(19) United States
(12) Patent Application Publication
(10) Pub. No.: US 2008/0293101 A1
(43) Pub. Date: Nov. 27, 2008

(54) Engineered microorganisms for increasing product yield in biotransformations, related methods and systems

(76) Inventors: Matthew W. Peters, Highlands Ranch, CO (US); Peter Meinhold, Highlands Ranch, CO (US); Thomas Buelter, Santa Monica, CA (US); Marco Landwehr, Pasadena, CA (US)

Correspondence Address: Enrica Bruno Patent Law, Ste 920, 150 S Los Robles Ave, Pasadena, CA, 91101, US

(21) Appl. No.: 11/881,748

(22) Filed: Jul. 27, 2007

Related U.S. Application Data
(60) Provisional application No. 60/833,932, filed on Jul. 27, 2006.

(51) Int. Cl. C12P 21/04; C12N 1/20; C12P 7/04; C12M 1/00

(52) U.S. Cl. 435/69.1; 435/252.3; 435/252.33; 435/157; 435/289.1

(57) ABSTRACT

There are disclosed recombinant microorganisms engineered to increase product yield in a biotransformation. In an embodiment, the microorganisms are engineered to increase the amount of NAD(P)H available for a NAD(P)H-requiring oxidoreductase involved in a biotransformation. There are also disclosed methods and systems for using recombinant microorganisms engineered to increase the amount of NAD(P)H available for a NAD(P)H-requiring oxidoreductase involved in a biotransformation. Other embodiments are also disclosed.

### CROSS REFERENCE TO RELATED APPLICATIONS

This application claims priority to U.S. Provisional Application Ser. No. 60/833,932 filed on Jul. 27, 2006, the disclosure of which is incorporated herein by reference it its entirety.

### FIELD

The present disclosure relates to engineered microorganisms. In particular, it relates to engineered microorganism for increasing product yield in biotransformations.

### BACKGROUND

Biotransformations, i.e. processes for the conversion of a substrate into a product within a host living organism, are known in the art. In particular, several processes are known in the art wherein the biotransformation results in the production of a desired compound in the host living organism. Specifically, processes are known wherein a substrate is converted into a final product within a living host cell via at least one oxidation-reduction reaction that requires transfer of electrons in order to occur.

A first example of such processes is provided by oxidations that involve insertion of oxygen atoms in a substrate molecule. In biological systems, oxygen is typically supplied to enzymatic systems as dioxygen and the reducing equivalents that regenerate oxygenase enzymes are usually derived from NADH or NADPH via proteins such as reductases. In particular, oxygenases stoichiometrically consume one molecule of NAD(P)H cofactor per molecule of product generated.

A further example of such processes is provided by a butanol-producing pathway as depicted in FIG. 1. This pathway is used by strains of the genus Clostridium, e.g. C. acetobutylicum to produce butanol (White, The physiology and Biochemistry of Prokaryotes. 2nd ed. 2000, New York: Oxford University Press, Inc.) and can be introduced into heterologous hosts, in which case the pathway requires 4 total NAD(P)H molecules to produce one molecule of butanol.

Longer chain alcohols can be theoretically produced by reducing acyl-ACP intermediates of the fatty acid biosynthetic pathway to the respective alcohols (White, The physiology and Biochemistry of Prokaryotes. 2nd ed. 2000, New York: Oxford University Press, Inc.) according to FIG. 2. The production of these alcohols also requires multiple NAD(P)H molecules to produce one molecule of alcohol.

Performance of such processes in a living host provides several advantages associated with a higher stability and/or activity shown by some enzymes involved in biotransformation (e.g. oxygenases, acylases) when expressed in a living host organism since the ‘packaging’ of the enzymes within the cellular membrane protects the enzyme from shear forces and other detrimental influences such as changes in pH. (W. A. Duetz, J. B. van Beilen, B. B. Witholt, Current opinion in biotechnology 12, 419 (2001). In addition, membrane-bound enzymes are oftentimes non-functional when not associated with the ability of cell membrane. Also, living cells have the ability to regenerate several of those enzymes when they become inactivated (see for example Ospina S. et al. Biotechnology Letters, 1995: 17(6)615-620).

Performance of biotransformation using whole cells, however, also presents a unique set of engineering challenges. For example, cells produce NAD(P)H cofactor for their own metabolic needs and not to supply it to a heterologously expressed biocatalyst. Additionally, facultative aerobes, such as E. coli, produce NAD(P)H in the presence of oxygen. However, in the process, the NAD(P)H is consumed by the respiratory pathway to ultimately reduce oxygen. This NAD(P)H and this oxygen can be required by some of the enzymes involved in the desired biotransformation.

The present disclosure relates to engineering whole cell microbial systems which address the above described challenges for the purpose of improving efficiency of the production of chemical products

### SUMMARY

The present disclosure relates to recombinant microorganisms engineered to increase the amount of NAD(P)H available for an NAD(P)H-requiring oxidoreductase involved in the biotransformation of a substrate into a desired product in the microorganisms. In the engineered microorganisms herein disclosed, an increased portion of the NAD(P)H produced by the microorganism is no longer processed by metabolic reactions of the microorganism and is instead channeled into the NAD(P)H-requiring oxidoreductase or NAD(P)H-requiring pathway involved in the biotransformation to drive the desired biotransformation of the substrate.

According to a first aspect, a recombinant microorganism is disclosed, wherein the recombinant microorganism has been engineered to inactivate a respiratory pathway in the microorganism. The recombinant microorganism can be further engineered to express an NAD(P)H-requiring oxidoreductase that is involved in the biotransformation of the substrate into the product.

According to a second aspect a recombinant microorganism is disclosed, wherein the recombinant microorganism has been engineered to activate a TCA cycle in the microorganism. The recombinant microorganism can be further engineered to express the NAD(P)H-requiring oxidoreductase that is involved in the biotransformation of the substrate into the product.

According to a third aspect, a method for performing a biotransformation of a substrate is disclosed, wherein the biotransformation is performed in any of the recombinant microorganisms herein disclosed where the NAD(P)H-requiring oxidoreductase involved in the biotransformation of the substrate is expressed.

According to a fourth aspect a system for performing a biotransformation of a substrate is disclosed, the system comprising any of the recombinant microorganisms herein disclosed and the substrate of the biotransformation. In some embodiments, where the recombinant microorganism is not engineered to express the NAD(P)H-requiring oxidoreductase involved in the biotransformation of the substrate, the NAD(P)H-requiring oxidoreductase involved in the biotransformation of the substrate can further be included in the system.

A first advantage of the recombinant microorganisms, methods and systems herein disclosed is that, due to the increased amount of NAD(P)H available for the biotransformation, an increased product yield of the biotransformation can be obtained.

A second advantage of the recombinant microorganism, methods and systems herein disclosed is that, higher activities of NAD(P)H-requiring enzyme(s) can be supported in the recombinant microorganism compared to unengineered microorganisms in which the intracellular metabolism is not sufficient to provide the required cofactors with the reduced equivalents. For example, in case of an NAD(P)H-requiring conversion of an exogenous substrate to a product requiring one NAD(P)H per reaction cycle, the product yield per carbon source can more than 4 and up to 10, depending on how many reducing equivalents generated during the TCA cycle are utilized to convert the substrate to the product. If the substrate is also the carbon and energy source for the cell, and the end product is derived from the substrate, then the recombinant microorganism disclosed herein makes biotransformations possible that require more NADH than the unengineered cells can produce.

An additional advantage of the recombinant microorganism in embodiments where the recombinant microorganisms are aerobes is that the cell does not respire oxygen and thus makes available oxygen that may be supplied to the culture medium to the overexpressed enzyme or pathway which may require oxygen as a substrate.

The details of one or more embodiments of the disclosure are set forth in the accompanying drawings and the description below. Other features, objects, and advantages will be apparent from the description and drawings, and from the claims.

### BRIEF DESCRIPTION OF THE DRAWINGS

The accompanying drawings, which are incorporated into and form a part of this specification, illustrate one or more embodiments of the present disclosure and, together with the detailed description, serve to explain the principles and implementations of the disclosure.

In the drawings:

FIG. 1 shows a butanol producing pathway known in the art;

FIG. 2 shows a fatty acid biosynthetic pathway known in the art;

FIG. 3 is a chart illustrating respiratory pathways of the microorganisms herein disclosed; Panel A shows a schematic representation of an aerobic respiratory pathway; Panel B shows a schematic representation of an anaerobic respiratory pathway.

FIG. 4 is a chart illustrating in more detail the aerobic respiratory pathway shown in FIG. 3 Panel A in a first exemplary microorganism herein disclosed;

FIG. 5 is a chart illustrating in more detail the anaerobic respiratory pathway shown in FIG. 3 Panel B in the first exemplary microorganism herein disclosed;

FIG. 6 is a chart illustrating in more detail the anaerobic respiratory pathway shown in FIG. 3 Panel B in a second exemplary microorganism herein disclosed;

FIG. 7 is a chart illustrating in more detail the respiratory pathways of FIGS. 4 and 5;

FIG. 8 is a chart illustrating in more detail the aerobic respiratory pathway shown in FIG. 3 Panel A in a third exemplary microorganism herein disclosed;

FIG. 9 is a chart illustrating a enzymatic system for the transport of NAD(P)H from a cellular compartment into another of the third exemplary microorganism herein disclosed,

FIG. 10 is a chart illustrating exemplary fermentative pathways in the microorganisms herein disclosed.

FIG. 11 is a chart illustrating in more detail the fermentative pathways shown in FIG. 10;

FIG. 12 is a chart schematically illustrating main variations of the tricarboxylic acid cycle (TCA) in microorganisms herein disclosed; dotted arrows indicate the glyoxylate shunt; block arrow indicate reactions catalyzed by enzymes that are inhibited by high level of NADH;

FIG. 13 illustrates expression of some enzymes involved in the TCA cycle in some recombinant microorganisms herein disclosed;

FIG. 14 illustrates expression of some enzymes involved in the glyoxylate shunt in some recombinant microorganisms herein disclosed;

FIG. 15 is a chart illustrating an exemplary engineered respiratory pathways in recombinant microorganism according to some embodiments herein disclosed;

FIG. 16 is a chart illustrating an exemplary engineered respiratory pathways in further recombinant microorganism according to some embodiments herein disclosed;

FIG. 17 is a chart illustrating an exemplary approach to produce the recombinant microorganisms herein disclosed that include the respiratory pathway described in FIG. 7.

FIG. 18 is a chart illustrating the exemplary approach of FIG. 9 performed under aerobic condition.

FIG. 19 is a schematic representation of the stoichiometry of butanol production using NADH made available by the TCA cycle;

FIG. 20 is a chart illustrating the exemplary approach of FIG. 9, performed in embodiments wherein the biotransformation is a metabolic pathway comprised of more than one reaction that utilize NAD(P)H has a cofactor;

FIG. 21 is a chart illustrating the level of propane oxidation in cell lysate and whole cells performed according to an embodiment of the present disclosure.

FIG. 22 shows levels of propanol produced in some embodiments of the recombinant microorganism herein disclosed compared with corresponding wild-type; Panel A is a diagram showing variation of the concentration of propanol and other metabolites at different times in wild-type microorganism expressing P450; Panel B is a diagram showing variation of the concentration of propanol and other metabolites at different times in the recombinant microorganism expressing P450; and

FIG. 23 shows product formation of ethyl 3-hydroxybytyrate in some embodiments of the recombinant microorganism herein disclosed compared with corresponding wild-type; Panel A is a diagram showing variation of the concentration of ethyl 3-hydroxybytyrate and glucose at different times in wild-type microorganism expressing ketoreductase; Panel B is a diagram showing variation of the concentration of ethyl 3-hydroxybytyrate and glucose at different times in the recombinant microorganism expressing ketoreductase; solid boxes indicate product concentration of ethyl 3-hydroxybytyrate, triangles indicate product concentration of glucose consumed.

### DETAILED DESCRIPTION

The present disclosure refers to a recombinant microorganism engineered to increase the amount of NAD(P)H available to a NAD(P)H-requiring oxidoreductase involved in a biotransformation.

The term “microorganism” is used herein interchangeably with the terms “cell,” “microbial cells” and “microbes” and refers to an organism of microscopic or ultramicroscopic size such as a prokaryotic or a eukaryotic microbial species. The term “prokaryotic” refers to a microbial species which contains no nucleus or other organelles in the cell, which includes but is not limited to Bacteria and Archaea. The term “eukaryotic” refers to a microbial species that contains a nucleus and other cell organelles in the cell, which includes but is not limited to Eukarya such as yeast and filamentous fungi, protozoa, algae, or higher Protista.

The term “bacteria” as used herein refers to several prokaryotic microbial species which include but are not limited to Gram-positive bacteria, Proteobacteria, Cyanobacteria, Spirochetes and related species, Planctomyces, Bacteroides, Flavobacteria, Chiamydia, Green sulfur bacteria, Green non-sulfur bacteria including anaerobic phototrophs, Radioresistant micrococci and related species, Thermotoga and Thermosipho thermophiles. More specifically, the wording “Gram positive bacteria” refers to cocci, nonsporulating rods and sporulating rods, such as, for example, Actinomyces, Bacillus, Clostridium, Corynebacterium, Erysipelothrix, Lactobacillus, Listeria, Mycobacterium, Myxococcus, Nocardia, Staphylococcus, Streptococcus and Streptomyces. The term “Proteobacteria” refers to purple photosynthetic and non-photosynthetic gram-negative bacteria, including cocci, nonenteric rods and enteric rods, such as, for example, Neisseria, Spirillum, Pasteurella, Brucella, Yersinia, Francisella, Haemophilus, Bordetella, Escherichia, Salmonella, Shigella, Klebsiella, Proteus, Pseudomonas, Bacteroides, Acetobacter, Aerobacter, Agrobacterium, Azotobacter, Spirilla, Serratia, Vibrio, Rhizobium, Chlamydia, Rickettsia, Treponema and Fusobacterium. Cyanobacteria, e.g., oxygenic phototrophs;

The term “Archaea” as used herein refers to prokaryotic microbial species of the division Mendosicutes, such as Crenarchaeota and Euryarchaeota, and include but is not limited to methanogens (prokaryotes that produce methane); extreme halophiles (prokaryotes that live at very high concentrations of salt (NaCl); and extreme (hyper) thermophiles (prokaryotes that live at very high temperatures).

The term “recombinant” as used herein with reference to a microorganism in alternative to “wild-type” or “native”, indicates a microorganism that has been engineered to modify the genotype and/or the phenotype of the microorganism as found in nature, e.g., by modifying the polynucleotides and/or polypeptides expressed in the microorganism as it exists in nature. A “wild-type microorganism” refers instead to a microorganism which has not been engineered and displays the genotype and phenotype of said microorganism as found in nature.

The term “engineer” refers to any manipulation of a microorganism that result in a detectable change in the microorganism, wherein the manipulation includes but is not limited to inserting a polynucleotide and/or polypeptide heterologous to the microorganism and mutating a polynucleotide and/or polypeptide native to the microorganism. A polynucleotide or polypeptide is “heterologous” to a microorganism if it is not part of the polynucleotides and polypeptides expressed in the microorganism as it exists in nature, i.e., it is not part of the wild-type of that microorganism. A polynucleotide or polypeptide is instead “native” to a microorganism if it is part of the polynucleotides and polypeptides expressed in the microorganism as it exists in nature, i.e., it is part of the wild-type of that microorganism. The term “mutation” as used herein indicates any modification of a nucleic acid and/or polypeptide which results in an altered nucleic acid or polypeptide. Mutations include, for example, point mutations, deletions, or insertions of single or multiple residues in a polynucleotide, which includes alterations arising within a protein-encoding region of a gene as well as alterations in regions outside of a protein-encoding sequence, such as, but not limited to, regulatory or promoter sequences.

The term “polynucleotide” is used herein interchangeably with the term “nucleic acid” and refers to an organic polymer composed of two or more monomers including nucleotides, nucleosides or analogs thereof, including but not limited to single stranded or double stranded, sense or antisense deoxyribonucleic acid (DNA) of any length and, where appropriate, single stranded or double stranded, sense or antisense ribonucleic acid (RNA) of any length, including siRNA. The term “nucleotide” refers to any of several compounds that consist of a ribose or deoxyribose sugar joined to a purine or a pyrimidine base and to a phosphate group, and that are the basic structural units of nucleic acids. The term “nucleoside” refers to a compound (as guanosine or adenosine) that consists of a purine or pyrimidine base combined with deoxyribose or ribose and is found especially in nucleic acids. The term “nucleotide analog” or “nucleoside analog” refers, respectively, to a nucleotide or nucleoside in which one or more individual atoms have been replaced with a different atom or with a different functional group. Accordingly, the term polynucleotide includes nucleic acids of any length, DNA, RNA, analogs and fragments thereof. A polynucleotide of three or more amino acids is also called nucleotidic oligomer or oligonucleotide.

The term “protein” or “polypeptide” as used herein indicates an organic polymer composed of two or more amino acidic monomers and/or analogs thereof. As used herein, the term “amino acid” or “amino acidic monomer” refers to any natural and/or synthetic amino acids including glycine and both D or L optical isomers. The term “amino acid analog” refers to an amino acid in which one or more individual atoms have been replaced, either with a different atom, or with a different functional group. Accordingly, the term polypeptide includes amino acidic polymer of any length including full length proteins, and peptides as well as analogs and fragments thereof. A polypeptide of three or more amino acids is also called a protein oligomer or oligopeptide

The term “enzyme” as used herein refers to any substance that catalyzes or promotes one or more chemical or biochemical reactions, which usually includes enzymes totally or partially composed of a polypeptide, but can include enzymes composed of a different molecule including polynucleotides.

The term “oxidoreductase” as used herein refers to an enzyme that catalyzes the transfer of electrons from one molecule (the reductant, also called the hydrogen or electron donor) to another (the oxidant, also called the hydrogen or electron acceptor). Electron donors include carrier molecules such as NADH or NAD(P)H that contain reducing equivalents wherein the term “reducing equivalents” refers to electrons usually generated through oxidation of a substrate during aerobic or anaerobic metabolism that are contained in the carrier molecule. Electron acceptors include the oxidized form of carrier molecules NADH and NADPH, i.e. NAD+ and NADP+. The term “substrate as used herein refers to any substance or compound that is converted or meant to be converted into another compound by the action of an enzyme catalyst.

An “NAD(P)H-requiring oxidoreductase” as used herein refers to an enzyme that catalyzes a reaction involving the transfer of reducing; equivalents directly or indirectly donated by NADH or NADPH. An “NAD(P)H producing oxidoreductase” as used herein refers to an enzyme that catalyzes a reaction involving the transfer of reducing equivalents directly or indirectly donated to an NAD⁺ or NADP⁺.

The term “biotransformation” as used herein refers to a process for the conversion of a substrate into a product within a living organism, which includes any modifications of the chemical and/or biological nature and/or properties of the substrate occurring within the living organism and resulting in the production of the product. Exemplary biotransformations can be performed by a single native or heterologous enzyme, by a plurality of native and/or heterologous enzymes, which in some embodiments can control one or more reactions of a chain of enzymatically controlled reactions for the production of the product.

The term “substrate” as used herein refers to any compound on which an enzyme can act, and in particular, any organic compound on which an enzyme can act in a microorganism herein disclosed.

An NAD(P)H-requiring oxidoreductase involved in the biotransformation of a substrate into a product is herein also referred as biotransformation NAD(P)H-requiring oxidoreductase.

In some embodiments of the recombinant microorganisms herein disclosed, the amount of NAD(P)H available for the biotransformation NAD(P)H-requiring oxidoreductase or NAD(P)H-requiring pathway is increased in the recombinant microorganism by engineering the microorganism to inactivate a respiratory pathway of the microorganism.

As used herein, the term “pathway” refers to a biological process including two or more enzymatically controlled chemical reactions by which a substrate is converted into a product. The wording “respiratory pathway” refers to a pathway wherein the conversion from the substrate to the product is associated with the production of energy in the microorganism and wherein at least one of the reactions in the pathway involves transfer of electrons from an electron donor to a carrier molecule such as NAD⁺ or NADP⁺, and transfer of the electrons from the carrier molecule to a final electron acceptor. The wording “respiratory pathway” refers to aerobic or anaerobic respiratory pathways.

The wording “aerobic respiratory pathway” refers to a respiratory pathway in which oxygen is the final electron acceptor and the energy is typically produced in the form of an ATP molecule. The wording “aerobic respiratory pathway” is used herein interchangeably with the wording “aerobic metabolism”, “aerobic respiration”, “oxidative metabolism” or “cell respiration”.

The wording “anaerobic respiratory pathway” refers to a respiratory pathway in which oxygen is not the final electron acceptor and the energy is typically produced in the form of an ATP molecule, which includes a respiratory pathway wherein an organic or inorganic molecule other than oxygen (e.g. nitrate, fumarate, dimethylsulfoxide, sulfur compounds such as sulfate, and metal oxides) is the final electron acceptor. The wording “anaerobic respiratory pathway” is used herein interchangeably with the wording “anaerobic metabolism” and “anaerobic respiration”.

The term “inactivated” or “inactivation” as used herein with reference to a pathway indicates a pathway in which any enzyme controlling a reaction in the pathway is biologically inactive, which includes but is not limited to inactivation of the enzyme is performed by deleting one or more genes encoding for enzymes of the pathway. The term “activated” or “activation”, as used herein with reference to a pathway, indicates a pathway in which any enzyme controlling a reaction in the pathway is biologically active. Accordingly, a respiratory pathway is inactivated when at least one enzyme controlling a reaction in the pathway is inactivated so that the reaction controlled by said enzyme does not occur. On the contrary, a respiratory pathway is activated when all the enzymes controlling a reaction in the pathway are activated. As a consequence in an inactivated respiratory pathway the transfer of electrons from the carrier molecule to the electron acceptor is not detectable while in an activated pathway transfer of electrons from the carrier molecule to the electron acceptor is detectable.

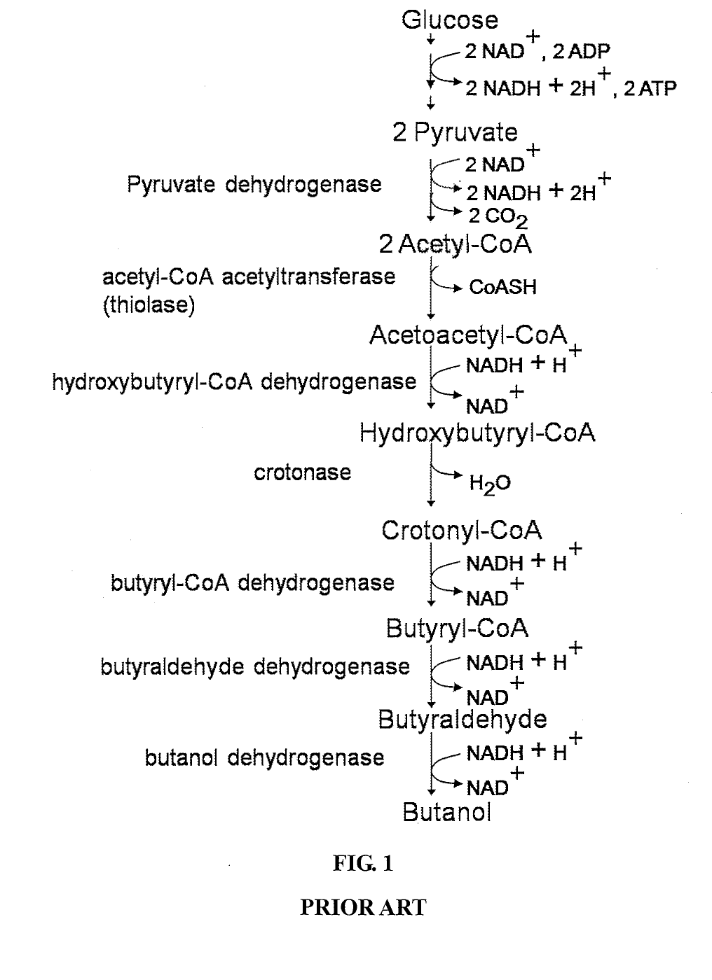

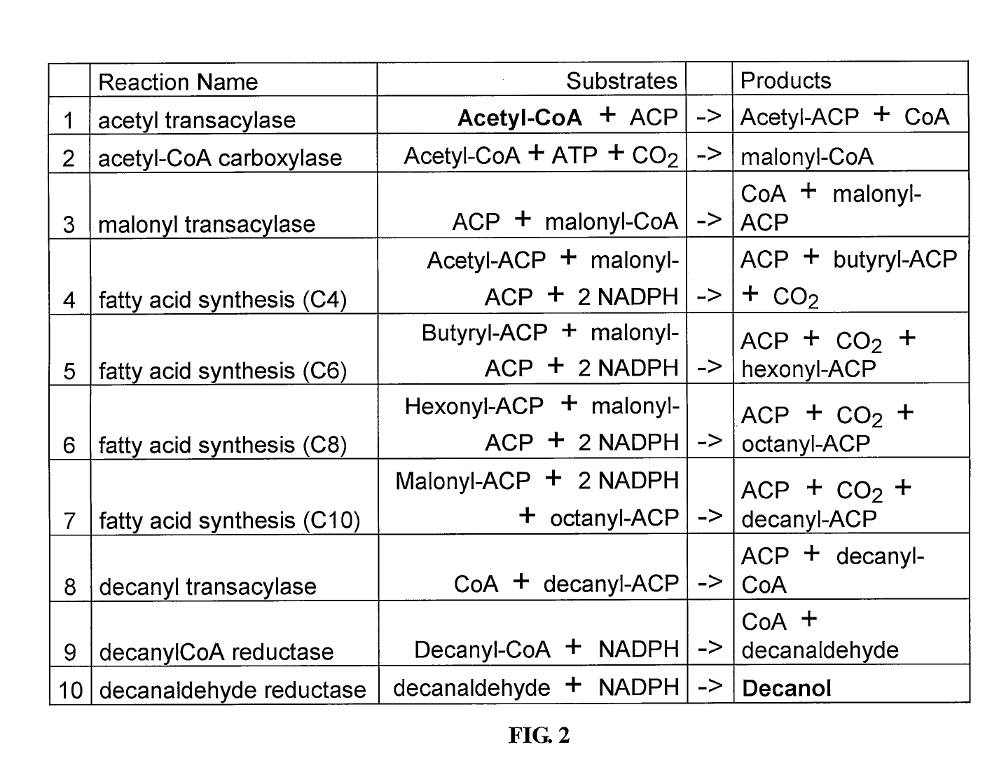

In some embodiments herein disclosed, the microorganisms herein disclosed, at least one of the above mentioned respiratory pathways is used by the microorganisms for the microorganisms' survival and growth. In “aerobes” or “aerobic microorganisms”, aerobic respiratory pathways are used by the microorganism wherein “facultative aerobes” can also use anaerobic respiratory pathways. In “anaerobes” or “anaerobic microorganisms”, anaerobic respiratory pathways are used, which includes both anaerobic respiration and/or fermentation. Exemplary respiratory pathways of the microorganisms herein disclosed are schematically shown in FIGS. 1 to 5, wherein NAD(P)H-requiring oxidoreductase of aerobic respiratory pathways and anaerobic respiratory pathways are illustrated in detail.

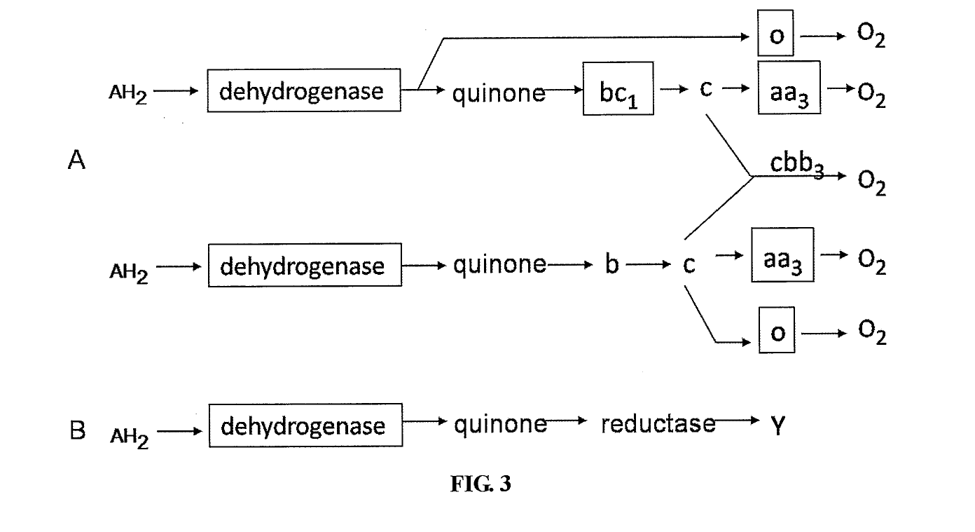

In particular, FIG. 3 provides a schematic representation of respiratory pathways wherein respiratory NAD(P)H-requiring oxidoreductase common to various pathways of different microorganisms are specifically identified while the specific reactions of the pathways that vary depending on the microorganism and the relevant growth conditions are omitted. In FIG. 3 redox active small molecules involved in the pathways are also specifically identified,

wherein the wording “redox active small molecule” refers to a chemical compound that is synthesized within the cell and that can accept electrons from an electron donor and subsequently transfer electrons to an electron acceptor within a respiratory pathway. Examples of redox active small molecules are provided by quinones and more specifically ubiquinone and menaquinone.

Both aerobic respiratory pathways (Panel A) and anaerobic respiratory pathways (Panel B) are illustrated in FIG. 3. In an aerobic respiratory pathway of the wild-type microorganisms herein disclosed, a dehydrogenase catalyzes the transfer of electrons from an electron donor, usually reducing equivalents in the form of NADH or NADPH (AH2), to a quinone (e.g. menaquinone and ubiquinone) (FIG. 3 Panel A). An oxidase complex then transfers these electrons to oxygen through a branched pathway. Branch points vary from organism to organism, but branching at the stage of quinone or cytochrome are usual. Some bacteria channel electrons from, the quinones directly to cytochrome o. Many bacteria funnel electrons along the path bc₁ complex→cytochrome c→cytochrome aa₃. This pathway is similar to mitochondria, as all contain cytochrome aa₃ as the terminal oxidase. Some bacteria do not contain a bc₁ complex or cytochrome aa₃. Other bacteria contain cytochrome b in place of the bc₁ complex. Still other bacteria contain alternate terminal oxidases, perhaps even two or three different ones per organism including cbb₃ which has a higher affinity for oxygen than other terminal oxidases, other oxidases that may or may not act as proton pumps, and still other oxidases that vary in structure (e.g. cytochrome bd oxidase).

In an anaerobic respiratory pathway of wild-type microorganisms herein disclosed, the electrons, after being transferred from a carrier molecule such as NADH or NADPH to a dehydrogenase and to a quinone (FIG. 3 panel B), are transferred to a reductase complex or complexes, which are synthesized anaerobically. A single microorganism may have a several reductases and each one is usually specific for a given electron acceptor. Y represents either an inorganic external electron acceptor other than oxygen, e.g. nitrate, or an organic electron acceptor, e.g. fumarate. The term “specific” or “specificity” as used herein with reference to an enzyme indicates recognition contact and reaction of the enzyme with a substrate together with substantially less recognition, contact and reaction of that enzyme with other substrates, which includes substrates that are similar in size, electrochemical properties or three-dimensional structure.

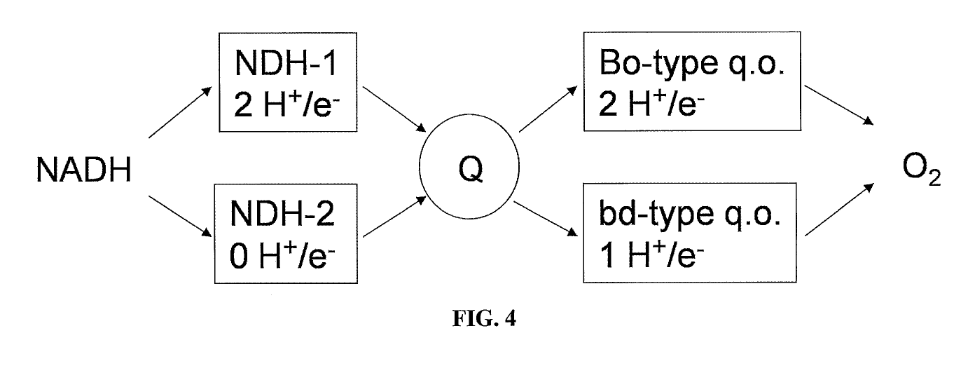

An additional and more detailed representation of an exemplary aerobic respiratory pathway is illustrated in FIG. 4 where the interactions between the NAD(P)H-requiring oxidoreductases of the aerobic respiratory pathway of FIG. 3, as occurring in a microorganism such as E. coli are schematically shown. In the respiratory pathway of FIG. 4, two electrons per NADH are transferred via an NADH dehydrogenase (NDH-1, NDH-2), a quinone (O) and a quinol oxidase (q.o.) complex (bo-type and bd-type q.o.) where oxygen is reduced to water. Redox reactions occurring at the NADH dehydrogenases and the quinol oxidase complexes are coupled to proton extrusion. Electrons are transferred to oxygen through one of four distinct pathways to translocate two (NDH-2/bd-type q.o.), four (NDH-2/bo-type q.o.), six (NDH-1/bo), or eight protons across the cell membrane, depending on the intra- and extracellular environment (see FIG. 4).

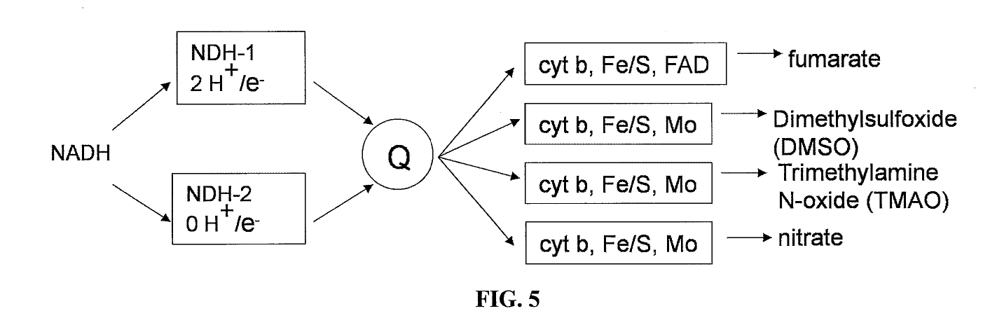

An additional and more detailed representation of an anaerobic respiratory pathway is illustrated in FIG. 5, wherein the interactions between the NAD(P)H-requiring oxidoreductases of the anaerobic respiratory pathway of FIG. 3, as occurring in a microorganism such as E. coli are schematically shown. In the respiratory pathway of FIG. 5, two electrons per NADH are transferred via an NADH dehydrogenase (NDH-1, NDH-2), a quinone (Q) and reductases to electron acceptors, such as fumarate, Dimethylsulfoxide, trimethylamine N-oxide and nitrate. Redox reactions occurring at the NADH dehydrogenases are coupled to proton extrusion. Electrons are transferred to the electron acceptor through distinct pathways (see FIG. 5).

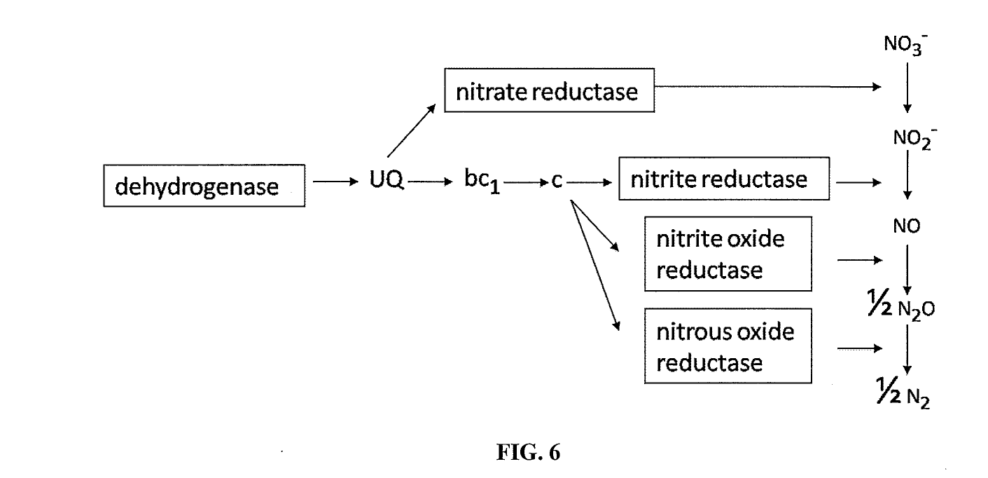

A further more detailed representation of an anaerobic respiratory pathway is illustrated in FIG. 6 (White, The physiology and Biochemistry of Prokaryotes. 2nd ed. 2000, New York: Oxford University Press, Inc.) wherein the interactions between the NAD(P)H-requiring oxidoreductases of the anaerobic respiratory pathway of FIG. 3 are schematically shown. In the respiratory pathway of FIG. 6, two electrons per NADH are transferred via an NADH dehydrogenase, a quinone (UQ) and/or Cytochrome (bc1 and c) and reductases (nitrate reductase, nitrite reductase, nitric oxide reductase and nitrous oxide reductase) to electron acceptors, such as nitrate, nitrite, nitrous oxide or nitric oxide. Redox reactions occurring at the NADH dehydrogenases are coupled to proton extrusion. Electrons are transferred to the electron acceptor through distinct pathways

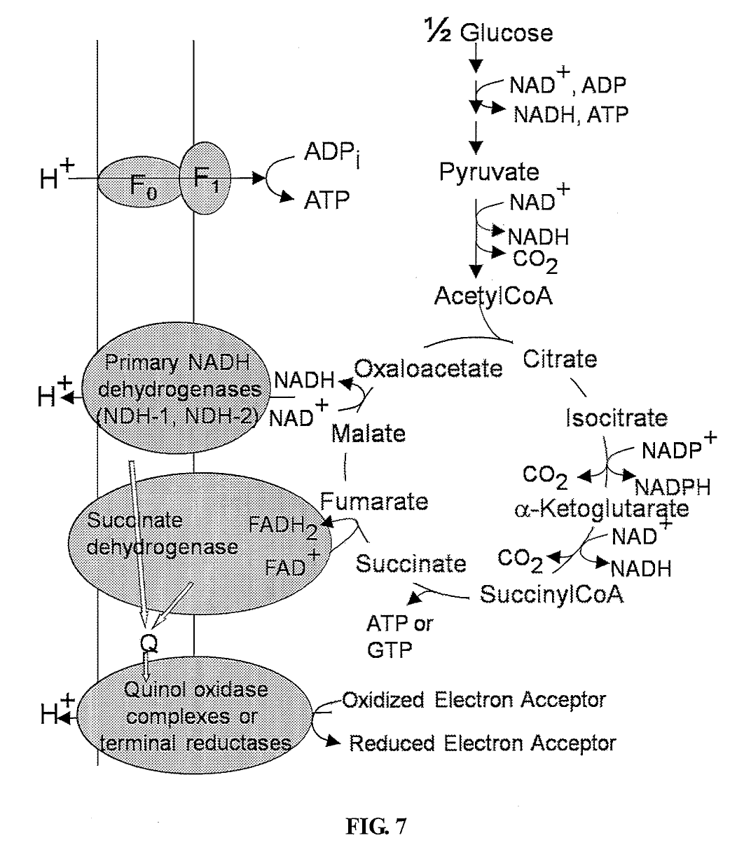

An even more detailed representation of the enzymatic reactions of some of the aerobic and anaerobic respiratory pathways-schematically described in FIGS. 3 to 5, is further illustrated in FIG. 7, which shows an exemplary aerobic or anaerobic respiratory pathway wherein glucose is the carbon source, carbon dioxide is the final product, and the pathway comprises activated glycolysis and TCA cycle pathways.

The term “glycolysis” refers to a pathway for the conversion of a glucose molecule into two pyruvate molecules within the microorganism, which in the microorganism is also associated with net production of two ATP molecule and two NAD(P)H molecule. Glycolysis may also be referred to as the “Embden-Meyerhof pathway”. The term “TCA cycle” as used herein refers to a pathway wherein the acetate is converted in a cyclical manner, into carbon dioxide and NAD(PH).TCA cycle may also be referred to as “tricarboxylic acid cycle” or “Krebs cycle.”

In the pathway illustrated in FIG. 7, a single glucose molecule is metabolized completely into carbon dioxide, wherein in aerobic respiration the final electron acceptor is oxygen and in anaerobic respiration the final electron acceptor is an exogenous electron acceptor other than oxygen.

In particular, in the pathway illustrate in FIG. 7, some of he NADH dehydrogenases, quinol oxidase complex (e.g. bo-type, bd-type) quinol cytochrome c oxidoreductases, cytochrome oxidases and reductases identified in FIGS. 3 to 5, translocate protons across the cell membrane as they pass electrons to their respective acceptor molecules. Those translocates protons generate a proton gradient across the membrane, called to the proton motive force, which is used by the cell to produce additional ATP for the cell by an ATP synthase. ATP synthase, consists of two components F0, which is the proton channel that spans the membrane and F1, which is the catalytic subunit on the inner membrane surface that catalyzed the reversible hydrolysis of ATP to ADP plus inorganic phosphate.

Some of the NADH dehydrogenases, quinol oxidase complexes quinol cytochrome c oxidoreductases, cytochrome oxidases, and reductases identified in FIGS. 3 to 5, may instead not translocate protons across the cell membrane. These enzymes or enzyme complexes provide a route for reoxidation of NAD(P)H that is uncoupled from the generation of proton motive force or ATP production, thus giving the cell an outlet to remove excess NADH without generating additional ATP.

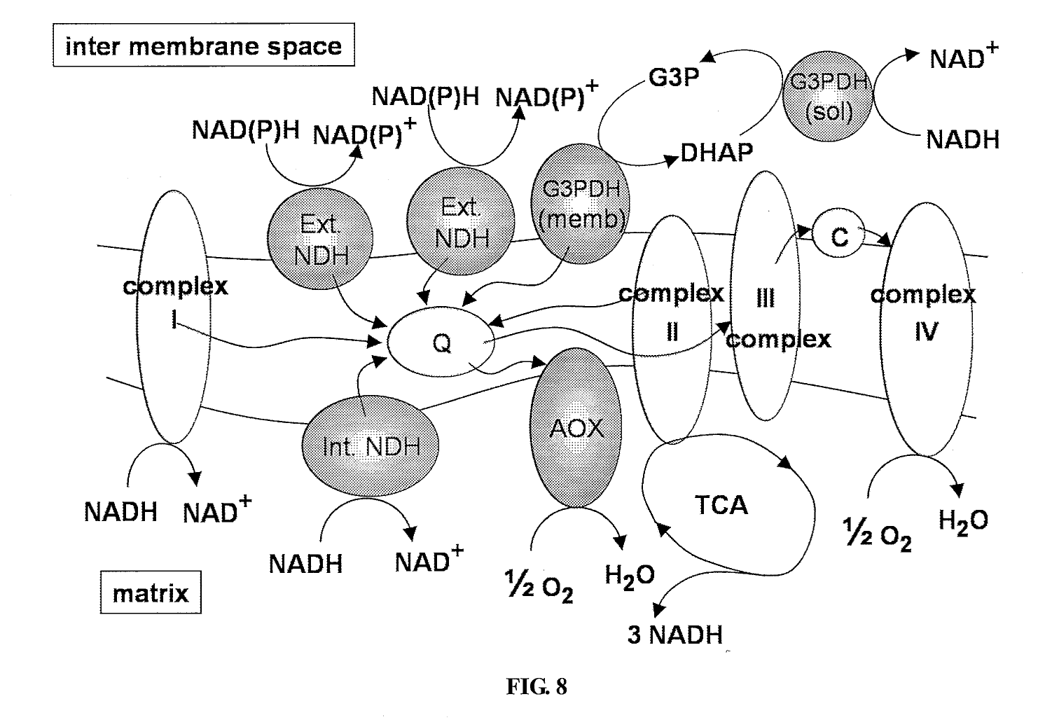

An additional and more detailed representation of an exemplary aerobic respiratory pathway is illustrated in FIG. 8 where the interactions between the NAD(P)H-requiring oxidoreductases of the aerobic respiratory pathway of FIG. 3, as occurring in a microorganism such as yeast are schematically shown

In yeast cells NAD+/NADH and NADP+/NADPH exist as separate pools in the cytoplasm and the mitochondria. During respiratory growth, the activated TCA cycle generates electrons that are transferred, directly or indirectly (via NAD(P)H molecules) to the ubiquinone pool (O) via succinate dehydrogenase (complex II), via the standard respiratory complex I (complex I), or via an internal NADH dehydrogenase (int. NDH), which is located on the matrix face of the inner mitochondrial membrane. The electrons from the cytoplasmic NAD(P)H (generated mainly through glycolysis and the pentose phosphate pathway) can be transferred to the ubiquinone pools via the external NAD(P)H dehydrogenases (ext. NDH) located on the cytoplasmic face of the inner mitochondrial membrane. In some yeasts such as Saccharomyces cerevisiae, these dehydrogenases are NADH-specific, while in other yeasts, such as Kluyveromyces lactis, these dehydrogenases utilize both NADH and NADPH.

As illustrated in FIG. 8, in addition to these external NAD(P)H dehydrogenases, NADH can be oxidized via a soluble glycerol-3-phosphate dehydrogenase (G3PDH) which converts dihydroxyacetone phosphate (DHAP) to glyceraldehyde-3-phophate (G3P). This reaction is reversed by a membrane bound G3PDH which in turn transfers electrons to the ubiquinone pool. The electrons from the ubiquinone pool can then be transferred to oxygen via the standard respiratory complexes III (ubiquinone:cytochrome c oxidoreductase) and IV (cytochrome c oxidase) or via an alternative oxidase (AOX). In some yeasts, such as the facultatively fermenting Saccharomyces cerevisiae and Kluyveromyces lactis, certain aspects of the respiratory pathway are absent. These strains do not encode for the respiratory complex I nor the alternative oxidase.

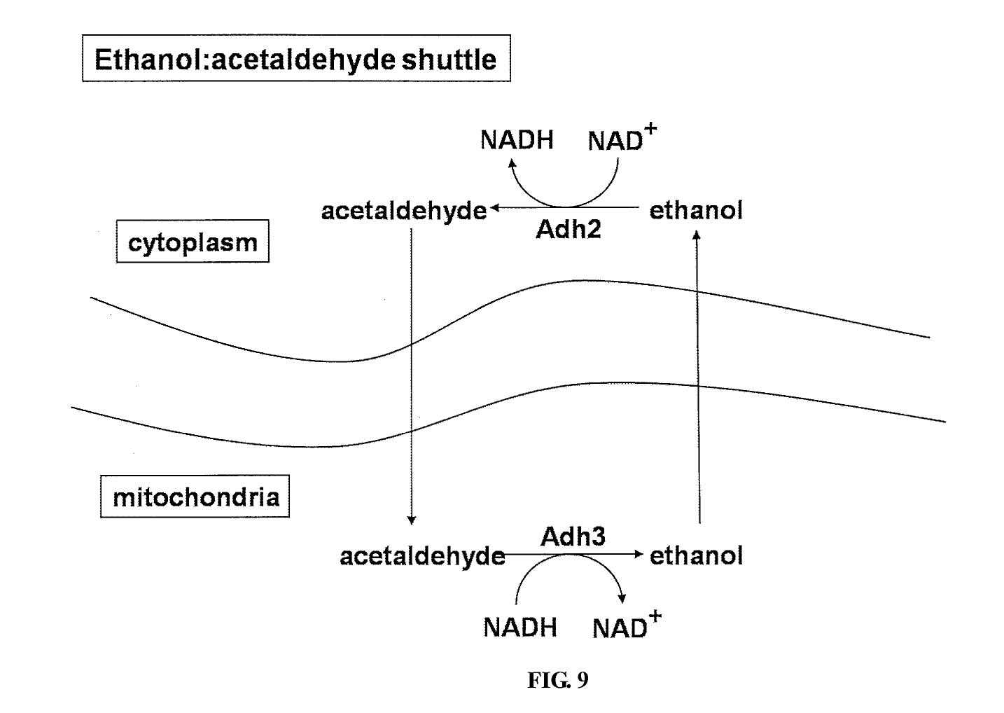

In yeast microorganisms, reducing equivalents produced by the TCA cycle can be transferred from the mitochondria to the cytoplasm via an acetaldehyde ethanol shuttle illustrated in FIG. 9. In particular, in the illustration of FIG. 9, an NADH donates the reducing equivalent in a reaction catalyzed by the dehydrogenase Adh3 to an acetaldehyde that is thus converted to ethanol; the ethanol pass in the cytoplasm where the reducing equivalents are transferred from ethanol to an NAD carrier in a reaction catalyzed by a alcohol dehydrogenase Adh2, where the ethanol is converted to acetaldehyde.

In some embodiments, a respiratory pathway of the microorganism can be inactivated by inactivating at least one or the biologically active molecules involved in the respiratory pathway and in particular an NAD(P)H-requiring oxidoreductase (herein also referred as respiratory NAD(PH) dependent oxidoreductase) a redox active small molecule and/or additional enzymes in the respiratory pathway whose biological activity is associated with downstream consumption of NAH(P)H through a respiratory NAD(P)H-requiring oxidoreductase.

The terms “inactivate” or “inactivation” as used herein with reference to a biologically active molecule, such as an enzyme or an electron carrier molecule, indicates any modification in the genome and/or proteome of a microorganism that prevents or reduces the biological activity of the biologically active molecule in the microorganism. Exemplary inactivations include but are not limited to modifications that results in the conversion of the molecule from a biologically active form to a biologically inactive form and from a biologically active form to a biologically less or reduced active form, and any modifications that result in a total or partial deletion of the biologically active molecule. For example, inactivation of a biologically active molecule can be performed by deleting or mutating the a native or heterologous polynucleotide encoding for the biologically active molecule in the microorganism, by deleting or mutating a native or heterologous polynucleotide encoding for an enzyme involved in the pathway for the synthesis of the biologically active molecule in the microorganism, by activating a further a native or heterologous molecule that inhibits the expression of the biologically active molecule in the microorganism.

The terms “activate” or “activation” as used herein with reference to a biologically active molecule, such as an enzyme, indicates any modification in the genome and/or proteome of a microorganism that increases the biological activity of the biologically active molecule in the microorganism. Exemplary activations include but are not limited to modifications that results in the conversion of the molecule from a biologically inactive form to a biologically active form and from a biologically active form to a biologically more active form, and modifications that result in the expression of the biologically active molecule in a microorganism wherein the biologically active molecule was previously not expressed. For example, activation of a biologically active molecule can be performed by expressing a native or heterologous polynucleotide encoding for the biologically active molecule in the microorganism, by expressing a native or heterologous polynucleotide encoding for an enzyme involved in the pathway for the synthesis of the biological active molecule in the microorganism, by expressing a native or heterologous molecule that enhances the expression of the biologically active molecule in the microorganism.

The term “native” or “endogenous” as used herein with reference to molecules, and in particular enzymes and polynucleotides, indicates molecules that are expressed in the organism in which they originated or are found in nature, independently on the level of expression that can be lower equal or higher than the level of expression of the molecule in the native microorganism.

The term “heterologous” or “exogenous” as used herein with reference to molecules and in particular enzymes and polynucleotides, indicates molecules that are expressed in a organism other than the organism from which they originated or are found in nature, independently on the level of expression that can be lower equal or higher than the level of expression of the molecule in the native microorganism. In some embodiments of the recombinant microorganisms herein disclosed, the recombinant microorganism is engineered to inactivate at least one of the respiratory NAD(P)H-requiring oxidoreductase illustrated in FIGS. 1 to 5. In particular in some embodiments, of the above mentioned dehydrogenase, oxidase, oxidoreductase and/or reductase involved in a respiratory pathway exemplarily illustrated in FIGS. 3 to 8.

More in particular, in some embodiments, the recombinant microorganism is engineered to inactivate at least one of an NDH-1 dehydrogenase, NDH-2 dehydrogenase, a quinol oxidase complex including a bo-type and/or a bd-type quinol oxidase complexes, a quinol:cytochrome c oxidoreductase, a cytochrome oxidase; a terminal reductase or an enzyme involved in a terminal reductase pathway including but not limited to iron-cytochrome-c reductase, respiratory arsenate reductase, nitrite reductase complex, trimethylamine n-oxide reductase, dimethyl sulfoxide reductase, dissimilatory sulfite reductase, adenylylsulfate reductase, atp sulfurylase, nitrous oxide reductase, nitric oxide reductase, nitrite reductase, periplasmic nitrate reductase and nitrate reductase.

In some embodiments, the inactivation of the respiratory NAD(P)H-requiring oxidoreductase is performed by inactivating an enzyme involved in the synthesis of the respiratory NAD(P)H oxidoreductase.

In some embodiments, the inactivation of the respiratory pathway is performed by inactivating a redox small molecule involved in the pathway, such as quinone including but not limited to ubiquinone and menaquinone. In some embodiments, the inactivation of the respiratory pathway is performed by inactivating an enzyme in the pathway that is not NAD(P)H-requiring. Should the microorganism activate alternative NAD(P)H-requiring respiratory enzymes or respiratory pathways that outcompete the NAD(P)H-requirement of the biotransformation, then these pathways are sequentially inactivated so that NAD(P)H is no longer utilized for respiration

Table 1 provides an exemplary list of NAD(P)H-requiring oxidoreductases involved in the respiratory pathway, enzymes involved in the synthesis of redox active small molecules of the respiratory pathway that can be inactivated in various embodiments of the recombinant microorganism herein disclosed

In particular, in Table 1 enzymes are shown along with their EC numbers and relevant substrates and products. EC number is the classification number designated by the Enzyme Commission. X=all child EC categories.

TABLE 1

| Relevant Reaction | Relevant Product | | |
|---|---|---|---|
| Substrate (s) | (s) | Enzyme name | EC number |
| NADH or NADPH | NAD+ or NADP+ | NADH dehydrogenase | 1.6.5.3, 1.6.99.3, |
| and Ubiquinone | and Ubiquinol | | 1.6.99.5, 1.6.99.X, |
| | | | 1.6.5.X, 1.16.1.X |
| NADH or NADPH | NAD+ or NADP+ | NADH oxidase or | 1.6.3.1 or 1.6.3.X |
| and oxygen | and water | NADPH oxidase | |
| Ubiquinol and | Ubiquinone and | ubiquinol-cytochrome c | 1.10.2.2 or |
| oxidized cytochrome c | reduced | reductase | 1.10.2.X |
| | Cytochrome c | | |
| Ubiquinol and oxygen | Ubiquinone and | Quinol oxidase complex | 1.10.3.X or |
| | water | | 1.10.2.X |
| reduced cytochrome c | Oxidized | Cytochrome c oxidase | 1.9.3.1, 1.9.3.X |
| and oxygen | cytochrome c and | | |
| | water | | |
| Chorismate | isochorismate | isochorismate synthase | 5.4.4.2 or 5.4.4.X |
| | | (menaquinone | |
| | | biosynthesis pathway) | |
| Chorismate | 4- | chorismate pyruvate- | 4.1.3.B1 or |
| | hydroxybenzoate | lyase (ubiquinone | 4.1.3.X |
| | and pyruvate | biosynthesis pathway) | |
| 4-hydroxybenzoate | 3-octaprenyl-4- | 4-hydroxybenzoate | 2.5.1.X |
| and octaprenyl | hydroxybenzoate | octaprenyltransferase | |
| diphosphate | and diphosphate | (ubiquinone biosynthesis | |
| | | pathway) | |
| Nitrate and a reduced | Nitrite and an | Nitrate reductase | 1.7.99.4 or |
| acceptor (e.g. NADH | acceptor (e.g. | | 1.7.99.X |
| or NADPH or quinol | NAD+ or NADP+ | | |
| or reduced cytochrome | or quinone or | | |
| c) | oxidized | | |
| | cytochrome c) and | | |
| | water | | |
| Nitrate and quinol | Nitrite and quinine | Periplasmic nitrate | 1.7.99.4 |
| | and water | reductase | |
| Nitrite and a reduced | Nitric oxide and an | Nitrite reductase | 1.7.2.1 or 1.7.2.X |
| acceptor (e.g. NADH | acceptor (e.g. | | |
| or NADPH or quinol | NAD+ or NADP+ | | |
| or reduced cytochrome | or quinone or | | |
| c) | oxidized | | |
| | cytochrome c) and | | |
| | water | | |
| Nitric oxide and a | Nitrous oxide and | Nitric oxide reductase | 1.7.99.7 or |
| reduced acceptor (e.g. | an acceptor (e.g. | | 1.7.99.X |
| NADH or NADPH or | NAD+ or NADP+ | | |
| quinol or reduced | or quinone or | | |
| Cytochrome c) | oxidized | | |
| | cytochrome c) and | | |
| | water | | |
| Nitrous oxide and a | Nitrogen gas and | Nitrous oxide reductase | 1.7.99.6 or |
| reduced acceptor (e.g. | an acceptor (e.g. | | 1.7.99.X |
| NADH or NADPH or | NAD+ or NADP+ | | |
| quinol or reduced | or quinone or | | |
| Cytochrome c) | oxidized | | |
| | cytochrome c) and | | |
| | water | | |
| Sulfate and ATP | adenosine 5′- | ATP sulfurylase | 2.7.7.4 or 2.7.7.X |
| | phosphosulfate | | |
| | and diphosphate | | |
| Adenosine 5′- | Sulfite and AMP | Adenylylsulfate | 1.8.99.2 or |
| phosphosulfate and a | and an acceptor | reductase | 1.8.99.X |
| reduced acceptor (e.g. | (e.g. NAD+ or | | |
| NADH or NADPH or | NADP+ or | | |
| quinol or reduced | quinone or | | |
| Cytochrome c) | oxidized | | |
| | Cytochrome c) | | |
| Sulfite and a reduced | Hydrogen sulfide | dissimilatory sulfite | 1.8.99.1 or |
| acceptor (e.g. NADH | and an acceptor | reductase | 1.8.99.X |
| or NADPH or quinol | (e.g. NAD+ or | | |
| or reduced cytochrome | NADP+ or | | |
| c) | quinone or | | |
| | oxidized | | |
| | cytochrome c) and | | |
| | water | | |
| Bisulfite and a reduced | Trithionate and | dissimilatory sulfite | 1.8.99.3 or |
| acceptor (e.g. NADH | water and an | reductase | 1.8.99.X |
| or NADPH or quinol | acceptor (e.g. | | |
| or reduced cytochrome | NAD+ or NADP+ | | |
| c) | or quinone or | | |
| | oxidized | | |
| | Cytochrome c) | | |
| Quinol and dimethyl | Quinone and | Dimethyl sulfoxide | 1.8.99.X |
| sulfoxide | dimethylsulfide | reductase | |
| Trimethylamine N- | Trimethylamine | Trimethylamine N-oxide | 1.6.6.9 or 1.6.6.X |
| oxide and NADH or | and NAD+ or | reductase | |
| NADPH | NADP+ and water | | |
| Trimethylamine N- | Trimethylamine | Trimethylamine N-oxide | 1.8.99.X |
| oxide and Quinol | and Quinone | reductase | |
| Trimethylamine N- | Trimethylamine | Trimethylamine N-oxide | 1.7.2.3 or 1.7.2.X |
| oxide and reduced | and oxidized | reductase | |
| Cytochrome c | cytochrome c and | | |
| | water | | |
| Nitrite and reduced | Ammonia and | Nitrite reductase | 1.7.2.2 or 1.7.2.X |
| Cytochrome c552 | oxidized | complex | |
| | Cytochrome c552 | | |
| Arsenate and a | Arsenite and an | Respiratory arsenate | 1.20.99.1 or |
| reduced acceptor (e.g. | acceptor (e.g. | reductase | 1.20.99.X or |
| NADH or NADPH or | NAD+ or NADP+ | | 1.20.98.1 or |
| quinol or reduced | or quinone or | | 1.20.98.X |
| Cytochrome c) | oxidized | | |
| | Cytochrome c) | | |
| Iron (III) and a | Iron (II) and an | Iron-cytochrome-c | 1.9.99.1 or |
| reduced acceptor (e.g. | acceptor (e.g. | reductase | 1.9.99.X |
| NADH or NADPH or | NAD+ or NADP+ | | |
| quinol or reduced | or quinone or | | |
| Cytochrome c) | oxidized | | |
| | Cytochrome c) | | |

In particular, in some embodiments, the recombinant microorganism is engineered to inactivate one or more of the NADH or NADPH dehydrogenase enzymes listed in Table 1.

In some embodiments, the recombinant microorganism is engineered to inactivate one or more quinone molecules or the enzymes that synthesize these molecules listed in Table 1.

In some embodiments, the recombinant microorganism is engineered to delete or inactivate one or more molecules of the quinol oxidase complexes, including bo-type and bd-type complexes, listed in Table 1. In some embodiments, the recombinant microorganism is engineered to delete or inactivate one or more of the quinol:cytochrome c oxidoreductases listed in Table 1.

In some embodiments, the recombinant microorganism is engineered to delete or inactivate one or more of the cytochrome oxidases listed in Table 1.

In embodiments wherein the recombinant microorganisms are engineered to inactivate a terminal reductase, inactivation can be performed in function of the terminal reductase pathways activated in the microorganism. In some embodiments, the recombinant microorganism is engineered to inactivate one terminal reductase enzyme of the terminal reductase pathway. In other embodiments the recombinant microorganism is engineered to inactivate a plurality of terminal reductase enzymes expressed in the terminal reductase pathway. In other embodiments, the recombinant microorganism is engineered to remove all of the said terminal reductase enzymes expressed in the terminal reductase pathway.

In some of the embodiments wherein the recombinant microorganism is engineered to inactivate a terminal reductase pathway, the recombinant microorganism might be further engineered to ensure activation of the TCA cycle, e.g. to express a NAD(P)H producing oxidoreductase.

In some embodiments, the recombinant microorganism is engineered to delete or inactivate various combinations of the enzymes listed in Table 1. In particular, in some embodiments, the microorganism is engineered to inactivate NDH-1 and a bo-type quinol oxidase complex and/or NDH-2 and a bd-type quinol oxidase complex.

In some embodiments, the recombinant microorganism is engineered to inactivate the primary NADH dehydrogenases, in combination with enzymes involved in the biosynthesis of a redox active small molecule involved in respiration, such as a quinone. In some of these embodiments, no enzyme of the TCA of the recombinant microorganism is dependent on using the inactivated redox active small molecules as electron carriers.

In some embodiments, the recombinant microorganism is engineered to delete or inactivate all the enzymes listed in Table 1.

In some embodiments, the recombinant microorganism can be additionally or alternatively engineered to delete or inactivate an ATP synthase. In particular, in the recombinant microorganism herein disclosed, deletion or inactivation of ATP synthase can replace or is added to inactivation or deletion of NDH-1, NDH-2 and both quinol oxidase complexes (Jensen, P. R. et al, 1992, J. Bacteriol., 174, 7635-41). The recombinant microorganisms of those embodiments significantly increase overflow metabolism due to an increase in intracellular NADH that results from inhibited NDH activity.

In some embodiments, where the recombinant microorganism is yeast, the recombinant microorganism can be additionally or alternatively engineered to inactivate the respiratory complex I and/or the internal NADH dehydrogenase. In particular, in embodiments where the recombinant microorganism is a yeast microorganism such as Aspergillus or Neurospora, the respiratory complex I and the internal NADH dehydrogenase are inactivated. In embodiments where the recombinant microorganism is a yeast such as S. cerevisiae or Kluyveromyces which do not have respiratory complex 1, only the internal NADH dehydrogenase is inactivated.

In further embodiments, where the recombinant microorganism is yeast, the recombinant microorganism can be additionally or alternatively engineered to inactivate the external NAD(P)H dehydrogenases to increase NAD(P)H availability in the cytoplasm, in a manner that would increase yield and help maintain a redox balance of a heterologous pathway such as the heterologous pathways herein described.

In some embodiments, the recombinant microorganism can be additionally or alternatively engineered to inactivate a fermentative respiratory pathway of the recombinant microorganism.

The wording “fermentation”, “fermentative pathways” or “fermentation metabolism” refers to a pathway wherein the conversion from the substrate to the product is associated with the production of energy in the microorganism and wherein at least one of the reactions in the pathway involves transfer of electrons from an electron donor to a carrier molecule such as NADH or NADPH in which the final electron acceptor is a metabolite produced within the pathway. For example, in one of the fermentative pathways of E. coli, NADH generated through glycolysis transfers its electrons to pyruvate, yielding lactate. Exemplary enzymes involved fermentative pathways. These enzymes include NAD(P)H-requiring oxidoreductases and also enzymes that divert acetyl-CoA or any metabolic intermediate of glycolysis, including pyruvate from the TCA cycle

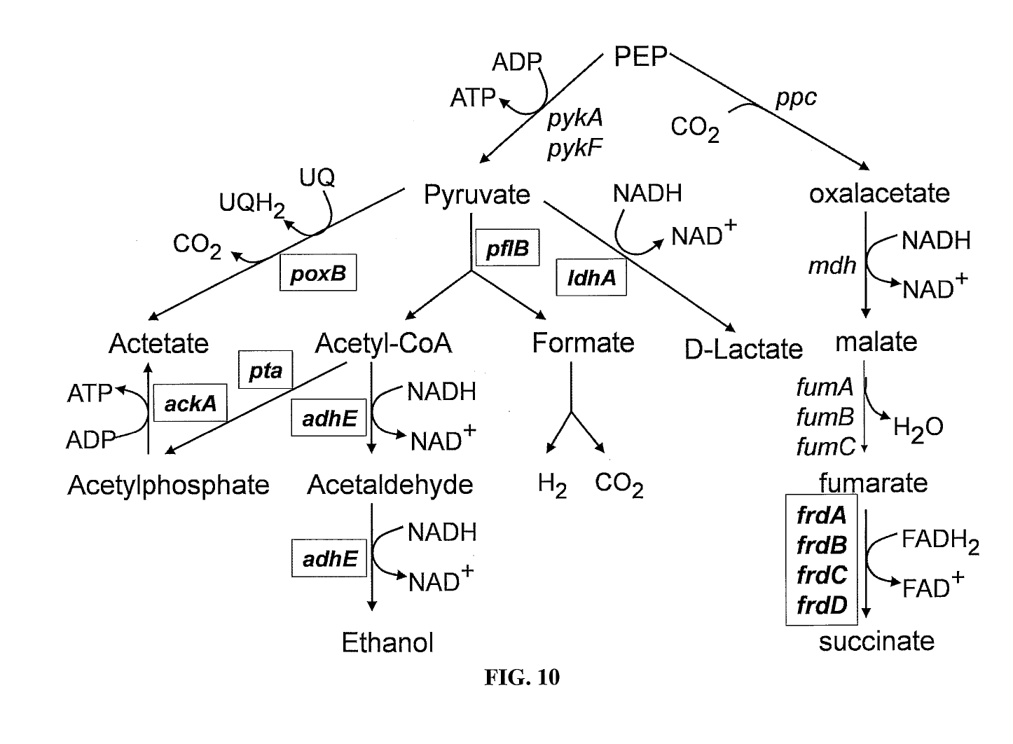

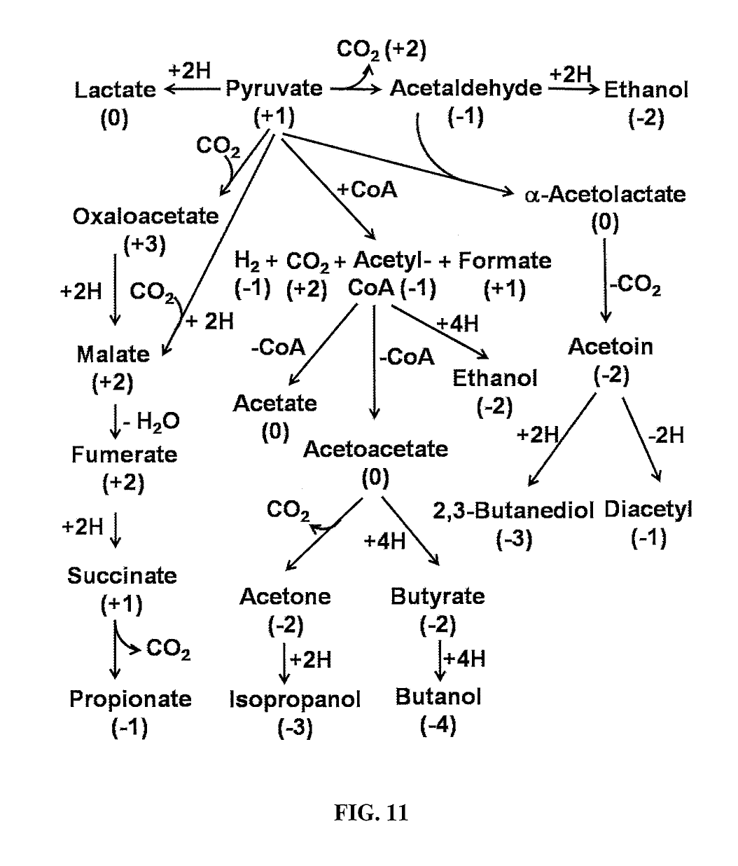

Exemplary fermentative pathways are illustrated in FIGS. 10 and 11. In particular, FIGS. 10 and 11 show exemplary fermentative pathways that are activated in a microorganism in particular when an excess of NADH or NAD(P)H is created. In the pathways shown in FIGS. 10 and 11, pyruvate is metabolized by the microorganism through several alternative metabolic pathways also identified as “overflow pathways” or “overflow metabolism” wherein NAD(P)H-requiring oxidoreductases transfer reducing equivalents from NADH or NADPH to another molecule in the pathway.

A first NAD(P)H oxidoreductase involved in the fermentative pathway shown in FIGS. 10 and 11. is D-lactate dehydrogenase (ldhA). This enzyme couples the oxidation of NADH to the reduction of pyruvate to D-lactate. Deletion of ldhA has previously been shown to eliminate the formation of D-lactate in a fermentation broth (Causey, T. B. et al, 2003, Proc. Natl. Acad. Sci., 100, 825-32).

A second NAD(P)H oxidoreductase involved in the fermentative pathway shown in FIGS. 10 and 11, is Acetaldehyde/alcohol dehydrogenase (adhE). Under aerobic conditions, pyruvate is also converted to acetyl-CoA, but this reaction is catalyzed by a multi-enzyme pyruvate dehydrogenase complex, yielding CO₂ and one equivalent of NADH. Acetyl-CoA fuels the TCA cycle but can also be oxidized to acetaldehyde and ethanol by acetaldehyde dehydrogenase and alcohol dehydrogenase, both encoded by the gene adhE. These reactions are each coupled to the reduction of one equivalents NADH.

A third NAD(P)H-requiring oxidoreductase involved in the fermentative pathway shown in FIGS. 10 and 11 is Fumarate reductase (frd). Under anaerobic conditions, phosphoenolpyruvate can be reduced to succinate via oxaloacetate, malate and fumarate, resulting in the oxidation of two equivalents of NADH to NAD⁺. Each of the enzymes could potentially be deleted to eliminate this pathway. For example, the final reaction catalyzed by fumarate reductase converts fumarate to succinate. The electron donor for this reaction is reduced menaquinone and each electron transferred results in the translocation of two protons. Deletion of this enzyme has proven useful for the generation of reduced pyruvate products.

A fourth NAD(P)H oxidoreductase involved in the fermentative pathway shown in FIGS. 10 and 11. is Pyruvate oxidase (poxB). Pyruvate can be oxidized by pyruvate oxidase to form acetate. This enzyme does not require NADH. However, upon decarboxylation of pyruvate, it transfers electrons from pyruvate to ubiquinone to form ubiquinol. Because of this electron transfer to the quinone pool, pyruvate oxidase indirectly increases the microorganism's need for oxygen. Removing pyruvate oxidase from the microorganism will prevent oxygen from being consumed by this pathway.

An additional enzyme involved in the fermentative pathway shown in FIGS. 10 and 11. is Phosphate acetyl transferase (pta)/acetate kinase A (ack4). These enzymes are involved in the conversion of acetyl-CoA via acetylphosphate to acetate. Deletion of ackA has previously been used to direct the metabolic flux away from acetate production (Underwood, S. A. et al, 2002, Appl. Environ. Microbiol., 68, 6263-72; Zhou, S. D. et al, 2003, Appl. Environ. Mirobiol., 69, 399-407), but deletion of pta should achieve the same result.

A still additional enzyme involved in the fermentative pathway shown in FIGS. 10 and 11, is Pyruvate formate lyase (pflB). This enzyme oxidizes pyruvate to acetyl-CoA and formate. Deletion of pflB has proven important for the overproduction of acetate (Causey, T. B. et al, 2003, Proc. Natl. Acad. Sci., 100, 825-32), pyruvate (Causey, T. B. et al, 2004, Proc. Natl. Acad. Sci., 101, 2235-40) and lactate (Zhou, S., 2005, Biotechnol. Lett., 27, 1891-96). Formate can be further be oxidized to CO₂ and hydrogen by a formate hydrogen lyase complex, but deletion of this complex should not be necessary in the absence of pflB.

Accordingly, in some embodiments, at least one or more of the above mentioned enzymes involved in fermentation pathways is inactivated. Those embodiments. These embodiments refer, in particular, to microorganisms, such as E. coli in which one or more of the above mentioned competing respiratory or fermentative pathways are present in the cell.

Additional NAD(P)H-requiring oxidoreductase or other enzymes involved in a fermentation pathway of a microorganism herein disclosed are listed Table 2, wherein the NAD(P) dependent oxidoreductases and the reactions they catalyze are indicated together with the respective EC number in which X=all child EC categories).

TABLE 2

| Relevant Reaction | Relevant Fermentation | | NAD(P)H- | EC |
|---|---|---|---|---|
| Substrate | Product | Enzyme name | requiring | number |
| Fumarate | Succinate | Fumarate Reductase | Yes | 1.3.1.6 or |
| | | | | 1.3.1.X, |
| | | | | 1.3.5.1 or |
| | | | | 1.3.5.X, |
| | | | | 1.3.99.1 |
| | | | | or |
| | | | | 1.3.99.X |
| Pyruvate | Lactate | Lactate Dehydrogenase | Yes | 1.1.1.27, |
| | | | | 1.1.1.28, |
| | | | | 1.1.1.X, |
| | | | | 1.1.2.3, |
| | | | | 1.1.2.4, |
| | | | | 1.1.2.5, |
| | | | | or |
| | | | | 1.1.2.X |
| Pyruvate | Acetate or acetyl | Pyruvate oxidase | | 1.2.2.2, |
| | phosphate | | | 1.2.2.X, |
| | | | | 1.2.3.3, |
| | | | | 1.2.3.X |
| Acetyl-CoA | Acetyl phosphate | Phosphate | | 2.3.1.8 or |
| | | transacetylase | | 2.3.1.X |
| Acetyl phosphate | Acetate | Acetate kinase | | 2.7.2.1 or |
| | | | | 2.7.2.X |
| Acetyl-CoA | Acetaldehyde or Ethanol | Aldehyde/Alcohol | Yes | 1.2.1.10, |
| | | dehydrogenase | | 1.2.1.X, |
| | | | | 1.1.1.X |
| | | | | or 1.1.1.1 |
| Pyruvate | Formate | Pyruvate-Formate | | 2.3.1.54, |
| | | lyase | | 2.3.1.X |
| 3- | 1,3-propanediol | 1,3-propanediol | Yes | 1.1.1.202 |
| hydroxypropionaldehyde | | dehydrogenase | | or |
| | | | | 1.1.1.X |
| Glycerol | 3- | Glycerol dehydratase | | 4.2.1.30 |
| | hydroxypropionaldehyde | | | or |
| | | | | 4.2.1.X |
| Pyruvate | Acetolactate | α-acetolactate synthase | | 2.2.1.6 or |
| | | | | 2.2.1.X |
| Acetoin | 2,3-butanediol | Acetoin reductase or | Yes | 1.1.1.4 or |
| | | 2,3,-butanediol | | 1.1.1.X |
| | | dehydrogenase | | |
| Acetolactate | Acetoin | α-acetolactate | | 4.1.1.5, |
| | | decarboxylase | | 4.1.1.X, |
| Propionyl-CoA | Propionate | propionyl- | | 2.8.3.X |
| | | CoA:succinate CoA | | |
| | | transferase | | |
| Pyruvate | Propionyl-CoA | methylmalonyl-CoA | | 2.1.3.1 or |
| | | carboxyltransferase | | 2.1.3.X |
| Butyryl-CoA | Butyrate | Acetate CoA- | | 2.8.3.8, |
| | | transferase | | 2.8.3.X, |
| Butyryl-CoA | Butyryl phosphate | phosphotransbutyrylase | | 2.3.1.19, |
| | | | | 2.3.1.X |
| Butyryl phosphate | Butyrate | Butyrate kinase | | 2.7.2.7 or |
| | | | | 2.7.2.X |
| Butyraldehyde | Butanol | Butanol dehydrogenase | Yes | 1.2.1.10, |
| | | | | 1.2.1.X, |
| | | | | 1.1.1.X |
| | | | | or 1.1.1.1 |
| Butyryl-CoA | Butyraldehyde | Butyraldehyde | Yes | 1.2.1.10, |
| | | dehydrogenase | | 1.2.1.X, |
| | | | | 1.1.1.X |
| | | | | or 1.1.1.1 |
| Crotonyl-CoA | Butyryl-CoA | Butyryl-CoA | Yes | 1.3.2.1 or |
| | | dehydrogenase | | 1.3.2.X |
| | | | | or |
| | | | | 1.3.99.2 |
| | | | | or |
| | | | | 1.3.99.X |
| β-hydroxybutyryl-CoA | Crotonyl-CoA | Crotonase | | 4.2.1.17 |
| | | | | or |
| | | | | 4.2.1.55 |
| | | | | or |
| | | | | 4.2.1.X |
| Acetoacetyl-CoA | β-hydroxybutyryl-CoA | Hydroxybutyryl-CoA | Yes | 1.1.1.157, |
| | | dehydrogenase | | 1.1.1.X, |
| Acetyl-CoA | Acetoacetyl-CoA | Thiolase | | 2.3.1.9 |
| Acetoacetate | Acetone | Acetoacetate | | 4.1.1.4 or |
| | | decarboxylase | | 4.1.1.X |
| Formate | Hydrogen gas | Formate hydrogen | | No EC |
| | | lyase complex | | number |
| | | | | assigned |
| Pyruvate | acetaldehyde or Carbon | Pyruvate | | 4.1.1.1, |
| | dioxide | decarboxylase | | 4.1.1.X, |
| Glyceraldehyde-3- | Glycerol | Glycerol-3-phosphate | | 3.1.3.21 |
| phosphate | | phosphohydrolase | | or |
| | | | | 3.1.3.X |
| Formate | carbon dioxide | Formate hydrogen | | No EC |
| | | lyase complex | | number |
| | | | | assigned |
| Formate | Hydrogen gas | Hydrogenase | Yes | 1.12.1.2 |
| | | | | or |
| | | | | 1.12.1.X, |
| | | | | 1.12.X.X |
| Formate | Carbon dioxide | Formate | Yes | 1.2.1.2 or |
| | | dehydrogenase | | 1.2.1.X |
| | | | | or |
| | | | | 1.2.1.43 |
| | | | | or 1.2.2.1 |
| | | | | or |
| | | | | 1.2.2.X |
| | | | | or 1.2.2.3 |

The fermentative pathways involving the enzymes listed in Table 2 lead to the production of fermentative products, wherein the wording “fermentative products” or “fermentation products” or “overflow products” refers to the final or intermediate products of fermentation metabolism. Fermentation products may include succinate, lactate, acetate, ethanol, formate, carbon dioxide, hydrogen gas, 1,3-propanediol, 2,3-butanediol, acetoin, propionate, butyrate, butanol, acetone, singly or mixtures thereof. Fermentative products include both oxidized and reduced products of fermentation.

When activated in the microorganism, fermentative NAD(P)H-requiring oxidoreductases or NAD(P)H-requiring pathways that contain one or more reactions controlled by at least one of the enzymes listed in Table 2 greatly decrease the amount of reducing equivalents that can be obtained by breaking down glucose.

Accordingly, in some embodiments, the recombinant microorganism is engineered to inactivate one or more of the enzymes indicated in Table 2. In particular, in some embodiments, the microorganism is engineered to inactivate at least one of Fumarate Reductase, Lactate Dehydrogenase, Pyruvate oxidase, Phosphate transacetylase, Acetate kinase, Aldehyde/Alcohol dehydrogenase, Pyruvate-Formate lyase, 1,3-propanediol dehydrogenase, Glycerol dehydratase, α-acetolactate synthase, Acetoin reductase, 2,3,-butanediol dehydrogenase, α-acetolactate decarboxylase or acetoin reductase, propionyl-CoA:succinate CoA transferase, methylmalonyl-CoA carboxyltransferase.

In some embodiments, the recombinant microorganism can be engineered to inactivate at least one of the following enzymes: Acetate CoA-transferase phosphotransbutyrylase, Butyrate kinase, Butanol dehydrogenase, Butyraldehyde dehydrogenase, Butyryl-CoA dehydrogenase, Crotonase, Hydroxybutyryl-CoA dehydrogenase, Thiolase, Acetoacetate decarboxylase, Formate hydrogen lyase complex, Pyruvate decarboxylase, alcohol dehydrogenase, Glycerol-3-phosphate phosphohydrolase, Formate hydrogen lyase complex, Hydrogenase, and Formate dehydrogenase.

Should the microorganism activate alternative NAD(P)H-requiring enzymes or pathways that outcompete the NAD(P)H-requirement of the biotransformation, then these pathways are sequentially inactivated until most (5 or more) or all (10) of the NAD(P)H produced by the cell is consumed by the enzyme or pathway of the biotransformation. At this point, the cell is dependent upon the enzyme or pathway for survival.

In some embodiments of this disclosure, one or more, of the above mentioned fermentative pathways may be reactivated if survival issues arise, to support survival to the extent that the related fermentative product should not accumulate at significant quantities.

In some embodiments, those competing fermentative pathways are deleted that remain most active and produce the most by-product. Methods to identify by-products generated by fermentative pathways are well established. For example, if ethanol is the main by-product of a biotransformation as measured by gas chromatography (GC) or high performance liquid chromatography (HPLC) analysis, then the gene responsible for the production of ethanol is inactivated, in particular by deletion. This process is preferably repeated until the amount of all by-product produced is less than 5% by weight per glucose.

In some embodiments, the amount of NAD(P)ii available for the NAD(P)H-requiring heterologous oxidoreductase, is increased in the recombinant microorganism by engineering the microorganism to express at least one heterologous NAD(P)H producing oxidoreductase enzyme of the TCA cycle.

The term “express” as used herein, with reference to a biologically active molecule, such as a protein, in a microorganism indicates activation of that biologically active molecule in the microorganism, which for enzymes include but is not limited to transcription and translation in the microorganism of a polynucleotide encoding for such as an enzyme together with any post-translational modifications, if any, necessary to convert the enzyme in its active form; for polynucleotides such as genes includes but is not limited to transcription of the polynucleotide sequence and, if the polynucleotides encodes for a protein, translation of the resulting transcript to a protein; for polynucleotide such as RNA includes but is not limited to the transcription of the polynucleotide.

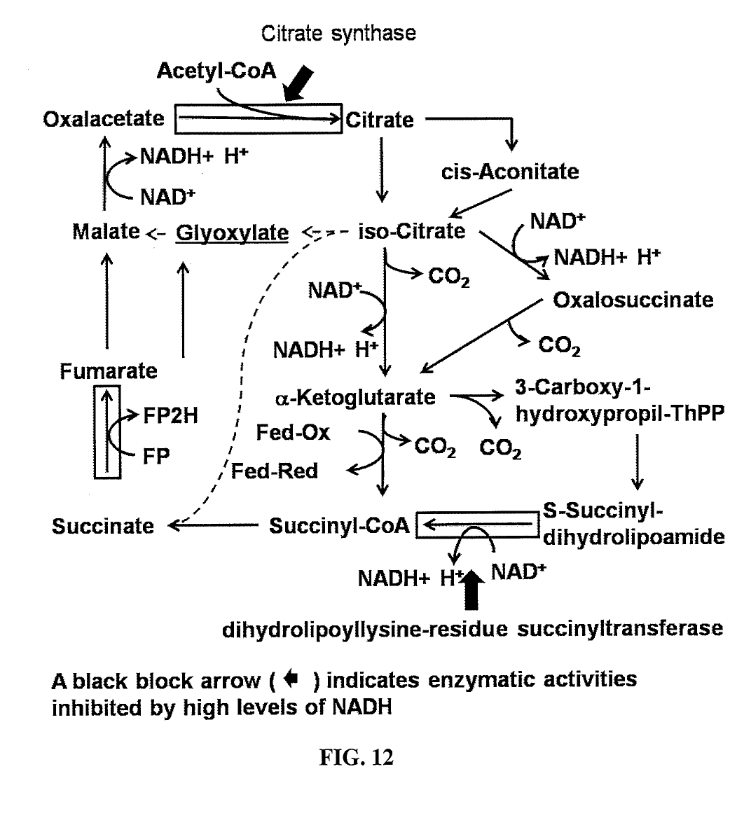

Reference is made to FIG. 12 where a detailed and comprehensive illustration of a TCA cycle that comprises the exemplary TCA cycle illustrated in FIG. 7, is depicted.

In particular FIG. 12 shows a schematic depiction of the metabolites involved in the TCA cycle. Each arrow represents and enzymatic reaction carried out by the enzymatic activities listed in table 4. The flow of reducing equivalents (from NAD+ to NADH+H⁺, from oxidized ferredoxin (Fed-Ox) to reduced ferredoxin (Fed-Red) and from reduced flavoprotein (FP) to reduced flavoprotein (FP2H)) and also the formation of carbon dioxide are shown.

A block arrow indicates reactions catalyzed by an enzyme that is inhibited by high levels of NAD(P)H. The name of the enzymes catalyzing these reactions is also shown in black next to the arrow. Enzymatic steps represented by an arrow encased in an box, indicate an E. coli enzymatic activity that should be modified to obtain a fully functional TCA cycle under anaerobic conditions.

The complete TCA cycle includes the following set of enzymatic reactions: conversion of oxalacetate plus acetyl-CoA into a molecule of citrate carried out by enzymatic activity EC 2.3.3.1 (known among other names as citrate synthase); conversion of citrate to iso-citrate as a single step catalyzed by EC 4.2.1.3 (known among other names a aconitase) or through the formation of cis-aconitate also catalyzed by EC 4.2.1.3 (known among other names a aconitase); conversion of iso-citrate into α-ketoglutarate either directly by using EC 1.1.1.41 (known among other names as isocitrate dehydrogenase) or through, the formation of oxalosuccinate EC 1.1.1.42 (known among other names as isocitrate dehydrogenase); conversion of α-ketoglutarate to succinyl-CoA either using EC 1.2.7.3 (known among other names as 2-ketoglutarate ferredoxin oxidoreductase) or through the formation of 3-carboxy-1-hydroxypropil-ThPP using EC 1.2.4.2 (known among other names as alpha-ketoglutarate dehydrogenase), its conversion into S-Succinyl-dihydrolipoamide using EC 1.2.4.2 (known among other names as alpha-ketoglutarate dehydrogenase) which in turn is converted into Succinyl-CoA using EC 2.3.1.61 (known among other names as dihydrolipoamide succinyltransferase); conversion of succinyl-CoA into succinate through the use of EC 6.2.1.4 (known among other names as succinyl-CoA synthetase), 6.2.1.5 (known among other names as succinyl-CoA synthetase) or 3.1.2.3 (known among other names as succinyl-CoA hydrolase); conversion of succinate into fumarate through the use of EC 1.3.5.1 (known among other names as succinate dehydrogenase) or EC 1.3.99.1 (known among other names as succinate dehydrogenase); conversion of fumarate into malate by using EC 4.2.1.2 (known among other names as fumarase); conversion of malate into oxalacetate by using EC 1.1.1.37 (known among other names as malate dehydrogenase). The additional enzymatic activities that constitute the glyoxylate shunt are indicated by a dashed arrow. As consequence of these activities a new metabolite, glyoxylate, appears. This fact has been indicated by underlining its name.

In some embodiments, the amount of NAD(P)H recombinant microorganism is engineered to express one or more heterologous NAD(P)H-producing oxidoreductase of the TCA cycle. In particular, in some of those embodiments, the recombinant microorganism is further engineered to inactivate corresponding native enzymes in the microorganism

The term “corresponding” as used herein with reference to enzymes or other biologically active molecules, indicates molecules having substantially the same biological activity, which for enzymes includes the ability to specifically act upon the same substrates

Accordingly, in some embodiments wherein a heterologous NAD(P)H producing oxidoreductase is expressed, said heterologous oxidoreductase replaces corresponding native enzymes in the TCA cycle of the microorganism.

The term “replace” as used herein with reference to biologically active molecules such as an enzyme indicates that the molecules substitute with respect to enzymatic activity or some property thereof for a native molecule or enzyme that has been removed or deleted from the wild-type organism.

In some of those embodiments, the recombinant microorganisms herein disclosed are microorganisms, such as E. coli, in which TCA cycle enzymes have low or no activity under conditions where the respiratory pathway has limited or no activity. In some of those embodiments, the recombinant microorganisms herein disclosed are microorganisms, such as E. coli, where TCA cycle enzymes are inhibited by the presence of high levels of NAD(P)H within the cell. These embodiments refer, in particular, to E. coli.

In some embodiments, citrate synthase is replaced by a corresponding enzyme such as the methyl citrate synthase.

In some embodiments, alpha-ketoglutarate dehydrogenase is replaced by a corresponding enzyme

In particular, in some embodiments, the recombinant microorganism herein disclosed is engineered to replace the native alpha-ketoglutarate dehydrogenase with an engineered alpha-ketoglutarate dehydrogenase. This includes, but is not limited to replace the lipoamide dehydrogenase of the alpha-ketoglutarate dehydrogenase with a lipoamide dehydrogenase that is not inhibited by NADH.

In some of those embodiments, a strain can be generated in E. coli, that does not show inhibition of the pyruvate dehydrogenase complex which also contains the lipoamide dehydrogenase and this complex is not inhibited by NADH. This strain is generated with suitable techniques such as by applying mutagenizing agents like MNNG (N-methyl-N′-nitro-N-nitrosoguanidine) to the cells and selecting for anaerobic growth with absent or decreased activity for the lactate dehydrogenase and pyruvate formate lyase enzymes and selecting for anaerobic growth on LB media containing 1% glucose. This method is similar to the method that has been reported by Kim Y. et al, 2007, Applied and Environmental Microbiology, 73(6), 1766-71. Alternatively, analogous mutations can be introduced to remove NADH inhibition of the pyruvate dehydrogenase and alpha ketoglutarate dehydrogenase in the genes of other microorganisms.

Recombinant microorganisms, such as E. coli that have no or decreased activity for the lactate dehydrogenase and pyruvate formate lyase enzymes, are engineered to remove the NADH inhibition of the pyruvate dehydrogenase and alpha ketoglutarate dehydrogenase and as a consequence show anaerobic growth on LB media containing 1% glucose. Aerobic growth is comparable to parental strain or wild-type E. coli strain W3110 when cultured in rich medium. In addition to these phenotypic characteristics these mutated strains generate ethanol as main fermentative product.

Furthermore the mutation will likely remove the NADH inhibition of the lipoamide dehydrogenase subunit of the alpha-ketotglutarate dehydrogenase enzyme complex and result in partial removal of the catabolite repression and activity of the therefore show increased TCA cycle activity while feeding glucose or other energy rich carbon sources to the cells. This is measurable by increased carbon dioxide production relative to levels generated by cells with inactive TCA cycle and would also results in increased NADH availability for biocatalysis. If the TCA cycle is active and the biocatalytic enzyme or pathway is active, one would see increased product per glucose yield that is greater than 4 (but less than 10)

In some embodiments, removing the NADH inhibition of the pyruvate dehydrogenase and the alpha-ketoglutarate dehydrogenase can be performed by mutagenizing the strain and deleting the ldh and pfl genes to be able to select for anaerobic growth on glucose. Strains that grow are expected to have a mutation in the lpdA gene (lipoamide dehydrogenase), or the aceE or the aceF gene based on report from the art.

In some embodiments, the recombinant microorganism is engineered to replace a citrate synthase (EC 2.3.3.1) with a dimeric citrate synthase mutant including at least one of the following amino acid mutations Y145A, R163L, K167A, and D362N.

In some of those embodiments, the citrate synthase is a type II citrate synthase enzyme, and the recombinant microorganism is a gram negative bacteria such as E. coli. In some of those embodiments, the type II citrate enzyme is engineered to introduce at least one of the following dimeric mutations Φ362N, Y145A, R163L, and K167A). The mutant D362N exhibits minimization of NADH inhibition (Patton A. J. et al., Eur J. Biochem. 1993 May 15; 214(1):75-81). The mutants Y145A, R163L and K167A have been shown to exhibit a reduced inhibition by NADH (Stokell, J Biol. Chem. 2003 Sep. 12; 278(37):35435-43). In some embodiments, the type citrate synthase is engineered to introduce all the dimeric mutations D362N, Y145A, R163L, and K167A. The site directed mutagenesis can be done using various techniques known in the art and in particular the technique described by Horton R. M., Mol. Biotechnol., 3(2), 93-99. In particular, some embodiments the mutation or mutations can be performed on the citrate synthase from E. coli (NP_{—}415248.1) whose sequence is indicated in the enclosed sequence listing with SEQ ID NO:1. In some embodiments the mutation or mutations can be performed on the methyl citrate synthase from E. Coli (NP_{—}414867.1) whose sequence is indicated in the enclosed sequence listing with SEQ ID NO:2.

In some of those embodiments, a type II citrate synthase is replaced by a type I citrate synthase. The type I citrate synthase is an enzyme expressed in animals, plants and some bacteria and appears to be a simple dimer that is not allosterically regulated. [Else A J, Danson M J, Weitzman P D (J988). “Models of proteolysis of oligomeric enzymes and their applications to the trypsinolysis of citrate synthases.” Biochem J 1988; 254(2); 437-42].

In some embodiments, the endogenous citrate synthase can be replaced by a methylcitrate synthase (EC 2.3.3.8) that can also catalyze the conversion of acetyl co-A and oxaloacetate to citrate and is not NAD(P) inhibited

In some embodiments, the endogenous citrate synthase can be replaced by an enzyme that can catalyze the conversion of acetyl co-A and oxaloacetate to citrate such as EC 2.3.3.1 citrate synthase, EC 2.3.3.8 methylcitrate synthase or others.

In some embodiments, the recombinant microorganism is engineered to replace a fumarate reductase/succinate dehydrogenase with an NADH independent fumarate reductase.

Fumarate reductases are a group of enzymes of the TCA cycles that usually include a NAD(H) binding domain, and in some cases a domain characterized as fumarate reductase/succinate dehydrogenase domain and/or an ApbE domain.

In some embodiments, the NADH dependant fumarate reductase is selected from the following fumarate reductase listed in Table 3, Table 3 also reports for each of the fumarate reductase, the sequence identifier of the corresponding gene and protein sequences listed in the enclosed sequence listing is also reported.

TABLE 3

| | | | Protein |
|---|---|---|---|
| Gene Sequence | Protein sequence | Organism | number/identity |
| SEQ ID NO: 3 | SEQ ID NO: 4 | Leishmania major | AAZ14310.1 |
| SEQ ID NO: 5 | SEQ ID NO: 6 | Leishmania major | AAZ14343.1 |
| SEQ ID NO: 7 | SEQ ID NO: 8 | Leishmania major | AAZ14344.1 |
| SEQ ID NO: 9 | SEQ ID NO: 10 | Trypanosoma brucei | AAX20162 |
| SEQ ID NO: 11 | SEQ ID NO: 12 | Trypanosoma brucei | AAN40014.1 |
| SEQ ID NO: 13 | SEQ ID NO: 14 | Trypanosoma brucei | AAX20164.1 |
| SEQ ID NO: 15 | SEQ ID NO: 16 | Leishmania | Emb\|CAM43326.1 |
| | | braziliensis | |
| SEQ ID NO: 17 | SEQ ID NO: 18 | Leishmania | emb\|CAM43299.1 |
| | | braziliensis | |
| SEQ ID NO: 19 | SEQ ID NO: 20 | Leishmania | emb\|CAM43327.1 |
| | | braziliensis | |
| SEQ ID NO: 21 | SEQ ID NO: 22 | Leishmania | emb\|CAM43328.1 |
| | | braziliensis | |
| SEQ ID NO: 23 | SEQ ID NO: 24 | Leishmania major | ref\|XP_843225.1 |
| | | strain Friedlin | |
| SEQ ID NO: 25 | SEQ ID NO: 26 | Leishmania major | ref\|XP_843226.1 |
| | | strain Friedlin | |
| SEQ ID NO: 27 | SEQ ID NO: 28 | Leishmania infantum | ref\|XP_001468931.1 |
| SEQ ID NO: 29 | SEQ ID NO: 30 | Leishmania infantum | ref\|XP_001468899.1 |
| SEQ ID NO: 31 | SEQ ID NO: 32 | Leishmania infantum | ref\|XP_001468932.1 |
| SEQ ID NO: 33 | SEQ ID NO: 34 | Trypanosoma cruzi | ref\|XP_807320.1 |
| | | strain CL Brener | |
| SEQ ID NO: 35 | SEQ ID NO: 36 | Trypanosoma cruzi | ref\|XP_803046.1 |
| | | strain CL Brener | |
| SEQ ID NO: 37 | SEQ ID NO: 38 | Trypanosoma cruzi | ref\|XP_810232.1 |
| | | strain CL Brener | |
| SEQ ID NO: 39 | SEQ ID NO: 40 | Trypanosoma cruzi | ref\|XP_804499.1 |
| | | strain CL Brener | |
| SEQ ID NO: 41 | SEQ ID NO: 42 | Trypanosoma cruzi | ref\|XP_810233.1 |
| | | strain CL Brener | |
| SEQ ID NO: 43 | SEQ ID NO: 44 | Trypanosoma cruzi | ref\|XP_811221.1 |
| | | strain CL Brener | |

In some embodiments, the recombinant microorganism where a heterologous NAD(P)H-producing oxidoreductase is expressed can be further engineered to further activate one or more enzymes of the TCA cycle, e.g., by inactivating enzymes that catalyze transcriptional repression of those enzymes. Exemplary embodiments of recombinant microorganisms so engineered include recombinant microorganisms wherein at least one of the following enzymes or transcription factors is deleted or inactivated, sdhCDAB-b0725-sucABCD operon by chromosomal promoter exchange as reported by (Veit, Polen, Wendisch 2007), and/or at least one of Fnr, ArcA, Cra, Crp (and others) (Shalel-Levanon, San, Bennett, 2005 Biotech and Bioeng, 89(5), Shalel-Levanon, San, Bennett, 2005 Biotech and Bioeng, 92(2); Perrenaud, Sauer 2005, J Bacteriology)

In some embodiments, the recombinant microorganism where a heterologous NAD(P)H-producing oxidoreductase is expressed can be further engineered to activate the soluble fraction of an ATPase in the microorganism, so to reduce ATP levels and increase NAD(P)H available in the cytoplasm.

In some embodiments, the recombinant microorganism where a heterologous NAD(P)H-producing oxidoreductase is expressed can be further engineered to further activate one or more enzymes of the TCA cycle, e.g., by inactivating or deleting enzymes that catalyze transcriptional repression of those enzymes. Exemplary embodiments of recombinant microorganisms so engineered include recombinant microorganisms wherein at least one of the following enzymes is deleted or inactivated, sdhCDAB-b0725-sucABCD operon by chromosomal promoter exchange as reported by (Veit, Polen, Wendisch 2007), and/or at least one of Fnr, ArcA, Cra, Crp (and others) (Shalel-Levanon, San, Bennett, 2005 Biotech and Bioeng, 89(5), Shalel-Levanon, San, Bennett, 2005 Biotech and Bioeng, 92(2); Perrenaud, Sauer 2005, J Bacteriology). This is applicable to all microorganisms that express a complete functional TCA cycle and whose TCA cycle enzymes are regulated by the above mentioned transcription factors.

In some embodiments, the recombinant microorganism where a heterologous NAD(P)H-producing oxidoreductase is expressed can be further engineered to activate the soluble fraction of an ATPase in the microorganism, so to reduce ATP levels and increase NAD(P)H available in the cytoplasm

In some embodiments, the activation, inactivation, replacement or expression of one or more of the above mentioned enzymes is performed by using standard molecular biology manipulation techniques. In particular, the recombinant microorganism can be engineered by transfection, transformation and other techniques identifiable by a skilled person upon reading of the present disclosure.

“Transfection,” or “transformation,” as used herein, refers to the insertion of an exogenous, endogenous, or heterologous polynucleotide into a host cell (eukaryotic or prokaryotic), irrespective of the method used for the insertion, for example, direct uptake, transduction, mating or electroporation, polymer-mediated, chemical-mediated, or viral.

Methods to express a polynucleotide, express at various levels include lower and higher levels compared to level of expression in a native microorganism, repress expression of, and delete genes in host cells are well known in the art and any such method is contemplated for use to construct the yeast strains of the present.

Any method can be used to activate an endogenous or exogenous nucleic acid molecule into a host cell and many such methods are well known to those skilled in the art. For example, transformation, electroporation, conjugation, and fusion of protoplasts are common methods for introducing nucleic acid into host cells. See, e.g., Ito et al., J. Bacteriol. 153:163-168 (1983); Durrens et al., Curr. Genet. 18:7-12 (1990); Becker and Guarente, Methods in Enzymology 194:182-187 (1991); and Sambrook, J., Fritsch, E. F. and Maniatis, T., Molecular Cloning: A Laboratory Manual, Second Edition, Cold Spring Harbor Laboratory Press, Cold Spring Harbor, N.Y. (1989).

In an embodiment, the integration of a gene of interest into a DNA fragment or target gene occurs according to the principle of homologous recombination. This embodiment results in an inactivated gene or enzyme. According to this embodiment, an integration cassette containing a module comprising at least one selective marker gene and/or the gene to be integrated (internal module) is flanked on either side by DNA fragments that act as recognition sequences (sequences of DNA to which recombinase enzymes bind and catalyze breakage and/or rearrangement of the DNA sequence) for a site-specific recombinase, such as cre-lox recombinase of bacteriophage lambda, RED recombinase from bacteriophage lambda, or FLP recombinase of Saccharomyces, and is further flanked on each side by DNA fragments that are homologous to those of the ends of the targeted integration site (recombinogenic sequences). Herein, “site-specific recombination” refers to a nucleic acid crossover event, such as the integration of bacteriophage lambda DNA into host chromosomal DNA that requires homology of only a very short region and uses an enzyme specific for that recombination, herein referred to as a “site-specific recombinase” enzyme. After transforming the host cell with the cassette by appropriate methods, a homologous recombination between the recombinogenic sequences may result in the internal module replacing the chromosomal region in between the two sites of the genome corresponding to the recombinogenic sequences of the integration cassette.

In an embodiment, for gene deletion, the integration cassette comprises an appropriate selective marker gene flanked by the recombinogenic sequences. In an embodiment, for integration of a heterologous gene into the host chromosome, the integration cassette comprises the heterologous gene under the control of an appropriate promoter and terminator together with the selectable marker flanked by recombinogenic sequences. In an embodiment, the heterologous gene comprises an appropriate native gene desired to increase the copy number of a native gene(s). The “selectable marker gene”, “marker”, or “selectable marker” can be any gene used in a host to express a protein or polynucleotide, including but not limited to, an antibiotic resistance gene such as tetracycline, erythromycin, ampicillin, chloramphenicol, kanamycin, spectinomycin, streptomycin, gentamycin, neomycin, ciprofloxacin, and/or a resistance gene for a toxic substance or compound, such as mercury, and/or a resistance gene for auxotrophy complementation, such as ScURA3 (for uracil) or an amino acid biosynthetic pathway gene. The recombinogenic sequences can be chosen at will, depending on the desired integration site suitable for the desired application.

Additionally, in an embodiment, certain introduced marker genes are removed or deleted from the genome using techniques well known to those skilled in the art. For example, ampicillin marker loss can be obtained by introduction of a recombinase enzyme through the aforementioned standard techniques. Host cells may then be screened for sensitivity to the antibiotic to confirm loss of the marker gene.

Additionally, in an embodiment, certain introduced marker genes are removed or deleted from the genome using techniques well known to those skilled in the art. For example, URA3 marker loss can be obtained by plating URA3 containing cells in FOA (5-fluoro-orotic acid) containing medium and selecting for FOA resistant colonies (Boeke. J. et al, 1984, Mol. Gen. Genet, 197, 345-47).

The exogenous or endogenous nucleic acid molecule contained within a host cell of the disclosure can be maintained within that cell in any form. For example, exogenous or endogenous nucleic acid molecules can be integrated into the genome of the cell or maintained in an episomal state, such as a plasmid, that can stably be passed on (“inherited”) to daughter cells. Such extra-chromosomal genetic elements (such as plasmids, etc.) can additionally contain selection markers that ensure the presence of such genetic elements in daughter cells. Moreover, the host cells can be stably or transiently transformed. In addition, the host cells described herein can contain a single copy, or multiple copies of a particular exogenous or endogenous nucleic acid molecule as described above.

Methods for expressing a polypeptide from an exogenous or endogenous nucleic acid molecule are well known to those skilled in the art. These methods may also be used to activate endogenous or native DNA sequences from a host. Such methods include, without limitation, constructing a nucleic acid such that a regulatory element promotes the expression of a nucleic acid sequence that encodes the desired polypeptide. Typically, regulatory elements are DNA sequences that regulate the expression of other DNA sequences at the level of transcription. Thus, regulatory elements include, without limitation, promoters, enhancers, and the like. For example, the exogenous or endogenous genes can be under the control of an inducible promoter or a constitutive promoter. Moreover, methods for expressing a polypeptide from an exogenous or endogenous nucleic acid molecule in bacteria or yeast are well known to those skilled in the art.

For example, nucleic acid-constructs that are capable of expressing exogenous or endogenous polypeptides within Kluyveromyces (see, e.g., U.S. Pat. Nos. 4,859,596 and 4,943,529, each of which is incorporated by reference herein in its entirety) and Saccharomyces (see, e.g., Gelissen et al., Gene 190(1):87-97 (1997)) are well known. In another embodiment, heterologous control elements can be used to activate or repress expression of endogenous or native genes. In yet another embodiment, endogenous or native control elements can be used to activate or repress expression of endogenous or native genes. Additionally, when expression is to be repressed or eliminated, the gene for the relevant enzyme, protein or RNA can be eliminated by known deletion techniques.

As described herein, hosts within the scope of the disclosure can be identified by selection techniques specific to the particular enzyme being expressed, over-expressed or repressed. Methods of identifying the strains with the desired phenotype are well known to those skilled in the art. Such methods include, without limitation, PCR and nucleic acid hybridization techniques such as Northern and Southern analysis, altered growth capabilities on a particular substrate or in the presence of a particular substrate, a chemical compound, a selection agent and the like. In some cases, immunohistochemistry and biochemical techniques can be used to determine if a cell contains a particular nucleic acid by detecting the expression of the encoded polypeptide. For example, an antibody having specificity for an encoded enzyme can be used to determine whether or not a particular host cell contains that encoded enzyme. Further, biochemical techniques can be used to determine if a cell contains a particular nucleic acid molecule encoding an enzymatic polypeptide by detecting a product produced as a result of the expression of the enzymatic polypeptide. For example, transforming a cell with a vector encoding acetyl-CoA synthetase and detecting increased acetyl-CoA concentrations indicates the vector is both present and that the gene product is active. Methods for detecting specific enzymatic activities or the presence of particular products are well known to those skilled in the art. For example, the presence of acetyl-CoA can be determined as described by Dalluge et al., Anal. Bioanal. Chem. 374(5):835-840 (2002).

A recombinant microorganism within the scope of the disclosure also can have inactivated enzymatic activity such as inactivated alcohol dehydrogenase activity. Thus recombinant microorganisms lacking alcohol dehydrogenase activity are considered to have reduced alcohol dehydrogenase activity since most, if not all, comparable host strains have at least some alcohol dehydrogenase activity. Such reduced enzymatic activities can be the result of lower enzyme concentration, lower specific activity of an enzyme, or a combination thereof. Many different methods can be used to make host having reduced enzymatic activity. For example, a host cell can be engineered to have a disrupted enzyme-encoding locus using common mutagenesis or knock-out technology. See, e.g., Methods in Yeast Genetics (1997 edition), Adams, Gottschling, Kaiser, and Stems, Cold Spring Harbor Press (1998). Include additional references here for bacteria . . . could use E. coli references for examples.

Alternatively, antisense technology can be used to reduce or inactivate one or more enzymatic activity. For example, a host cell can be engineered to contain a cDNA that encodes an antisense molecule that prevents an enzyme from being made. The term “antisense molecule” as used herein encompasses any nucleic acid molecule that contains sequences that correspond to the coding strand of an endogenous polypeptide. An antisense molecule also can have flanking sequences (e.g., regulatory sequences). Thus antisense molecules can be ribozymes or antisense oligonucleotides. A ribozyme can have any general structure including, without limitation, hairpin, hammerhead, or axhead structures, provided the molecule cleaves RNA.

Recombinant microorganisms having an inactive enzyme according to the present disclosure can be identified using any method. For example, recombinant microorganisms having reduced alcohol dehydrogenase activity can be easily identified using common methods, for example, by measuring ethanol formation via gas chromatography.

Further exemplary techniques that can be used to inactivate enzymes or polynucleotides that encode enzymes in accordance with the present disclosure include but are not limited to the techniques described in Calhoun, M. W. et al, 1993, J. Bacteriol., 175, 3013-19; Calhoun, M. W. et al, 1993, J. Bacteriol., 175, 3020-25; Teixeira de Mattos, M. J. et al, 1997, J. Biotechnol., 59, 117-26; Jensen, P. R. et al, 1992, J. Bacteriol., 174, 7635-41.

In some embodiments, the recombinant microorganism where a heterologous NAD(P)H-producing oxidoreductase is expressed can be further engineered to further activate one or more enzymes of the TCA cycle, e.g., by inactivating or deleting enzymes that catalyze transcriptional repression of those enzymes. Exemplary embodiments of recombinant microorganisms so engineered include recombinant microorganisms wherein at least one of the following enzymes is inactivated, sdhCDAB-bO725-sucABCD operon by chromosomal promoter exchange as reported by (Veit, Polen, Wendisch 2007), and/or at least one of Fnr, ArcA, Cra, Crp (and others) (Shalel-Levanon, San, Bennett, 2005 Biotech and Bioeng, 89(5), Shalel-Levanon, San, Bennett, 2005 Biotech and Bioeng, 92(2); Perrenaud, Sauer 2005, J Bacteriology). This can apply to all recombinant microorganisms herein disclosed.

In some embodiments, where a heterologous NAD(P)H-producing oxidoreductase is expressed, the heterologous NAD(P)H-producing oxidoreductase is an enzyme of the TCA cycle and is expressed in a microorganism that in its wild-type does not include a TCA cycle. In those embodiments, expression in the recombinant microorganism of the heterologous NAD(P)H-producing oxidoreductase of the TCA cycle, is performed to activate the TCA cycle.

In some of those embodiments the recombinant microorganism, is a microorganism such as Clostridium acetobutylicum, C. tetani, C. perfringens, C. thermocellum, C. difficile, C. botulinum, C. beijerinckii and C. novyi. In some of those embodiments the recombinant microorganism, is a microorganism such as yeast wherein the TCA cycle is activated in a compartment of the cell (mitochondria) to the extent that an activated TCA cycle is desired in a different compartment of the cell (cytoplasm)

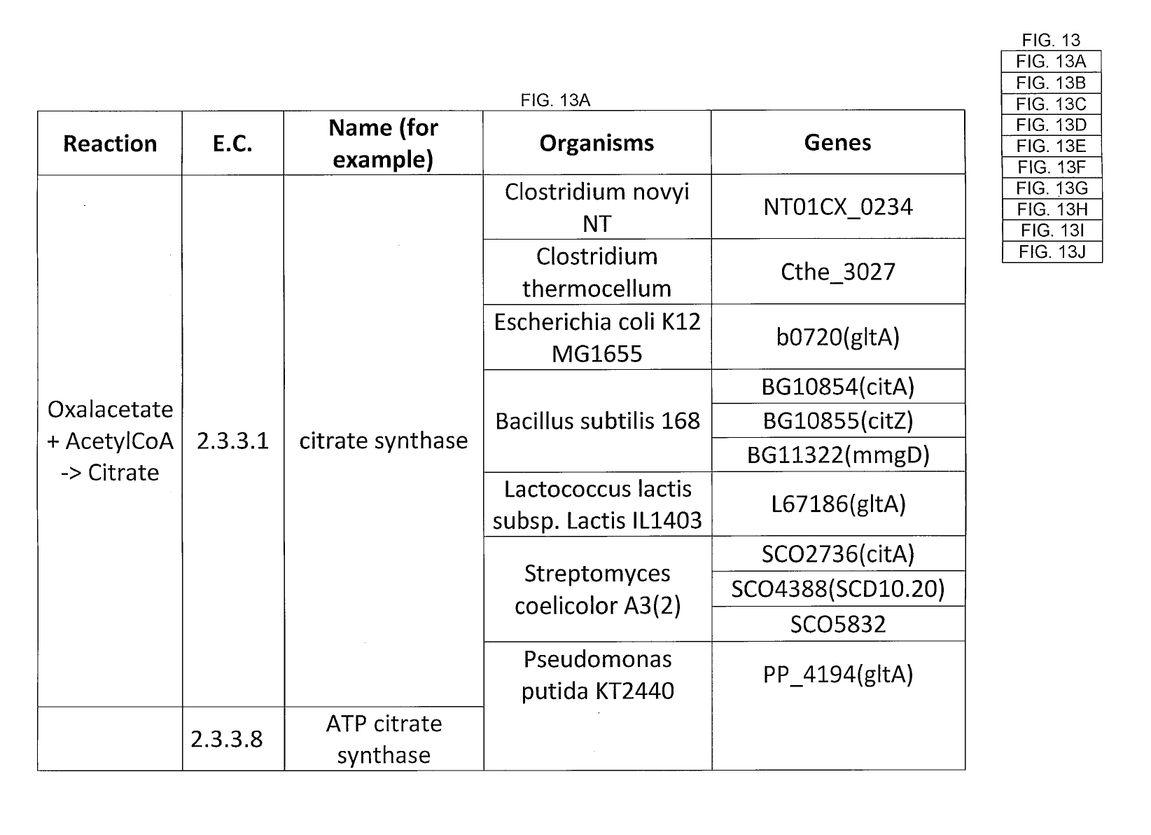

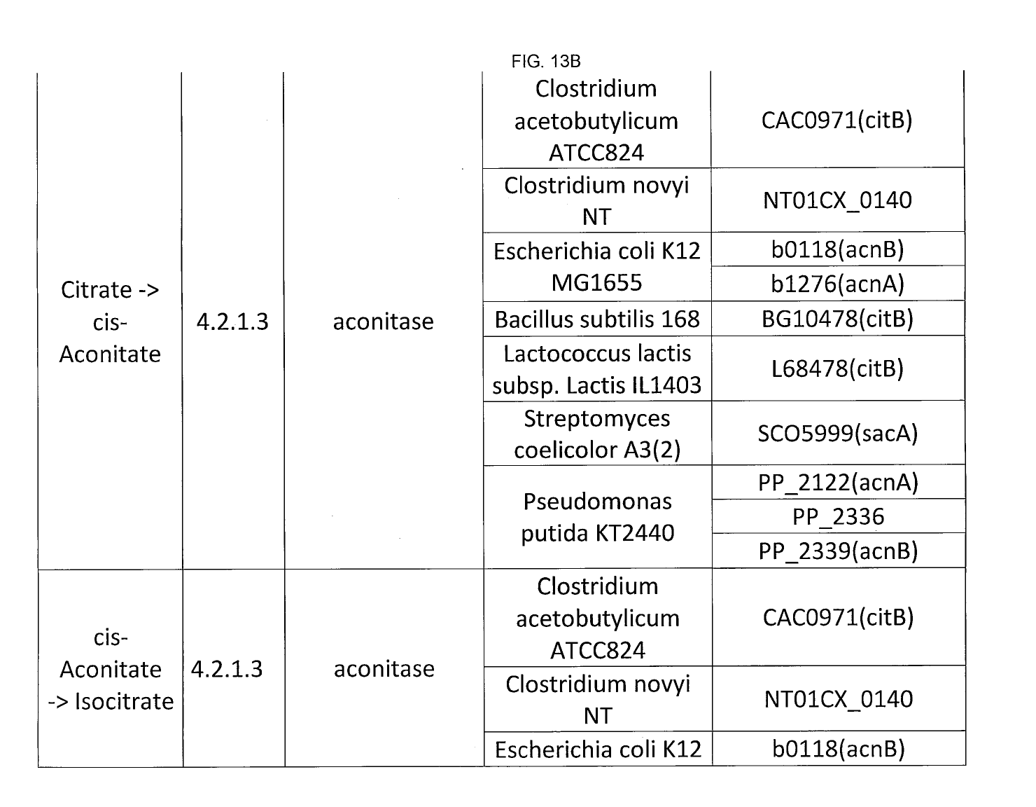

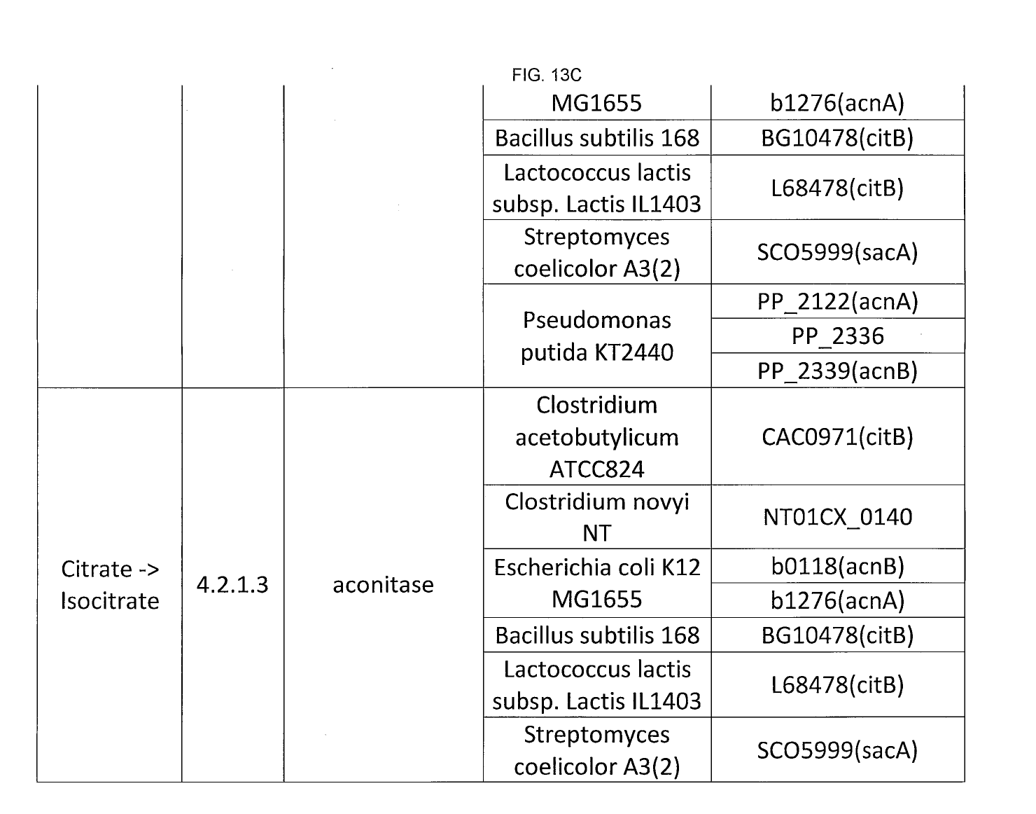

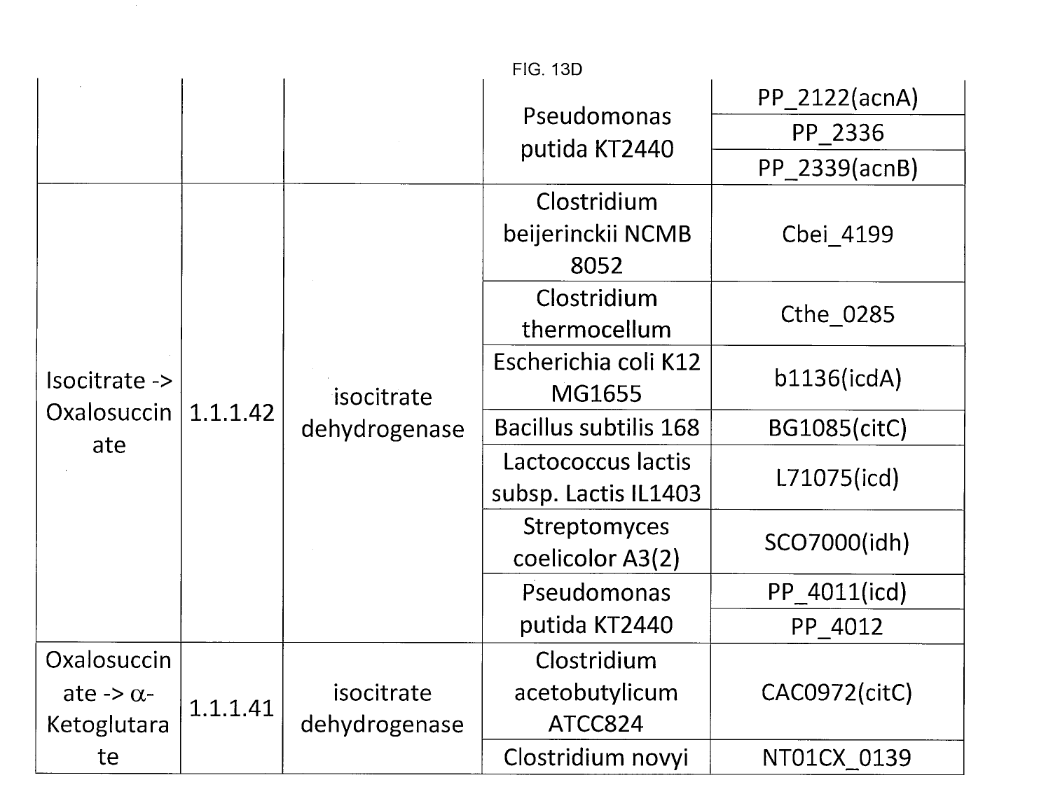

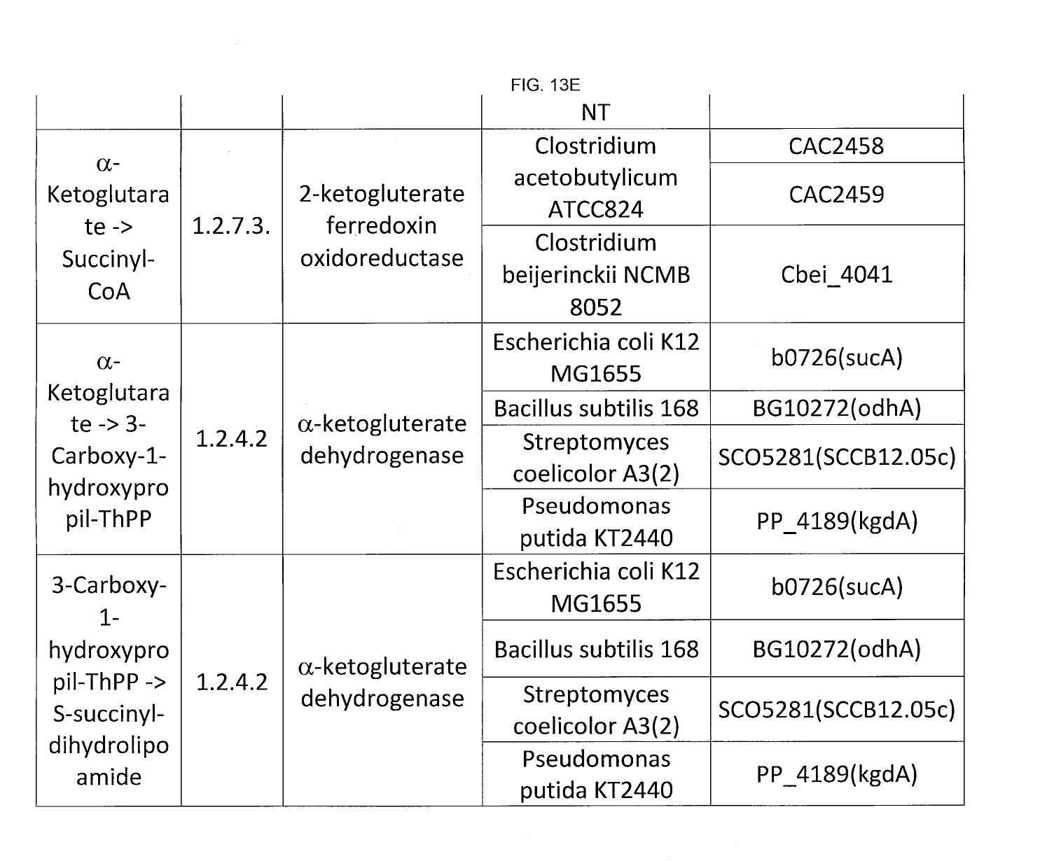

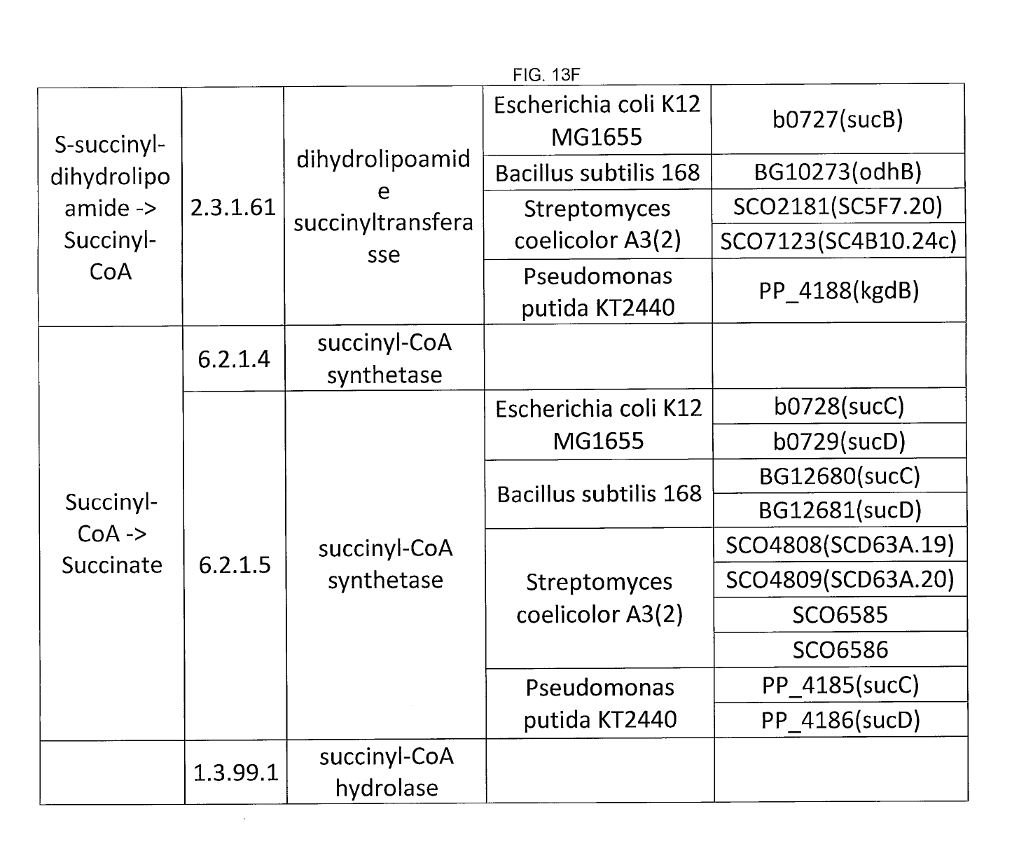

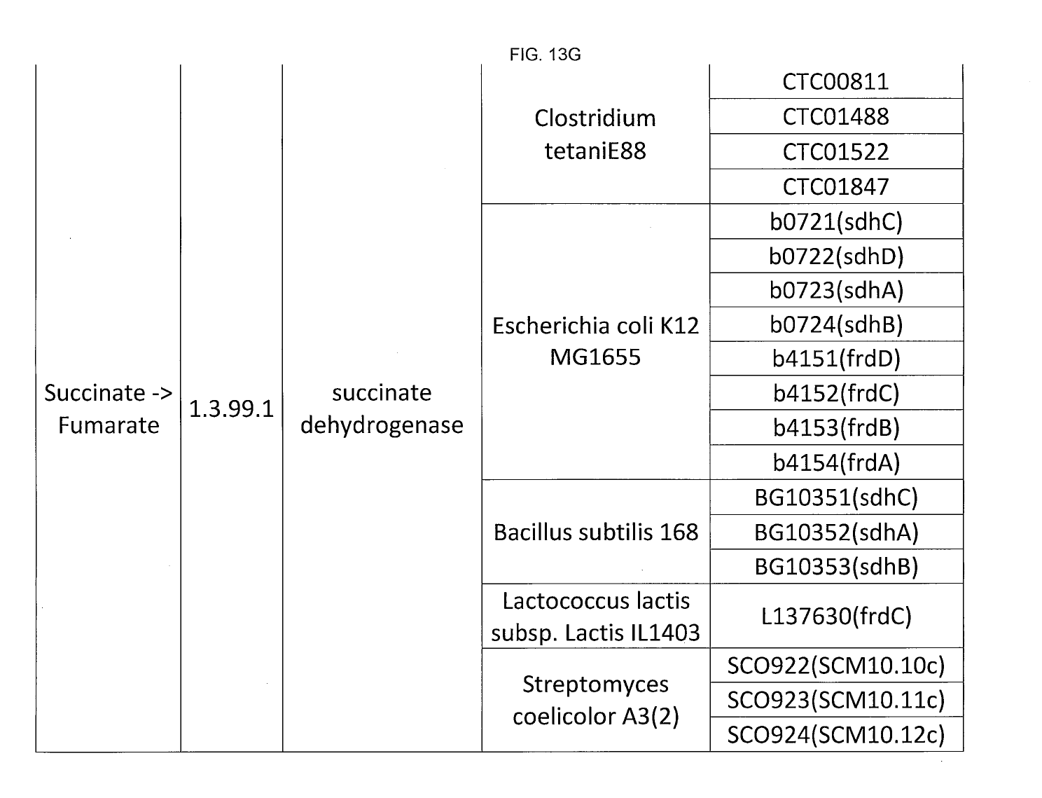

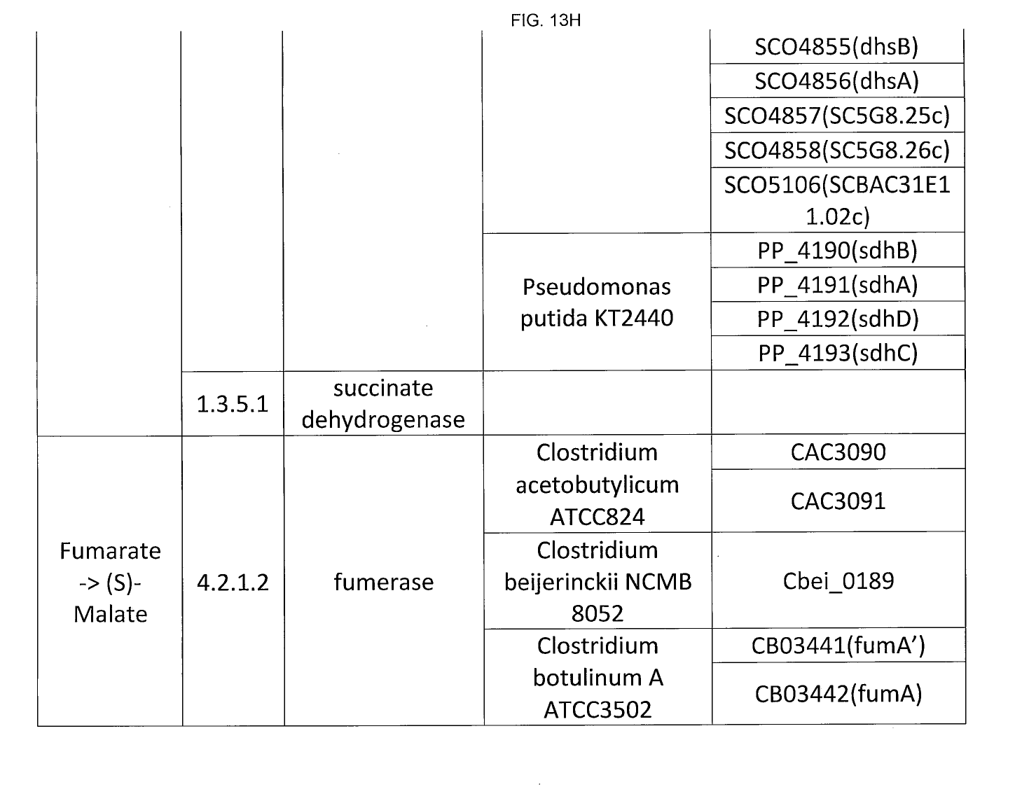

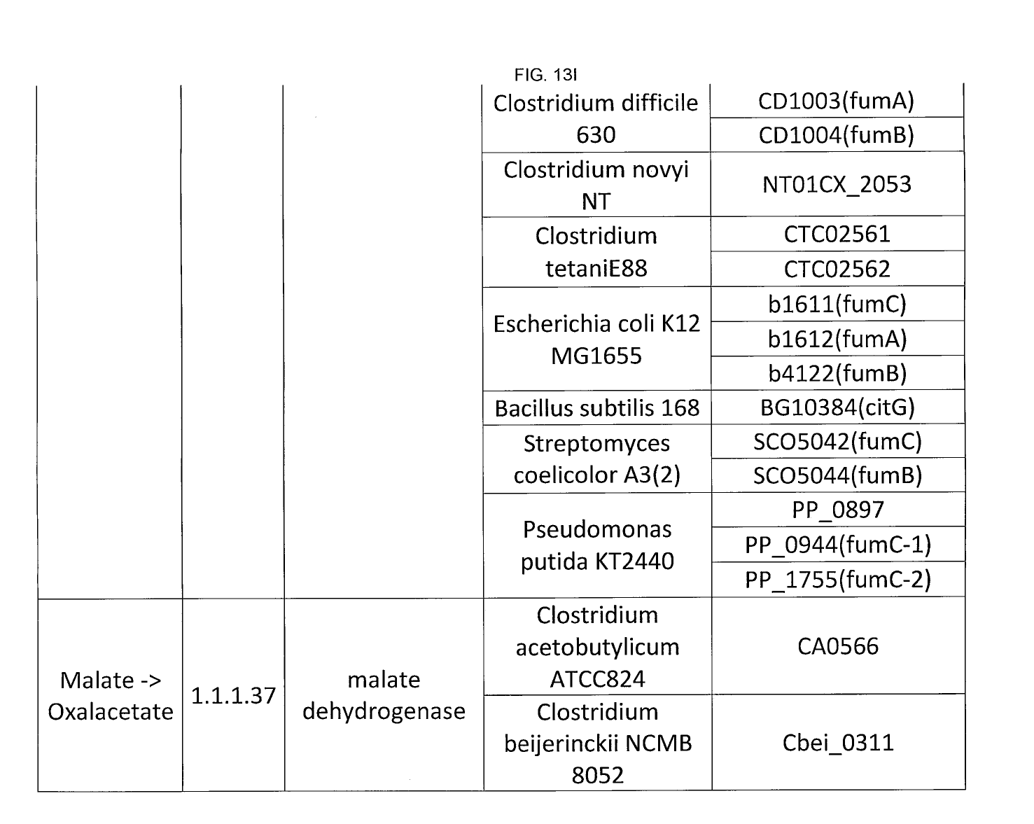

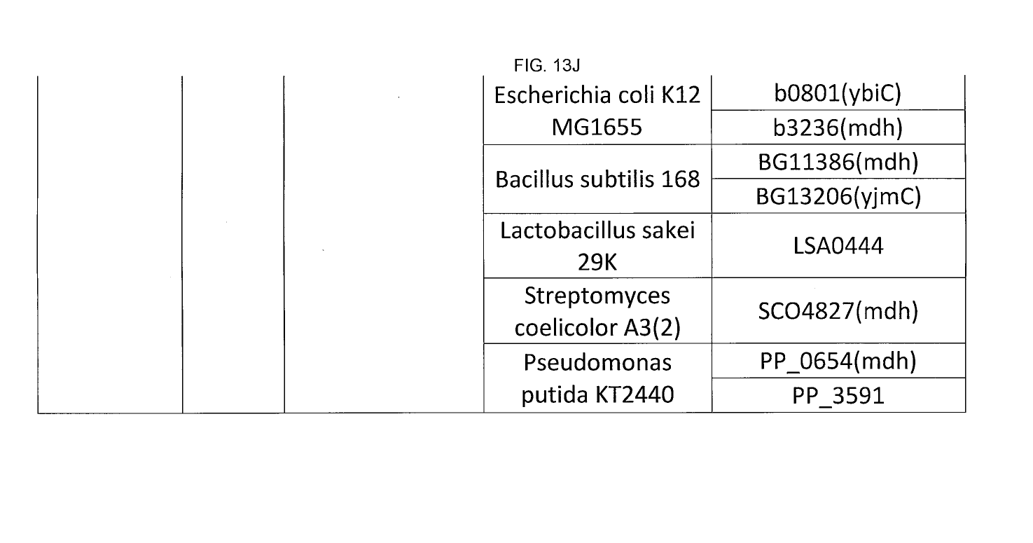

Reference is made to the schematic representation of the TCA cycle shown in FIG. 12 and to the related FIG. 13.

FIG. 13 shows the presence or absence of active enzymes controlling the reactions involved in the TCA cycle pathway as depicted in FIG. 12 for several microorganisms (Bacillus subtilis 168, Clostridium acetobutylicum ATCC 824, Clostridium beijerinckii NCIMB 8052, Clostridium botulinum A ATCC 3502, Clostridium difficile 630, Clostridium novyi NT, Clostridium perfringens 13, Clostridium perfringens SM101, Clostridium tetani E88, Clostridium thermocellum ATCC 27405, Escherichia coli K-12 MG1655, Lactococcus lactis subsp. Lactis IL1403, Lactobacillus sakei 23K, Streptomyces coelicolor A3(2), Pseudomonas putida KT2440) according to the KEGG database.

In particular, FIG. 13 shows TCA cycles and related enzymes in several organisms made through computational predictions based on sequence similarity to known proteins with the enzymatic activity of interest, performed according to KEGG (Kanehisa, M., et al.; From genomics to chemical genomics: new developments in KEGG. Nucleic Acids Res. 34, D354-357 (2006))

In view of the particular, once the enzymes that are expressed in a microorganism have been identified, the missing functionalities necessary to activate the TCA cycle can be introduced.

A person skilled in the art will be able to identify possible additional enzymatic activities of the TCA cycle in any of the microorganisms listed in FIG. 13 by identifying additional proteins, among the proteins not yet experimentally characterized in those organisms, that although not sequence homologs are functional homologs of an enzyme of the TCA cycle.

Once the enzymatic activities of the TCA cycle pathway already present in a specific microorganism have been identified using the computational approach and/or experimental characterization, it is possible to craft a strategy directed to introduce the missing functionality in the microorganism. In particular, the enzymatic activities that need to be introduced to complete an oxidative TCA cycle in a predetermined microorganism are initially identified. After that, a source of enzymes providing the missing functionality is identified, and finally the relevant functionality is introduced in the microorganism. In some embodiments, the deletion, inactivation or down-regulation of one or more native enzymes and/or pathways that interfere with the TCA cycle pathways can also be performed to activate the desired cycle.

In some embodiments, the recombinant microorganism is engineered to introduce the enzymatic functionalities necessary to activate a complete TCA cycle in the microorganism (see FIG. 12)

In some embodiments, a complete TCA cycle include the following set of enzymatically controlled reactions: conversion of oxalacetate acetyl-CoA into a molecule of citrate carried out by enzymatic activity EC 2.3.3.1; conversion of citrate to iso-citrate as a single step catalyzed by EC 4.2.1.3 or through the formation of cis-aconitate also catalyzed by EC 4.2.1.3; conversion of iso-citrate into α-ketoglutarate either directly by using EC 1.1.1.41 or through the formation of oxalosuccinate EC 1.1.1.42; conversion of α-ketoglutarate to succinyl-CoA either using EC 1.2.7.3 or through the formation of 3-carboxy-1-hydroxypropil-ThPP using EC 1.2.4.2 its conversion into S-Succinyl-dihydrolipoamide using EC 1.2.4.2 which in turn is converted into Succinyl-CoA using EC 2.3.1.61; conversion of succinyl-CoA into succinate through the used of EC 6.2.1.4, 6.2.1.5 or 3.1.2.3; conversion of succinate into fumarate through the use of EC 1.3.5.1 or EC 1.3.99.1; conversion of fumarate into malate by using EC 4.2.1.2; conversion of malate into oxalacetate by using EC 1.1.1.37.

In some embodiments, the recombinant microorganism is engineered to introduce enzymatic functionalities necessary to activate the glyoxylate cycle or glyoxylate shunt in the microorganism (see FIG. 12). Those embodiments have the advantage to bypass the requirement for isocitrate dehydrogenase and α-ketoglutarate dehydrogenase activities otherwise required (see FIG. 12)

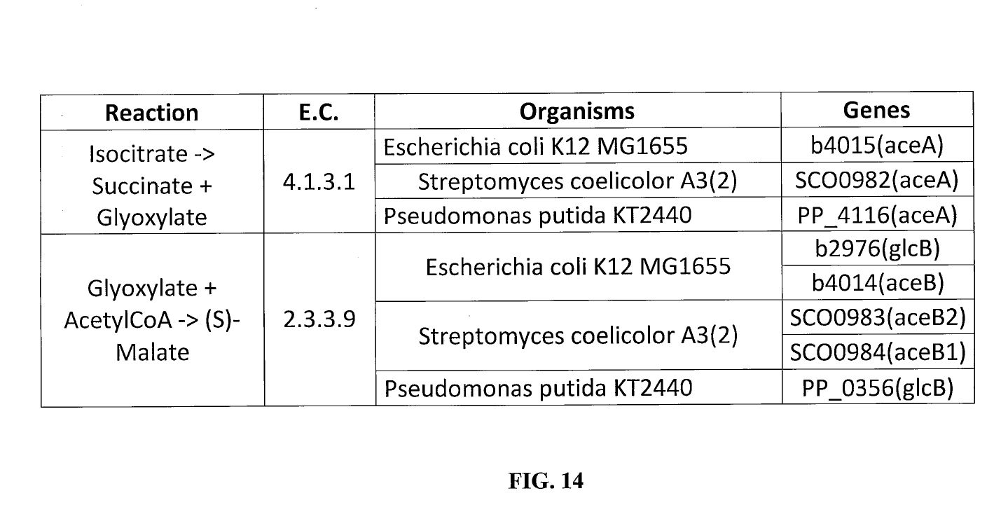

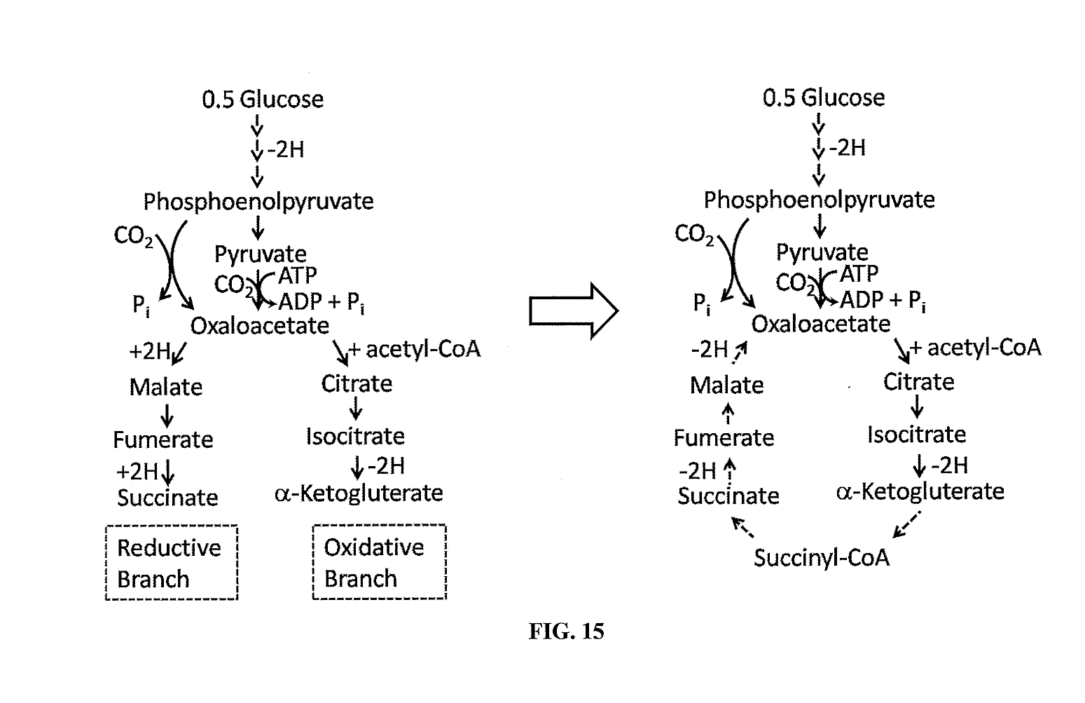

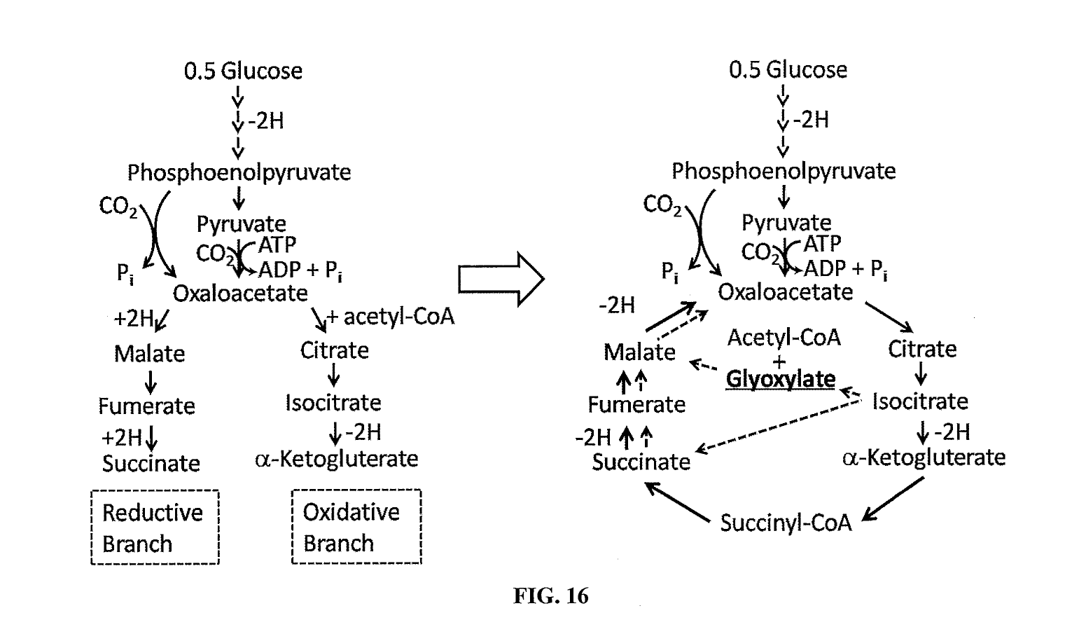

FIG. 14_illustrates the presence of the enzymatic activities of the glyoxylate cycle in the organisms identified in FIG. 13 according to KEGG (Kanehisa, M., et al.; From genomics to chemical genomics: new developments in KEGG. Nucleic Acids Res. 34, D354-357 (2006)). In particular, FIG. 14 shows a list of enzymatically controlled reactions and related enzymes that required for the production of isocitrate using oxalacetate and acetyl-CoA and for the reaction steps and enzymatic activities required for the conversion of succinate into oxalacetate for these organism are shown in FIG. 13

The enzymatic activities required for the activation of the TCA cycle or Glyoxylate shunt could be provided by introducing one or more enzymes that perform the activities in other organisms. In some embodiments, the enzymes introduced in the recombinant microorganisms are obtained from other organisms such E. coli, Streptomyces coelicolor or Pseudomonas putida and introduced into any of the recombinant microorganisms of FIG. 13 and FIG. 14.

In some embodiments, the recombinant microorganism to be engineered to introduce a TCA cycle or a glyoxylate shunt is a Clostridium, and, in particular, Clostridium acetobutylicum ATCC 824 or Clostridium Novyi NT, which have the advantage of requiring introduction of a lower number of enzymatic activities of the TCA cycle to activate the TCA cycle.

The introduction of heterologous enzymes and the possible deletion, inactivation or downregulation of native enzymes or pathway in the microorganism, can be performed by techniques identifiable by a skilled person and described, for example, in Mermelstein L D, Welker N E, Bennett G N, Papoutsakis E T., Expression of cloned homologous fermentative genes in Clostridium acetobutylicum ATCC 824. Biotechnology (NY). February; 10(2):190-5. (1992); Lee, S. Y., Bennett. G. N. and Papoutsakis, E. T., “Construction of E. coli-Clostridium acetobutylicum shuttle vectors and transformation of C. acetobutylicum strains”, Biotechnol. Lett. 14: 427-432 (1992); Lee, S. Y., Mermelstein, L. D., Bennett, G. N. and Papoutsakis, E. T., “Vector construction, transformation and gene amplification in Clostridium acetobutylicum ATCC 824”, Ann. N.Y. Acad. Sci. 665: 39-51 (1992); Mermelstein, L. D. and Papoutsakis, E. T., “In vivo methylation in Escherichia coli by the Bacillus subtilis phage φ3T I. Methyltransferase to protect plasmids from restriction upon transformation of Clostridium acetobutylicum ATCC 824”, Appl. Environ. Microbiol. 59: 1077-1081 (1993); Lee, S. Y., Mermelstein, L. D., Bennett, G N., and Papoutsakis, E. T. “Determination of plasmid copy number and stability in Clostridium acetobutylicum ATCC 824”, FEMS Microbiol. Lett. 108: 319-324 (1993); Papoutsakis, E. T. and Bennett, G. N., “Cloning, structure, and expression of acid and solvent pathway genes of Clostridium acetobutylicum”, Chapter 8 in: Clostridia and Biotechnology (Woods, D. R., ed.), pp. 157-199, Butterworth-Heinemann, Stoneham, Mass. (1993); Mermelstein, L. D., Bennett, G. N. and Papoutsakis, E. T., “Amplification of homologous fermentative genes in Clostridium acetobutylicum ATCC 824”, In: Bioproducts and Bioprocesses: Third Conference to Promote Japan/US Joint Projects and Cooperation in Biotechnology” (Tanner, R. D., ed.), pp. 317-343, Springer Verlag, New York (1993); Mermelstein, L. D., Papoutsakis, E. T., Petersen, D. J. and Bennett, G. N., “Metabolic engineering of Clostridium acetobutylicum for increased solvent production by enhancement of acetone formation enzyme activities using a synthetic acetone operon” Biotechnol. Bioeng. 42: 1053-1060 (1993); Mermelstein, L. D., and Papoutsakis, E. T., “Evaluation of macrolide and lincosamide antibiotics for plasmid maintenance in low Ph Clostridium acetobutylicum ATCC 824 fermentations”, FEMS Microbiol. Lett. 113: 71-75 (1993); Mermelstein, L. D., Welker, N. E., Petersen, D. J., Bennett, G N., and Papoutsakis, E. T, “Genetic and metabolic engineering of Clostridium acetobutylicum ATCC 824”, Ann. N.Y. Acad. Sci. 721: 54-68 (1994); Walter, K. A., Mermelstein, L. D. and Papoutsakis, E. T., “Host-plasmid interactions in recombinant strains of Clostridium acetobutylicum ATCC 824”, FEMS Microbiol. Lett., 123: 335-342 (1994); Desai, R. P. and Papoutsakis, E. T., “Antisense RNA strategies for the metabolic engineering of Clostridium acetobutylicum”, Appl. Environ. Microbiol. 65: 936-945 (1999); Tummala, S. B., Welker, N. E., and Papoutsakis, E. T., “Development and characterization of a gene-expression reporter system for Clostridium acetobutylicum ATCC 824”, Appl. Environ. Microbiol. 65: 3793-3799 (1999); L. M. Harris, N. E. Welker, and E. T. Papoutsakis “Northern, morphological and fermentation analysis of spo0A inactivation and overexpression in Clostridium acetobutylicum ATCC 824”, J. Bacteriol. 184: 3586-3597 (2002); Tummala, S. B., Welker, N. E., and Papoutsakis, E. T., “Design of antisense RNA constructs for the downregulation of the acetone formation pathway of Clostridium acetobutylicum,” J. Bacteriol., 185: 1923-1934 (2003)

In some embodiments, the introduction of enzymatic activities into clostridia is performed by the use of a suitable vector capable of either autonomous replication or homologous recombination with the chromosome. Introduction of this vector can be done through the use of electroporation techniques. The screening for clones with the desired enzymatic activity can be greatly facilitated by the inclusion of a selection marker (e.g. an antibiotic resistance) in the vector. Otherwise, the identification of a clone with the desired enzymatic activity can be carried out by performing a plasmid DNA extraction of each colony. Alternatively the presence of a clone carrying out the polypeptide of interest can also be detected through the use of a PCR reaction or southern blot of the DNA sequence encoding for the enzymatic activity, its mRNA by using Q-RT-PCR or Northern Blot, or its product (a polypeptide) by using Western Blot, an ELISA assay and/or using an enzyme activity assay. These methods could also prove that the polypeptide is transcribed (Q-RT-PCR and Northern Blot), its expressed (Western Blot or ELISA assay), and it is active in vitro (enzyme activity assay). Determination of the activity in vivo could be carried out by comparative analysis of the levels of the reaction products between the plasmid control strain (i.e. a strain transformed with a plasmid without the DNA encoding the polypeptide of interest) and the successful clone. The verification of the activity of the engineered TCA can be carried out by in vivo fluorimetry and or by the use of substrates labeled with ¹⁴C (radioactive) or ¹³C substrates and then analyzing the incorporation of the labeled carbon into the intermediates of the TCA.

In some embodiments, where the recombinant microorganism is yeast, the recombinant microorganism can be also engineered to introduce a TCA cycle, in the cytoplasm of the yeast. In those embodiments, the external NADH dehydrogenases, glycerol-3-phosphate dehydrogenases, as well as other competing enzymes that would oxidize NADH would be inactivated as described above. To engineer a TCA cycle in the cytoplasm of a yeast such as Saccharomyces cerevisiae, each gene that are required for the enzymes, citrate synthase EC 2.3.3.1, aconitase EC 4.2.1.3, isocitrate dehydrogenase EC 1.1.1.41, alpha-ketoglutarate dehydrogenase EC 1.2.4.2, succinyl CoA synthetase EC 6.2.1.4 or EC 6.2.1.5, succinate dehydrogenase EC 1.3.5.1 or EC 1.3.99.1, fumarase EC 4.2.1.2 and malate dehydrogenase EC 1.1.1.37, are cloned into an yeast expression plasmid. Multiple genes can be expressed off of a single plasmid using different promoters, such as the promoters for TEF2, TDH3, ENO2, and PGKI. Multiple plasmids can also be used with different auxotrophic markers (HIS3, TRP1, LEU2, or URA3) or antibiotic markers (kan, ble, bar, or hph). Furthermore, the sequences would be analyzed for an N-terminal mitochondrial localization signal peptide and any such sequence will be removed. Such prediction can be performed by prediction software on the web, such as MITOPROT (http://ihg.gsf.de/ihg/mitoprot.html). To make the NADH from this engineered TCA cycle to be available for a cytoplasmic biocatalyst, specifically in the yeast, S. cerevisiae, the following genes would be deletes: the external NADH dehydrogenases, NDE1 and NDE2, the soluble glycerol-3-phosphate dehydrogenases, GPD1 and GPD2, and the alcohol dehydrogenases ADH1, ADH2, ADH4, ADH5, and SFA1.

In some embodiments where the recombinant microorganism is yeast, the recombinant microorganism can be further engineered to increase the activity of a mitochondrial redox shuttle (FIG. 9) such as the ethanol-acetaldehyde shuttle so to increase the reducing equivalents available for use in the cytoplasm. In some of those embodiments, the activity of the redox-shuttle is increased by engineering the microorganism so that the expression of both the cytoplasmic alcohol dehydrogenase (and specifically Adh2) and the mitochondrial alcohol dehydrogenase (specifically Adh3) is increased (see FIG. 9).

It should be noted here that any combination of deletion or inactivation of the above enzymes results in viable cells. In particular, in any of those embodiments, cell growth is expected at various growth rates as expected in view of previous reports concerning inactivation and in particular deletions of some of those enzymes in microorganism such as E. coli (Calhoun, M. W. et al, 1993, J. Bacteriol., 175, 3013-25).

In any of the above mentioned embodiments, the recombinant microorganism is capable of supplying a heterologous oxidoreductase with more NAD(P)H when compared to the wild-type organism and, when the respiration is aerobic respiration, also supplies more O₂ when compared to the wild-type organism.

In particular, in some embodiments, the recombinant microorganism herein disclosed is expected to provide said heterologous oxidoreductase with up to 1.5-fold more NAD(P)H and possibly O₂ when compared to the wild-type organism.

In particular, in some embodiments, the recombinant microorganism herein disclosed is expected to provide said heterologous oxidoreductase with up to 2-fold more NAD(P)H and possibly O₂ when compared to the wild-type organism.

In particular, in some embodiments, the recombinant microorganism herein disclosed is expected to provide said heterologous oxidoreductase with up to 2.5 fold more NAD(P)H and possibly O₂ when compared to the wild-type organism.

In particular, in some embodiments, the recombinant microorganism herein disclosed is expected to provide said heterologous oxidoreductase with up to 3.0-fold more NAD(P)H and possibly O₂ when compared to the wild-type organism.

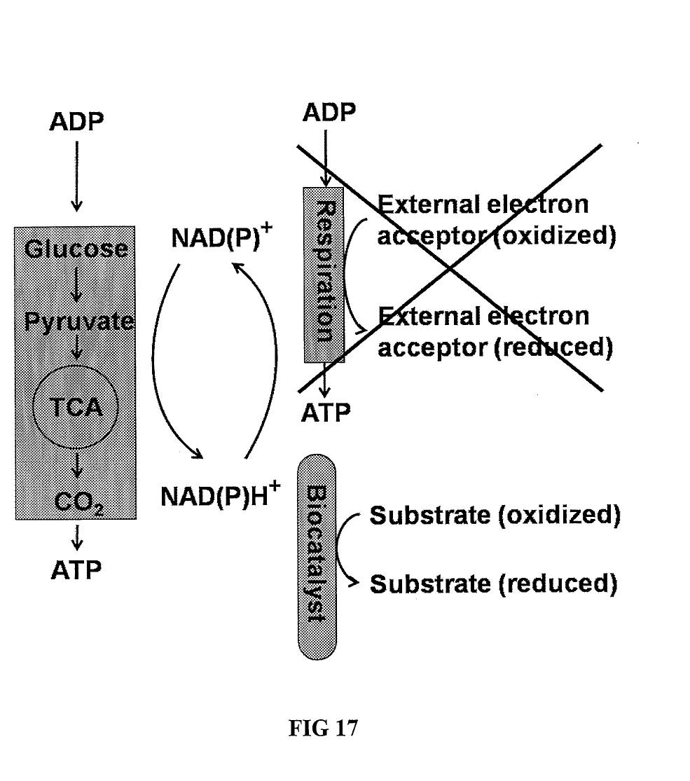

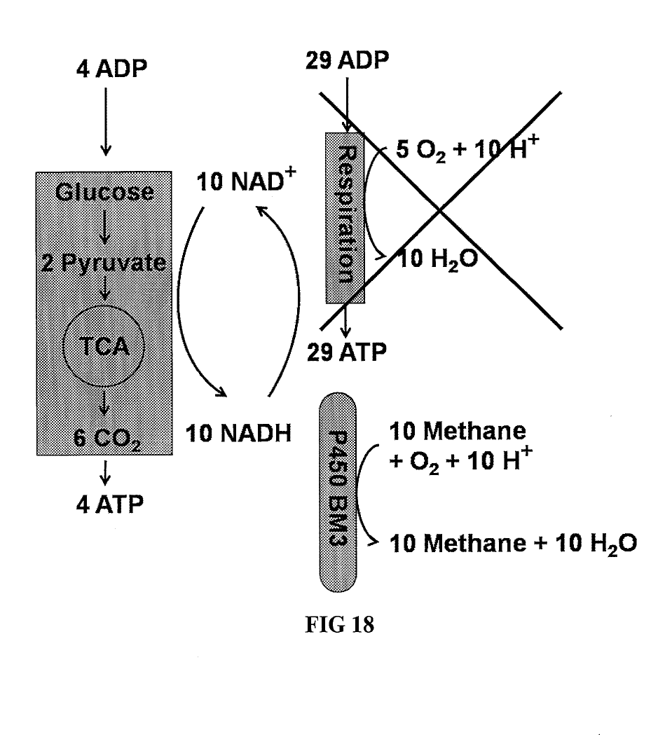

An exemplary biotransformation performed according to some embodiments of the present disclosure is schematically exemplified in FIG. 17 and FIG. 18. In particular, as shown in FIGS. 17 and 18, in the engineered microorganism herein disclosed, the metabolization of NAD(P)H by the respiratory pathway of the microorganism is replaced by a heterologous NAD(P)H-requiring oxidoreductase that uses the reducing equivalents in NAD(P)H to perform a biotransformation.

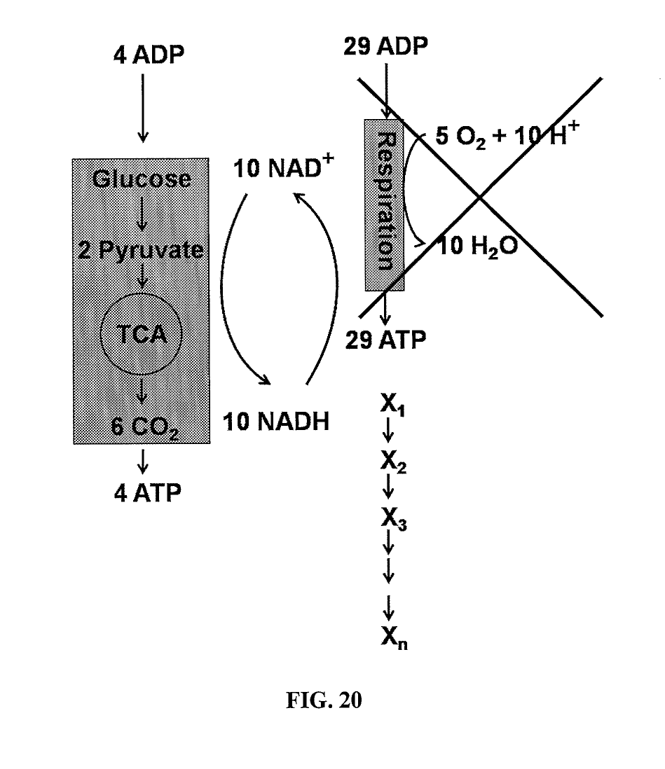

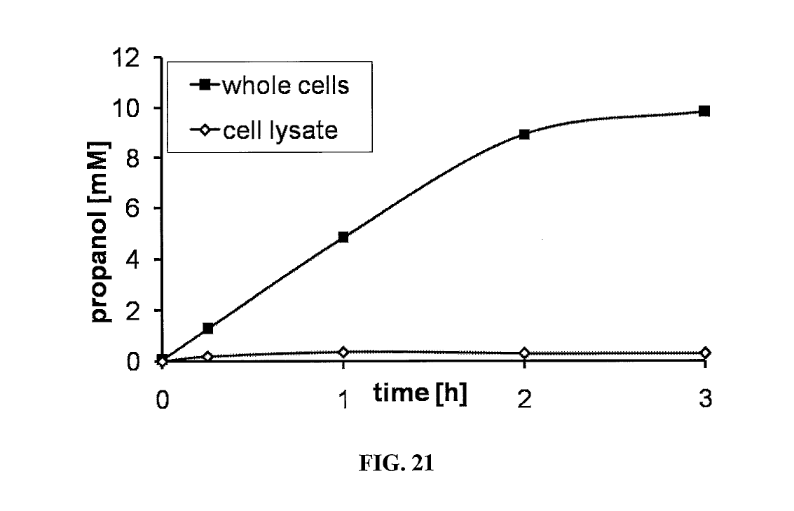

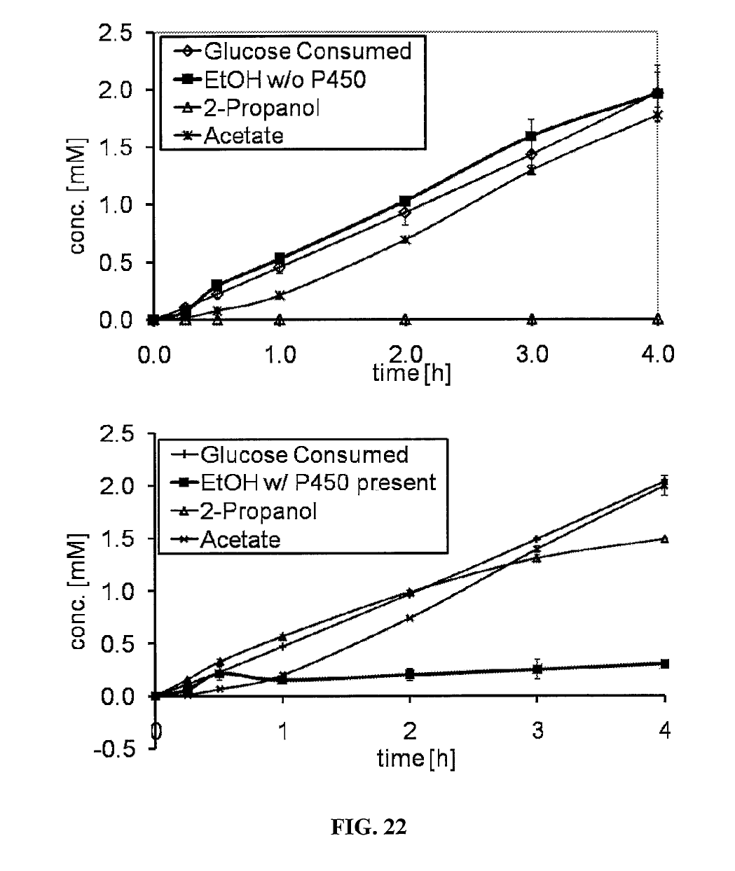

In some embodiments, the biotransformation is performed by a NAD(P)H-requiring oxidoreductase that catalyzes the direct conversion of the substrate into the product. An exemplary representation of those embodiments is illustrated in FIG. 18 schematically showing the NAD(P)H-requiring oxidoreductase cytochrome P450. In some embodiments, the biotransformation is performed by a heterologous pathway, wherein at least one of the reactions is catalyzed by the heterologous NAD(P)H-requiring oxidoreductase (FIG. 20).

In both cases, the net result of the respiratory pathway expected following the engineering of the microorganism is the generation of up to 10 molecules of reduced product or other product per molecule of glucose.

The heterologous NAD(P)H-requiring oxidoreductase, can be expressed in the recombinant microorganism using techniques identifiable by a skilled person upon reading of the present disclosure.

In some embodiments expression of the above mentioned enzyme is performed by using standard gene manipulation techniques. Standard recombinant DNA and molecular cloning techniques used here are well known in the art and are described by Sambrook, J., Fritsch, E. F. and Maniatis, T., Molecular Cloning: A Laboratory Manual, Second Edition, Cold Spring Harbor Laboratory Press, Cold Spring Harbor, N.Y. (1989) (hereinafter “Maniatis”); and by Silhavy, T. J., Bennan, M. L. and Enquist, L. W., Experiments with Gene Fusions, Cold Spring Harbor Laboratory Press, Cold Spring Harbor, N.Y. (1984); and by Ausubel, F. M. et al., Current Protocols in Molecular Biology, published by Greene Publishing Assoc. and Wiley-Interscience (1987).

Methods commonly used to introduce an endogenous or exogenous nucleic acid molecule into a host cell include but are not limited to, transformation, electroporation, conjugation, transduction, transfection and fusion of protoplasts. See, e.g., Ito et al., J. Bacteriol. 153:163-168 (1983); Durrens et al., Curr. Genet. 18:7-12 (1990); and Becker and Guarente, Methods in Enzymology 194:182-187 (1991); Maniatis; Silhavy, T. J., Bennan, M. L. and Enquist, L. W., Experiments with Gene Fusions, Cold Spring Harbor Laboratory Press, Cold Spring Harbor, N.Y. (1984); Ausubel, F. M. et al., Current Protocols in Molecular Biology, published by Greene Publishing Assoc. and Wiley-Interscience (1987); Current Protocols in Molecular Biology, Copyright © 2007 by John Wiley and Sons, Inc., Last updated: 24 Jul. 2007, online resource, found at: http://www.mrw.interscience.wiley.com/emrw/0471142727/home.

In an embodiment, the integration of a gene of interest into a DNA fragment or target gene occurs according to the principle of homologous recombination. This embodiment results in the gene being integrated into the host genome. According to this embodiment, an integration cassette containing a module comprising at least one selective marker gene and/or the gene to be integrated (internal module) is flanked on either side by DNA fragments that act as recognition sequences (sequences of DNA to which recombinase enzymes bind and catalyze breakage and/or rearrangement of the DNA sequence) for a site-specific recombinase, such as cre-lox recombinase of bacteriophage lambda, RED recombinase from bacteriophage lambda, or FLP recombinase of Saccharomyces, and is further flanked on each side by DNA fragments that are homologous to those of the ends of the targeted integration site (recombinogenic sequences). Herein, “site-specific recombination” refers to a nucleic acid crossover event, such as the integration of bacteriophage lambda DNA into host chromosomal DNA, that requires homology of only a very short region and uses an enzyme specific for that recombination, herein referred to as a “site-specific recombinase” enzyme. After transforming the host cell with the cassette by appropriate methods, a homologous recombination between the recombinogenic sequences may result in the internal module replacing the chromosomal region in between the two sites of the genome corresponding to the recombinogenic sequences of the integration cassette.

The exogenous or endogenous nucleic acid molecule contained within a host cell of the disclosure can be maintained within that cell in any form. For example, exogenous or endogenous nucleic acid molecules can be integrated into the genome of the cell or maintained in an episomal state, such as a plasmid, that can stably be passed on (“inherited”) to daughter cells. Such extra-chromosomal genetic elements (such as plasmids, etc.) can additionally contain selection markers that ensure the presence of such genetic elements in daughter cells. The “selectable marker gene”, “marker”, or “selectable marker” can be any gene used in a host to express a protein or polynucleotide, including but not limited to, an antibiotic resistance gene such as tetracycline, erythryomycin, ampicillin, chloramphenicol, kanamycin, spectinomycin, streptomycin, gentamycin, neomycin, ciprofloxacin, and/or a resistance gene for a toxic substance or compound, such as mercury, and/or a resistance gene for auxotrophy complementation, such as ScURA3 (for uracil) or an amino acid biosynthetic pathway gene. Moreover, the host cells can be stably or transiently transformed. In addition, the host cells described herein can contain a single copy, or multiple copies of a particular exogenous or endogenous nucleic acid molecule as described above.

Methods for expressing a polypeptide from an exogenous or endogenous nucleic acid molecule are well known to those skilled in the art. These methods may also be used to activate endogenous or native DNA sequences from a host. Such methods include, without limitation, constructing a nucleic acid such that a regulatory element promotes the expression of a nucleic acid sequence that encodes the desired polypeptide. Typically, regulatory elements are DNA sequences that regulate the expression of other DNA sequences at the level of transcription. Thus, regulatory elements include, without limitation, promoters, enhancers, and the like. For example, the exogenous or endogenous genes can be under the control of an inducible promoter or a constitutive promoter. Moreover, methods for expressing a polypeptide from an exogenous or endogenous nucleic acid molecule in bacteria or yeast are well known to those skilled in the art. For example, see references Maniatis; Silhavy, T. J., Bennan, M. L. and Enquist, L. W., Experiments with Gene Fusions, Cold Spring Harbor Laboratory Press, Cold Spring Harbor, N.Y. (1984); Ausubel, F. M. et al., Current Protocols in Molecular Biology, published by Greene Publishing Assoc. and Wiley-Interscience (1987); Current Protocols in Molecular Biology, Copyright © 2007 by John Wiley and Sons, Inc., Last updated: 24 Jul. 2007, online resource, found at: http://www.mrw.interscience.wiley.com/emrw/0471142727/home. For example, nucleic acid constructs that are capable of expressing exogenous or endogenous polypeptides within Kluyveromyces (see, e.g., U.S. Pat. Nos. 4,859,596 and 4,943,529, each of which is incorporated by reference herein in its entirety) and Saccharomyces (see, e.g., Gelissen et al., Gene 190(1):87-97 (1997)) are well known. In another embodiment, heterologous control elements can be used to activate or repress expression of endogenous or native genes. In yet another embodiment, endogenous or native control elements can be used to activate or repress expression of endogenous or native genes. Additionally, when expression is to be repressed or eliminated, the gene for the relevant enzyme, protein or RNA can be eliminated by known deletion techniques.

As described herein, hosts within the scope of the disclosure can be identified by selection techniques specific to the particular enzyme being expressed or over-expressed. Methods of identifying strains with the desired gene of interest are well known to those skilled in the art. Such methods include, without limitation, PCR and nucleic acid hybridization techniques such as Northern and Southern analysis, polyacrylamide gel electrophoresis, altered growth capabilities on a particular substrate or in the presence of a particular substrate, a chemical compound, a selection agent, labeling with a fluorescent tagging and the like. In some cases, immunohistochemistry and biochemical techniques can be used to determine if a cell contains a particular nucleic acid by detecting the expression of the encoded polypeptide. For example, an antibody having specificity for an encoded enzyme can be used to determine whether or not a particular host cell contains that encoded enzyme. Further, biochemical techniques can be used to determine if a cell contains a particular nucleic acid molecule encoding an enzymatic polypeptide by detecting a product produced as a result of the expression of the enzymatic polypeptide. For example, transforming a cell with a vector encoding a NADH dependent oxidoreductase and detecting reduction/oxidation of NAD+/NADH in the presence of a specific substrate indicates that the vector is both present and that the gene product is active. Methods for detecting specific enzymatic activities or the presence of particular products are well known to those skilled in the art. For example, the activity of a NADH dependent oxidoreductase can be determined as described by

As described here, the heterologous NAD(P)H-requiring oxidoreductase can be expressed in a host strain and its expression verified by techniques known to those skilled in the art. In particular, in some embodiments where the host strain is E. coli, the heterologous NAD(P)H-requiring oxidoreductase can be stably transformed in a high copy vector such as pUC18 together with a gene encoding ampicillin resistance(such as β-lactamase) as a selection marker. The vector can be of either high, low or medium copy number. The selection marker could include but is not limited to tetracycline, erythryomycin, ampicillin, chloramphenicol, kanamycin, and spectinomycin. The expression of the gene can be constitutive, or regulated by a promoter such as lac, tac, trp, ara or the like. In some embodiments where the host strain is Saccharomyces cerevisiae the heterologous NAD(P)H-requiring oxidoreductase can be stably transformed in a vector under a promoter such as AOX1 together with the expression of a selection marker gene such as ARG4

In some embodiments, the desired product of the biotransformation is an alcohol-based product. The term “alcohol product” or “alcohol-based product” refers to a chemical compound that, at a minimum, consists of the elements carbon (C), oxygen (O) and hydrogen (H). “Alcohol products” are of the general formula R—OH, wherein R may be any carbon-based backbone. There are essentially two types of alcohol products: (1) aromatic alcohol products, which contain at least one C—OH bond in an aromatic ring (R=aromatic ring) and (2) aliphatic alcohol products, which may be either saturated or unsaturated (R=aliphatic group). It should be noted that the carbon-based backbone, R, may additionally be substituted with a variety of elements and functional groups.

Accordingly, in particular in some embodiments, the heterologous NAD(P)H-requiring oxidoreductase is an enzyme capable of oxidizing a hydrocarbon substrate to an alcohol product or reducing a ketone substrate to an alcohol product. The product can be produced by a single substrate or a plurality of substrates which can be administered to the host in various forms such as solutions, mixtures and other materials which contain at least one substrate.

In particular, in some embodiments, the heterologous NAD(P)H-requiring oxidoreductase is an oxidase or a reductase, including but not limited to oxidases or reductases that carry out regioselective and stereoselective chemical transformations. More in particular, the heterologous NAD(P)H-requiring oxidoreductase can catalyze reactions such as hydroxylation, epoxidation, Baeyer-Villiger oxidation and ketone reduction. Accordingly, in some embodiments, the heterologous NAD(P)H-requiring oxidoreductase can be an enzyme of class EC 1.1.X.X., e.g. EC 1.1.1.1. alcohol dehydrogenase, EC 1.1.1.28 lactate dehydrogenase; enzyme class EC 1.4.X.X., for e.g. 1.4.1.9. leucine dehydrogenase; enzyme class 1.5.X.X., for e.g. 1.5.1.13. nicotinic acid hydroxylase; enzyme class EC 1.13.X.X., for e.g. 1.13.11.1. oxygenase, 1.13.11.11. naphthalene dioxygenase; enzyme class EC 1.14.X.X, for e.g. EC 1.14.12.10 benzoate dioxygenase, EC 1.14.13.X. monooxygenase, EC 1.14.13.16 cyclopentanone monooxygenase, EC 1.14.13.22 cyclohexanone monooxygenase, 1.14.13.44. oxygenase, EC 1.14.13.54 steroid monooxygenase, EC 1.14.14.1. monooxygenase.

In particular, in some embodiments, the NAD(P)H-requiring oxidoreductase can be a recombinantly expressed cytochrome P450. Cytochromes P450 are a large superfamily of heme proteins found in all domains of life that, as a whole, perform a diverse array of redox chemistries on an extremely wide variety of substrates. Most P450s are NADPH-dependent monooxygenases which introduce an oxygen atom from dioxygen into non activated carbon atoms to yield often optically pure products according to the reaction:

RH+O₂+NAD(P)H+H⁺→ROH+H₂O+NAD(P)⁺

Less common reactions catalyzed by these versatile enzymes include, but are not limited to, alkene epoxidation, amine and thiother oxidations, dealkylation of amines, ethers and thioethers, oxidative and reductive dehalogenations and dehydrogenations. P450s are used for the catabolic degradation of alkanes and aromatics in bacteria, drugs and xenobiotics in animals and herbicides (both natural and synthetic) in plants. Additionally, key steps in the biosynthesis of physiologically important compounds, such as steroids, fatty and bile acids, eicosanoids and fat-soluble vitamins, are catalyzed by P450s.

Cytochrome P450 BM3 from Bacillus megaterium is a fast, water soluble, single-component fatty acid hydroxylase readily expressed in laboratory strains of Escherichia coli, making it an ideal candidate for protein engineering. To extend the use of this fast and efficient enzyme for biotechnology applications, various groups have focused on engineering P450 BM3 to accept and hydroxylate a variety of substrates (Urlacher, V. et al, 2006, Curr. Opin. Chem. Biol., 10, 156-61). BM3 has provided an evolvable protein framework for obtaining modified or new activities. Rational design and directed evolution approaches have created BM3 variants with activity on medium-chain fatty acids (Li, Q. S. et al, 2001, Biochim. Biophys. Acta, 1545), selectivity for terminal (Meinhold, P. et al, 2006, Advanced Synthesis & Catalysis, 348, 763-72) and 2-hydroxylation of n-alkanes (Peters, M. W. et al, 2003, J. Am. Chem. Soc., 125, 13442-50), alpha-hydroxylation of organic acid derivatives (Landwehr, M. et al, 2006, J. Am. Chem. Soc., 128, 6058-59) activity on aromatic compounds (Appel, D. et al, 2001, J. Biotechnol., 88, 167-71; Li, Q. S. et al, 2001, Appl. Environ. Mirobiol., 67, 5735-39) and the ability to oxidize ethane to ethanol (Meinhold, P., 2005, Department of Biochemistry and Molecular Biophysics, 266). For example, P450 BM3 variant 4E10 (mutations A82L, A328V) efficiently converts propane to propanol.

Suitable substrates for biotransformation catalyzed by P450 include decanoic acid, styrene, myristic acid, lauric acid and other fatty acids and fatty acid-derivatives. Alkane/alkene-substrates, including, but not limited to, propane, propene, ethane, ethene, butane, butene, pentane, pentene, hexane, hexene, cyclohexane, octane, octene, p-nitrophenoxyoctane (8-pnpane) and various derivatives thereof, can also be used. The term “derivative” refers to the addition of one or more functional groups to a substrate, including, but not limited to, alcohols, amines, halogens, thiols, amides, carboxylates, etc.

The requirement for reduced cofactors (NADH or NADPH) in cytochrome P450 monooxygenase catalyzed reactions severely limits the practical use of this class of enzymes for large-scale conversions. Intact cells synthesize these cofactors and the reduced form can be regenerated from exogenously added carbohydrates such as glucose. Cells harboring an active cytochrome P450 monooxygenase can therefore be used as self-contained biocatalysts oxygenation reactions.

In some embodiments, the heterologous NAD(P)H-requiring oxidoreductase is a methane monooxygenases (MMO). Methane monooxygenases are the only enzymes naturally capable of efficiently catalyzing the oxidative conversion of methane to methanol according to the following reaction scheme:

CH₄+O₂+NADH+H⁺→CH₃OH+H₂O+NAD⁺

All methanotrophs can produce a membrane-bound, particulate form of MMO (pMMO), while only a subset of methanotrophs can produce a soluble form of MMO (sMMO which is produced under conditions of copper limitation (Lipscomb, J. D., 1994, Annu. Rev. Microbiol., 48, 371-99).

Soluble methane monooxygenase has been purified and is well characterized. Attempts to recombinantly express this enzyme in E. coli have failed so far. Evidence exists for expression in other heterologous hosts such as Pseudomonas sp. (Jahng, D. J. et al, 1994, Appl. Environ. Mirobiol., 60, 2473-82).

There are several, suitable sources of methane monooxygenases. In some embodiments, said sources include, but are not limited to Methylococcus, Methylosinus, Methylobacter, Methylomicrobium, Methylocystis, Methylomonas, Methylosinus or Methylocella. In a further embodiment, the methane monooxygenase is from Methylococcus capsulatus.

In embodiments where said microorganism, comprises a heterologous DNA sequence encoding a methane monooxygenase, substrates for the biocatalyst include, but are not limited to, alkanes. In a further embodiment, said alkane substrates are saturated or unsaturated alkanes having a carbon atom content limited to between about 1 and about 20 carbon atoms. In another embodiment, said alkane substrates have a carbon atom content limited to between about 1 and about 10 carbon atoms. In yet another embodiment, said alkane substrates have a carbon atom content limited to between about 11 and about 20 carbons. In yet another embodiment, said alkane substrates are methane, ethane and propane.

In some embodiments, the heterologous NAD(P)H-requiring oxidoreductase is a dioxygenase. Dioxygenases catalyze the regioselective and stereoselective insertion of two oxygen atoms from molecular oxygen into a substrate. One family of dioxygenases, the Rieske dioxygenases, are non-heme containing enzymes involved in the synthesis of key secondary metabolites such as flavonoids and alkaloids. These are multi-component systems that have together with the oxygenase component, an iron-sulfur flavoprotein reductase and iron-sulfur ferredoxin (Li, Z., J. B. van Beilen, et al. (2002)., Curr Opin Chem Biol 6(2): 136-44). Two of the well-characterized members of this family are the naphthalene and toluene dioxygenases. Quantitative conversion of naphthalene to cis-(1R,2S)-1,2-dihydro-1,2 dihydroxynaphthalene was achieved in recombinant E. coli carrying the naphthalene dioxygenase from Pseudomonas fluorescens (Urlacher, V. B. and R. D. Schmid (2006), Curr Opin Chem Biol 10(2): 156-61). While novel members of this family continue to be isolated, directed evolution and recombination strategies have helped isolate members with high activity and stable expression in heterologous hosts.

There are several, suitable dioxygenases useful in this disclosure. In some embodiments, said sources include, but are not limited to, benzene 1,2-dioxygenase, naphthalene 1,2-dioxygenase, toluene 2,3-dioxygenase and toluene 1,2-dioxygenase from Pseudomonas putida; biphenyl dioxygenase from Burkholderia cepacia; benzoate 1,2-dioxygenase from Acinetobacter sp. ADP1; phthalate 4,5-dioxygenase from Burkholderia cepacia; 4-chlorophenylacetate 3,4-dioxygenase from Pseudomonas sp. CBS; 4-sulfobenzoate 3,4-dioxygenase and terephthalate 1,2-dioxygenase from Comamonas Testosteroni T-2; dibenzofuran 4,4-dioxygenase dibenzothiophene 1,2-dioxygenase and ethylbenzene 2,3-dioxygenase.

In embodiments where such microorganism comprises a heterologous DNA sequence encoding a dioxygenase, substrates for the biocatalyst include, but are not limited to, benzene, naphthalene, toluene, xylenes, biphenyls, benzoates, phthalates, substituted benzenes and substituted benzoates.

In some embodiments, the heterologous NAD(P)H-requiring oxidoreductase is a styrene monooxygenases. Styrene monooxygenases catalyze the stereoselective epoxidation of styrene to styrene oxide. Styrene monooxygenases have been recombinantly expressed in E. coli (Panke, S. et al, 1998, Appl. Environ. Mirobiol., 64, 2032-43), permitting biocatalytic synthesis of enantiopure styrene oxide in whole-cells. To overcome the toxicity of styrene oxide, a two-liquid phase process for the production of enantiopure (S)-styrene oxide can be utilized (Panke, S. et al, 2000, Biotechnol. Bioeng., 69, 91-100), and this process has been scaled up to a 30 L scale (Panke, S. et al, 2002, Biotechnol. Bioeng., 80, 33-41). However, since the enzyme is cofactor dependent, an increased product yield per glucose would be desirable.

In some embodiments of this disclosure, the styrene monooxygenase is from Pseudomonas putida or Pseudomonas fluorescens.

In embodiments where such microorganism comprises a heterologous DNA sequence encoding a styrene monooxygenase, substrates for the biocatalyst include, but are not limited to, styrene.

In some embodiments, the heterologous NAD(P)H-requiring oxidoreductase is a Baeyer-Villiger monooxygenase. Baeyer-Villiger monooxygenases have been identified in a variety of bacteria and fungi (Stewart, J. D., 1998, Curr. Org. Chem., 2, 195-216), in which they play a vital role in catabolizing non-carbohydrate ketones, such as camphor(Ougham, H. J. et al, 1983, J. Bacteriol., 153, 140-52; Taylor, D. G. et al, 1986, J. Bacteriol., 165, 489-97) and cyclohexanone(Brzostowicz, P. C. et al, 2000, J. Bacteriol., 182, 4241-48; Donoghue, N. A. et al, 1976, Eur. J. Biochem., 63, 175-92) as sources of carbon and energy. Cyclohexanone monooxygenase, originally isolated from Acinetobacter sp. NCIB 9871(Donoghue, N. A. et al, 1976, Eur. J. Biochem., 63, 175-92) is a flavoprotein monooxygenase which converts cyclohexanone stereoselectively to ε-caprolactone in the presence of oxygen and NADPH (Ryerson, C. C. et al, 1982, Biochemistry, 21, 2644-55). The enzyme accepts a broad array of substrates and often exhibits high stereoselectivities in ketone oxidations, making it well-suited for synthetic applications (Stewart, J. D., 1998, Curr. Org. Chem., 2, 195-216). Early difficulties associated with enzyme supply were solved by overexpressing cyclohexanone monooxygenase in easily handled heterologous hosts such as Saccharomyces cerevisiae (Cheesman, M. J. et al, 2001, Protein Expr. Purif., 21, 81-86; Stewart, J. D. et al, 1996, Journal of the Chemical Society-Perkin Transactions 1, 755-57) and Escherichia coli (Chen, Y. C. J. et al, 1988, J. Bacteriol., 170, 781-89; Doig, S. D. et al, 2001, Enz. Microb. Technol., 28, 265-74; Mihovilovic, M. D. et al, 2001, J. Org. Chem., 66, 733-38). However, the NADPH-dependence of enzyme-mediated Baeyer-Villiger oxidations presents the major problem for preparative bioconversions.

There are several, suitable Baeyer-Villager monooxygenases useful for the purposes of the present disclosure. In some embodiments, said sources include, but are not limited to, cyclohexanone monooxygenase from Acinetobacter, cyclopentanone monooxygenase from Comamonas, cyclododecanone monooxygenase from Rhodococcus ruber and Rhodococcus rubber, steroid monooxygenase s from Cylindrocarbon radicola and Rhodococcus rhodochrous, 4-hydroxyacetophenome monooxygenase from Pseudomonas fluorescens and Pseudomonas putida. In a further embodiment, the Baeyer-Villager monooxygenase is from Acinetobacier.

In embodiments where such microorganism comprises a heterologous DNA sequence encoding a Baeyer-Villager monooxygenase, substrates for the biocatalyst include, but are not limited to, cyclic ketones such as cyclohexanone, cyclopentanone, cyclododecanone, 4-hydroxyacetophenone and progesterone. In embodiments where such Baeyer-Villager monooxygenase is a cyclohexanone monooxygenase, a possible substrate is cyclohexanone.

In some embodiments, the heterologous NAD(P)H-requiring oxidoreductase is a ketoreductase. The biocatalytic reduction of ketones can be utilized for the synthesis of chiral alcohols from a broad range of ketone, ketoacid and ketoester substrates. They also catalyze the reduction of a number of aldehydes. Ketoreductases are cofactor dependent and commonly used in vitro using a cofactor regeneration system (Kaluzna, I. A. et al, 2005, Tetrahedron: Asymmetry, 16, 3682-89). In some embodiments of this disclosure, the ketoreductase is Gre2p, an NADPH-dependent short-chain dehydrogenase from Saccharomyces cerevisiae, that reduces a variety of ketones with high stereoselectivity.

Many heterogeneously-expressed biocatalysts may not be initially optimized for use as a metabolic enzyme inside a host microorganism. However, these enzymes can usually be improved using directed evolution to enhance activity or expression levels in a given host. Alternatively, enzymes can usually be improved by codon optimization through modifying the coding sequence of a given enzyme to enhance expression in a given host. In other words, even if the activity of a biocatalyst enzyme or pathway is low initially, it is possible to improve upon this pathway. Codons can be substituted to reflect the preferred codon usage of the host, a process sometimes called “codon optimization” or “controlling for species codon bias.” Optimized coding sequences containing codons preferred by a particular prokaryotic or eukaryotic host can be prepared (see also Murray et al. (1989) Nucl. Acids Res. 17:477-508).

In some embodiments, the heterologous NAD(P)H-requiring oxidoreductase is an enzyme of an NAD(P)H-requiring heterologous pathway and, in particular, a heterologous pathway resulting in the production of an alcohol.

The wording “NAD(P)H-requiring pathway” as used herein refers to a pathway wherein the conversion from the substrate to the product requires reducing equivalents directly or indirectly provided by NAD(P)H at some catalytic step within said pathway or by some or one enzyme or biologically active molecule within said pathway.

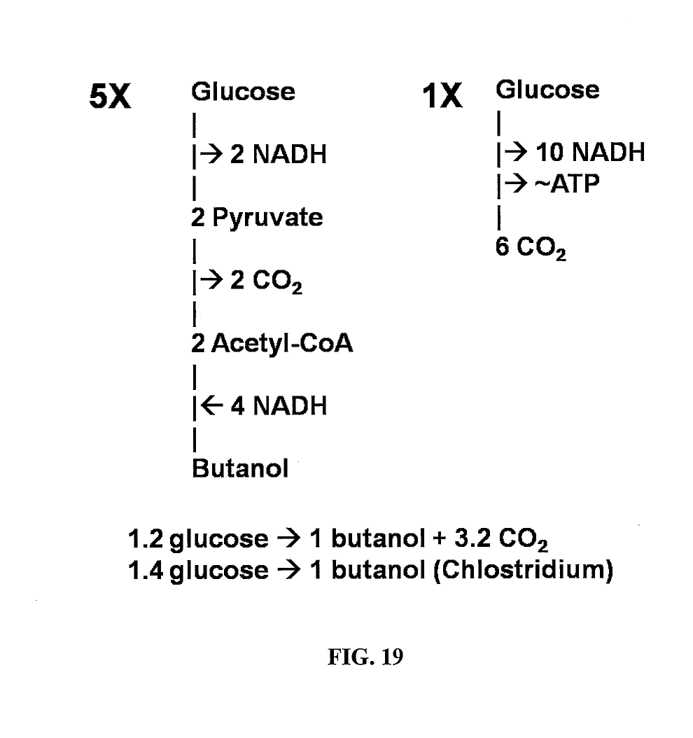

In some embodiments, the heterologous NAD(P)H-requiring pathway is a pathway wherein reducing equivalents from at least two NAD(P)H molecules are required for the conversion of the substrate to a product. An exemplary NAD(P)H-requiring heterologous pathway is the pathway for the production of Butanol schematically illustrated in FIG. 19, wherein the pathway is shown in comparison with glycolysis in a representation illustrating the stoichiometry of the reactions.

As illustrated in FIG. 19, for the production of butanol from glucose, four NAD(P)H molecules are required and thus four NAD(P)H molecules need to be derived from glucose to form this product. In a wild-type microorganism herein disclosed, the conversion of glucose to acetyl-CoA via pyruvate yields two molecules of NAD(P)H under anaerobic conditions. Therefore, two additional molecules of NAD(P)H are required to form butanol under anaerobic conditions.

In the recombinant microorganism herein disclosed, the additional NAD(P)H molecules can be made available by one or more of the inactivation of NAD(P)H-requiring native oxidoreductase and/or by the activation, replacement or introduction of one or more of the NAD(P)H producing oxidoreductase herein disclosed.

Other exemplary heterologous pathways that convert glucose to an end product that require more than two NAD(P)H molecules can include the production of chain alcohols longer than butanol. These alcohols can be biosynthesized using enzymes activities commonly found in fatty acid biosynthetic pathways and are listed in Table 4:

TABLE 4

| | Reaction Name | Substrates | | Products |
|---|---|---|---|---|
| 1 | acetyl transacylase | Acetyl-CoA + ACP | -> | Acetyl-ACP + CoA |
| 2 | Acetyl-CoA | Acetyl-CoA + CO₂ + ATP | -> | Malonyl-CoA |
| | carboxylase | | | |
| 3 | malonyl transacylase | ACP + malonyl-CoA | -> | CoA + malonyl-ACP |
| 4 | Fatty acid synthesis | Acetyl-ACP + malonyl-ACP + 2 | -> | ACP + butyryl-ACP + |
| | (C4) | NADPH | | CO2 |
| 5 | Fatty acid synthesis | Butyryl-ACP + malonyl-ACP + 2 | -> | ACP + CO2 + |
| | (C6) | NADPH | | hexonyl-ACP |
| 6 | Fatty acid synthesis | Hexonyl-ACP + malonyl-ACP + 2 | -> | ACP + CO2 + |
| | (C8) | NADPH | | octanyl-ACP |
| 7 | fatty acid synthesis | Malonyl-ACP + 2 NADPH + octanyl- | -> | ACP + CO2 + |
| | (C10) | ACP | | decanyl-ACP |
| 8 | decanyl transacylase | CoA + decanyl-ACP | -> | ACP + decanyl-CoA |
| 9 | decanylCoA reductase | decanylCoA + NADPH | -> | CoA + decanaldehyde |
| 10 | decanaldehyde | decanaldehyde + NADPH | -> | Decanol |
| | reductase | | | |

In some embodiments, where the recombinant microorganism herein disclosed has at least one of the NAD(P)H-requiring oxidoreductase such as NADH dehydrogenase, NDH-1 dehydrogenase, NDH-2 dehydrogenase, a quinone molecule such as ubiquinone and menaquinone, a quinol oxidase complex including a bo-type and/or a bd-type quinol oxidase complexes, a quinol:cytochrome c oxidoreductase, a cytochrome oxidase, and a terminal reductase or terminal reductase pathways, the stoichiometry for the production of alcohols of various chain lengths according to the above outlined pathway is illustrated in the following Table 5.

TABLE 5

| Chain | | | | | | | |
|---|---|---|---|---|---|---|---|
| length | Glucose | -> | ATP | CO2 | product | yield (g/g) | NADH used |
| 4 | 1 | -> | 2.00 | 2 | 1.00 | 0.41 | 4 |
| 6 | 1 | -> | 0.67 | 2 | 0.67 | 0.38 | 4 |
| 8 | 1 | -> | 0.50 | 2 | 0.50 | 0.36 | 4 |
| 10 | 1 | -> | 0.40 | 2 | 0.40 | 0.35 | 4 |
| 12 | 1 | -> | 0.33 | 2 | 0.33 | 0.34 | 4 |
| 14 | 1 | -> | 0.29 | 2 | 0.29 | 0.34 | 4 |
| 16 | 1 | -> | 0.25 | 2 | 0.25 | 0.34 | 4 |
| 18 | 1 | -> | 0.22 | 2 | 0.22 | 0.33 | 4 |
| 20 | 1 | -> | 0.20 | 2 | 0.20 | 0.33 | 4 |
| 22 | 1 | -> | 0.18 | 2 | 0.18 | 0.33 | 4 |
| 24 | 1 | -> | 0.17 | 2 | 0.17 | 0.33 | 4 |
| 26 | 1 | -> | 0.15 | 2 | 0.15 | 0.33 | 4 |
| 28 | 1 | -> | 0.14 | 2 | 0.14 | 0.33 | 4 |
| 30 | 1 | -> | 0.13 | 2 | 0.13 | 0.32 | 4 |

In some embodiments, the recombinant microorganism herein disclosed is engineered to express an heterologous NAD(P)H-producing oxidoreductase of the TCA cycle. In those embodiments, the production of the molecules listed in Table 4 is expected according to the stoichiometry as illustrated in the following Table 6:

TABLE 6

| | | | | | | | yield | NADH |
|---|---|---|---|---|---|---|---|---|
| C | M (g/mol) | Glucose | -> | ATP | CO2 | product | (g/g) | used |
| 4 | 74 | 1 | -> | 1.50 | 2.67 | 0.83 | 0.34 | 3.33 |
| 6 | 102 | 1 | -> | 1.22 | 2.67 | 0.56 | 0.32 | 3.33 |
| 8 | 130 | 1 | -> | 1.08 | 2.67 | 0.42 | 0.30 | 3.33 |
| 10 | 158 | 1 | -> | 1.00 | 2.67 | 0.33 | 0.29 | 3.33 |
| 12 | 186 | 1 | -> | | 2.67 | 0.28 | 0.29 | 3.33 |
| 14 | 214 | 1 | -> | | 2.67 | 0.24 | 0.28 | 3.33 |
| 16 | 242 | 1 | -> | | 2.67 | 0.21 | 0.28 | 3.33 |
| 18 | 270 | 1 | -> | | 2.67 | 0.19 | 0.28 | 3.33 |
| 20 | 299 | 1 | -> | | 2.67 | 0.17 | 0.28 | 3.33 |
| 22 | 327 | 1 | -> | | 2.67 | 0.15 | 0.27 | 3.33 |
| 24 | 355 | 1 | -> | | 2.67 | 0.14 | 0.27 | 3.33 |
| 26 | 383 | 1 | -> | | 2.67 | 0.13 | 0.27 | 3.33 |
| 28 | 411 | 1 | -> | | 2.67 | 0.12 | 0.27 | 3.33 |
| 30 | 439 | 1 | -> | | 2.67 | 0.11 | 0.27 | 3.33 |

In some embodiments, a heterologous NAD(P)H-requiring pathway is comprised of one or more oxidoreductase enzymes, including but not limited to oxidases or reductases that carry out regioselective and stereoselective chemical transformations. More in particular, the heterologous NAD(P)H-requiring oxidoreductase can catalyze reactions such as hydroxylation, epoxidation, Baeyer-Villiger oxidation and ketone reduction. Accordingly, in some embodiments, the heterologous NAD(P)H-requiring oxidoreductase can be an enzyme of class EC 1.1.X.X., e.g. EC 1.1.1.1. alcohol dehydrogenase, EC 1.1.1.28 lactate dehydrogenase; enzyme class EC 1.4.X.X., for e.g. 1.4.1.9. leucine dehydrogenase; enzyme class 1.5.X.X., for e.g. 1.5.1.13. nicotinic acid hydroxylase; enzyme class EC 1.13.X.X., for e.g. 1.13.11.1. oxygenase, 1.13.1.1.11. naphthalene dioxygenase; enzyme class EC 1.14.X.X, for e.g. EC 1.14.12.10 benzoate dioxygenase, EC 1.14.13.X. monooxygenase, EC 1.14.13.16 cyclopentanone monooxygenase, EC 1.14.13.22 cyclohexanone monooxygenase, 1.14.13.44. oxygenase, EC 1.14.13.54 steroid monooxygenase, EC 1.14.14.1. monooxygenase.

In another embodiment, a heterologous NAD(P)H-requiring pathway is comprised of one or more oxidoreductase enzymes, including but not limited to oxidases or reductases, including but not limited to the ones listed above, and one or more enzymes that do not require NAD(P)H as a cofactor.

In yet another embodiment, the preferred substrate is an alkane which is regioselectively or enantioselectively converted to an alcohol by an alkane monooxygenase.

Microorganisms, in general as herein described, are suitable as hosts for the production of any of the above products if they possess inherent properties, for example solvent resistance, that will allow them to function when the product is produced in less than ideal environments.

The terms “host cells” and “recombinant host cells” are used interchangeably herein and refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

Useful hosts for producing oxidized or reduced products may be either eukaryotic or prokaryotic microorganisms.

While in some embodiments E. coli is the usual host, in other embodiments hosts include aerobes such as Pseudomonas strains, which can metabolize other carbon sources such as petroleum and which can be tolerant to substrates/products that are toxic to E. coli, or are able to import or export substrates and products naturally. Still, in other embodiments, hosts include anaerobes, such as Bacillus subtilis or Shewanella oneidensis, which can metabolize other carbon sources such as carbohydrates or aromatic compounds and which can be tolerant to substrates/products that are toxic to E. coli, or are able to import or export substrates and products naturally. Therefore, in some embodiments said hosts include, but are not limited to, Saccharomyces, Pichia, Hanensula, Yarrowia, Aspergillus and Candida species. In some embodiments, the host can be Aspergillus, or Penicillium or Kluyveromyces.

In some embodiments, said hosts are bacterial hosts. In another embodiment said hosts include Arthrobacter, Bacillus, Brevibacterium, Clostridium, Corynebacterium, Escherichia, Gluconobacter, Lactobacillus, Nocardia, Pseudomonas, Rhodococcus, Saccharomyces, Shewanella, Streptomyces, Xanthomonas, Zymomonas. In another embodiment, such hosts are E. coli or Pseudomonas. In another embodiment, such hosts are E. coli W3110, E. coli B, Pseudomonas oleovorans, Pseudomonas fluorescens, or Pseudomonas putida.

In some embodiments, the hosts is engineered, where possible to inactivate all relevant/present NAD(P)H-requiring oxidoreductases that are not otherwise required for the desired biocatalytic reaction.

The host recombinant microorganism herein disclosed can use carbon sources as substrates for the biotransformation and/or metabolic reactions in the microorganism.

The term “carbon source” generally refers to a substance suitable to be used as a source of carbon for prokaryotic or simple eukaryotic cell growth. Carbon sources include, but are not limited to, biomass hydrolysates, starch, cellulose, hemicellulose, xylose, and lignin. Carbon sources can comprise various organic compounds in various forms, including, but not limited to polymers, carbohydrates, acids, alcohols, aldehydes, ketones, amino acids, peptides, etc. These include, for example, various monosaccharides such as glucose, dextrose, maltose, oligosaccharides, polysaccharides, saturated or unsaturated fatty acids, succinate, lactate, acetate, ethanol, etc., or mixtures thereof. Photosynthetic organisms can additionally produce a carbon source as a product of photosynthesis. The term “carbon source” may be used interchangeably with the term “energy source” since in chemoorganotrophic metabolism the carbon source is used both as an electron donor during catabolism as well as a source of carbon during cell growth. In some embodiments, carbon sources may be selected from biomass hydrolysates and glucose.

The term “biomass” as used herein refers primarily to the stems and leaves of green plants, and is mainly comprised of lignin, cellulose and hemicellulose. The term “lignin” as used herein refers to a polymer material, mainly composed of linked phenolic monomeric compounds, such as p-coumaryl alcohol, coniferyl alcohol, and sinapyl alcohol, that forms the basis of structural rigidity in plants and is frequently referred to as the woody portion of plants. Lignin is also considered to be the noncarbohydrate portion of the cell wall of plants. The term “cellulose” as used herein refers is a long-chain polymer polysaccharide carbohydrate, of beta-glucose of formula (C₆H₁₀O₅)ₙ. usually found in plant cell walls in combination with lignin and any hemicellulose. The term “hemicellulose” refers to a class of plant cell-wall polysaccharides that can be any of several heteropolymers. These include xylane, xyloglucan, arabinoxylan, arabinogalactan, glucuronoxylan, glucomannan and galactomannan. This class of polysaccharides is found in almost all cell walls along with cellulose. Hemicellulose is lower in weight than cellulose and cannot be extracted by hot water or chelating agents, but can be extracted by aqueous alkali. Polymeric chains bind pectin and cellulose, forming a network of cross-linked fibers.

Biomass can be decomposed by either chemical or enzymatic treatment to the monomeric sugars and phenols of which it is composed (Wyman, C. E., 2003, Biotechnol. Prog., 19, 254-62). This resulting material, called biomass hydrolysate, is neutralized and treated to remove trace amounts of organic material that may adversely affect the host cell, and is then used as a carbon source for the biotransformations.

The monosaccharide glucose is the basic unit of carbon energy in most metabolisms. Glucose is metabolized via glycolysis to acetyl-CoA, which is the precursor to all carbon metabolites in both aerobic and anaerobic metabolism. Glucose is a six carbon sugar and is fed to the cells during biotransformations, according to this disclosure, typically in concentrations of 1-50 mM glucose, or approximately 6-300 mM per carbon. Other monosaccharide aldo- and keto-sugars, e.g. the six carbon sugars galactose and mannose and the five carbon sugars xylose and arabinose, can also be used as carbon sources for the biotransformations using the engineered cells described in this disclosure. All of these sugars are metabolized by the cell via their conversion into compounds, such as fructose-6-phosphate or glyceraldehyde-3-phosphate, that are intermediates metabolites in the glycolysis pathway (Kotrba, P. et al, 2001, Journal of Bioscience and Bioen gineering, 92, 502-17). Reduced sugars, e.g. sorbitol, mannitol and xylitol, are similarly metabolized by first oxidizing each sugar to its corresponding aldo- or keto-sugar and then conversion of the oxidized sugar into a glycolytic pathway intermediate (Kotrba, P. et al, 2001, Journal of Bioscience and Bioengineering, 92, 502-17). The amount of energy, i.e. NADH reducing equivalents, that can be extracted from each of these sources to support the biotransformations described herein depends upon the amount of energy required to uptake the sugar into the host cell and convert it into a glycolysis intermediate.

Five carbon sugars can be used as carbon sources with microorganism strains that are capable of processing these sugars, for example E. coli B. In some embodiments, glycerol, a three carbon carbohydrate, may be used as a carbon source for the biotransformations. Glycerol is metabolized by its conversion into the glycolysis intermediate glyceraldehyde-3-phosphate Lin, E. C. C., 1976, Annu. Rev. Microbiol., 30, 535-78). The amount of energy that can be derived from glycerol depends upon the energy requirements of the pathway in the host microorganism that is used to convert it into glyceraldehyde-3-phosphate. In other embodiments, glycerin, or impure glycerol obtained by the hydrolysis of triglycerides from plant and animal fats and oils, may be used as a carbon source, as long as any impurities do not adversely affect the host microorganisms.

Additional carbon sources can be used by the recombinant microorganisms of the present disclosure including, but not limited to, alkanes, alkenes, alkynes, dienes, isoprenes, aldehydes, carboxylic acids, styrene, cyclic ketones, wax esters and combinations thereof.

Using the above mentioned carbon sources for energy and/or substrate of biotransformation, the host recombinant microorganisms of this disclosure can be used to produce a an extended range of products. In some embodiments, the generated products are oxidized relative to the substrate. In other embodiments, the generated products are reduced relative to the substrate. In one embodiment, the products are saturated or unsaturated alcohols having a carbon atom content limited to between about 1 and about 20 carbon atoms. In another embodiment, the products have a carbon atom content limited to between about 1 and about 10 carbon atoms. In another embodiment, the products have a carbon atom content limited to between about 11 and about 20 carbons. In some embodiments such products include, for example, methanol, ethanol, propanol, butanol, styrene oxide, diols, lactones, alcohols and epoxides.

In some embodiments the recombinant microorganisms are fed at a constant rate or express an heterologous NAD(P)H-requiring oxidoreductase under preferred process conditions, so that these microorganisms do not require alternative metabolic pathways to survive.

In some embodiments, the recombinant microorganism is fed a constant feed rate compatible with the activity of the NAD(P)H-requiring enzyme or metabolic pathway so that all of the NAD(P)H produced in the microorganism is consumed by the NAD(P)H-requiring enzyme or metabolic pathway without accumulation of NAD(P)H.

In some embodiments, the heterologous NAD(P)H-requiring oxidoreductase or metabolic pathways can have oxygen as a substrate. In order for such biocatalysts or metabolic pathways to be effectively used in whole-cell processes, the host organism should provide energy to the system via aerobic metabolism. Under aerobic conditions a single glucose molecule generates 10 NADH molecules as it is broken down into carbon dioxide in facultative aerobes such as E. coli. Generally, NADH and NADPH in aerobes can be considered to be interchangeable due to transhydrogenases which are capable of inter-converting the two cofactors. The transhydrogenation reaction, however, requires an energy source and costs the system one proton from the gradient per cofactor processed. In the presence of oxygen, approximately 6 of the NADH cofactors are consumed by respiration enzymes to produce energy, i.e. ATP, for the cell, leaving a theoretical maximum of 4 reduced cofactors for each glucose consumed available to an NADH or NADPH dependent oxidoreductase in the wild-type microorganism. However, the highest value of NAD(P)H equivalents per glucose used by a heterologous NAD(P)H oxidoreductase expressed in the recombinant microorganism as a whole cell system is expected to be ˜2.3, and ˜1 for oxygen-utilizing oxidoreductase as reported by Walton and Stewart (Walton, A. Z. et al, 2002, Biotechnol. Prog., 18, 262-68; Walton, A. Z. et al, 2004, Biotechnol. Prog., 20, 403-11).

When placed in oxygen-rich media, aerobes metabolize carbon sources such as glucose and glycerol to produce as much ATP as possible for the cells. In addition, the presence of nitrogen and other mineral nutrients provides cells with the means to support the biosynthesis of the polypeptides and polynucleotides needed for rapid cell growth. In the absence of a nitrogen source, however, the cell is unable to manufacture biomolecules and accumulates and then shuttles unused NADH and ATP. For the purposes of the present disclosure, such a metabolic state is ideal, as it supplies the energy driven oxidation reaction with ample amounts of NADH.

Thus, in some embodiments of this disclosure, host aerobes microorganisms are cultured in an environment where the nitrogen supply is controlled so as to modulate biomolecule synthesis. Nitrogen sources which serve as appropriate starting materials for protein production include, but are not limited to: ammonium chloride, ammonium sulfate, ammonium phosphate, ammonia gas, aqueous ammonia, urea, glutamic acid and soybean protein hydrolysate.

In some embodiments of this disclosure, host anaerobes microorganisms are cultured when placed in media such as LB or TB and metabolize carbon sources such as glucose and glycerol to produce as much ATP as possible for the cells. The usage of anaerobic hosts which can metabolize other carbon sources such as petroleum and which can be tolerant to substrates/products that are toxic to aerobic hosts, or are able to import or export substrates and products naturally.

In certain embodiments, the carbon source is selected from the group consisting of: biomass hydrolysates, glucose, starch, cellulose, hemicellulose, xylose, lignin, dextrose, fructose, glycerol, glycerin, galactose and maltose. In certain embodiments, the carbon source is selected from biomass hydrolysates and glucose.

In some embodiments, the heterologous NAD(P)H-requiring enzyme or pathway does not utilize oxygen as a substrate. In order for such biocatalysts to be effectively used in whole-cell processes, the host organism should provide NAD(P)H to the NAD(P)H-requiring enzyme of pathway via the TCA cycle that is modified to replace one or more of the enzymes involved in the TCA cycle as disclosed herein. Under conditions in which the TCA cycle is active, a single glucose molecule generates 10 NADH molecules as it is broken down into carbon dioxide.

In certain embodiments, host microorganisms can be cultured in an oxygen-free environment. This is the case if the enzyme that is overexpressed within the microorganism does not require oxygen and preferably when the substrate is also the carbon source.

Generally, NADH and NADPH in aerobes can be considered to be interchangeable due to transhydrogenases which are capable of inter-converting the two cofactors. The transhydrogenation reaction, however, requires an energy source and costs the system one proton from the gradient per cofactor processed.

In the absence of a nitrogen source, the cell is unable to manufacture biomolecules and accumulates unused NADH and ATP. For the purposes of the present disclosure, such a metabolic state is ideal, as it supplies the NAD(P)H-driven reaction with ample amounts of NADH.

Thus, in some embodiments of this disclosure, host microorganisms are cultured in an environment where the nitrogen supply is controlled so as to modulate biomolecule synthesis. Nitrogen sources which serve as appropriate starting materials for protein production include, but are not limited to: ammonium chloride, ammonium sulfate, ammonium phosphate, ammonia gas, aqueous ammonia, urea, glutamic acid and soybean protein hydrolysate.

In some embodiments, the recombinant microorganism uses glucose as a carbon source and said microorganism produces greater than 4 moles of product per mole of metabolized glucose.

In some embodiments, the recombinant microorganism uses glucose as a carbon source and said microorganism produces greater than 5 moles of product per mole of metabolized glucose.

In some embodiments, the recombinant microorganism uses glucose as a carbon source and said microorganism produces greater than 6 moles of product per mole of metabolized glucose.

In some embodiments, the recombinant microorganism uses glucose as a carbon source and said microorganism produces greater than 7 moles of product per mole of metabolized glucose.

In some embodiments, the recombinant microorganism uses glucose as a carbon source and said microorganism produces greater than 8 moles of product per mole of metabolized glucose.

In some embodiments, the recombinant microorganism uses glucose as a carbon source and said microorganism produces greater than 9 moles of product per mole of metabolized glucose.

In some embodiments, the recombinant microorganism uses glucose as a carbon source and said microorganism produces greater than 10 moles of product per mole of metabolized glucose.

In some embodiments, the recombinant microorganism can be used to optimize the heterologous NAD(P)H-requiring oxiddreductase, and in particular of oxygenases. In those embodiments, directed evolution of the most effective variant of a desired oxygenase, can be performed to obtain improved biocatalysts. As shown in the following examples, using error prone PCR and other mutagenesis techniques, a library of biocatalyst genes can be inserted into an appropriate expression plasmid and transformed into an E. coli strain deficient in both NADH dehydrogenases. The microorganisms containing the library may be transferred onto agar plates containing an appropriate medium spiked with antibiotic and inducing agent and grown in the presence of a substrate. Replicas of each plate may be made and grown without the substrate present to identify mutants that consume NADH without making product, i.e. are uncoupled. All mutants that grow well on the substrate, but not in its absence, may be isolated and characterized to identify the biocatalyst variants with the most improved activity.

In some embodiments, host organisms expressing oxygenases will require oxygen not only as a substrate for the enzyme but also as a terminal electron acceptor for endogenous respiration. Since oxygenases typically have a higher Km for oxygen than the terminal oxidases (bo and bd-type quinol oxidases), relatively high oxygen concentrations need to be maintained during biocatalysis. In standard industrial biocatalytic whole-cell oxygenation processes, typical oxygen transfer coefficients of ca. 200 h⁻¹ are achieved. This is equivalent to an oxygen transfer rate of 1500 U L⁻¹ (1 U=1 μmol min⁻¹), assuming an average air pressure of 2.5 atm in the bioreactor and a desired residual oxygen concentration of 100 Um. Endogenous oxygen consumption due to respiration of ca. 6 mmol g⁻¹ h⁻¹ (=100 U g⁻¹) limits the oxygen available to the catalyst. Therefore, 10 g of cells would consume 10×100 U L⁻¹g⁻¹ oxygen, leaving only 500 U L⁻¹ available to the catalyst. Due to oxygen limitations, cell densities higher than 15 g L⁻¹ are not suitable for such a process.

Using the engineered microorganisms described above for the same process, the oxygen concentration available to the catalyst is drastically increased since endogenous respiration is reduced. A low amount of oxygen is still required to regenerate FADH2 which is produced during the TCA cycle by succinate dehydrogenase. Because 2 mols of FADH are generated per 10 mols of NAD(P)H, the oxygen demand due to endogenous respiration is ⅙ that of an non-engineered microorganism.

In some embodiments, in the recombinant microorganism herein disclosed, 1,5-fold more oxygen is made available to an heterologous oxygenase or other oxygen-requiring NAD(P)H-requiring oxidoreductase in said microorganism as compared to the wild-type microorganism. Therefore in some of those embodiments the recombinant microorganism can produce substantially the same amount of substrate in a culture medium containing 1.5-fold less oxygen, compared to an unengineered microorganism.

In some embodiments, in the recombinant microorganism herein disclosed, 2-fold more oxygen is made available to an heterologous oxygenase or other oxygen-requiring NAD(P)H-requiring oxidoreductase in said microorganism as compared to the wild-type microorganism. Therefore in some of those embodiments the recombinant microorganism can produce substantially the same amount of substrate in a culture medium containing 2-fold less oxygen, compared to an unengineered microorganism.

In some embodiments, in the recombinant microorganism herein disclosed, 2.5-fold more oxygen is made available to an heterologous oxygenase or other oxygen-requiring NAD(P)H-requiring oxidoreductase in said microorganism as compared to the wild-type microorganism. Therefore in some of those embodiments the recombinant microorganism can produce substantially the same amount of substrate in a culture medium containing 2.5-fold less oxygen, compared to an unengineered microorganism.

In some embodiments, in the recombinant microorganism herein disclosed, 3-fold more oxygen is made available to an heterologous oxygenase or other oxygen-requiring NAD(P)H-requiring oxidoreductase in said microorganism as compared to the wild-type microorganism. Therefore in some of those embodiments the recombinant microorganism can produce substantially the same amount of substrate in a culture medium containing 3-fold less oxygen, compared to an unengineered microorganism.

### EXAMPLES

The present disclosure is also illustrated in the following examples, which are provided by way of illustration and are not intended to be limiting.

The following examples relate to metabolically engineered microorganisms capable of converting a substrate to a product. Such microorganisms are characterized in that select proteins of the respiratory chain have been replaced by recombinantly expressed NAD(p)H-requiring oxidoreductase, including oxidases and reductases. The oxidases and reductases utilize oxygen and the nicotinamide cofactors NADH and NADPH which, in the wild-type microorganism, are normally consumed by the respiratory chain to produce ATP. Biocatalytic processes utilizing the metabolically engineered microorganisms of this disclosure characterized by reduced endogenous respiration provide increased yields of product per carbon and oxygen metabolized.

In particular, the following examples relate to engineering metabolic pathways in microorganisms, such as E. coli, such that the microorganism uses one or more NADH or NADPH dependent oxidoreductase biocatalyst biocatalysts that can be part of a metabolic pathway in place of key, endogenous metabolic enzymes. Such modifications render the microorganism dependent upon the engineered oxidoreductase enzyme(s) or metabolic pathway the oxidoreductase is part of, and channel most of the energy from any available energy source to the enzyme. The engineered microorganism no longer metabolizes NADH and instead channels NADH directly into the oxidoreductase enzyme(s) or metabolic pathway the oxidoreductase is part of to drive a desired reaction.

In addition, in embodiments where the oxidoreductase enzyme is an oxygenase or where the this oxygenase is part of a metabolic pathway, and the process is an aerobic process, much of the oxygen that a wild-type microorganism would normally utilize to operate its aerobic metabolism is instead used by the oxygenase in the cells of the engineered microorganisms of the current disclosure. This accommodates implementation of oxygenase enzymes in oxidation processes where hydrophobic oxygen is usually the limiting reagent. As a whole, the microorganisms are modified such that: a) the host comprises a heterologous DNA sequence encoding an enzyme capable of regioselectively and stereoselectively modifying a variety of substrates and b) DNA sequences encoding one or more proteins involved in the respiratory pathway are deleted from the host's genome so as to increase the amount of NADH and NADPH available to the engineered enzyme.

In particular, genes that are deleted or knocked out to produce the microorganisms of this disclosure are exemplified for E. coli. However, the corresponding, homologous or analogous genes can easily be identified in other microorganisms by one skilled in the art and deleted, removed, inhibited, mutated, inactivated, or knocked out in these organisms according to well established molecular biology methods, for example in Pseudomonas putida.

The term “homologue” or “homologous” refers to nucleic acid or protein sequences or protein structures that are related to each other by descent from a common ancestral sequence or structure. All members of a gene family are homologues or homologous, by definition.

The terms “analogue” or “analogous” refers to nucleic acid or protein sequences or protein structures that are related to one another in function only and are not from common descent or do not share a common ancestral sequence. Analogues may differ in sequence but may share a similar structure, due to convergent evolution. For example, two enzymes that catalyze the same reaction of conversion of a substrate to a product but are unrelated in sequence or structure are analogues or analogous. For example, two enzymes that catalyze the same reaction of conversion of a substrate to a product, are unrelated in sequence, but share a similar structure are analogues or analogous.

A polynucleotide or polypeptide has a certain percent “sequence identity” to another polynucleotide or polypeptide, meaning that, when aligned, that percentage of bases or amino acids is the same when comparing the two-sequences. Sequence similarity can be determined in a number of different manners. To determine sequence identity, sequences can be aligned using the methods and computer programs, including BLAST, available over the World Wide Web at ncbi.nlm.nih.gov/BLAST. See, e.g., Altschul et al. (1990), Mol. Biol. 215:403-10.

“Sequence similarity” takes into account (1) the functional impact of amino acid substitutions, (2) amino acid insertions and deletions and (3) the length and structural complexity of a sequence. A “sequence similarity score” is determined by means of a sequence alignment as described above. The “protein similarity score” “S” is a value calculated based on scoring matrix and gap penalty. The higher the score, the more significant the alignment, and the higher the degree of similarity between the queried sequences.

“Sequence alignment” indicates the process of lining up two or more sequences to achieve maximal levels of identity (and, in the case of amino acid sequences, conservation) for the purpose of assessing the degree of similarity. Numerous methods for aligning sequences and assessing similarity/identity are known in the art such as, for example, the Cluster Method, wherein similarity is based on the MEGALIGN algorithm, as well as BLASTN, BLASTP, and FASTA (Lipman and Pearson, 1985; Pearson and Lipman, 1988). When using all of these programs, the preferred settings are those that results in the highest sequence similarity. For example, the “identity” or “percent identity” with respect to a particular pair of aligned amino acid sequences can refer to the percent amino acid sequence identity that is obtained by ClustalW analysis (version W 1.8 available from European Bioinformatics Institute, Cambridge, UK), counting the number of identical matches in the alignment and dividing such number of identical matches by the greater of (i) the length of the aligned sequences, and (ii) 96, and using the following default ClustalW parameters to achieve slow/accurate pairwise alignments—Gap Open Penalty: 10; Gap Extension Penalty: 0.10; Protein weight matrix: Gonnet series; DNA weight matrix: IUB; Toggle Slow/Fast pairwise alignments=SLOW or FULL Alignment. Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Of particular interest are alignment programs that permit gaps in the sequence. The Smith-Watennan is one type of algorithm that permits gaps in sequence alignments. See Meth. Mol. Biol. 70: 173-187 (1997). Also, the GAP program using the Needleman and Wunsch alignment method can be utilized to align sequences. See Mol. Biol. 48: 443-453 (1970).

Two sequences are “optimally aligned” when they are aligned for similarity scoring using a defined amino acid substitution matrix (e.g., BLOSUM62), gap existence penalty and gap extension penalty so as to arrive at the highest score possible for that pair of sequences. Amino acid substitution matrices and their use in quantifying the similarity between two sequences are well known in the art and described, e.g., in Dayhoff et al. (1978) “A model of evolutionary change in proteins” in “Atlas of Protein Sequence and Structure,” Vol. 5, Suppl. 3 (ed. M. O. Dayhoff), pp. 345-352. Natl. Biomed. Res. Found., Washington, D.C. and Henikoff et al. (1992) Proc. Nat'l. Acad. Sci. USA 89: 10915-10919. The BLOSUM62 matrix is often used as a default scoring substitution matrix in sequence alignment protocols such as Gapped BLAST 2.0. The gap existence penalty is imposed for the introduction of a single amino acid gap in one of the aligned sequences, and the gap extension penalty is imposed for each additional empty amino acid position inserted into an already opened gap. The alignment is defined by the amino acids positions of each sequence at which the alignment begins and ends, and optionally by the insertion of a gap or multiple gaps in one or both sequences so as to arrive at the highest possible score. While optimal alignment and scoring can be accomplished manually, the process is facilitated by the use of a computer-implemented alignment algorithm, e.g., gapped BLAST 2.0, described in Altschul et al. (1997) Nucl. Acids Res. 25: 3389-3402, and made available to the public at the National Center for Biotechnology Information (NCBI) Website (www.ncbi.nlm.nih.gov). Optimal alignments, including multiple alignments, can be prepared using, e.g., PSI-BLAST, available through the NCBI website and described by Altschul et al. (1997) Nucl. Acids Res. 25:3389-3402.

Generally, homologous or similar genes and/or homologous or similar enzymes can be identified by functional, structural, and/or genetic analysis and in most cases will have functional, structural, or genetic similarities. Techniques suitable to identify homologous genes and homologous enzymes are known to one skilled in the art. Generally, analogous genes and/or analogous enzymes can be identified by functional analysis and in most cases will have functional similarities. Techniques suitable to identify analogous genes and analogous enzymes are known to one skilled in the art. Techniques to identify homologous or analogous genes, proteins, or enzymes include cloning a first gene using PCR primers based on a known gene/enzyme and PCR, and performing sequencing, genomic mapping and/or functional assays to identify the cloned gene as homologous to the second gene/enzyme. Further, techniques to identify homologous or analogous genes, proteins, or enzymes include examining a cell or cell culture for the catalytic activity of an enzyme through in vitro enzyme assays for said activity, then isolating the enzyme through purification, determining the protein sequence of the enzyme through techniques such as Edman degradation, design of PCR primers to the likely nucleic acid sequence, amplification of said DNA sequence through PCR, and cloning of said nucleic acid sequence would permit one skilled in the art to identify likely alternatives based on functional homology or similarity. Those techniques to identify homologous or similar genes and/or homologous or similar enzymes, analogous genes and/or analogous enzymes or proteins also include comparison of data concerning a candidate gene or enzyme with databases such as BRENDA, KEGG, or MetaCYC. A person skilled in the art can identify the candidate gene or enzyme within the above mentioned databases upon reading of the present disclosure.

The strains described in the following examples are listed in the following Table 7. Mut* refers to additional mutations.

TABLE 7

| | | source or |
|---|---|---|
| Strain | Relevant characteristics | reference |
| Strains | | |
| W3110 | F-L-rph-1 INV(rrnD, rrnE) | DSMZ#613 |
| B | Wt | DSMZ#5911 |
| WA837 | E. coli B, gal-151, met-100, [malB + (LamS)], hsdR11, Δ46 | CGSC#90266 |
| GEVO711 | E. coli W3110, Δ(nuoA-N), P_{nuoA}::BM3(4E10)::FRT-kan-FRT | |
| GEVO713 | E. coli W3110, Δndh::FRT-kan-FRT | |
| GEVO715 | E. coli W3110, Δ(nuoA-N)::FRT-kan-FRT | |
| GEVO717 | E. coli B, Δ(nuoA-N), P_{nuoA}::BM3(4E10)::FRT-kan-FRT | |
| GEVO734 | E. coli W3110, Δ(nuoA-N), P_{lactactac}::BM3(4E10)::FRT-kan-FRT | |
| GEVO736 | E. coli W3110, Δndh, lactactacp::bm3(4E10)::FRT-kan-FRT | |
| GEVO738 | E. coli W3110, Δ(nuoA-N), P_{nuoA}::BM3(4E10)::FRT | |
| GEVO740 | E. coli W3110, Δndh::FRT | |
| GEVO741 | E. coli W3110, Δ(nuoA-N)::FRT | |
| GEVO746 | E. coli WA837, Δ(nuoA-N), P_{nuoA}::BM3(4E10)::FRT-kan-FRT | |
| GEVO747 | E. coli WA837, Δndh, P_{ndh}::BM3(4E10)::FRT-kan-FRT | |
| GEVO748 | E. coli WA837, Δ(nuoA-N), P_{lactactac}::BM3(4E10)::FRT-kan-FRT | |
| GEVO749 | E. coli WA837, Δ(nuoA-N)::FRT-kan-FRT | |
| GEVO750 | E. coli W3110, Δndh::FRT, Δ(nuoA-N)::FRT-kan-FRT | |
| GEVO751 | E. coli W3110, Δ(nuoA-N), P_{nuoA}::BM3(4E10)::FRT, Δndh::FRT-kan- | |
| | FRT | |
| GEVO752 | E. coli WA837, Δndh::P_{lactactac}::BM3(4E10)::FRT-kan-FRT | |
| GEVO756 | E. coli WA837, Δndh::FRT-kan-FRT | |
| GEVO757 | E. coli W3110, Δndh, P_{ndh}::BM3(4E10)::FRT-kan-FRT, Δ(nuoA- | |
| | N)::FRT | |
| GEVO759 | E. coli W3110, Δndh, P_{ndh}::bm3(4E10)::FRT-kan-FRT, Δ(nuoA-N), | |
| | P_{nuoA}::BM3(4E10)::FRT | |
| GEVO761 | E. coli W3110, Δndh, P_{lactactac}::BM3(4E10)::FRT-kan-FRT, | |
| | P_{nuoA}::BM3(4E10)::FRT | |
| GEVO763 | E. coli W3110, Δ(nuoA-N), P_{lactactac}::BM3(4E10)::FRT-kan-FRT, | |
| | Δndh::FRT | |
| GEVO765 | E. coli W3110, Δndh, P_{lactactac}::BM3(4E10)::FRT-kan-FRT, Δ(nuoA- | |
| | N)::FRT | |
| GEVO784 | E. coli B, Δndh, P_{ndh}::BM3(4E10)::FRT-kan-FRT | |
| GEVO785 | E. coli B, Δndh::P_{lactactac}::BM3(4E10)::FRT-kan-FRT | |
| GEVO786 | E. coli B, Δndh::FRT-kan-FRT | |
| GEVO787 | E. coli B, Δ(nuoA-N)::FRT-kan-FRT | |
| GEVO788 | E. coli W3110, ΔldhA:: FRT-kan-FRT | |
| GEVO789 | E. coli WA837, ΔldhA:: FRT-kan-FRT | |
| GEVO1182 | E. coli W3110, ΔldhA::FRT, Δfrd::FRT, ΔfocApflB::FRT | |
| GEVO1283 | E. coli W3110, ΔldhA::FRT, Δfrd::FRT, ΔfocApflB::FRT mut* | |
| GEVO1284 | E. coli W3110, ΔldhA::FRT, Δfrd::FRT, ΔfocApflB::FRT mut* | |
| GEVO1285 | E. coli W3110, ΔldhA::FRT, Δfrd::FRT, ΔfocApflB::FRT mut* | |
| GEVO1286 | E. coli W3110, ΔldhA::FRT, Δfrd::FRT, ΔfocApflB::FRT mut* | |
| GEVO1317 | E. coli B, Δndh::FRT-kan-FRT, Δ(nuoA-N)::FRT | |
| GEVO1318 | E. coli B, Δ(nuoA-N), P_{lactactac}::BM3(4E10)::FRT-kan-FRT | |
| GEVO1319 | E. coli B, Δndh, P_{ndh}::bm3(4E10)::FRT-kan-FRT, Δ(nuoA-N), | |
| | P_{nuoA}::BM3(4E10)::FRT | |
| GEVO1320 | E. coli W3110, Δ(nuoA-N), P_{lactactac}::BM3(4E10)::FRT | |
| GEVO1321 | E. coli W3110, Δ(nuoA-N), P_{lactactac}::BM3(4E10)::FRT, | |
| | Δndh::P_{lactactac}::BM3(4E10)::FRT-kan-FRT | |
| GEVO1322 | E. coli B, Δ(nuoA-N), P_{lactactac}::BM3(4E10)::FRT, | |
| | Δndh::P_{lactactac}::BM3(4E10)::FRT-kan-FRT | |
| GEVO793 | E. coli B, Δndh::FRT | |
| GEVO1323 | E. coli B, Δ(nuoA-N), P_{nuoA}::BM3(4E10)::FRT-kan-FRT, Δndh::FRT | |
| GEVO1324 | E. coli B, Δndh, P_{ndh}::BM3(4E10)::FRT-kan-FRT, Δ(nuoA-N)::FRT | |
| GEVO1325 | E. coli B, Δ(nuoA-N), P_{lactactac}::BM3(4E10)::FRT-kan-FRT, | |
| | Δndh::FRT | |
| GEVO1326 | E. coli B, Δndh, P_{lactactac}::BM3(4E10)::FRT-kan-FRT, Δ(nuoA- | |
| | N)::FRT | |
| GEVO1327 | E. coli W3110, Δndh::FRT, Δ(nuoA-N)::FRT | |
| GEVO1328 | E. coli W3110, Δndh::FRT, Δ(nuoA-N)::FRT, ΔldhA:: FRT-kan-FRT | |
| GEVO1329 | E. coli B, Δndh::FRT, Δ(nuoA-N)::FRT | |
| GEVO1330 | E. coli B, Δndh::FRT, Δ(nuoA-N)::FRT, ΔldhA:: FRT-kan-FRT | |
| GEVO800 | E. coli W3110, ΔadhE:: FRT-kan-FRT | |
| GEVO803 | E. coli WA837, ΔadhE:: FRT-kan-FRT | |
| GEVO1331 | E. coli W3110, Δndh::FRT, Δ(nuoA-N)::FRT, ΔldhA:: FRT | |
| GEVO831 | E. coli W3110, Δndh::FRT, Δ(nuoA-N)::FRT, ΔldhA:: FRT, ΔadhE:: | |
| | FRT-kan-FRT | |
| GEVO1332 | E. coli B, Δndh::FRT, Δ(nuoA-N)::FRT, ΔldhA:: FRT | |
| GEVO1333 | E. coli B, Δndh::FRT, Δ(nuoA-N)::FRT, ΔldhA:: FRT, ΔadhE:: FRT- | |
| | kan-FRT | |
| GEVO802 | E. coli W3110, ΔfocA-pflB:: FRT-kan-FRT | |
| GEVO805 | E. coli WA837, ΔfocApflB:: FRT-kan-FRT | |
| GEVO1334 | E. coli W3110, Δndh::FRT, Δ(nuoA-N)::FRT, ΔldhA:: FRT, ΔadhE:: | |
| | FRT | |
| GEVO1335 | E. coli W3110, Δndh::FRT, Δ(nuoA-N)::FRT, ΔldhA:: FRT, ΔadhE:: | |
| | FRT, ΔfocApflB:: FRT-kan-FRT | |
| GEVO1336 | E. coli B, Δndh::FRT, Δ(nuoA-N)::FRT, ΔldhA:: FRT, ΔadhE:: FRT | |
| GEVO1337 | E. coli B, Δndh::FRT, Δ(nuoA-N)::FRT, ΔldhA:: FRT, ΔadhE:: FRT, | |
| | ΔfocA-pflB:: FRT-kan-FRT | |
| GEVO818 | E. coli W3110, ΔfrdABCD:: FRT-kan-FRT | |
| GEVO822 | E. coli WA837, ΔfrdABCD:: FRT-kan-FRT | |
| GEVO1338 | E. coli W3110, Δndh::FRT, Δ(nuoA-N)::FRT, ΔldhA:: FRT, ΔadhE:: | |
| | FRT, ΔfocA-pflB:: FRT | |
| GEVO1339 | E. coli W3110, Δndh::FRT, Δ(nuoA-N)::FRT, ΔldhA:: FRT, ΔadhE:: | |
| | FRT, ΔfocA-pflB:: FRT, ΔfrdABCD:: FRT-kan-FRT | |
| GEVO1340 | E. coli B, Δndh::FRT, Δ(nuoA-N)::FRT, ΔldhA:: FRT, ΔadhE:: FRT, | |
| | ΔfocA-pflB:: FRT | |
| GEVO1341 | E. coli B, Δndh::FRT, Δ(nuoA-N)::FRT, ΔldhA:: FRT, ΔadhE:: FRT, | |
| | ΔfocA-pflB:: FRT, ΔfrdABCD:: FRT-kan-FRT | |
| GEVO817 | E. coli W3110, ΔackA:: FRT-kan-FRT | |
| GEVO821 | E. coli WA837, ΔackA::: FRT-kan-FRT | |
| GEVO1342 | E. coli W3110, Δndh::FRT, Δ(nuoA-N)::FRT, ΔldhA:: FRT, ΔadhE:: | |
| | FRT, ΔfocA-pflB:: FRT; ΔfrdABCD:: FRT | |
| GEVO1343 | E. coli W3110, Δndh::FRT, Δ(nuoA-N,)::FRT, ΔldhA:: FRT, ΔadhE:: | |
| | FRT, ΔfocA-pflB:: FRT, ΔfrdABCD:: FRT, ΔackA:: FRT-kan-FRT | |
| GEVO1344 | E. coli B, Δndh::FRT, Δ(nuoA-N)::FRT, ΔldhA:: FRT, ΔadhE:: FRT, | |
| | ΔfocA-pflB:: FRT, ΔfrdABCD:: FRT | |
| GEVO1345 | E. coli B, Δndh::FRT, Δ(nuoA-N)::FRT, ΔldhA:: FRT, ΔadhE:: FRT, | |
| | ΔfocA-pflB:: FRT, ΔfrdABCD:: FRT, ΔackA::: FRT-kan-FRT | |
| GEVO801 | E. coli W3110, ΔpoxB:: FRT-kan-FRT | |
| GEVO804 | E. coli WA837, ΔpoxB:: FRT-kan-FRT | |
| GEVO1346 | E. coli W3110, Δndh::FRT, Δ(nuoA-N)::FRT, ΔldhA:: FRT, ΔadhE:: | |
| | FRT, ΔfocA-pflB:: FRT, ΔfrdABCD:: FRT, ΔackA:: FRT | |
| GEVO1347 | E. coli W3110, Δndh::FRT, Δ(nuoA-N)::FRT, ΔldhA:: FRT, ΔadhE:: | |
| | FRT, ΔfocA-pflB:: FRT, ΔfrdABCD:: FRT, ΔackA:: FRT, ΔpoxB:: | |
| | FRT-kan-FRT | |
| GEVO1348 | E. coli B, Δndh::FRT, Δ(nuoA-N)::FRT, ΔldhA:: FRT, ΔadhE:: FRT, | |
| | ΔfocA-pflB:: FRT, ΔfrdABCD:: FRT, ΔackA::: FRT | |
| GEVO1349 | E. coli B, Δndh::FRT, Δ(nuoA-N)::FRT, ΔldhA:: FRT, ΔadhE:: FRT, | |
| | ΔfocA-pflB:: FRT, ΔfrdABCD:: FRT, ΔackA::: FRT, ΔpoxB:: FRT- | |
| | kan-FRT | |

The plasmids described in the in the following examples are listed in the following Table 8.

TABLE 8

| Plasmids | Relevant characteristics | Source or reference |
|---|---|---|
| Pkd13 | bla FRT-kan-FRT | (Datsenko, K. A. et al, |
| | | 2000, Proc. Nat. Acad. Sci. |
| | | USA, 97, 6640-45) |
| Pkd46 | bla γ β exo (red recombinase), | (Datsenko, K. A. et al, |
| | temperature conditional | 2000, Proc. Nat. Acad. Sci. |
| | replicon | USA, 97, 6640-45) |
| Pcp20 | FLP⁺, λ Ci857⁺, λ_{PR} Rep^{ts}, | (Cherepanov, P. P. et al, |
| | Ap^{R}, Cm^{R} | 1995, Gene, 158, 9-14) |
| pRK415 | tetracycline resistance, Plac | Keen N. T. et al., 1988, |
| | promoter | Gene, 70: 191-7 |

The primers used are disclosed in the following examples and listed in the following Table 9.

TABLE 9

| | | |
|---|---|---|
| Name | Primer | Sequence |

| | | |
|---|---|---|
| SEQ ID NO: 45 | 1nuoA_NR | CATCAGCGGCATTGCCAAACG |
| | | CACAATGCTAATCAGCGGTat |
| | | tccggggatccgtcgacc |
| SEQ ID NO: 46 | 2nuoA_NF | TTCATCGCATCGGACGATAGA |
| | | TAATTCCTGAGACAATAGTgt |
| | | gtaggctggagctgcttc |
| SEQ ID NO: 47 | 3ndhF | Atacacccctcactctatatc |
| | | actctcacaaattcgctcagt |
| | | gtaggctggagctgcttc |
| SEQ ID NO: 48 | 4ndhR | ATGCAACTTCAAACGCGGACG |
| | | GATAACGCGGTTAATACTCat |
| | | tccggggatccgtcgacc |
| SEQ ID NO: 49 | 5ndh_BM3F | Cattaattaacaattggttaa |
| | | taaatttaagggggtcacgat |
| | | gacaattaaagaaatgcctca |
| | | gc |
| SEQ ID NO: 50 | 6nuo_BM3F | Gaagagcagtgaatctggcgc |
| | | tacttttgatgagtaagcaat |
| | | gacaattaaagaaatgcctca |
| | | gc |
| SEQ ID NO: 51 | 7nuo_tacBM3F | TTCATCGCATCGGACGATAGA |
| | | TAATTCCTGAGACAATAGTGC |
| | | TTCCGGCTCGTATAATGT |
| SEQ ID NO: 52 | 8ndh_tacBM3F | atacacccctcactctatatc |
| | | actctcacaaattcgctcaGC |
| | | TTCCGGCTCGTATAATGT |
| SEQ ID NO: 53 | 9nuoA_NF | AGACGTGTGGGCTGGGTAAGg |
| | | tgtaggctggagctgcttc |
| SEQ ID NO: 54 | 10BM3R | GAAGCAGCTCCAGCCTACACC |
| | | TTACCCAGCCCACACGTCT |
| SEQ ID NO: 55 | 41ldhA_ko_f | TGTGATTCAACATCACTGGAG |
| | | AAAGTCTTATGAAACTCGCgt |
| | | gtaggctggagctgcttc |
| SEQ ID NO: 56 | 42ldhA_ko_r | TTGCAGCGTAGTCTGAGAAAT |
| | | ACTGGTCAGAGCTTCTGCTat |
| | | tccggggatccgtcgacc |
| SEQ ID NO: 57 | 47focApflB_ko_f | ACCATGCGAGTTACGGGCCTA |
| | | TAAGCCAGGCGAGATATGATg |
| | | tgtaggctggagctgcttc |
| SEQ ID NO: 58 | 48focApflB_ko_r | CATAGATTGAGTGAAGGTACG |
| | | AGTAATAACGTCCTGCTGCat |
| | | tccggggatccgtcgacc |
| SEQ ID NO: 59 | 49adhE_ko_f | GTTATCTAGTTGTGCAAAACA |
| | | TGCTAATGTAGCCACCAAATC |
| | | gtgtaggctggagctgcttc |
| SEQ ID NO: 60 | 50adhE_ko_r | GCAGTTTCACCTTCTACATAA |
| | | TCACGACCGTAGTAGGTATCa |
| | | ttccggggatccgtcgacc |
| SEQ ID NO: 61 | 51poxB_ko_f | GATGGAGAACCATGAAACAAA |
| | | CGGTTGCAGCTTATATCGCgt |
| | | gtaggctggagctgcttc |
| SEQ ID NO: 62 | 52poxB_ko_r | CTGAAACCTTTGGCCTGTTCG |
| | | AGTTTGATCTGCGGTGGAAca |
| | | tatgaatatcctccttag |
| SEQ ID NO: 63 | 53ackA_ko_f | Ataggtacttccatgtcgagt |
| | | aagttagtactggttctgagt |
| | | gtaggctggagctgcttc |
| SEQ ID NO: 64 | 54ackA_ko_r | TGCCGAAACGTGCAGCCAGGT |
| | | TGCGTTCATGATCAACTTCca |
| | | tatgaatatcctccttag |
| SEQ ID NO: 65 | 55frd_ko_f | GACTTATCCATCAGACTATAC |
| | | TGTTGTACCTATAAAGGAGCg |
| | | tgtaggctggagctgcttc |
| SEQ ID NO: 66 | 56frd_ko_r | GAGCTTCATTGGTCGCGTATT |
| | | CCTGTTCCTGATGATCGTTat |
| | | tccggggatccgtcgacc |
| SEQ ID NO: 67 | 773Tryp_kpn_f | CGGGTACCATGGTAGACGGGC |
| | | GATCTTC |
| SEQ ID NO: 68 | 775Tryp_sal_r | GAGTCGACAATTTTGGATGAG |
| | | CCGCTCG |

The recombinant microorganisms, methods and systems herein disclosed are further illustrated in the following actual examples 2-3 and 7-10 and prophetic examples 1, 4-6 and 11-24.

### Example 1

### Deletion of the NADH Dehydrogenase for Aerobic NADH Consumption from Pseudomonas putida Genome

The recombinant microorganisms disclosed herein are organism in which NAD(P)H-requiring pathways not involved in the biotransformation are inactivated to increase the supply of NAD(P)H to the biotransformation. The following is an exemplary embodiment wherein the recombinant microorganism is bacterium, such as Pseudomonas putida wherein the inactivated NAD(P)H consuming pathway is the respiratory pathway containing primary NADH dehydrogenase.

Both NADH dehydrogenases of the respiratory pathway are knocked out and replaced with the enzyme biocatalyst. The parent strain used for the metabolic engineering of Pseudomonas putida towards overproduction of redox cofactors are P. putida KT2440. During strain construction, cultures are grown on Luria-Bertani medium or agar (Sambrook, J., Russel, D. W. Molecular Cloning, A Laboratory Manual. 3 ed. 2001, Cold Spring Harbor, N.Y.: Cold Spring Harbor Laboratory Press) and the medium contains antibiotics, where appropriate. Standard methods are used for transduction with pf16h2 phage to combine mutations, for PCR and for sequencing nucleic acids (Miller, J. H., 1992,; Sambrook, J. et al, 2001; Dáz R et al., 1976, Microbiol Esp., 29, 33-45). DNA for the insertion of genes and expression cassettes into the P. putida chromosome is constructed with Splicing by Overlap Extension (SOE) PCR (Horton, R. M., 1995, Mol Biotechnol, 3, 93-9). Cloning plasmids, chromosomal deletions, insertions and gene disruptions are constructed using the methods developed by (Keen N. T., Tamaki, S., Kabayashi, D., and Trollinger, D. 1988, Gene, 70, 191-7; Marx C. J. et al., Biotechniques 2002, 33(5)1062-7; Kanter-Smoler G et al., Biotechniques 1994, 16(5)800-2; Hoang T. T. et al., Gene 1998, 212, 77-86; Quandt, J., 1993, Gene 127, 15-21).

Homologous recombination in the presence of Cre recombinase is used as the primary method of gene disruption. (Marx C. J. et al., Biotechniques 2002, 33(5)1062-7). Plasmid presence, chromosomal integrations, and deletions are verified by using the appropriate antibiotic markers such as ampicillin, gentamycin, kanamycin, spectinomycin, or tetracycline, and PCR analysis and in case of integrations by sequencing. The genes coding for the NADH dehydrogenases NDH1 and NDH2 are nuoA-N and ndh respectively (Table 10). The operon nuoA-N is deleted completely including all of its promoters as well as upstream regulator binding sites (nucleotides 4655799-4671135 of the genomic sequence are deleted). Ndh is also deleted completely including all of its promoters as well as upstream regulator binding sites (nucleotides 734019-735398 of the genomic sequence are deleted).

Table 10 shows the gene name, genomic location, and sequence of each of the NADH dehydrogenases in Pseudomonas putida KT2440. Table 10 also shows the sequence identifier of the related DNA and protein sequences reported in the enclosed sequence listing.

TABLE 10

| | Gene | Gene | Protein | Common | EC | | | Organism |
|---|---|---|---|---|---|---|---|---|
| Locus | Symbol | sequence | sequence | Name | Number | 5′ End | 3′ End | Name |
| PP_4119 | nuoA | SEQ ID | SEQ ID | NADH | 1.6.5.3 | 4656182 | 4656595 | Pseudomonas |
| | | NO: 69 | NO: 70 | dehydrogenase | | | | putida |
| | | | | I, A subunit | | | | KT2440 |
| PP_4120 | nuoB | SEQ ID | SEQ ID | NADH | 1.6.99.5 | 4656605 | 4657282 | Pseudomonas |
| | | NO: 71 | NO: 72 | dehydrogenase | | | | putida |
| | | | | I, B subunit | | | | KT2440 |
| PP_4121 | nuoCD | SEQ ID | SEQ ID | NADH | N/A | 4657362 | 4659143 | Pseudomonas |
| | | NO: 73 | NO: 74 | dehydrogenase | | | | putida |
| | | | | I, C, D | | | | KT2440 |
| | | | | subunit | | | | |
| PP_4122 | nuoE | SEQ ID | SEQ ID | NADH | 1.6.99.5 | 4659146 | 4659643 | Pseudomonas |
| | | NO: 75 | NO: 76 | dehydrogenase | | | | putida |
| | | | | I, E subunit | | | | KT2440 |
| PP_4123 | nuoF | SEQ ID | SEQ ID | NADH | 1.6.99.5 | 4659640 | 4661001 | Pseudomonas |
| | | NO: 77 | NO: 78 | dehydrogenase | | | | putida |
| | | | | I, F subunit | | | | KT2440 |
| PP_4124 | nuoG | SEQ ID | SEQ ID | NADH | 1.6.99.5 | 4661134 | 4663848 | Pseudomonas |
| | | NO: 79 | NO: 80 | dehydrogenase | | | | putida |
| | | | | I, G subunit | | | | KT2440 |
| PP_4125 | nuoH | SEQ ID | SEQ ID | NADH | 1.6.99.5 | 4663845 | 4664852 | Pseudomonas |
| | | NO: 81 | NO: 82 | dehydrogenase | | | | putida |
| | | | | I, H subunit | | | | KT2440 |
| PP_4126 | nuoI | SEQ ID | SEQ ID | NADH | 1.6.99.5 | 4664864 | 4665412 | Pseudomonas |
| | | NO: 83 | NO: 84 | dehydrogenase | | | | putida |
| | | | | I, I subunit | | | | KT2440 |
| PP_4127 | nuoJ | SEQ ID | SEQ ID | NADH | 1.6.99.5 | 4665423 | 4665923 | Pseudomonas |
| | | NO: 85 | NO: 86 | dehydrogenase | | | | putida |
| | | | | I, J subunit | | | | KT2440 |
| PP_4129 | nuoL | SEQ ID | SEQ ID | NADH | 1.6.99.5 | 4666232 | 4668085 | Pseudomonas |
| | | NO: 87 | NO: 88 | dehydrogenase | | | | putida |
| | | | | I, L subunit | | | | KT2440 |
| PP_4130 | nuoM | SEQ ID | SEQ ID | NADH | 1.6.99.5 | 4668126 | 4669658 | Pseudomonas |
| | | NO: 89 | NO: 90 | dehydrogenase | | | | putida |
| | | | | I, M subunit | | | | KT2440 |
| PP_4131 | nuoN | SEQ ID | SEQ ID | NADH | 1.6.99.5 | 4669666 | 4671135 | Pseudomonas |
| | | NO: 91 | NO: 92 | dehydrogenase | | | | putida |
| | | | | I, N subunit | | | | KT2440 |
| PP_0626 | Ndh | SEQ ID | SEQ ID | NADH | 1.6.99.3 | 735314 | 734019 | Pseudomonas |
| | | NO: 93 | NO: 94 | dehydrogenase | | | | putida |
| | | | | | | | | KT2440 |

Using unmodified and engineered P. putida strains and a plasmid expression system, pRK415 containing cytochrome P450 BM3 from Bacillus megaterium or a variant thereof, the amount of NADH made available to an overexpressed oxygenase catalyst or metabolic pathway is determined. Cytochrome P450 BM3 from Bacillus megaterium is used as the model oxygenase enzyme for this purpose. This enzyme is a fast, water soluble, single-component fatty acid hydroxylase readily expressed in laboratory strains of Pseudomonas. Recently, this enzyme was engineered to hydroxylate linear alkanes, such as octane (Peters, M. W. et al, 2003, J. Am. Chem. Soc., 125, 13442-50), propane (Peters, M. W. et al, 2003, J. Am. Chem. Soc., 125, 13442-50) and ethane (Meinhold, P. et al, 2005, Chembiochem, 6, 1765-68). A variant of BM3, 4E10, which catalyzes the efficient conversion of propane to propanol is used for the measurements, in which cells containing 4E10 are placed in a fermenter containing varying concentrations of glucose, propane and oxygen and allowed to react over several hours. For each reactor condition, the rate of glucose consumption is compared to the rate of product formation to determine the amount of available NAD(P)H utilized by the catalyst.

As described above, the amount of NAD(P)H available to an overexpressed biocatalyst is determined. The host microorganisms containing the plasmid to express the biocatalyst are first grown to high density in a rich medium. Biocatalyst expression is then induced using IPTG and, after an optimum amount of active biocatalyst is accumulated inside the hosts, the cells are removed from the rich medium and placed in an oxygenated fermenter containing a nitrogen-free, glucose-rich medium wherein the glucose present is converted into NADH and ATP by the microorganisms. In the presence of a substrate, the biocatalyst consumes this NADH to produce oxygenated products.

P. putida microorganisms expressing BM3 variant 4E10 are used to perform whole-cell reactions under different reaction conditions. In particular, oxygen concentration and pH are monitored during the course of the reaction and correlated to changes in biocatalyst productivity. Parameters such as temperature, biocatalyst expression levels and concentrations of oxygen, glucose, carbon dioxide, substrate and products are measured over the course of the whole cell reactions and the data is used to fine tune process conditions. For example, lower NAD(P)H/glucose ratios reported for whole cell oxygenase reactions might be caused by other enzymes in the aerobic metabolic pathways outcompeting the biocatalysts for oxygen. Therefore, reactor configurations that maximize oxygen transfer to the microorganisms are of high interest. Additionally, these studies help identify biocatalyst properties, such as substrate and oxygen binding affinity, which can be improved via protein engineering techniques such as site-directed mutagenesis and directed evolution (Panke, S. et al, 2004, Curr. Opin. Biotech., 15, 272-79).

Whole-cell reactions are performed in temperature-controlled DasGip fedbatch pro 400 Ml fermenters (DASGIP, Germany). The cell pellet is resuspended in 250 Ml of nitrogen-free, minimal salts medium. Dissolved oxygen and Ph are measured in real time using electrodes attached to the fermentation vessels. The dissolved oxygen concentration is maintained at 100% by a combination of an automated gas mixer (mixing oxygen, air and nitrogen) and an automated mass flow controller (up to a maximum of 50 L/h). The temperature is maintained at 30° C. and the pH is kept constant at 7.0 (by automatic addition of 2 M NaOH or 2 M HCL). Glucose is added via a peristaltic pump to maintain a concentration of ca. 10 mM. Samples are taken periodically and analyzed for a variety of properties. Cell density is measured at a wavelength of 600 nm. Samples of the reaction medium are centrifuged for 10 min at 20,000 g in a microcentrifuge. The supernatant is filtered through a 0.2-μm syringe filter and stored chilled prior to analysis. The concentration of glucose and organic metabolites in the reaction medium is determined by high performance liquid chromatography (HPLC) according to standard protocols. Active P450 concentration is determined using an established carbon monoxide (CO) binding assay on cell lysates removed from the fermenter according to standard procedures (Omura, T., Sato R., 1964, Biol. Chem., 239, 2370-78) adjusted for high throughput format in 2 mL-96-well plates (Qtey, C., 2003, in: Screening and Selection for Directed Enzyme Evolution, Humana Press, Inc., Totowa, N.J.). The concentrations of the resulting products are measured using gas chromatography according to established procedures. (Bell S. G. et al., 2003, Dalton Transactions, 11, 2133-40). Ratios of product molecules formed per glucose molecule consumed are calculated from this data.

With NDH1 and NDH2 removed, more than 4 NADH per glucose that are normally processed by these enzymes are made available to a biocatalyst. By leaving at least one of the quinol oxidase complexes in place, electrons from FADH₂ still result in the generation of ATP.

With the biocatalyst removing NADH from the hosts as it is produced, the microorganisms grow faster than microorganisms without NADH dehydrogenases and no NADH-dependent overexpressed enzyme biocatalyst. These microorganisms are also able to produce a small amount of ATP via oxidative phosphorylation since the biocatalyst consumes one proton per NADH while inside the cell, causing a proportional net difference in the proton gradient. Hence, the engineered microorganisms containing the biocatalyst are expected to grow more efficiently than the microorganisms with both NADH dehydrogenases removed.

Since glucose flux is primarily regulated by ATP production, the lower ATP levels in the microorganisms without NDH activity will increase the flow of glucose-through the microorganism, resulting in NADH formation rates inside the cell that are higher than those found in wild-type cells. The increase in glycolytic flux might be limited by the availability of ADP (Koebmann, B. J. et al, 2002, J. Bacteriol., 184, 3909-16). This limitation is especially important in non-growing cells, since biosynthesis reactions that use the bulk of the ATP are drastically reduced. The availability of ADP can be increased by the introduction of futile cycles that consume ATP (Patnaik, R. et al, 1992, J. Bacteriol., 174, 7527-32) or by expression of cytoplasmic ATPase (Causey, T. B. et al, 2003, Proc. Natl. Acad. Sci., 100, 825-32; Koebmann, B. J. et al, 2002, J. Bacteriol., 184, 3909-16).

As long as the biocatalyst is expressed in sufficient amounts to process the corresponding increase in intracellular NADH, the engineered microorganisms will produce more product of the biotransformation compared to the unengineered microorganism and process much more material per cell than unaltered cells. Should the microorganism activate alternative NAD(P)H-requiring enzymes or pathways that outcompete the NAD(P)H-requirement of the biotransformation, then these pathways are identified and sequentially inactivated until most (5 or more) or all (10) of the NAD(P)H produced by the cell is consumed by the enzyme or pathway of the biotransformation. At this point, the cell is dependent upon the enzyme or pathway for survival.

### Example 2

### Determination of NADH Availability in E. coli

Using unmodified E. coli strains and a plasmid expression system, the amount of NADH made available to an overexpressed oxygenase catalyst was determined. Cytochrome P450 BM3 from Bacillus megaterium was used as the model oxygenase enzyme for this purpose. This enzyme is a fast, water soluble, single-component fatty acid hydroxylase readily expressed in laboratory strains of Escherichia coli. Recently, this enzyme was engineered to hydroxylate linear alkanes, such as octane (Peters, M. W. et al, 2003, J. Am. Chem. Soc., 125, 13442-50), propane (Peters, M. W. et al, 2003, J. Am. Chem. Soc., 125, 13442-50) and ethane (Meinhold, P. et al, 2005, Chembiochem, 6, 1765-68). A variant of BM3, 4E 10, which catalyzes the efficient conversion of propane to propanol was used for the measurements, in which cells containing 4E10 were placed in a fermenter containing varying concentrations of glucose, propane and oxygen and allowed to react over several hours. For each reactor condition, the rate of glucose consumption was compared to the rate of product formation to determine the amount of available NADH utilized by the catalyst.

As described above, the amount of NAD(P)H available to an overexpressed biocatalyst was determined. The host microorganisms containing the plasmid (as detailed below) to express the biocatalyst were first grown to high density in a rich medium. Biocatalyst expression was then induced and, after an optimum amount of active biocatalyst was accumulated inside the hosts, the cells were removed from the rich medium and placed in an oxygenated fermenter containing a nitrogen-free, glucose-rich medium wherein the glucose present was converted into NADH and ATP by the microorganisms. In the presence of a substrate, the biocatalyst consumed this NADH to produce oxygenated products.

The stress of removing cells from a rich medium and placing them into a minimal medium reduces their effectiveness as biocatalysts. The healthiest cells were obtained when M9Y (M9 supplemented with 2% yeast extract) rich medium was used to grow the cells. A 3MI overnight culture of E. coli BL21 or DH5a cells, previously transformed with a plasmid containing the cytochrome P450 oxygenase biocatalyst 4E10 was grown in 3 M1 LB medium containing 100 μg/Ml at 37° C./250 rpm. This culture was used to inoculate 500 Ml of M9 medium (Sambrook, J., Russel, D. W. Molecular Cloning, A Laboratory Manual. 3 ed. 0.2001, Cold Spring Harbor, N.Y.: Cold Spring Harbor Laboratory Press) containing 0.4% glucose, 100 μg/Ml ampicillin and 2% (w/v) yeast extract and the culture was incubated at 30° C./250 rpm. IPTG (1 Mm) was added after 12 h and the culture was grown for an additional 12 h or until the P450 concentration was greater than 1 μM. The cells were then centrifuged at 3000×g for 15 min. After removal of the medium supernatant, the cells were resuspended in M9 medium (Sambrook, J., Russel, D. W. Molecular Cloning, A Laboratory Manual. 3 ed. 2001, Cold Spring Harbor, N.Y.: Cold Spring Harbor Laboratory Press), were allowed to acclimate for three hours at 30° C./250 rpm and were harvested by centrifugation and stored at 4° C. Expression of the biocatalyst at this stage was controlled using an appropriate plasmid (as detailed below) and an expression strain of E. coli (such as DH5α or BL21)

As described below, however, strains such as W3110 and B were used for metabolic engineering. Because most plasmid expression systems impart antibiotic resistance to the host microorganism and expression of the gene for the biocatalyst is regulated using inducing agents, said agents were used in the M9Y medium to accumulate both cell mass and biocatalyst. Active P450 oxygenase expression levels as high as 0.1 g/g cell dry weight, are easily obtainable under these conditions. For reactions lasting one day or less, neither antibiotic nor inducing agent is required in the minimal medium used in the fermenter. These compounds may be required to support longer whole cell reactions, especially if a small amount of cell growth and biocatalyst expression is required to replace dead cells and inactivated biocatalyst over time. However, the metabolic engineering of the host microorganisms described below ultimately increases the long term viability of the whole cell process better than can be done by controlling the feeding rates of antibiotic and inducing agents.

E. coli microorganisms expressing BM3 variant 4E10 were used to perform whole-cell reactions under different reaction conditions. In particular, oxygen concentration and pH (an indirect measure of the use of overflow metabolic pathways inside the cell) were monitored during the course of the reaction and correlated to changes in biocatalyst productivity. Parameters such as temperature, biocatalyst expression levels and concentrations of oxygen, glucose, carbon dioxide, substrate and products were measured over the course of the whole cell reactions and the data used to fine tune process conditions. For example, the lower NAD(P)H/glucose ratios reported for whole cell oxygenase reactions might be caused by other enzymes in the aerobic metabolic pathways outcompeting the biocatalysts for oxygen. Therefore, reactor configurations that maximize oxygen transfer to the microorganisms are of high interest. Additionally, these studies have helped identify biocatalyst properties, such as substrate and oxygen binding affinity, which can be improved via protein engineering techniques such as site-directed mutagenesis and directed evolution (Panke, S. et al, 2004, Curr. Opin. Biotech., 15, 272-79).

Whole-cell reactions were performed in temperature-controlled DasGip fedbatch pro 400 mL fermenters (DASGIP, Germany). The cell pellet was resuspended in 250 Ml of nitrogen-free, minimal salts medium (M9 medium without ammonium chloride added), and supplemented with 10 Ml/L VA solution (Neidhardt, F. C., et al., 1974, J. Bacteriol., 119, 736-47), 1 Ml/L micronutrient stock (Neidhardt, F. C., et al., 1974, J. Bacteriol., 119, 736-47), and 5 Ml/L of 0.0325% thiamine. Dissolved oxygen and Ph was measured in real time using electrodes attached to the fermentation vessels. The dissolved oxygen concentration was maintained at 100% by a combination of an automated gas mixer (mixing oxygen, air and nitrogen) and an automated mass flow controller (up to a maximum of 50 L/h). The temperature was maintained at 30° C. and the Ph was kept constant at 7.0 (by automatic addition of 2 M NaOH or 2 M HCL. Glucose was added via a peristaltic pump to maintain a concentration of ca. 10 Mm. Samples were taken periodically and analyzed for a variety of properties. Cell density was measured at a wavelength of 600 nm. Samples of the reaction medium were centrifuged for 3 min at 14,000 g in a microcentrifuge.

The supernatant was filtered through a 0.2-μm syringe filter and stored chilled prior to analysis. The concentration of glucose and organic metabolites (e.g. lactate, ethanol) in the reaction medium was determined by high performance liquid chromatography (HPLC) according to standard protocols (Causey, T. B. et al, 2003, Proc. Natl. Acad. Sci., 100, 825-32). Active cytochrome P450 concentration was determined using an established carbon monoxide (CO) binding assay on cell lysates removed from the fermenter according to standard procedures (Omura, T., Sato R., 1964, Biol. Chem., 239, 2370-78) adjusted for high throughput format in 2MI-96-well plates (Otey, C., 2003, in: Screening and Selection for Directed Enzyme Evolution, Humana Press, Inc., Totowa, N.J.). The concentrations of the resulting products were measured using gas chromatography according to established procedures (Bell S. G. et al., 2003, Dalton Transactions, 11, 2133-40). Ratios of product molecules formed per glucose molecule consumed were calculated from this data.

### Example 3

### Deletion of the NADH Dehydrogenase for Aerobic NADH Consumption from Host Microorganism Genome

Both NADH dehydrogenases of the respiratory chain were knocked out and replaced with the biocatalyst. Parent strains used for the metabolic engineering of E. coli towards overproduction of redox cofactors were E. coli W3110 (ATCC 27325) and E. coli B. For the transfer of genomic deletions, insertions and gene disruptions from E. coli K12 to E. coli B strain WA837 (CGSC 90266) was used as an intermediate host. During strain construction, cultures were grown on Luria-Bertani medium or agar (Sambrook, J., Russel, D. W. Molecular Cloning, A Laboratory Manual. 3 ed. 2001, Cold Spring Harbor, N.Y.: Cold Spring Harbor Laboratory Press). Standard methods were used for transduction with phage P1, PCR and sequencing (Miller, J. H., 1992,; Sambrook, J. et al, 2001). DNA for the insertion of genes and expression cassettes into the E. coli chromosome was constructed with Splicing by Overlap Extension (SOE) (Horton, R. M., 1995, Mol Biotechnol, 3, 93-9). Chromosomal deletions, insertions and gene disruptions were constructed using the methods developed by Datsenko and Wanner (Datsenko, K. A. et al, 2000, Proc. Nat. Acad. Sci. USA, 97, 6640-45). Chromosomal integrations and deletions were verified by using the appropriate antibiotic markers and, PCR analysis and in case of integrations by sequencing.

Homologous recombination in the presence of Red recombinase was used as the primary method of gene disruption. (Datsenko, K. A. et al, 2000, Proc. Nat. Acad. Sci. USA, 97, 6640-45). The gene(s) coding for the NADH dehydrogenases NDH1 and NDH2 are nuoA-N and ndh respectively. The operon nuoA-N was deleted completely including its two promoters nuoAp1 and nuoAp2 as well as regulator binding sites upstream of nuoAp1 (nucleotides −716(nuoA)-1234(nuoN) were deleted). Pkd13 was used as the template for the PCR with 2nuoA_NF and 1nuoA_NR as forward and reverse primers (Table 9). W3110(Pkd46) was transformed with the PCR product. The resulting strain, GEVO715, contained FRT-kan-FRT in place of the nuoA_N operon. A phage P1 lysate of GEVO715 was prepared and the deletion was transferred into WA837, an E. coli B strain which is r_{B}⁻m_{B}⁺. From the resulting strain, GEVO749, the deletion was transduced into E. coli B yielding GEVO787. Ndh was deleted (including its promoter) with Pkd13 as template and 3ndhF and 4ndhR as the forward and reverse primers (nucleotides −2,9-1272 were deleted). W3110 (Pkd46) was transformed with the PCR product. The resulting strain, GEVO713, contained FRT-kan-FRT in place of the ndh gene. A phage P1 lysate of GEVO713 was prepared and the deletion was transferred into WA837. From the resulting strain, GEVO756, the deletion was transduced into E. coli B yielding GEVO786. To remove both NADH dehydrogenases from E. coli the deletions of ndh and nuoA_N were combined. The kan^{R} cassette was removed from the chromosome of GEVO713 with FLP recombinase using a temperature conditional helper plasmid (Pcp20). The resulting strain GEVO740 was transduced with the lysate of GEVO715 and the resulting double deletion strain was designated GEVO750. The same procedure is used to construct the double deletion of nuoA_N and ndh in E. coli B (GEVO1317).

With NDH-1 and NDH-2 removed, most of the 10 NADH per glucose that are normally processed by these enzymes is expected to be made available to a biocatalyst. By leaving at least one of the quinol oxidase complexes in place, FADH₂ was still allowed to be converted into ATP. The bd-type quinol oxidase has a higher affinity for oxygen than the bo-type—Km of 0.1 μM vs. 1-2 μM respectively. This may require the removal of the bd-type quinol oxidase in order to lessen the competition for oxygen between the attenuated respiration pathway and the biocatalyst.

With the biocatalyst removing NADH from the hosts as it is produced, the microorganisms grow faster than microorganisms without NADH dehydrogenases and no outlet for NADH other than overflow metabolism (which produces toxic metabolites, such as acetate). These microorganisms might also be able to produce a small amount of ATP via oxidative phosphorylation since the biocatalyst consumes one proton per NADH while inside the cell, causing a proportional net difference in the proton gradient. Hence, the engineered microorganisms containing the biocatalyst are expected to grow more efficiently than the microorganisms with both NADH dehydrogenases removed.

Since glucose flux is primarily regulated by ATP production, the lower ATP levels in the microorganisms without NDH activity will increase the flow of glucose through the microorganism, resulting in NADH formation rates inside the cell that are higher than those found in wild-type cells. The increase in glycolytic flux might be limited by the availability of ADP (Koebmann, B. J. et al, 2002, J. Bacteriol., 184, 3909-16). This limitation is especially important in non-growing cells, since biosynthesis reactions that use the bulk of the ATP are drastically reduced. The availability of ADP can be increased by the introduction of futile cycles that consume ATP (Patnaik, R. et al, 1992, J. Bacteriol., 174, 7527-32) or by expression of cytoplasmic ATPase (Causey, T. B. et al, 2003, Proc. Natl. Acad. Sci., 100, 825-32; Koebmann, B. J. et al, 2002, J. Bacteriol., 184, 3909-16). As long as the biocatalyst is expressed in sufficient amounts to process the corresponding increase in intracellular NADH, the engineered microorganisms will be able to process much more material per cell than unaltered cells—significantly reducing the reaction volume of the final device.

### Example 4

### Deletion of the Cytochromes for Aerobic NADH Consumption from Host Microorganism Genome

Escherichia coli has three terminal oxidases: cytochrome bo encoded by the cyo operon, cytochrome bd-1 encoded by cydA and cydB, and cytochrome bd-11 encoded by the appC and appB genes. The cytochrome bo terminal oxidase complex is a terminal oxidase in the respiratory chain used under high oxygen growth conditions. The enzyme catalyzes the two-electron oxidation of ubiquinol within the membrane and the four-electron reduction of molecular oxygen to water. In the cell the enzyme functions as a proton pump, with a net movement of 2H+/e− across the cytoplasmic membrane, thereby generating a proton-motive force (Puustinen A, Finel M, Haltia T, Gennis R B, Wikstrom M (1991). “Properties of the two terminal oxidases of Escherichia coli.” Biochemistry 30(16); 3936-42. PMID: 1850294). There are four subunits, three of which are responsible for the enzyme activity. Those subunits are coded for by the cyoB, cyoA, cyoc and cyoD genes, all of which are necessary for a functional enzyme. Cytochrome bd-I is one of three terminal oxidases in the respiratory chain of E. coli. It is used under conditions of limited oxygen and catalyzes the two-electron oxidation of ubiquinol and the four-electron reduction of oxygen to water. Unlike cytochrome bo, it is not a proton pump (Puustinen A, Finel M, Haltia T, Gennis R B, Wikstrom M (1991). “Properties of the two terminal oxidases of Escherichia coli.” Biochemistry 30(16); 3936-42. PMID: 1850294). Cytochrome bd-I has two subunits.

The appC-encoded subunit of cytochrome bd-II is 60% homologous with CydA and the appB-encoded subunit with CydB (Dassa J, Fsihi H, Marck C, Dion M, Kieffer-Bontemps M, Boquet P L (1991). “A new oxygen-regulated operon in Escherichia coli comprises the genes for a putative third cytochrome oxidase and for pH 2.5 acid phosphatase (appA).” Mol Gen Genet. 1991; 229(3); 341-52. PMID: 1658595). However, under normal conditions of growth, cytochrome bd-II is apparently not expressed because strains in which cytochrome bo and cytochrome bd-I have been mutationally inactivated are unable to grow aerobically with succinate as a sole source of carbon and energy. However, if such a strain is complemented with a chromosomal fragment from Bacillus firmus, cytochrome bd-II is expressed and the strain can grow in a cytochrome bd-II-dependent manner, aerobically on succinate (Sturr M G, Krulwich T A, Hicks D B (1996). “Purification of a cytochrome bd terminal oxidase encoded by the Escherichia coli app locus from, a delta cyo delta cyd strain complemented by genes from Bacillus firmus OF4.” J Bacteriol 178(6); 1742-9. PMID: 8626304). The appCB-appA operon is under the control of the transcriptional activator AppY. It is induced upon entry into the stationary phase, as well as starvation for carbon or phosphate (Atlung T, Knudsen K, Heerfordt L, Brondsted L (1997). “Effects of sigmaS and the transcriptional activator AppY on induction of the Escherichia coli hya and cbdAB-appA operons in response to carbon and phosphate starvation.” J Bacteriol 1997; 179(7); 2141-6. PMID: 9079897). The physiological role of cytochrome bd-II terminal oxidase in wild-type strains of E. coli is obscure. The strategy includes knocking out these three-terminal oxidases.

Homologous recombination in the presence of Red recombinase was used as the primary method of gene disruption. (Datsenko, K. A. et al, 2000, Proc. Nat. Acad. Sci. USA, 97, 6640-45). CyoB, cyoA, cyoC and cyoD genes were deleted completely. Pkd13 was used as the template for the PCR with corresponding primers and then W3110(Pkd46) was transformed with the PCR product. The resulting strain, contained FRT-kan-FRT in place of the cyoABCD genes. A phage P1 lysate prepared and the deletion was transferred into WA837, an E. coli B strain which is r_{B}⁻m_{B}⁺. From the resulting strain, the deletion was transduced into E. coli B. The kan^{R} cassette was removed from the chromosome with FLP recombinase using a temperature conditional helper plasmid (Pcp20), when necessary.

CydA and cydB were deleted completely including its promoter sites and upstream regulator binding sites. Pkd13 was used as the template for the PCR with corresponding primers and then W3110(Pkd46) was transformed with the PCR product. The resulting strain, contained FRT-kan-FRT in place of the cydAB operon. A phage P1 lysate prepared and the deletion was transferred into WA837, an E. coli B strain which is r_{B}⁻ m_{B}⁺. From the resulting strain, the deletion was transduced into E. coli B. The kan^{R} cassette was removed from the chromosome with FLP recombinase using a temperature conditional helper plasmid (Pcp20), when necessary.

AppC and appB were deleted completely including its promoter site. Pkd13 was used as the template for the PCR with corresponding primers and then W3110(Pkd46) was transformed with the PCR product. The resulting strain, contained FRT-kan-FRT in place of the appCB operon. A phage P1 lysate prepared and the deletion was transferred into WA837, an E. coli B strain which is r_{B}⁻ m_{B}⁺. From the resulting strain, the deletion was transduced into E. coli B. The kan^{R} cassette was removed from the chromosome with FLP recombinase using a temperature conditional helper plasmid (Pcp20), when necessary.

The same procedure is used to construct the combination of deletions mentioned above in E. coli.

### Example 5

### Deletion of the Ubiquinone for Aerobic NADH Consumption from Host Microorganism Genome

Another example for generating respiratory negative strains is to delete genes of the ubiquinone synthesis pathway.

Bacterial respiratory quinones can be divided into two groups, ubiquinone (UQ) or coenzyme Q and the naphthoquinones menaquinone (MK) or demethylmenaquinone (DMK). MK plays an additional role in the anaerobic biosynthesis of pyrimidines (Gibson & Cox, 1973). The quinone structure has isoprenoid side chains of various length depending on the species. E. coli has usually a chain length of 8 isoprenoid molecules (UQ-8). In E. coli, the composition of the quinone pool is highly influenced by the degree of oxygen availability: aerobically grown E. coli cells contain significantly more UQ-8 than MK-8 and DMK-8, whereas in anaerobic cells the profile is reversed (Meganathan, 1996; Ingledew & Poole, 1984; Wissenbach et al., 1990, 1992; Shestopalov et al., 1997). The menaquinone biosynthesis pathway supplies two of the three major quinones in E. coli, demethylmenaquinone (DMK) and menaquinone (MK). The third major quinone, ubiquinone (Q), is synthesized from the same precursor, chorismate, but using a different pathway (see Alexander K, Young I G (1978). “Alternative hydroxylate for the aerobic and anaerobic biosynthesis of ubiquinone in Escherichia coli.” Biochemistry 17(22); 4750-5; Meganathan R (2001). “Ubiquinone biosynthesis in microorganisms.” FEMS Microbiol Lett 203(2); 131-9; Neidhardt F C, Curtiss III R. Ingraham J L, Lin E C C, Low Jr K B, Magasanik B, Reznikoff W S, Riley M, Schaechter M, Umbarger H E “Escherichia coli and Salmonella, Cellular and Molecular Biology, Second Edition.” American Society for Microbiology, Washington, D.C., 1996. Soballe B, Poole R K (1999). “Microbial ubiquinones: multiple roles in respiration, gene regulation and oxidative stress management.” Microbiology 145 (Pt 8); 1817-30. PMID: 10463148; See also FIGS. 10 and 11 illustrating ubiquinone biosynthesis I and regulation (aerobic)).

Deleting ubi C,A,X,D,B,G,H,F (one or all genes) is expected to generate a respiratory negative strain.

UbiA and ubiC were deleted completely including its promoter site. Pkd13 was used as the template for the PCR with corresponding primers and then W3110(Pkd46) was transformed with the PCR product. The resulting strain, contained FRT-kan-FRT in place of the ubiAC operon. A phage P1 lysate prepared and the deletion was transferred into WA837, an E. coli B strain which is r_{B}⁻m_{B}⁺. From the resulting strain, the deletion was transduced into E. coli B. The kan^{R} cassette was removed from the chromosome with FLP recombinase using a temperature conditional helper plasmid (Pcp20), when necessary.

The same procedure is used to construct the combination of deletions mentioned above in E. coli.

A very interesting finding is that mutations in ubiF (and other ubi synthesis pathways genes) lead to resistance of high temperature (Collis C M, Grigg G W. J. Bacteriol. (1989) 171(9):4792-8). Since our final strain will be respiratory negative this deletions are very beneficial. Deleting these genes could potentially create a production strain with increases temperature resistance.

### Example 6

### Overexpression of ATPase in a Host Microorganism

ATP is competitive inhibitor of mammalian CS. Yeast CS is also inhibited by ATP as are others microbial CS. A study of CS purified from the facultatively photosynthetic bacterium Rhodospirillum rubrum (Gram negative) and the thermophile Bacillus stearothermophilus (Gram positive) are both inhibited by ATP. Based on these results it is conclusive to assume that the E. coli enzyme might be inhibited by ATP, too. Though BRENDA (enzyme database) does not list the E. coli enzyme to be inhibited by ATP we address the potential problem of ATP inhibition in this example.

Control by the ATP-hydrolyzing reaction was convincingly shown in a study in which the F1 part of the H⁺-ATPase was overexpressed in the cytosol, thereby increasing ATP hydrolysis without affecting the rest of metabolism (Koebmann, B. J., H. V. Westerhoff, J. L. Snoep, D. Nilsson, and P. R. Jensen. 2002. The glycolytic flux in Escherichia coli is controlled by the demand for ATP. J. Bacteriol. 184:3909-3916). The authors worked in the framework of Metabolic Control Analysis and showed that more than 75% of the control of the glycolytic flux resides in the ATP-consuming steps. Independently, but by use of a similar strategy, these results were confirmed by Causey et al. (Causey, T. B., S. Zhou, K. T. Shanmugam, and L. O. Ingram. 2003. Engineering the metabolism of Escherichia coli W3110 for the conversion of sugar to redox-neutral and oxidized products: homoacetate production. Proc. Natl. Acad. Sci. USA, 100:825-832). Such a control distribution was predicted on basis of theoretical considerations in a supply-demand analysis of the system (Hofmeyr, J. S., and A. Cornish-Bowden. 2000. Regulating the cellular economy of supply and demand. FEBS Lett. 476:47-51). The distribution of control over the supply or demand reactions appears to be dependent on the organism and the growth conditions (Koebmann, B. J., H. V. Westerhoff, J. L. Snoep, C. Solem, M. B. Pedersen, D. Nilsson, O. Michelsen, and P. R. Jensen. 2002. The extent to which ATP demand controls the glycolytic flux depends strongly on the organism and conditions for growth. Mol. Biol. Rep. 29:41-46).

The nature of the control of glycolytic flux is one of the central, as-yet-uncharacterized issues in cellular metabolism. Genes encoding the F(1) part of the membrane-bound (F(1)F(0)) H(+)-ATP synthase were expressed in steadily growing Escherichia coli cells, which lowered the intracellular [ATP]/[ADP] ratio. This resulted in a strong stimulation of the specific glycolytic flux concomitant with a smaller decrease in the growth rate of the cells. By optimizing additional ATP hydrolysis, we increased the flux through glycolysis to 1.7 times that of the wild-type flux.

Glycolytic flux can be limited by ATP utilization during the oxidative metabolism of glucose which limits the amount of NADH that can be generated. Glycolytic flux increases in a dose dependent manner with controlled expression of F1-ATPase genes from a plasmid since ATP is hydrolysed (Koebmann, B. J., Westerhoff, H. V., Snoep, J. L., Nilsson, D. & Jensen, P. R. (2002) J. Bacteriol. 184, 3909-3916). This is further supported by Ingram et al. (Causey T. B., Zhou S., Shanmugam K. T., Ingram L. O. (2003) Proc. Natl. Acad. Sci., USA. 100(3):825-32). During the oxidative metabolism of glucose, glycolytic flux is limited by the metabolic ability to use ATP (availability of ADP) rather than by glucose transport or the catalytic capacities of central glycolytic enzymes. With this in mind, similar strategies that delete subunits concerned with the membrane assembly of the (F₁F₀)H⁺ ATP synthase, create futile cycles for ATP consumption, or increase cytoplasmic levels of the ATPase activities may prove useful to decrease cell yield, increase metabolic flux, and increase product yield in many other bioconversion processes (Causey T. B., Zhou S., Shanmugam K. T., Ingram L. O. (2003) Proc. Natl. Acad. Sci., USA. 100(3):825-32)

### Example 7

### Genetic Insertion of the Biocatalyst into Host Microorganism Genome

Once the microorganisms were placed in a minimal medium inside the fermenter, expression of the biocatalyst was stopped, allowing the activity and lifetime of the biocatalyst in the microorganism to be easily measured. For a continuous process where biocatalyst activity is required for extended periods of time, however, plasmid expression systems are not desired. Forced overexpression with an inducing agent is damaging to the long term viability of a microorganism—a reason why microorganisms expressing industrially important proteins are usually harvested fairly quickly after induction. Additionally, plasmids are relatively unstable in a population of growing cells unless they impart the cell with an advantage such as antibiotic resistance. To increase the stability of the biocatalyst gene over a long period of time, the gene of the biocatalyst was inserted into the host cell genome. The resulting host strain is then used as a starting point for metabolic engineering as described below.

To avoid the presence of a plasmid carrying the gene coding for the NAD(P)H consuming enzyme in the engineered strain, the gene encoding an NAD(P)H-requiring oxidoreductase was integrated into the E. coli chromosome. While this was accomplished by replacing the genes of NDH1 and NDH2 with the 4E10 gene, any other oxidoreductase enzyme overexpressed in the engineered strain has the same effect.

Once inserted, the expression of P450 BM3 variant 4E10 was verified. Calculations based on the expression levels obtained by the plasmid expression system showed that in the final engineered microorganism, consumption of NAD(P)H by the biocatalyst was the rate limiting step in glucose metabolism. In order to determine optimal expression levels, strains expressing varying levels of biocatalyst were prepared and used. With the biocatalyst gene incorporated into the host microorganism genome, antibiotic and inducing agents were no longer required to produce biocatalyst during cell growth. Instead, biocatalyst production was controlled initially by the presence of nitrogen in the medium and later by modifications in the metabolic regulation machinery in the cell (described below). For a process to function unassisted for extended periods of time, enough nitrogen was fed to the process over time to replace inactivated biocatalysts and other cell components. The presence of too much nitrogen, however, leads to accumulated biomass which may adversely affect the process. With each strain, a nitrogen feed rate was determined that keeps the whole cells functioning as biocatalysts that generate the alcohol product at a constant rate.

To determine the rate at which the biocatalyst should be replaced inside the microorganism over time, engineered E. coli strains expressing P450 BM3 variant 4E10 were first grown in a nitrogen-containing rich medium and then placed into minimal medium in a fermenter as described above. The microorganisms were then used to transform propane into propanol. Periodically, aliquots of cells were removed from the fermenter and the amount of active P450 BM3 variant 4E10 inside these aliquots was measured using an activity assay and compared to the total amount of expressed biocatalyst determined from SDS-PAGE gels. (which measure both active and inactive protein). A nitrogen feed rate that just replaces the inactivated biocatalyst over time was then determined empirically by similarly measuring active biocatalyst concentrations in the fermenter over time.

The transcriptional unit from the Pbm3-4E10 plasmid was amplified with the primers 7nuo_tacBM3F and 10BM3R. The km resistance cassette was amplified from Pkd13 using primers 9nuoA_NF and 1nuoA_NR. The two PCR products were used in an overlap extension reaction with the primers 7nuo_tacBM3F and 1nuoA_NR. The product of the SOE reaction was transformed into W3110(Pkd46) and the resulting strain, GEVO734, contained Plactactac::BM3(4E10)::FRT-kan-FRT in place of the nuoA_N operon. The SOE product was also transformed into WA837(Pkd46). From the resulting strain, GEVO748, the replacement was transduced into E. coli B yielding GEVO1318.

For the construction of the replacement of ndh with lactactacBM3(4E10) the transcription unit was amplified from Pbm3(4E10) using the primers 8ndh_tacBM3F and 10BM3R. The km resistance cassette was amplified from Pkd13 using primers 9nuoA_NF and 4ndhR. The two PCR products were used in an overlap extension reaction with the primers 8ndh_tacBM3F and 4ndhR. The product of the SOE reaction was transformed into W3110(Pkd46). The resulting strain GEVO736 contained Plactactac::BM3(4E10)::FRT-km-FRT in place of the ndh gene. A phage P1 lysate of GEVO736 was prepared and the deletion was transferred into WA837. From the resulting strain, GEV-0752, the replacement was transduced into E. coli B yielding GEVO785.

To enable the expression of BM3(4E10) under the regulatory control of the host strain, the gene coding for 4E10 was fused to the promoters of the nuoA_N operon and of the ndh gene thereby replacing the genes coding for NDH1 and NDH2. The 4E10 gene was amplified from the plasmid Pbm3(4E10) with the primers 6nuo_BM3F and 10BM3R. The km resistance cassette was amplified from Pkd13 using primers 9nuoA_NF and 1nuoA_NR. The two PCR products were used in an overlap extension reaction with the primers 6nuo_BM3F and 1nuoA_NR. The product of the SOE reaction was transformed into W3110(Pkd46). The resulting strain GEVO711 contained PnuoA::BM3(4E10)::FRT-ka-FRTin place of the nuoA_N operon. The SOE product was also transformed into, WA837(Pkd46). From the resulting strain, GEVO746, the replacement was transduced into E. coli B yielding GEVO717. For the corresponding replacement of ndh the 4E10 gene was amplified from the plasmid Pbm3(4E10) with the primers 5ndh_BM3F and 10BM3R. The km resistance cassette was amplified from Pkd13 using primers 9nuoA_NF and 4ndhR. The two PCR products were used in an overlap extension reaction with the primers 5ndh_BM3F and 4ndhR. The product of the SOE reaction was transformed into W3110(Pkd46). The resulting strain, GEVO744, contained Pndh::BM3(4E10)::FRT-kan-FRT in place of the ndh gene. The SOE product was also transformed into WA837(Pkd46). From the resulting strain GEVO747 the replacement was transduced into E. coli B yielding GEVO784.

To remove both NADH dehydrogenases from E. coli and replace them with BM3(4E10), the replacements of ndh and nuoA_N with BM3(4E10) were combined. GEVO738 was transduced with the lysate of GEVO744 and the resulting double replacement strain with 4E10 under native control was designated GEVO759. The same procedure was used to construct the double replacement of nuoA_N and ndh in E. coli B (GEVO1319). For the construction of the double replacement strain with 4E10 under control of the lactactac promoter, the kan^{R} cassette was removed from the chromosome of GEVO734 with FLP recombinase. The resulting strain, GEVO1320, was transduced with the lysate of GEVO736 and the resulting double replacement strain was designated GEVO1321. The same procedure was used to construct the double replacement of nuoA_N and ndh in E. coli B (GEVO1322).

Strains featuring the deletion of both NDH1 and NDH2 (and either one of these replaced with 4E10) were constructed. GEVO738 was transduced with a P1 lysate prepared from GEVO713 and the resulting double deletion strain with nuoA_N replaced with PnuoA::BM3(4E10)::FRT-kan-FRT was named GEVO751. The kan^{R} cassette was removed from the chromosome of GEVO715 with FLP recombinase. The resulting strain, GEVO741, was transduced with a P1 lysate of GEVO744 and the resulting double deletion strain with ndh replaced with Pndh::BM3(4E10)::FRT-kan-FRT was named GEVO757. GEVO740 was transduced with a P1 lysate prepared from GEVO734 and the resulting strain with nuoA_N replaced with Plactactac::BM3(4E10)::FRT-kan-FRT was named GEVO763. GEVO741 was transduced with a P1 lysate prepared from GEVO736 and the resulting strain with ndh replaced with Plactactac::BM3(4E10)::FRT-kan-FRT was named GEVO765.

The same strategy was used to make the strains with double deletion and single replacement in E. coli B (GEVO1323, GEVO1324, GEVO1326, GEVO1325).

### Example 8

### Removal of Overflow Pathways from Host Microorganism Genome

The alternative NADH overflow pathways lead to the production of compounds such as succinate, lactate, acetate, ethanol, formate, carbon dioxide and hydrogen gas and, when activated, greatly decrease the amount of NADH that can be obtained by breaking down glucose. The engineered microorganisms described above accumulate NADH unless the biocatalyst is present to remove the cofactor as it is produced. If the biocatalyst activity is not high enough to consume all of the NADH that is generated through normal aerobic metabolism, the overflow pathways are activated by the increased NADH levels and compete with the biocatalyst. Once the overflow pathways are activated, glucose is converted into other substances, such as acetate, that will adversely affect the microorganisms and limit the yield of a whole-cell biocatalytic process. Methods for removing the key enzymes from each pathway are described below.

D-lactate dehydrogenase (ldhA): Most of the gene coding for the lactate dehydrogenase in E. coli (ldhA) was deleted (nucleotides 11-898 were deleted). Pkd13 was used as the template for the PCR with 411dhA_ko_f and 42ldhA_ko_r as forward and reverse primers (Table 9). W3110(Pkd46) was transformed with the PCR product. The resulting strain, GEVO788, contained FRT-kan-FRT in place of the ldhA gene. The PCR product was also transformed into WA837(Pkd46) yielding GEVO789. The deletion of ldhA was combined with the deletions of nuoA_N and ndh. The kan^{R} cassette was removed from the chromosome of GEVO750 with FLP recombinase. The resulting strain, GEVO1327, is transduced with a P1 lysate prepared from GEVO788 and the resulting strain is designated GEVO1328. For the construction of the corresponding E. coli B strain, the kan^{R} cassette is removed from the chromosome of GEVO1317 with FLP recombinase. The resulting strain, GEVO1329, is transduced with a P1 lysate prepared from GEVO789 and the transduced strain is designated GEVO 1330.

Acetaldehyde/alcohol dehydrogenase (adhE): The gene coding for the alcohol dehydrogenase in E. coli (adhE) is disrupted with a deletion (nucleotides-308-2577 are deleted). Pkd13 is used as the template for the PCR with 49adhE_ko_f and 50adhE_ko_r as forward and reverse primers (Table 9). W3110(Pkd46) is transformed with the PCR product and the resulting strain, GEVO800, contains FRT-kan-FRT in place of the adhE gene. The PCR product is also transformed into WA837(Pkd46) yielding GEVO803. The deletion of adhE is combined with the deletions of nuoA_N, ndh and ldhA. The kan^{R} cassette is removed from the chromosome of GEVO1328 with FLP recombinase. The resulting strain, GEVO1331, is transduced with a P1 lysate prepared from GEVO800 and the resulting strain is designated GEVO 831. For the construction of the corresponding E. coli B strain the kan^{R} cassette is removed from the chromosome of GEVO1330 with FLP recombinase. The resulting strain, GEVO1332, is transduced with a P1 lysate prepared from GEVO803 and the transduced strain is designated GEVO 1333.

Pyruvate formate lyase (pflB): The gene coding for the pyruvate formate lyase in E. coli (pflB) is disrupted by the deletion of focA and pflB (nucleotides −69(focA)-2240(pflB) are deleted). Pkd13 is used as the template for the PCR with 47focApflB_ko_f and 48focApflB_ko_r as forward and reverse primers (FIG. 12). W3110(Pkd46) is transformed with the PCR product. The resulting strain, GEVO802, contains FRT-kan-FRT in place of the focA-pflB operon. The PCR product is also transformed into WA837(Pkd46) yielding GEVO805. The deletion of pflB is combined with the deletions of nuoA_N, ndh, ldhA and adhE. The kan^{R} cassette is removed from the chromosome of GEVO831 with FLP recombinase. The resulting strain GEVO1334 is transduced with a P1 lysate prepared from GEVO802 and the resulting strain is designated GEVO 1335. For the construction of the corresponding E. coli B strain the kan^{R} cassette is removed from the chromosome of GEVO1333 with FLP recombinase. The resulting strain, GEVO1336, is transduced with a P1 lysate prepared from GEVO805 and the transduced strain is designated GEVO 1337.

Fumarate reductase (frd): The genes coding for the fumarate reductase in E. coli (frdABCD) are disrupted with a deletion of frdABCD (nucleotides −86(frdA)-178(frdD) are deleted). Pkd13 is used as the template for the PCR with 55frd_ko_f and 56frd_ko_r as forward and reverse primers (Table 9). W3110(Pkd46) is transformed with the PCR product and the resulting strain, GEVO818, contains FRT-kan-FRT in place of the frdABCD operon. The PCR product is also transformed into WA837(Pkd46) yielding GEVO822. The deletion offrdABCD is combined with the deletions of nuoA_N, ndh, ldhA, adhE and focA-pflB. The kan^{R} cassette is removed from the chromosome of GEVO1335 with FLP recombinase. The resulting strain, GEVO1338, is transduced with a P1 lysate prepared from GEVO818 and the resulting strain is designated GEVO1339. For the construction of the corresponding E. coli B strain the kan^{R} cassette is removed from the chromosome of GEVO1337 with FLP recombinase. The resulting strain, GEVO1340, is transduced with a P1 lysate prepared from GEVO822 and the transduced strain is designated GEVO1341.

Acetate kinase A (ackA): The gene coding for acetate kinase in E. coli (ackA) is disrupted with a deletion (nucleotides 29-1062 are deleted). Pkd4 is used as the template for the PCR with 53ackA_ko_f and 54ackA_ko_r as forward and reverse primers (Table 9). W3110(Pkd46) is transformed with the PCR product and the resulting strain, GEVO817, contains FRT-kan-FRT in place of the ackA gene. The PCR product is also transformed into WA837(Pkd46) yielding GEVO821. The deletion of ackA is combined with the deletions of nuoA_N, ndh, ldhA, adhE, focA-pflB and frdABCD. The kan^{R} cassette is removed from the chromosome of GEVO1339 with FLP recombinase. The resulting strain, GEVO1342, is transduced with a P1 lysate prepared from GEVO817 and the resulting strain is designated GEVO1343. For the construction of the corresponding E. coli B strain the kan^{R} cassette is removed from the chromosome of GEVO1341 with FLP recombinase. The resulting strain, GEVO1344, is transduced with a P1 lysate prepared from GEVO821 and the transduced strain is designated GEVO 1345.

Pyruvate oxidase (poxB): The gene coding for pyruvate oxidase in E. coli (poxB) is disrupted with a deletion in poxB (nucleotides 30-1600 are deleted). Pkd4 is used as the template for the PCR with 51poxB_ko_f and 52poxB_ko_r as forward and reverse primers (Table 9). W3110(Pkd46) is transformed with the PCR product and the resulting strain, GEVO801, contains FRT-kan-FRT replacing part of the poxB gene. The PCR product is also transformed into WA837(Pkd46) yielding GEVO804. The deletion of poxB is combined with the deletions of nuoA_N, ndh, ldhA, adhE, focA-pflB, frdABCD and ackA. The kan^{R} cassette is removed from the chromosome of GEVO1343 with FLP recombinase. The resulting strain, GEVO1346, is transduced with a P1 lysate prepared from GEVO801 and the resulting strain is designated GEVO1347. For the construction of the corresponding E. coli B strain, the kan^{R} cassette is removed from the chromosome of GEVO1345 with FLP recombinase. The resulting strain, GEVO1348, is transduced with a P1 lysate prepared from GEVO804 and the transduced strain is designated GEVO1349.

### Example 9

### Cytochrome P450 Catalyzed Monooxygenation Reactions

Bioconversions were performed with E. coli BL21 microorganisms transformed with a plasmid carrying BM3 variant 4E10. Propane was chosen as a substrate. A culture of E. coli BL21 harboring plasmid Pbm3_{—}4E10 was grown in 500 Ml of M9 medium containing 0.4% glucose, 100 μg/Ml ampicillin and 2% (w/v) yeast extract. IPTG (1 Mm) was added after 12 h and the culture was grown for an additional 24 h. Microorganisms were then acclimated in M9 medium for three hours, harvested by centrifugation and stored at 4° C. before starting a bioconversion. Biotransformations were then carried out as described above. Bioconversions with cell lysate were carried out with the lysate from exactly the same amount of cells. Propane and oxygen were bubbled through the cell suspension as described above. The concentration of propanol, glucose and organic metabolites in the fermentation broth was assayed as described above.

Nitrogen was omitted from the biotransformation medium to limit microorganism growth and to thereby channel reducing equivalents away from biosynthetic pathways and into the conversion of propane to propanol. Oxygen and propane were bubbled through the cell suspension (ca. 5 g of cells per L) and propanol concentrations were determined over time.

The results illustrated in FIG. 12, show that the propanol formation rate was approximately 300 mg/L (5 Mm) of propanol for the first hour and then slowly decreased to yield a maximum propanol concentration of 600 mg/L (10 Mm) after three hours. Even though neither the cells nor the reaction conditions were optimized, these numbers are 20 times higher than those measured using cell lysate containing the same amount of P450 catalyst. The decline in propanol formation after three hours can be attributed to a number of factors, including limited glucose feed, evaporation of product and stability of the catalyst. The results nevertheless show that a recombinantly expressed cytochrome P450 can be used in whole E. coli cells for biotransformations.

These cells, however, perform as expected in terms of the ratio of product formation over glucose consumption and yield about 1 product molecule per molecule of glucose consumed. In the engineered microorganism GEVO 1349, recombinantly expressing a cytochrome P450, the product yield per glucose consumed is at least five times higher than in non-engineered E. coli cells.

### Example 10

### Compared NADH Availability in Recombinant Microorganism and Wild-Type

To test the influence of P450 catalysis on metabolite distribution we expressed the catalyst from a plasmid, fed glucose and propane and measured ethanol metabolite and propanol product formation. The expression experiment in FIG. 12 shows that expression of the P450 biocatalyst can redirect the NADH flux from the alcohol dehydrogenase towards the biocatalyst. Per mole of glucose one mol of acetate and one mol of ethanol are produced when there is no P450 expressed. Upon expression of the biocatalyst the majority of the reducing equivalents are redirected towards propanol formation. Ethanol fermentation was observed in GEVO750 (Δndh, Δnuo) as the strain is respiratory negative and regenerates reducing equivalents by ethanol fermentation. Results are illustrated in FIG. 12a and show that P450 biocatalysis can effectively compete with fermentation.

Since plasmid-based P450 expression proved detrimental for cell viability (for detailed discussion see section below), one copy of the P450 4E10 (a P450 variant with comparable in vitro activity than 19A12) variant including a promoter was inserted into the genome resulting in GEVO829(Δndh, Δnuo, Δadh, Δldh, +P450 4E10). GEVO829 yielded about four times higher propanol yields when compared to plasmid-based P450 expression. We compared this strain with a wild-type strain expressing 4E10 from a plasmid and measured product per glucose ratios. Comparative propane to propanol conversion could not detect significant differences in productivity and metabolites of whole-cell biocatalysis of wild-type (GEVO706) and Gevo engineered strain (GEVO 829).

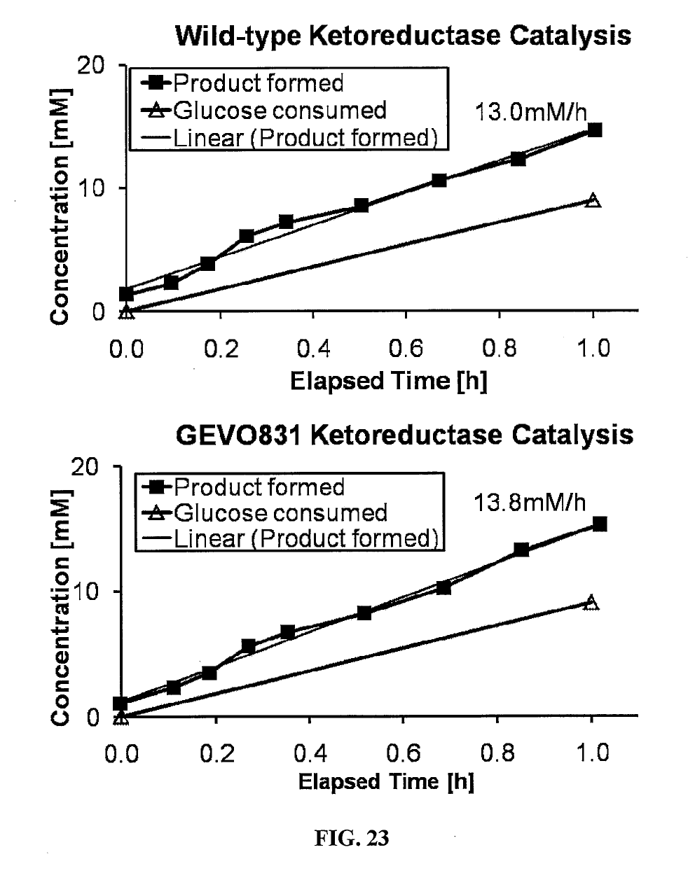

In order to verify these results and the effect of the metabolic engineering strategy we chose a different biocatalyst that is also NADPH-dependent and well-characterized {Walton, 2004 #1166}. This biocatalyst, a ketoreductase, reduces ethyl acetoacetate to ethyl 3-hydroxybutyrate. There are several advantages using this biocatalyst as test system for verifying the metabolic engineering strategy. For once this catalyst does not require a gaseous substrate and second does not require oxygen feeding. This approach eliminates experimental difficulties associated with gas supply, but nevertheless allows the determination of a product per glucose ratio to validate the metabolic engineering strategy. The catalyst is also very well expressed and does not have coupling problems—that is using reducing equivalents without performing the desired catalysis reaction—that might lead to highly reactive intermediates harmful for the cell. Improving coupling efficiency has also been one goal of the catalyst engineering and its progress is documented in FIG. 23.

Whole cell comparative biocatalysis experiments using the ketoreductase confirmed that the engineered quadruple knock-out (GEVO831, Δndh, Δnuo, Δadh, Δldh) and wild-type strain (GEVO706) do not exhibit significantly different product formation rates or product/glucose ratios. The ketoreductase biocatalyst yielded product/glucose ratios for wild-type and engineered strains of 4

GEVO831 was designed to eliminate the primary NADH sinks to produce up to 10 product molecules per glucose. These engineered cells do not exhibit statistically significant differences in their product/glucose ratio compared to unmodified cells and exhibit respiratory activity as measured by oxygen consumption.

Therefore, the microorganism has activated alternative NAD(P)H dehydrogenase-like enzymes or pathway that outcompetes the NAD(P)H-requirement of the biotransformation.

### Example 11

### Methane Monooxygenases Catalyzed Conversion of Methane to Methanol

In the engineered E. coli GEVO 1349 cells, the selective pressure for expression of an NADH utilizing enzyme allows for expression of soluble methane monooxygenase. In addition, at least five times more methanol per molecule of glucose is produced than in non-engineered E. coli cells.

### Example 12

### Styrene Monooxygenase

In the engineered GEVO 1349 microorganisms, recombinantly expressed styrene monooxygenase yields at least five product molecules per molecules of glucose consumed.

### Example 13

### Baeyer-Villiger Monooxygenases

In non-growing E. coli cells (i.e. without a nitrogen source in the medium) expressing this cyclohexanone Baeyer-Villiger monooxygenase, the ratio of product formation over glucose consumption is ca. 1.0 (Walton, A. Z. et al, 2002, Biotechnol. Prog., 18, 262-68). In the engineered GEVO 1349 microorganism, the same recombinantly expressed Baeyer-Villiger monooxygenase (Walton, A. Z. et al, 2002, Biotechnol. Prog., 18, 262-68) yields at least 5 product molecules per molecules of glucose consumed.

### Example 14

### Ketoreductases

Gre2p; an NADPH-dependent short-chain dehydrogenase from Saccharomyces cerevisiae that reduces a variety of ketones with high stereoselectivity, was engineered in E. coli. The enzyme was overexpressed in E. coli using standard procedures (Walton, A. Z. et al, 2004, Biotechnol. Prog., 20, 403-11), and the biotransformation of ethyl acetoacetate to ethyl-3-hydroxybutyrate carried out using the same procedure as described above. This whole cell biocatalytic conversion proceeded with a yield of at least five product molecules per molecule of glucose and may be achieved using the engineered GEVO 1349 E. coli cells of this disclosure.

### Example 15

### Replacement of Alpha-Ketoglutarate Dehydrogenase in E. coli

The recombinant microorganisms disclosed in the following exemplary embodiment are organisms in which the native E. coli alpha-ketoglutarate dehydrogenase is replaced by an alternative alpha-ketoglutarate dehydrogenase that is inhibited by higher NADH-levels than the native enzyme. This removes one of the bottlenecks that would prevent the TCA cycle from functioning in a manner that allows the biocatalyst to consume greater than four NADH molecules per glucose. Since alpha-ketoglutarate dehydrogenase shares the same lpdA subunit responsible for NADH inhibition of pyruvate dehydrogenase, a similar mutation in lpdA that allows for growth under anaerobic conditions will allow alpha-ketoglutarate dehydrogenase to function under high NADH concentrations. GEVO1182 was generated by GEVO788, GEVO802 and GEVO818 by subsequent removing of the resistance cassette and transduction using the homologous recombination in the presence of Red recombinase according to Datsenko, K. A. et al, 2000, Proc. Nat. Acad. Sci. USA, 97, 6640-45. This strain does not grow under anaerobic condition on LB plates containing 1% glucose. Mutagenizing agent MNNG(N-methyl-N′-nitro-N-nitrosoguanidine) was applied to the cells and mutagenized cells selected for growth, under anaerobic condition on LB plates containing 1% glucose. Four colonies were isolated after restreaking, GEVO1283, GEVO1284, GEVO1285 and GEVO1286. These strains were characterized by metabolite analysis and showed Ethanol: Acetate ratios>15.

These results are in accordance with Kim, Ingram. Y. et al. 2007, Applied and Environmental Microbiology, 73(6), 1766-71

### Example 16

### Replacement of Citrate Synthase in a E. coli

The recombinant microorganisms disclosed in the following exemplary embodiment are organisms in which the native E. coli citrate synthase is replaced by an alternative citrate synthase that is inhibited by higher NADH-levels than the native enzyme. This removes one of the bottlenecks that would prevent the TCA cycle from functioning in a manner that allows the biocatalyst to consume greater than four NADH molecules per glucose.

A first approach to mutate the citrate synthase is the following; After gene deletion of the endogenous enzyme according to methods described elsewhere) the alternative enzyme can either be expressed from an expression plasmid (e.g. by pZ vector system described by Lutz, R. and H. Bujard (1997) Nucleic Acids Res 25(6): 1203-10.) or it can be integrated into the genome using commonly used methods.

The gene coding for the modified citrate synthase that includes one or more of the mutations, Y145A, R163L, K167, and D362N, was generated by SOE PCR using primers that encode the desired mutations. As template the genes encoding the above mentioned enzymes were used. The gene will be later integrated into the E. coli chromosome.

Once inserted, the expression of the citrate synthase variant can be verified by enzymatic assays (according to Srere P. A., Brooks G. C., Arch Biochem Biophys. 1969 February; 129(2):708-10.) measured by absorbance change at 412 nm upon the formation of citrate. With the gene incorporated into the host microorganism genome, antibiotic and inducing agents were no longer required to produce citrate synthase during cell growth.

For genomic insertion the transcriptional unit from a plasmid encoding for the mutated citrate synthase was amplified with corresponding primers. The km resistance cassette was amplified from Pkd13 using with corresponding primers. The two PCR products were used in an overlap extension reaction. The product of the SOE reaction would be transformed into W3110(Pkd46) and after selecting for km resistance and verifying the genomic insertion the resulting strain would express the modified citrate synthase that is no longer NADH inhibited

Furthermore this mutation or mutations or this replacement will likely remove the NADH inhibition of the citrate synthase) is activity and result in partial removal of the catabolite repression and activity of the therefore show increased TCA cycle activity while feeding glucose or other energy rich carbon sources to the cells. This is measurable by increased carbon dioxide production relative to levels generated by cells with inactive TCA cycle and would also results in increased NADH availability for biocatalysis. If the TCA cycle is active and the biocatalytic enzyme or pathway is active, one would see increased product per glucose yield that is greater than 4 (but less than 10).

To avoid the presence of a plasmid carrying the gene coding for the modified citrate synthase in the engineered strain, the gene encoding the modified citrate synthase that includes the mutations Y145A, R163L and K167 was generated by using primers that encode the desired mutations by SOE. The gene will be later integrated into the E. coli chromosome.

Once inserted, the expression of the citrate synthase variant is verified by enzymatic assays measuring according to Srere P. A., Brooks G. C., Arch Biochem Biophys. 1969 February; 129(2):708-10.) by following absorbance change, at 412 nm upon the formation of citrate. With the gene incorporated into the host microorganism genome, antibiotic and inducing agents were no longer required to produce biocatalyst during cell growth.

For genomic insertion the transcriptional unit from a plasmid encoding for the mutated citrate synthase was amplified with corresponding primers. The km resistance cassette was amplified from Pkd13 using with corresponding primers. The two PCR products were used in an overlap extension reaction. The product of the SOE reaction would be transformed into W3110(Pkd46) and after selecting for km resistance and verifying the genomic insertion the resulting strain would express the modified citrate synthase that is no longer NADH inhibited.>

The expected effect (only in addition to the removal of the NADH inhibition of citrate synthase and alpha-ketoglutarate dehydrogenase is the partial removal of catabolite repression and activity of the TCA cycle while feeding glucose or other energy rich carbon sources. In the presence of a pathway accepting reducing equivalents this activity would also be true under anaerobic condition or absence of other electron acceptors. The effect would be increased carbon dioxide production through the TCA cycle and would also results in increased NADH availability for biocatalysis. If TCA cycle is active one would see increased product per glucose yield that is greater than 4 (but smaller than 12).

### Example 17

### Replacement of Fumarate Reductase (Succinate Dehydrogenase) in a E. coli

The enzyme has two catalytic subunits (SdhA, SdhB) plus two membrane subunits (SdhC, SdhD). The succinate oxidation reaction, which is part of the aerobic respiratory chain and part of the Krebs cycle, oxidizes succinate to fumarate while reducing ubiquinone to ubiquinol. It is closely related to fumarate reductase, which carries out the reverse reaction. The succinate dehydrogenase and fumarate reductase can replace each other [Guest, J. R. J. Gen. Microbiol. (1981) 122, 171.]. 110110-Succinate dehydrogenase is made under aerobic conditions with succinate or acetate as a carbon source. Enzyme synthesis is regulated by catabolite repression [Wilde R J, Guest J R (1986). “Transcript analysis of the citrate synthase and succinate dehydrogenase genes of Escherichia coli K12.” J Gen Microbiol 1986; 132 (Pt 12); 3239-51.]. Activation of the enzyme by covalent attachment of FAD to the SdhA enzyme subunit is promoted by intermediates of the TCA cycle [Brandsch R, Bichler V (1989). “Covalent cofactor binding to flavoenzymes requires specific effectors.” Eur J Biochem 1989; 182(1); 125-8]. 110110Fumarate reductase is made under anaerobic conditions with glucose as a carbon source. Succinate dehydrogenase and fumarate reductase functions are partially interchangeable if their regulation is manipulated such that succinate dehydrogenase is produced under anaerobic conditions or fumarate reductase is produced aerobically [Guest J R (1981). “Partial replacement of succinate dehydrogenase function by phage- and plasmid-specified fumarate reductase in Escherichia coli.” J Gen Microbiol 1981; 122(Pt 2); 171-9; Maklashina E, Berthold D A, Cecchini G (1998). “Anaerobic expression of Escherichia coli succinate dehydrogenase: functional replacement of fumarate reductase in the respiratory chain during anaerobic growth.” J Bacteriol 1998; 180(22); 5989-96]. We cloned the glyoxysomal fumarate from Trypanosoma brucei into an expression vector pZ21. One sequence was obtained from amplification of genomic DNA with specific primers (773Tryp_kpn_f and 775Tryp_sal_r).

The gene was also synthesized artificially and cloned into the pZ21 expression vector. Functional heterologous expression in E. coli is verified by enzyme assays measuring NADH consumption when converting fumarate to succinate. Then this gene will be inserted genomically to replace the endogenous succinate dehydrogenase as described for other enzymes in example above using homologs recombination according to Datsenko, K. A. et al, 2000, Proc. Nat. Acad. Sci. USA, 97, 6640-45endogenous.

### Example 18

### Removal of NADH Dehydrogenase in Yeast

Mitochondrial NADH is oxidized and its electrons transferred to the ubiquinone chain via the action of respiratory complex I or an internal NADH dehydrogenase. In the yeast, S. cerevisiae, respiratory complex I is not present. The internal NADH dehydrogenase is encoded by the NDI1 gene. This gene can be deleted by directed double homologous recombination using a PCR product containing from 5′ to the 3′ end, a 70 bp targeting homology region to 70 bp of endogenous sequence just upstream of the start of the NDI1 coding region, the K. lactis URA3 marker, a region (200-300 bp) of homology to the promoter region of NDI1 that is upstream of the targeting homology region, and 70 bp of endogenous sequence just downstream of the stop of the NDI1 coding region. The marker (K lactis URA3) can be amplified from pGV1299 by PCR where the 5′ primer introduces 70 bp homology region upstream of the NDI1 start codon. The 200-300 bp region of homology to the NDI1 promoter sequence is amplified from S. cerevisiae genomic DNA using a 5′ primer that introduces and overlap with the 3′ end of the amplified K. lactis URA3 marker. These two PCR fragments are combined by SOE using the 5′ primer above that introduces 70 bp homology upstream of the NDI1 start codon and a 3′ primer that introduces 70 bp homology downstream of the NDI1 stop codon. The resulting product is transformed into S. cerevisiae and integrations are selected on media lacking uracil. The disruption of the gene can be confirmed by colony PCR using a primer directed to the K. lactis URA3 and a primer directed to a sequence outside the NDI homology region. Also, the resulting integration would result in the K. lactis URA3 marker being flanked by 200-300 bp region of homology. This will result, at a low but significant frequency, in the K. lactis URA3 marker being removed by recombination between these homologous sequences. These events can be detected by growth on 5-FOA, which allows for selection for strains that have lost the K. lactis URA3 gene.

### Example 19

### Removal of External NADH Dehydrogenases and Glycerol-3-Phosphate Dehydrogenases in Yeast

In the yeast S. cerevisiae, Cytoplasmic NADH is oxidized and its electrons transferred to the ubiquinone pool via external NADH dehydrogenases (NDE1 and NDE2) and indirectly via soluble glycerol-3-phosphate dehydrogenases (GPD1 and GPD2) and a membrane bound glycerol-3-phosphate dehydrogenase (GUT2) (Figure). To prevent NADH from entering the respiratory chain, the two NADH dehydrogenases, NDE1 and NDE2, and the soluble glycerol-3-phosphate dehydrogenases, GPD1 and GPD2, are deleted as described above for NDI1. As the marker is reusable in this method, the genes are deleted in series.

### Example 20

### Additional Deletions

In addition to the above NADH reductases, other reductases that would compete with the biocatalyst need to be inactivated. Specifically, cytoplasmic alcohol dehydrogenase genes, which include ADH1, ADH2, ADH4, ADH5, and SFA1, can be deleted as described above.

### Example 21

### Heterologous Pathway/Oxygenase in Yeast

The additional NADH generated by the engineered TCA cycle can be utilized by the cytochrome P450 BM3. This gene is cloned into a yeast expression vector and placed under the control of a constitutive yeast promoter, such as the promoters for the TEF2 or TDH3 genes. This plasmid is transformed into the engineered yeast strain and the activity is tested by assessing the ability of this transformed strain to convert propane to propanol as described above.

### Example 22

### Insertion TCA in Clostridia

Based on FIG. 12 and the description of the enzymatic reactions that comprise a complete TCA cycle (paragraph 138), it is trivial to deduce that Clostridium acetobutylicum ATCC 824 could harbor a complete TCA cycle by providing it with the capability of convert converting succinyl-CoA to succinate and then succinate to fumarate. It will also require the capability of converting oxalacetate plus acetyl-CoA to citrate. The first of these conversions can be carried out either by introducing the enzymatic activities EC 6.2.1.4 (known among other names as succinyl-CoA synthetase), EC 6.2.1.5 (known among other names as succinyl-CoA synthetase) or EC 3.1.2.3 (known among other names as succinyl-CoA hydrolase). This step can be carried out by the combined action of the alpha and beta subunits of the succinyl-CoA synthetase encoded by the E. coli genes sucD (NCBI-GeneID: 945314) and sucC (NCBI-GeneID: 945312) respectively. These two genes are contiguous in the E. coli genome and the distance from the start codon of sucC to the stop codon of the preceding gene (sucB) is of 275 bp. The facts strongly suggest that sucC and sucD are transcribed as single mRNA. The second conversion (succinate to fumarate) requires the introduction of the enzymatic activities EC 1.3.5.1 (known among other names as succinate dehydrogenase) or EC 1.3.99.1 (known among other names as succinate dehydrogenase). In this example, the conversion of succinate into fumarate will be carried out by the same engineered, enzyme used for the E. coli example (paragraph 00281). The conversion of oxalacetate plus acetyl-CoA into citrate can be carried out by the citrate synthase of Clostridium kluyveri DSM 555 (Li F et al. “Re-citrate synthase from Clostridium kluyveri is phylogenetically related to homocitrate synthase and isopropylmalate synthase rather than to Si-citrate synthase.” J. Bacteriol. June; 189(11):4299-304. 2007). The sequences for these three genes could be easily obtained from public databases and primers for their amplification can the generation of sucC and sucD as a single PCR product can be easily be designed for performed by somebody skilled in the art. The same applies for the generation of the PCR fragment for the coding region of the citrate synthase of C. kluyveri.

For the obtention of the polypeptide carrying out the conversion of succinate to fumarate refer to the E. coli example. The PCR fragment containing sucC and sucD could then be introduced into the plasmid pSOS95 (Tummala, S. B., et al “Development and characterization of a gene-expression reporter system for Clostridium acetobutylicum ATCC 824”, Appl. Environ. Microbiol. 65: 3793-3799 (1999)). Also in the same plasmid, we could then introduce the PCR fragment comprising the ptb promoter from plasmid pHT4 (Tummala, S. B., et al “Development and characterization of a gene-expression reporter system for Clostridium acetobutylicum ATCC 824”, Appl. Environ. Microbiol: 65: 3793-3799 (1999)) to control the expression of the PCR fragment of the coding region of the citrate synthase of C. kluyveri. We will refer to this construct as plasmid A. The PCR product containing the polypeptide responsible for the conversion of succinate into fumarate could be introduced into plasmid pTLH1 (Harris, L. M. et al “Characterization of Recombinant Strains of the Clostridium acetobutylicum Butyrate Kinase Inactivation Mutant: Need for New Phenomenological Models for Solventogenesis and Butanol Inhibition? ” Biotechnology and Engineering, 67 (1): 1-11 (2000). However this plasmid will require the insertion of a suitable promoter in front the polypeptide of interest. This promoter could be the ptb promoter contained in plasmid pSOS94 (Tummala, S. B., et al “Development and characterization of a gene-expression reporter system for Clostridium acetobutylicum ATCC 824”, Appl. Environ. Microbiol. 65: 3793-3799 (1999)). We will refer to this engineered plasmid as plasmid B.

These two new plasmids then used to independently transform the recombinant E. coli strain ER2275 (pAN1) which harbors the Φ3T I methyltransferase encoded by the Bacillus subtilis phage Φ3T as documented in Mermelstein, L. D. and Papoutsakis, E. T., “In vivo methylation in Escherichia coli by the Bacillus subtilis phage Φ3T I Methyltransferase to protect plasmids from restriction upon transformation of Clostridium acetobutylicum ATCC 824”, Appl. Environ. Microbiol. 59: 1077-1081 (1993). This step will ensure the proper methylation of the plasmid to avoid its degradation by the action of C. acelobutylicum ATCC 824 DNAases, especially Cac824I. After the successful extraction of plasmid A and plasmid B from the independent cultures, they can be introduced into Clostridium acetobutylicum ATCC 824 by electroporation following the protocols described in Mermelstein, L. D. and Papoutsakis, E. T., “In vivo methylation in Escherichia coli by the Bacillus subtilis phage. Φ3T I Methyltransferase to protect plasmids from restriction upon transformation of Clostridium acetobutylicum ATCC 824”, Appl. Environ. Microbiol. 59: 1077-1081 (1993). After selection of a clone resistant to both erythromycin (i.e. carrying plasmid A) tetracycline (i.e. carries plasmid B), the expression and activity of the polypeptides should be checked. The transcription of the polypeptides could be checked by Q-RT-PCR and Northern Blot, their expression by a Western Blot or ELISA assay, and their in vitro activity could be checked by performing an in vitro activity assay. Determination of the activity in vivo could be carried out by comparative analysis of the levels of the reaction products between the plasmid control strain (i.e. a strain transformed with a plasmid without the DNA encoding the polypeptide of interest) and the successful clone. The verification of the activity of the engineered TCA can be carried out by in vivo fluorimetry and or by the use of substrates labeled with ¹⁴C (radioactive) or ¹³C substrates and then analyzing the incorporation of the labeled carbon into the intermediates of the TCA.

### Example 23

### Insertion of Glvoxylate Shunt in Clostridia

Based on tables 4 and 5 and the description of the enzymatic reactions that comprise the glyoxylate shunt (paragraph 140), it is trivial to deduce that Clostridium acelobutylicum ATCC 824 could harbor glyoxylate shunt by introducing the capability of converting iso-citrate to glyoxylate, glyoxylate to malate and then succinate to fumarate. It will also require the capability of converting oxalacetate plus acetyl-CoA to citrate. The first of these conversion isocitrate to glyoxylate requires the presence of the enzymatic activity EC 4.1.3.1 (known among other names as isocitrate lyase). In E. coli this enzymatic activity is encoded by the isocitrate lyase gene (aceA or icl, NCBI-GenelD: 948517). The second step (conversion of glyoxylate into malate) requires the introduction of the enzymatic activity EC 2.3.3.9 (known among other names as malate synthase) which in E. coli is encoded by the product of the malate synthase G gene (glcB or glc, NCBI-GeneID: 948857). The third step (conversion of succinate to fumarate) requires the introduction of the enzymatic activities EC 1.3.5.1 (known among other names as succinate dehydrogenase) or EC 1.3.99.1 (known among other names as succinate dehydrogenase). The third step (conversion of succinate to fumarate) requires the introduction of the enzymatic activities EC 1.3.5.1 or EC 1.3.99.1. In this example, the conversion of succinate into fumarate will be carried out by the same engineered enzyme used for the E. coli example (paragraph 00281).

The conversion of oxalacetate plus acetyl-CoA into citrate can be carried out by the citrate synthase of Clostridium kluyveri DSM 555 (Li F et al. “Re-citrate synthase from Clostridium kluyveri is phylogenetically related to homocitrate synthase and isopropylmalate synthase rather than to Si-citrate synthase.” J. Bacteriol. June; 189(11):4299-304. 2007). The sequences for the first two genes could be easily obtained from public databases and primers for the generation of a specific PCR for each of these genes can be easily performed by somebody skilled in the art. The same applies for the generation of the PCR fragment for the coding region of the citrate synthase of C. kluyveri

For the obtention of the polypeptide carrying out the conversion of succinate to fumarate refer to the E. coli example. The PCR fragment comprising the coding region of the aceA gene could then be introduced into the plasmid pSOS95 (Tummala, S. B., et al “Development and characterization of a gene-expression reporter system for Clostridium acetobutylicum ATCC 824”, Appl. Environ. Microbiol. 65: 3793-3799 (1999)). Also in the same plasmid, we could then introduce the PCR fragment comprising the ptb promoter from plasmid pHT4 (Tummala, S. B., et al “Development and characterization of a gene-expression reporter system for Clostridium acetobutylicum ATCC 824”, Appl. Environ. Microbiol. 65: 3793-3799 (1999)) to control the expression of the PCR fragment of the coding region of gene g/cB. We will refer to this construct as plasmid A. The PCR product containing the polypeptide responsible for the conversion of succinate into fumarate could be introduced into plasmid pTLH1 (Harris, L. M. et al “Characterization of Recombinant Strains of the Clostridium acetobutylicum Butyrate Kinase Inactivation Mutant: Need for New Phenomenological Models for Solventogenesis and Butanol Inhibition?” Biotechnology and Engineering, 67 (1): 1-11 (2000). However this plasmid will require the insertion of a suitable promoter in front the polypeptide of interest. This promoter could be the ptb promoter contained in plasmid pSOS94 (Tummala, S. B., et al “Development and characterization of a gene-expression reporter system for Clostridium acetobutylicum ATCC 824”, Appl. Environ. Microbiol. 65: 3793-3799 (1999)).

The same strategy can be applied to control the expression of the PCR fragment of the coding region of the citrate synthase of C. kluyveri that will be also included in this plasmid. We will refer to this engineered plasmid as plasmid B. These two new plasmids then used to independently transform the recombinant E. coli strain ER2275 (pANI) which harbors the Φ3T I methyltransferase encoded by the Bacillus subtilis phage Φ3T as documented in Mermelstein, L. D. and Papoutsakis, E. T., “In vivo methylation in Escherichia coli by the Bacillus subtilis phage Φ3T I Methyltransferase to protect plasmids from restriction upon transformation of Clostridium acetobutylicum ATCC 824”, Appl. Environ. Microbiol. 59: 1077-1081 (1993). This step will ensure the proper methylation of the plasmid to avoid its degradation by the action of C. acetobutylicum ATCC 824 DNAases, especially Cac824I. After the successful extraction of plasmid A and plasmid B from the independent cultures, they can be introduced into Clostridium acetobutylicum ATCC 824 by electroporation following the protocols described in Mermelstein, L. D. and, Papoutsakis, E. T., “In vivo methylation in Escherichia coli by the Bacillus subtilis phage Φ3T I Methyltransferase to protect plasmids from restriction upon transformation of Clostridium acetobutylicum ATCC 824”, Appl. Environ. Microbiol. 59: 1077-1081 (1993). After selection of a clone resistant to both erythromycin (i.e. carrying plasmid A) tetracycline (i.e. carries plasmid B), the expression and activity of the polypeptides should be checked.

The transcription of the polypeptides could be checked by Q-RT-PCR and Northern Blot, their expression by a Western Blot or ELISA assay, and their in vitro activity could be checked by performing an in vitro activity assay. Determination of the activity in vivo could be carried out by comparative analysis of the levels of the reaction products between the plasmid control strain (i.e. a strain transformed with a plasmid without the DNA encoding the polypeptide of interest) and the successful clone. The verification of the activity of the engineered glyoxylate cycle can be carried out by the use of substrates labeled with ¹⁴C (radioactive) or ¹³C substrates and then analyzing the incorporation of the labeled carbon into the reaction intermediates.

### Example 24

### Expressing P450 BM3 Variant 4E10 in an Engineered Clostridium acetobutylicum ATCC 824 with a Functional TCA Cycle to Convert Propane to Propanol

Based on table 4 and the description of the enzymatic reactions that comprise a complete TCA cycle (paragraph 138), it is trivial to deduce that Clostridium acetobutylicum ATCC 824 could harbor a complete TCA cycle by providing it with the capability of convert succinyl-CoA to succinate and then succinate to fumarate. It will also require the capability of converting oxalacetate plus acetyl-COA to citrate. The first of these conversions can be carried out either by introducing the enzymatic activities EC 6.2.1.4 (known among other names as succinyl-CoA synthetase), EC 6.2.1.5 (known among other names as succinyl-CoA synthetase) or EC 3.1.2.3 (known among other names as succinyl-CoA hydrolase).

This step can be carried out by the combined action of the alpha and beta subunits of the succinyl-CoA synthetase encoded by the E. coli genes sucD (NCBI-GeneID: 945314) and sucC (NCBI-GeneID: 945312) respectively. These two genes are contiguous in the E. coli genome and the distance from the start codon of sucC to the stop codon of the preceding gene (sucB) is of 275 bp. The facts strongly suggest that sucC and sucD are transcribed as single mRNA. The second conversion (succinate to fumarate) requires the introduction of the enzymatic activities EC 1.3.5.1 or EC 1.3.99.1. In this example, the conversion of succinate into fumarate will be carried out by the same engineered enzyme used for the E. coli example (paragraph 00281).

The conversion of oxalacetate plus acetyl-CoA into citrate can be carried out by the citrate synthase of Clostridium kluyveri DSM 555 (Li F et al. “Re-citrate synthase from Clostridium kluyveri is phylogenetically related to homocitrate synthase and isopropylmalate synthase rather than to Si-citrate synthase.” J. Bacteriol. June; 189(11):4299-304. 2007). The sequences for these genes could be easily obtained from public databases and primers for the generation of sucC and sucD as a single PCR product can be easily performed by somebody skilled in the art. The same applies for the generation of the PCR fragment for the coding region of the citrate synthase of C. kluyveri. For the obtention of the polypeptide carrying out the conversion of succinate to fumarate refer to the E. coli example. The PCR fragment containing sucC and sucD could then be introduced into the plasmid pSOS95 (Tummala, S. B., et al “Development and characterization of a gene-expression reporter system for Clostridium acetobutylicum ATCC 824”, Appl. Environ. Microbiol. 65: 3793-3799 (1999)).

The citrate synthase activity will also be included in this plasmid. To control the expression of the PCR fragment of the coding region of the citrate synthase of C. kluyveri we will use the ptb promoter contained in plasmid pSOS94 (Tummala, S. B., et al “Development and characterization of a gene-expression reporter system for Clostridium acetobutylicum ATCC 824”, Appl. Environ. Microbiol. 65: 3793-3799 (1999)). We will refer to this construct as plasmid A. The PCR product containing the polypeptide responsible for the conversion of succinate into fumarate could be introduced into plasmid pTLH1 (Harris, L. M. et al “Characterization of Recombinant Strains of the Clostridium acetobutylicum Butyrate Kinase Inactivation Mutant: Need for New Phenomenological Models for Solventogenesis and Butanol Inhibition?” Biotechnology and Engineering, 67 (1): 1-11 (2000).

However this plasmid will require the insertion of a suitable promoter in front the polypeptide of interest. This promoter could be the ptb promoter contained in plasmid pSOS94 (Tummala, S. B., et al “Development and characterization of a gene-expression reporter system for Clostridium acetobutylicum ATCC 824”, Appl. Environ. Microbiol. 65: 3793-3799 (1999)). P450 BM3 variant 4E10 will be introduced into the same plasmid under the control of the ptb promoter from plasmid pHT4 (Tummala, S. B., et al “Development and characterization of a gene-expression reporter system for Clostridium acetobutylicum ATCC 824”, Appl. Environ. Microbiol. 65: 3793-3799 (1999)) We will refer to this engineered plasmid as plasmid B. These two new plasmids then used to independently transform the recombinant E. coli strain ER2275 (pAN 1) which harbors the Φ 3T I methyltransferase encoded by the Bacillus subtilis phage Φ 3T as documented in Mermelstein, L. D. and Papoutsakis, E. T., “In vivo methylation in Escherichia coli by the Bacillus subtilis phage Φ 3T I Methyltransferase to protect plasmids from restriction upon transformation of Clostridium acetobutylicum ATCC 824”, Appl. Environ. Microbiol. 59: 1077-1081 (1993). This step will ensure the proper methylation of the plasmid to avoid its degradation by the action of C. acetobutylicum ATCC 824 DNAases, especially Cac824I. After the successful extraction of plasmid A and plasmid B from the independent cultures, they can be introduced into Clostridium acetobutylicum ATCC 824 by electroporation following the protocols described in Mermelstein, L. D. and Papoutsakis, E. T., “In vivo methylation in Escherichia coli by the Bacillus subtilis phage Φ3T I Methyltransferase to protect plasmids from restriction upon transformation of Clostridium acetobutylicum ATCC 824”, Appl. Environ. Microbiol. 59: 1077-1081 (1993). After selection of a clone resistant to both erythromycin (i.e. carrying plasmid A) tetracycline (i.e. carries plasmid B), the expression and activity of the polypeptides should be checked. The transcription of the polypeptides could be checked by Q-RT-PCR and Northern Blot, their expression by a Western Blot or ELISA assay, and their in vitro activity could be checked by performing an in vitro activity assay.

Determination of the activity in vivo could be carried out by comparative analysis of the levels of the reaction products between the plasmid control strain (i.e. a strain transformed with a plasmid without the DNA encoding the polypeptide of interest) and the successful clone. The verification of the activity of the engineered TCA can be carried out by in vivo fluorimetry and or by the use of substrates labeled with ¹⁴C (radioactive) or ¹³C substrates and then analyzing the incorporation of the labeled carbon into the intermediates of the TCA. The activity of the P450 BM3 variant 4E10 polypeptide can be measured by the conversion of propane to propanol.

It is to be understood that the disclosures are not limited to particular compositions or biological systems, which can, of course, vary. It is also to be understood that the terminology used herein is for the purpose of describing particular embodiments only, and is not intended to be limiting. As used in this specification and the appended claims, the singular forms “a,” “an,” and “the” include plural referents unless the content clearly dictates otherwise. Thus, for example, reference to “a biosynthetic intermediate” includes a plurality of such intermediates, reference to “a nucleic acid” includes a plurality of such nucleic acids and reference to “the genetically modified host cell” includes reference to one or more genetically-modified host cells and equivalents thereof known to those skilled in the art and so forth. As used in this specification the term a “plurality” refers to two or more references as indicated unless the content clearly dictates otherwise.

Unless defined otherwise, all technical and scientific terms used herein have the same meaning as commonly understood by one of ordinary skill in the art to which the disclosure pertains. Although any methods and materials similar or equivalent to those described herein can be used in the practice for testing of the disclosure(s), specific examples of appropriate materials and methods are described herein. All publications mentioned herein are incorporated herein by reference to disclose and describe the methods and/or materials in connection with which the publications are cited.

The examples set forth above are provided to give those of ordinary skill in the art a complete disclosure and description of how to make and use the embodiments of the devices, systems and methods of the disclosure, and are not intended to limit the scope of what the inventors regard as their disclosure. Modifications of the above-described modes for carrying out the disclosure that are obvious to persons of skill in the art are intended to be within the scope of the following claims. All patents and publications mentioned in the specification are indicative of the levels of skill of those skilled in the art to which the disclosure pertains. All references cited in this disclosure are incorporated by reference to the same extent as if each reference had been incorporated by reference in its entirety individually.

The entire disclosure of each document cited (including patents, patent applications, journal articles, abstracts, laboratory manuals, books, or other disclosures) in the Background of the Invention, Detailed Description, and Examples is hereby incorporated herein by reference. Further, the hard copy of the sequence listing submitted herewith and the corresponding computer readable form are both incorporated herein by reference in their entireties.

While specific embodiments of the subject disclosures are explicitly disclosed herein, the above specification and examples herein are illustrative and not restrictive. It will be understood that various modifications may be made without departing from the spirit and scope of the disclosure. Many variations of the disclosures will become apparent to those skilled in the art upon review of this specification and the embodiments below. The full scope of the disclosures should be determined by reference to the embodiments, along with their full scope of equivalents and the specification, along with such variations. Accordingly, other embodiments are within the scope of the following claims.

What is claimed is:

1. A recombinant microorganisms engineered to increase the amount of NAD(P)H available for a biotransformation NAD(P)H-requiring oxidoreductase, the biotransformation NAD(P)H-requiring oxidoreductase involved in a biotransformation of a substrate to a product in the recombinant microorganism, wherein the recombinant microorganism is engineered to inactivate a respiratory pathway in the microorganism.

2. The recombinant microorganism of claim 1, further engineered to express the biotransformation NAD(P)H-requiring oxidoreductase.

3. The recombinant microorganism of claim 1, wherein inactivation of a respiratory pathway is performed by inactivating a respiratory NAD(P)H-requiring oxidoreductase, the respiratory NAD(P)H-requiring oxidoreductase involved in the respiratory pathway to be inactivated.

4. The recombinant microorganism of claim 3, wherein the respiratory NAD(P)H-requiring oxidoreductase is a dehydrogenase, an oxidase, oxidoreductase and/or reductase.

5. The recombinant microorganism of claim 4, wherein the native NAD(P)H-requiring oxidoreductase is selected from the group consisting of NDH-1 dehydrogenase, NDH-2 dehydrogenase, a quinol oxidase complex, a quinol oxidase complex, a quinol:cytochrome c oxidoreductase, a cytochrome oxidase, and a terminal reductase.

6. The recombinant microorganism of claim 5, wherein the native NAD(P)H-requiring oxidoreductase is selected from the group consisting of NADH dehydrogenase, NADH oxidase, NADPH oxidase, ubiquinol-cytochrome c reductase, quinol oxidase complex, Cytochrome c oxidase, Nitrate reductase, Periplasmic nitrate reductase, Nitrite reductase, Nitric oxide reductase, Nitrous oxide reductase, ATP sulfurylase, Adenylylsulfate reductase, dissimilatory sulfite reductase, dissimilatory sulfite reductase, Dimethyl sulfoxide reductase, Trimethylamine N-oxide reductase, Trimethylamine N-oxide reductase, Trimethylamine N-oxide reductase, Nitrite reductase complex, Respiratory arsenate reductase, Iron-cytochrome-c reductase.

7. The recombinant microorganism of claim 1, wherein inactivation of a respiratory pathway is performed by inactivating a redox active small molecule, involved in the respiratory pathway to be inactivated.

8. The recombinant microorganism of claim 7, wherein the redox active small molecule is a quinone,

9. The recombinant microorganism of claim 8, wherein the redox active small molecule is a ubiquinone or menaquinone

10. The recombinant microorganism of claim 1, wherein the recombinant microorganism is engineered to further inactivate one or more fermentation pathways in the microorganism

11. The recombinant microorganism of claim 10, wherein the one or more fermentation pathways are inactivated by inactivating one or more enzymes selected from the group consisting of Fumarate Reductase, Lactate Dehydrogenase, Pyruvate oxidase, Phosphate transacetylase, Acetate kinase, Aldehyde/Alcohol dehydrogenase, Pyruvate-Formate lyase, 1,3-propanediol dehydrogenase, Glycerol dehydratase, α-acetolactate synthase, Acetoin reductase, 2,3,-butanediol dehydrogenase, α-acetolactate decarboxylase or acetoin reductase, propionyl-CoA:succinate CoA transferase, methylmalonyl-CoA carboxyltransferase, Acetate CoA-transferase phosphotransbutyrylase, Butyrate kinase, Butanol dehydrogenase, Butyraldehyde dehydrogenase, Butyryl-CoA dehydrogenase, Crotonase, Hydroxybutyryl-CoA dehydrogenase, Thiolase, Acetoacetate decarboxylase, Formate hydrogen lyase complex, Pyruvate decarboxylase, alcohol dehydrogenase, Glycerol-3-phosphate phosphohydrolase, Formate hydrogen lyase complex, Hydrogenase, Formate dehydrogenase, D-lactate dehydrogenase, Pyruvate formate lyase, Acetaldehyde/alcohol dehydrogenase, Phosphate acetyl transferase/acetate kinase A, Fumarate reductase, and Pyruvate oxidase.

12. The recombinant microorganism of claim 1, wherein the substrate is a carbon source, the product is a alcohol and the biotransformation NAD(P)H-requiring oxidoreductase is an enzyme that catalyzes direct conversion of the carbon source into the alcohol.

13. The recombinant microorganism of claim 12, wherein the biotransformation NAD(P)H-requiring oxidoreductase is an oxidase or a reductase.

14. The recombinant microorganism of claim 12, wherein the biotransformation NAD(P)H-requiring oxidoreductase is an oxidase or a reductase selected from the group consisting of alcohol dehydrogenase, lactate dehydrogenase, leucine dehydrogenase, nicotinic acid hydroxylase, naphthalene dioxygenase, benzoate dioxygenase, cyclopentanone monooxygenase, cyclohexanone monooxygenase, steroid monooxygenases.

15. The recombinant microorganism of claim 12, wherein the biotransformation NAD(P)H-requiring oxidoreductase is a P450 cytochrome, a methane monooxygenases, a dioxygenase, styrene monooxygenases, a Baeyer-Villiger monooxygenases or a ketoreductase.

16. The recombinant microorganism of claim 15, wherein the substrate is an alkane and the product is an alcohol

17. The recombinant microorganism of claim 16, wherein the recombinant microorganism is E. coli.

18. The recombinant microorganism of claim 1, wherein the biotransformation is performed by an NAD(P)H requiring pathway.

19. The recombinant microorganism of claim 18, wherein the NAD(P)H requiring pathway is for the production of butanol

20. The recombinant microorganism of claim 16, wherein said microorganism is selected from the group consisting of: GEVO711, GEVO713, GEVO715, GEVO717, GEVO734, GEVO736, GEVO738, GEVO740, GEVO741, GEVO746, GEVO747, GEVO748, GEVO749, GEVO750, GEVO751, GEVO752, GEVO756, GEVO757, GEVO759, GEVO761, GEVO763, GEVO765, GEVO784, GEVO785, GEVO786, GEVO787, GEVO788, GEVO789, GEVO1317, GEVO1318, GEVO1319, GEVO1320, GEVO1321, GEVO1322, GEVO1323, GEVO1324, GEVO1325, GEVO1326, GEVO1327, GEVO1328, GEVO1329, GEVO1330, GEVO800, GEVO803, GEVO1331, GEVO831, GEVO1332, GEVO1333, GEVO802, GEVO805, GEVO1334, GEVO1335, GEVO1336, GEVO1337, GEVO818, GEVO822, GEVO1338, GEVO1339, GEVO1340, GEVO1341, GEVO817, GEVO821, GEVO1342, GEVO1343, GEVO1344, GEVO1345, GEVO801, GEVO804, GEVO1346, GEVO1347, GEVO1348 and GEVO1349

21. A recombinant microorganisms engineered to increase the amount of NAD(P)H available for a biotransformation NAD(P)H-requiring oxidoreductase, biotransformation NAD(P)H-requiring oxidoreductase involved in a biotransformation of a substrate to a product in the recombinant microorganism, wherein the recombinant microorganism is engineered to activate TCA cycle pathway in the microorganism.

22. The recombinant microorganism of claim 21, further engineered to express the biotransformation NAD(P)H-requiring oxidoreductase.

23. The recombinant microorganism of claim 21, wherein the TCA cycle has been enabled by activating one or more of enzymes selected from the group consisting of alpha-ketoglutarate dehydrogenase, an NADH dependant fumarate reductase, and a dimeric citrate synthase including at least one of the mutations selected from the group consisting of Y145A, R163L, K167A, and D362N.

24. The recombinant microorganism of claim 23, wherein the recombinant microorganism is further engineered to inactivate at least one of the native enzymes selected from the group consisting of fumarate reductase/succinate dehydrogenase, citrate synthase and alpha-ketoglutarate dehydrogenase.

25. A method for performing a biotransformation of a substrate, the method comprising performing the biotransformation in a recombinant microorganism according to claim 2.

26. A method for performing a biotransformation of a substrate, the method comprising performing the biotransformation in a recombinant microorganism according to claim 22.

27. The method of claim 25, wherein the biotransformation is the conversion of a carbon source into an alcohol.

28. The method of claim 26, wherein the biotransformation is the conversion of a carbon source into an alcohol.

29. A system for performing a biotransformation of a substrate, the system comprising at least one of the recombinant microorganisms of claim 1 and the substrate of the biotransformation.

30. The system of claim 27, further comprising a heterologous NAD(P)H-requiring oxidoreductase involved in the biotransformation of the substrate.


SEQUENCE LISTING (98 sequences)

SEQ ID NO: 1 - protein, 427 aa, E. Coli
  MISC_FEATURE (1)..(427): citrate synthase NP_415248.1
MADTKAKLTLNGDTAVELDVLKGTLGQDVIDIRTLGSKGVFTFDPGFTSTASCESKITFIDGDEGILLHRGFPIDQLATDSNYLEVCYILLNGEKPTQEQYDEFKTTVTRHTMIHEQITRLFHAFRRDSHPMAVMCGITGALAAFYHDSLDVNNPRHREIAAFRLLSKMPTMAAMCYKYSIGQPFVYPRNDLSYAGNFLNMMFSTPCEPYEVNPILERAMDRILILHADHEQNASTSTVRTAGSSGANPFACIAAGIASLWGPAHGGANEAALKMLEEISSVKHIPEFVRRAKDKNDSFRLMGFGHRVYKNYDPRATVMRETCHEVLKELGTKDDLLEVAMELENIALNDPYFIEKKLYPNVDFYSGIILKAMGIPSSMFTVIFAMARTVGWIAHWSEMHSDGMKIARPRQLYTGYEKRDFKSDIKR

SEQ ID NO: 2 - protein, 389 aa, E. Coli
  MISC_FEATURE (1)..(389): methyl citrate synthase NP_414867.1
MSDTTILQNSTHVIKPKKSVALSGVPAGNTALCTVGKSGNDLHYRGYDILDLAKHCEFEEVAHLLIHGKLPTRDELAAYKTKLKALRGLPANVRTVLEALPAASHPMDVMRTGVSALGCTLPEKEGHTVSGARDIADKLLASLSSILLYWYHYSHNGERIQPETDDDSIGGHFLHLLHGEKPSQSWEKAMHISLVLYAEHEFNASTFTSRVIAGTGSDMYSAIIGAIGALRGPKHGGANEVSLEIQQRYETPDEAEADIRKRVENKEVVIGFGHPVYTIADPRHQVIKRVAKQLSQEGGSLKMYNIADRLETVMWESKKMFPNLDWFSAVSYNMMGVPTEMFTPLFVIARVTGWAAHIIEQRQDNKIIRPSANYVGPEDRPFVALDKRQ

SEQ ID NO: 3 - DNA, 3585 bp, Leishmania major
  CDS (1)..(3585): Sequence coding for a fumarate reductase
atgggtgggtgcgcaacggcaacgcagcgacgttgcgcagccaccgactcgcataccggcgcctccgtagtcgttgtggaccccgaaaaggccgctcgcgagcgcgatcgcattgctcgcgacctgctcaccaccaacttccccgagttgcacgtcaatcagcgctgggtgctgctgtataaggacgtgatgcacacagtgccgtacacgctcaccattgctgtagacggcagcgtcgctcgccaagatgcggatcctgtcgtcaaggcaattctgagcgactgcttcgcgatggtggacaagcacctcaactccttcaacccggacagcgaggtgtcgcaggtcaacaggatgccggtgggaaagaagcacgtcatgtcagagcacctcttcgaggtagtgaagtgctgcgaggaggtctaccaccgtagcggcagctgctttgacccggccgcagcaccgctagtgcacaagctgcgcgatgccgctcgccggcaggactccaccgagggggacttcgccatcactgcggaggaggcggggcgtttcaccctgacgaacagctttgccattgacatcaaagaagggaccatcgcgcgcaagcacgaagatgcgacgctagacctgggtggcctgaacaagggctacactgtcgactgcgtggtggatcagctgaatgcagccaattttgccgacgtgctgttcgagtggggcggcgactgccgcgcctcgggtgtgaacgtgcagcgccagccgtgggcagtcggcgttgtgcgtccgccatcggtcgacgagatcgtcgcggctgccaagtccggcaagtcgatgacaatgaatgcacacagccttggggatcacacggatgaaccggcgccgtccacgttggccgccgacggggcggccaagcctgcgcacaaggcgttcctgcgcgtcatgtcgctcagcaatgaggcgctctgcacgagcggcgactacgagaacgtgctcttcgccaacgcgctcggctgcgctctctcgagcacatacaactggcgtcgccgctgcctcatcgaaccctgccagaacgaactggcccaggttagcatcaaatgctactcgtgcctatacgccgacgcactcgccacggcgagcttcgtgaagcgcgaccccgtgcgcgtgcggtacatgctcgagccctaccgtcacgattacaaccgcgtgaccgactacgccgcctacacgcgtgagggggagcggctggcgcacatgtacgagatcgcgcacgagagcccggcatgtcggatagagcgcattgccggctcgctgccggcgcgtgttgttgtgatcggcggcggccttgctggttgcgcggccgccattgaggcagccagctgcggcgctaccgtcattctcctggagaaggaagcccggctgggtggcaatagcgccaaggccacctctggcatcaacggctggggcacccgcacgcaagccgtgaatcacgtcctcgataactgcaagttttttgagcgggacacgttcctctccggcaagggtggtcactgcgaccctggactcgtgcgcaccctctctgtaaaatccgctgaagcgattagctggctcgagtccttcggcatcccactaaccgtcctctaccagcttggtggcgcgagccgcaggcgctgccaccgcgcgccggatcagaaagacggcaccccggtgcccgttggcttcacaatcatgcgtcacctggaggaccacatccgcaccaagctgcaaggcaaggtaacgatcttgaacgagatggcggtggtgagcctcatgcacgacgtgagcgcgatgccggacggaaaccgcgagattcgcgtgcacggtgtccgctacacgtcgatgaccgatgcgtcgggaacggtgatggatctgccggcggacgccgtcgtgcttgccaccggcggcttctcgaacgaccgcacgcccaactcgctgctgcgcgagtacgcgccaaacgtgtacggcacccccaccaccaacggcacgttcgccaccggcgacggtgtgaagatggcgcgcaagcttggcgccacgctggtggacatggacaaggtgcagctgcacccgaccggtctcattgaccccaaggacccgtcgaatcgcaccaagtacctcggccccgaggcgctgcgcgggtccggcggcatcctgctgaacaagaacggcgagcgcttcgtgaacgagctggacctgcgctccgtcgtgtcgcaggcgattatcgcgcaggacaacgagtacccgaactcgggtggcagcaagttcgcatactgcgtgctgaacgaagaggcagcgacgctcttcggcaagaactccctcacgtactactggaagtcgcagggcctgttcacccgcgtggatgacatgaaggcgctcgccgagctcatcggctgctcggttgaaagcctacatcgaaccctcgagacgtacgagcgccagagcacggggaagaaggcctgcccgctgactggcaagctcgtgttccccagtgtggtgggcaccaaggggccctattacgtggcgtacgtcacgccgtcgatccactacaccatgggtggctgcttcatctctccggcggcggagctgctcatggaggatcactccgtcaacatattcgacgacatgcgccccatccttggcctcttcggtgcgggtgaggtaaccggcggcgtgcacggccgcaaccgtctcggcggcaactctctgctggagtgcgtcgtgttcggcaagatcgctggcgaccgcgcggccacaattctgcagaaggagaagcacgggctcagcaaggataagtgggtgccggtggtggtgcgggagtcgagggcgagtgatcagttcggtgttggctcgcgcgtcctgcgcttcaacctgcccggcgcgacgcagacatccggattgaccgttggcgagttcatcggtatccgcggtgactgggacggccagcagttgatcggatactacagccccatcaatatgcccgacgacaagggccgcatctcgattctggcgcgtggtgacaagggcaacctgcaggaatggatctcgtccatgcgtccgggcgactcggtcgaaatgaaggcctgcggcggtctccgtatcgagctcaagccccaccagaagcagatggtgtaccgcaagacggttatccgaaaactgggcctcatcgccggcggctctggtgtggcgccgatgctgcagattattaaggccgcgctcaatcgcccatatgtggacagcatcgagacaatccgcctcgtttacgccgccgaggacgagtatgaactaacctaccgcttgctgctaaagcaataccgcaccgacaacccgggcaagttcgactgcggcttcgtgctcaataaccctcccgaaggctggacagagggtgtgggctacgtcgaccgtgccacgctgcagagccttctcccgcctccgtcgaagggcttgctcgtggccatttgcggcccgccggtgatgcagcgttccgttgtggcggacctgctggcactaggctataacgccgaaatggtgcgcacagtggatgaggatggcgcgctctag

SEQ ID NO: 4 - protein, 1194 aa, Leishmania major
MGGCATATQRRCAATDSHTGASVVVVDPEKAARERDRIARDLLTTNFPELHVNQRWVLLYKDVMHTVPYTLTIAVDGSVARQDADPVVKAILSDCFAMVDKHLNSFNPDSEVSQVNRMPVGKKHVMSEHLFEVVKCCEEVYHRSGSCFDPAAAPLVHKLRDAARRQDSTEGDFAITAEEAGRFTLTNSFAIDIKEGTIARKHEDATLDLGGLNKGYTVDCVVDQLNAANFADVLFEWGGDCRASGVNVQRQPWAVGVVRPPSVDEIVAAAKSGKSMTMNAHSLGDHTDEPAPSTLAADGAAKPAHKAFLRVMSLSNEALCTSGDYENVLFANALGCALSSTYNWRRRCLIEPCQNELAQVSIKCYSCLYADALATASFVKRDPVRVRYMLEPYRHDYNRVTDYAAYTREGERLAHMYEIAHESPACRIERIAGSLPARVVVIGGGLAGCAAAIEAASCGATVILLEKEARLGGNSAKATSGINGWGTRTQAVNHVLDNCKFFERDTFLSGKGGHCDPGLVRTLSVKSAEAISWLESFGIPLTVLYQLGGASRRRCHRAPDQKDGTPVPVGFTIMRHLEDHIRTKLQGKVTILNEMAVVSLMHDVSAMPDGNREIRVHGVRYTSMTDASGTVMDLPADAVVLATGGFSNDRTPNSLLREYAPNVYGTPTTNGTFATGDGVKMARKLGATLVDMDKVQLHPTGLIDPKDPSNRTKYLGPEALRGSGGILLNKNGERFVNELDLRSVVSQAIIAQDNEYPNSGGSKFAYCVLNEEAATLFGKNSLTYYWKSQGLFTRVDDMKALAELIGCSVESLHRTLETYERQSTGKKACPLTGKLVFPSVVGTKGPYYVAYVTPSIHYTMGGCFISPAAELLMEDHSVNIFDDMRPILGLFGAGEVTGGVHGRNRLGGNSLLECVVFGKIAGDRAATILQKEKHGLSKDKWVPVVVRESRASDQFGVGSRVLRFNLPGATQTSGLTVGEFIGIRGDWDGQQLIGYYSPINMPDDKGRISILARGDKGNLQEWISSMRPGDSVEMKACGGLRIELKPHQKQMVYRKTVIRKLGLIAGGSGVAPMLQIIKAALNRPYVDSIETIRLVYAAEDEYELTYRLLLKQYRTDNPGKFDCGFVLNNPPEGWTEGVGYVDRATLQSLLPPPSKGLLVAICGPPVMQRSVVADLLALGYNAEMVRTVDEDGAL

SEQ ID NO: 5 - DNA, 3444 bp, Leishmania major
  CDS (1)..(3444): Sequence coding for a fumarate reductase
atggcggatggaaagacctctgcatctgtggtggcggtcgatgccgagagcgcggcaaaggagcgcgacgcagctgcccgcgcgatgctgcaggacggcggcgtctcaccagtcggaaaggctcagctgttaaagaaaggcctcgtgcacacggttccgtacaccctcaaagtcgtcgtggcggaccccaaggagatggagaaggccactgcagatgcggaggaagtgctccagagtgccttccaggtggtcgacaccctcctcaacagcttcaacgagaacagcgaggtgtcccgcatcaaccgaatgccggtcggtgaggaacaccagatgtctgcggctctgaagcatgtgatggcctgctgtcagaaagtctacaactcgtcgcgcggcgccttcgaccccgccgtcggcccgctcgtccgagagctgcgcgaggctgcccataaaggcaagacggtgccggcggagcgcgtcaacgacctcctcagcaagtgcacgctgaacgctagcttctccattgacatgaaccgtggcatgatcgcccgcaagcacgcggacgcgatgctggaccttggtggtgtgaacaagggctacggcatcgactacaccgtcgagcgcctcaacagcctcggctacgacgacgtcttcttcgagtggggcggtgatgtgcgtgccagtggcaagaaccagtcgattcagccctgggccgtcggcatcgtgcgcccacccgctttggcggacattcgcactgtggtgccgaaggacaagcggtcctttatccgggtggtgcacctcaacaacgaggccatcgccaccagcggtgactacgagaacctgattgagacccccgcctccaaagtgtactcgtcgacctttgatcgggcatccaagaacctgctggagccgaccgaggcgggcatggcgcaggtctccgtgaagtgctacagctgcatgtacgccgatgccctggccaccgccgcgctcctcaagaacgacccggcggccgtccgccgcatgctggacaactggcgctacgtgcgcgataccgtcaccgactacaccacctacactcgtgagggcgagcgtgttgccaagatgctcgaaatcgctacggaggatgcggagatgcgcgcgaagcgcatcaagggctcgctgccggcgcgtgtgatcatcgttggtggaggcctggccggttgctcggctgcgatcgaggcggccaactgtggcgcgcaggtgattctgctggagaaggagccaaagctcggtggcaacagcgccaaagcaacgtccggcatcaacgcctggggaacccgtgcgcaggcgaagcagggcatcatggacggcggcaagttctttgagcgcgacacgcaccgctccggcaagggtggcaactgcgatccatgcctcgtcaagacgctatctgtaaagagctccgacgcggtgaagtggctgtccgagctgggcgtgccgctgacggtgctgtcgcagctcggcggcgcgagccgcaagcgttgccaccgcgcgccagataagtcggacggtaccccggtcccagtcggcttcaccatcatgaagaccctggagaaccatatcgtcaacaacctcagtcgccatgttactgtgatgacgggcattaccgtaacagcgctggagagcacgagccatgtccgccctgatggcgtccttgtgaagcacgtgacgggcgtccgtctcatccaggccagcgggcagtccatggtgctgaacgccgacgctgtcattctcgccaccggcggcttctcgaacgaccacacgccaaactcgctcctgcagcagtacgcgccgcaactgtcgtccttccccaccaccaacggtgtatgggccaccggtgatggcgtgaagatggcgagcaagctgggcgtggcgctggtagacatggataaggtgcagctgcacccgaccggtctcattgaccccaaagacccgtcgaatcgcaccaagtacctcggccccgaggcgctgcgtggctccggtggcgtgctgctgaacaagaacggcgagcgcttcgtgaacgagctggacctgcgctccgtcgtgtcacaggcgattatcgcgcaggacaacgtctaccccgggtccggcggcagcaaattcgcgtactgtgtgctgaatgagaccgcggcgaagctcttcggcaagaacttcctcggcttctactggaatcgcctcggtctcttccagaagtcagatagcgtcgctggcctggcgaaactgattggctgccctgaggcgaatgtgatggcaaccctgaagcagtacgaggaactctcatccaagaagttaaacccctgcccgctgactggcaagaacgtgttcccttgtgtgctgggtactcaaggaccctactacgtcgccctcatcacgccgtcgatccactacaccatgggtggctgtctcatctcgccctcggcggagatgcagacgaaagacaatagcggtgtaacccccgttcgtcgtccgattctgggcctctttggcgctggcgaggtgacgggcggcgtgcacggtggcaaccgtctcggcggcaactcgctgctggagtgcgtcgtgtttggcaagatcgccggcgaccgcgcggccacgattctgcagaagaagaacacggggctatccatgacggagtggtcgaccgtggtgttgcgtgaggtgcgcgagggtggcgtgtacggtgccggctcacgcgtgctgcgcttcaacatgcccggcgcgctgcagaagactggccttgctcttggtcagtttatcggtattcgcggtgactgggacggccaccgactgatcggctactacagccccatcacgctgccggacgatgtcggtgtgattggcatcctcgcccgcgccgacaagggccgcctggcggagtggatctctgccctgcagcctggagacgcggtggagatgaaggcgtgcggtggcctcatcatcgagcgccgcttcgctgaccgccacttctttttccgtggccacaagattcgcaagctcgccctcatcggtggcggcacgggtgtcgcgccgatgctgcagattgtgcgggctgcggtgaagaaacccttcgtggactcgatcgagagcattcagttcatctacgccgccgaggacgtatcggagctgacgtaccgcacgctgcttgagagctacgagaaggagtatggctctgagaagttcaagtgtcacttcgttctcaacaaccctcccgctcagtggaccgacggcgtcggcttcgttgataccgctttgctacgctccgccgtgcaggcgccatccaacgaccttctggtggccatctgcggtccgccgatcatgcagcgtgcggtcaaaggtgccctcaagagtctcggctacaacatgaacctcgtgcgcacggtggatgaaacagaacccacctcggccaagatttaa

SEQ ID NO: 6 - protein, 1147 aa, Leishmania major
MADGKTSASVVAVDAESAAKERDAAARAMLQDGGVSPVGKAQLLKKGLVHTVPYTLKVVVADPKEMEKATADAEEVLQSAFQVVDTLLNSFNENSEVSRINRMPVGEEHQMSAALKHVMACCQKVYNSSRGAFDPAVGPLVRELREAAHKGKTVPAERVNDLLSKCTLNASFSIDMNRGMIARKHADAMLDLGGVNKGYGIDYTVERLNSLGYDDVFFEWGGDVRASGKNQSIQPWAVGIVRPPALADIRTVVPKDKRSFIRVVHLNNEAIATSGDYENLIETPASKVYSSTFDRASKNLLEPTEAGMAQVSVKCYSCMYADALATAALLKNDPAAVRRMLDNWRYVRDTVTDYTTYTREGERVAKMLEIATEDAEMRAKRIKGSLPARVIIVGGGLAGCSAAIEAANCGAQVILLEKEPKLGGNSAKATSGINAWGTRAQAKQGIMDGGKFFERDTHRSGKGGNCDPCLVKTLSVKSSDAVKWLSELGVPLTVLSQLGGASRKRCHRAPDKSDGTPVPVGFTIMKTLENHIVNNLSRHVTVMTGITVTALESTSHVRPDGVLVKHVTGVRLIQASGQSMVLNADAVILATGGFSNDHTPNSLLQQYAPQLSSFPTTNGVWATGDGVKMASKLGVALVDMDKVQLHPTGLIDPKDPSNRTKYLGPEALRGSGGVLLNKNGERFVNELDLRSVVSQAIIAQDNVYPGSGGSKFAYCVLNETAAKLFGKNFLGFYWNRLGLFQKSDSVAGLAKLIGCPEANVMATLKQYEELSSKKLNPCPLTGKNVFPCVLGTQGPYYVALITPSIHYTMGGCLISPSAEMQTKDNSGVTPVRRPILGLFGAGEVTGGVHGGNRLGGNSLLECVVFGKIAGDRAATILQKKNTGLSMTEWSTVVLREVREGGVYGAGSRVLRFNMPGALQKTGLALGQFIGIRGDWDGHRLIGYYSPITLPDDVGVIGILARADKGRLAEWISALQPGDAVEMKACGGLIIERRFADRHFFFRGHKIRKLALIGGGTGVAPMLQIVRAAVKKPFVDSIESIQFIYAAEDVSELTYRTLLESYEKEYGSEKFKCHFVLNNPPAQWTDGVGFVDTALLRSAVQAPSNDLLVAICGPPIMQRAVKGALKSLGYNMNLVRTVDETEPTSAKI

SEQ ID NO: 7 - DNA, 1488 bp, Leishmania major
  CDS (1)..(1488)
atgtctcgagtagccccttcagtaaatcgtgtcgtcatcgtcggcagcggacttgcagggcagtccgcggcgatcgaggccgcccgcgagggcgctaaggaagttgtcctcattgagaaggaagggcggctgggcggcaacagtgccaaggccacgtctggcatcaatggctggggcacggcagtgcagaaggccgccggcgtgcacgacagcggtgaactctttgaaaaggatacgttcgtctctggcaagggtggcacctgtcagccagagttggtgcggacgctgtccgaccacagtgcagaagccatcgagtggctttcttcatttggcatcccgctgaccgccatcacgcaactcggcggtgcgagtcgcaagcgctgccaccgtgccccagacaagccggacgggactccgctgccgatcggtttcaccattgtgcgtgcgctggagaactacattcgcacaaacctgtccggcaccgtgctcatcgaaagcaatgcgcgtctcatctcactgatacacagaaaggagagcgacgtggaggtggtgcaaggcatcacgtatgccacgcaaactggaagcggcgaggagcagactcgcgaactgcaggcccgtgccgtcattctcgccaccggcggcttctcgaacgatcacacgcccaactcgctcctgcagcagtacgcgccgcaactgtcgtccttccccaccaccaacggtgtatgggccaccggcgacggtgtaaaggcggcacgtgagctgggcgtggcgctggtagacatggataaggtgcagctgcacccgaccggcctgctgaatccgaaagatccgaacgccaagacactcttcctcggccccgaggcgctgcgtggctccggtggcgtgctgctgaacaagaacggcgagcgcttcgtgaacgagctggacctgcgctccgtcgtgtcacaggcgattatcgcgcaggacaacgtctaccccggcacggagagtcgccgctacgcgtattgcgtactgaacgacgcggctgctgacgcgtttgggcgcagttcgctgaacttctactggaaaaagatggggctctttgctgaggctgccgacgtcgccgcgcttgcggctctcattggatgtccggaagaaaccttgaagcacacgctctccgagtacgagaagatctccagcggccaaaaaccgtgcccgaagactggaaaagaagtgttcccttgtgtgctgggtactcaagggccctactacgtcgccctcgtcacgccgtcgatccactacaccatgggtggctgcctcatctcacctgcagcagagatcctggatgagcagatgcacccgattccgggcctctttggcgctggcgaggtgacgggcggcgtgcacggtggcaaccgtctcggcggcaactcgctgctggagtgcgtcgtgttcggtcgtattgctgggcgtcaagctgcacgccacctcggcacaccggtgtcttag

SEQ ID NO: 8 - protein, 495 aa, Leishmania major
MSRVAPSVNRVVIVGSGLAGQSAAIEAAREGAKEVVLIEKEGRLGGNSAKATSGINGWGTAVQKAAGVHDSGELFEKDTFVSGKGGTCQPELVRTLSDHSAEAIEWLSSFGIPLTAITQLGGASRKRCHRAPDKPDGTPLPIGFTIVRALENYIRTNLSGTVLIESNARLISLIHRKESDVEVVQGITYATQTGSGEEQTRELQARAVILATGGFSNDHTPNSLLQQYAPQLSSFPTTNGVWATGDGVKAARELGVALVDMDKVQLHPTGLLNPKDPNAKTLFLGPEALRGSGGVLLNKNGERFVNELDLRSVVSQAIIAQDNVYPGTESRRYAYCVLNDAAADAFGRSSLNFYWKKMGLFAEAADVAALAALIGCPEETLKHTLSEYEKISSGQKPCPKTGKEVFPCVLGTQGPYYVALVTPSIHYTMGGCLISPAAEILDEQMHPIPGLFGAGEVTGGVHGGNRLGGNSLLECVVFGRIAGRQAARHLGTPVS

SEQ ID NO: 9 - DNA, 3699 bp, Trypanosoma brucei
  CDS (1)..(3699): Sequence coding for a fumarate reductase
atgctctcaacgaagcaacttctccttcgagccacatctgcattagtggcgggaagctctggagttgcgcgagacagcccttcgcttgtcggcgacccttgcgactcggtttcaccaacgcgggtcgtatgggggcgcttcttcaaatccctagcgccacccgctccctcggttgtttcatgtcaaaagcgttttacgtcccatggcgccgatggtatttcctcggcttcgattgttgtcactgacccggaggcggcagcaaagaagcgtgaccgcatggcgcgcgagttgctctcaagtaatagtggtctttgtcaagaagatgaacccactatcattaacttaaaggggttggagcacacgattccgtacaggctcgccgtggttctttgtaactcgcgctctacaggtgaattcgaagcaaaggcagctgagattttgcgaaaggcatttcacatggtggactactccctcaattgtttcaatcctgaaagcgagttgtcgcgtgtcaactctctgccggtgggtgagaagcatcaaatgtcggaggatctccggcacgtgatggagtgcaccatcagtgtacatcactccagcggaatgggcttcgacccggcggcaggtccaattatcagccgacttcggggggcaatgagggaccacaacgacatgtccgacatttccgtaacggaagccgaggtagagctcttctccttagcgcaaagttttgacgtggacctcgaggagggaacaatagctcgcaagcactctgaagcgaggcttgatcttggtggtgtgaacaaaggctacacagttgattatgtagtggatcatcttcgtgcggccggtatgccaaacgtgctctttgagtggggcggggatattcgagcgtcgggtaggaacatcaaaggaaacctatgggcagttgctatcaaacgaccgccatctgtggaggaggtgattcggcgcgccaaagggaaaatgttaaaaatgggggaggaggagcaggaagagaaggacgatgattctccatccctgcttcatgtggtggagcttgatgatgaagccctttgcaccagtggtgactacgaaaacgttttgtatcatccaaagcatggagtggcggggagcatttttgactggcagcgaagggggctactatctcctgaggaaggggcactcgctcaagtgtctgtgaaatgttatagcgcaatgtacgctgatgctctggcaacagtgtgccttgtgaagcgtgatgctgtgaggattcgctacttattagagggctggcgttacgttcgaagtcgtgtgacgaattactttgcctatacccgtcagggcgagcggttagcacatatgcacgagatagcgcaagaaacacgggagctacgtgaaatacggattgccgggagtttgccctccagaattgttattgtgggtggaggtctagcgggcctttcagcggccatcgaagccgcaagttgtggtgcacaagtcatactcatggaaaaggaaggaagaatcggggggaacagcgcaaaggctacatcaggtattaatgggtgggggacgcgtacgcaggcaaagtcagatattctcgacggtggaaagtattttgagcgtgacacttttctctctggcgttggcggtactaccgatcctgccctcgtcaaagtgctctcagttaagagtggggacgcaattggttggcttacttctcttggtgtgccactcagtgtcctctcgcaacttggtggccacagtttcaagcgaacccaccgtgccccggacaaaacggacgggacacccctaccaattggtcatacgatcatgagaaccctcgaggatcacatccgtaacaacctctctgagcgagtaacgattatgacacatgtgtccgtgaccgagttattgcacgaaaccgatacaacacctgatggcgcctccgaagtgcgtgttacgggtgtaagatacagggacctctccgatgtggatggccagccatcaaaattgcttgcggatgccgtcgttcttgcaactggtggtttctccaatgaccgtgaagaaaattcactgctctgcaagtatgcgcctcacctggccagttttccaacgacaaatggcccctgggcgaccggtgacggggttaaactcgcaacatcggttggtgcaaagcttgtggatatggataaggttcagctacaccccacagggcttatcgatccaaaggatcccgcgaacacaacgaagattctcggcccggaggcactccgaggttcaggtgggatattactcaacaagcaaggaaagcgcttcgtgaatgaacttgacctccgctctgttgtatccaaggcaattaatacgcagggtaatgaataccctggatccggtggatgttactttgcgtactgcgtgctcaacgaagatgcaacaaacctcttctgtggcggtgcactggggttctacggaaagaagcttggtttgttccagcgtgctgagactgtggaagagttggccaaactgattggctgtgacgaaggtgaattacgggatacgcttgaaaagtatgaaacttgcagcaaggccaaagttgcgtgccctgtgacggggaaggtagtattcccttgtgtggtgggtacaagggggccgtacaatgttgcttttgtcacgccttccattcattacacaatgggtggctgcctcatttcaccggctgctgaagttcttcaggagtacaaaggtttaaatattctggaaaaccatagaccgattcgatgcttgtttggtgccggtgaagtgacgggtggtgtgcacggtggtaaccgccttggtggtaattcgctcttggaatgtgtggtattcgggaaaattgcgggtgaccgtgccgcaacaatacttcaaaaacgtgagatagccctctccaagacgagttggacttccgttgttgtacgtgagtcccgctccggcgaacagttcgggaccggctctcgtgttcttcgttttaacctacctggggcgctgcagcgcacaggtctcaatctgggcgaatttgtggccatccgtggcgagtgggacggccaacaacttgttggttacttcagtccaattacactaccagaggaccttggcactatctcccttctggttcgtgccgacaagggcacattgaaggaatggatctgcgccttgcgaccgggcgactccgtcgaaatcaaagcgtgtggaggtcttcgtattgatcaagacccggtaaagaagtgtctgctgtttcgtaaccggcctattacgcggtttgctcttgtcgcggcagggactggtgtcgcgcccatgttgcaggttattcgtgcggcactcaagaagccttacgtggacacgttggaaagcatccgtcttatatacgccgcagaagagtacgacacattgacgtatcgctcaattttgcagcggtttgcggaagagttccccgacaagttcgtctgcaacttcgttcttaacaacccacccgaagggtggacaggtggagtggggtttgtcaacaaaaaatccctgcagaaggtgctgcaaccgccatcgagtgagccgctgattgttgtgtgtggaccgcccgtgatgcagcgcgacgtgaagaatgagttactgagcatgggttatgacaaagagctcgttcatacggttgacggcgagtcgggaacgctgtga

SEQ ID NO: 10 - protein, 1232 aa, Trypanosoma brucei
MLSTKQLLLRATSALVAGSSGVARDSPSLVGDPCDSVSPTRVVWGRFFKSLAPPAPSVVSCQKRFTSHGADGISSASIVVTDPEAAAKKRDRMARELLSSNSGLCQEDEPTIINLKGLEHTIPYRLAVVLCNSRSTGEFEAKAAEILRKAFHMVDYSLNCFNPESELSRVNSLPVGEKHQMSEDLRHVMECTISVHHSSGMGFDPAAGPIISRLRGAMRDHNDMSDISVTEAEVELFSLAQSFDVDLEEGTIARKHSEARLDLGGVNKGYTVDYVVDHLRAAGMPNVLFEWGGDIRASGRNIKGNLWAVAIKRPPSVEEVIRRAKGKMLKMGEEEQEEKDDDSPSLLHVVELDDEALCTSGDYENVLYHPKHGVAGSIFDWQRRGLLSPEEGALAQVSVKCYSAMYADALATVCLVKRDAVRIRYLLEGWRYVRSRVTNYFAYTRQGERLAHMHEIAQETRELREIRIAGSLPSRIVIVGGGLAGLSAAIEAASCGAQVILMEKEGRIGGNSAKATSGINGWGTRTQAKSDILDGGKYFERDTFLSGVGGTTDPALVKVLSVKSGDAIGWLTSLGVPLSVLSQLGGHSFKRTHRAPDKTDGTPLPIGHTIMRTLEDHIRNNLSERVTIMTHVSVTELLHETDTTPDGASEVRVTGVRYRDLSDVDGQPSKLLADAVVLATGGFSNDREENSLLCKYAPHLASFPTTNGPWATGDGVKLATSVGAKLVDMDKVQLHPTGLIDPKDPANTTKILGPEALRGSGGILLNKQGKRFVNELDLRSVVSKAINTQGNEYPGSGGCYFAYCVLNEDATNLFCGGALGFYGKKLGLFQRAETVEELAKLIGCDEGELRDTLEKYETCSKAKVACPVTGKVVFPCVVGTRGPYNVAFVTPSIHYTMGGCLISPAAEVLQEYKGLNILENHRPIRCLFGAGEVTGGVHGGNRLGGNSLLECVVFGKIAGDRAATILQKREIALSKTSWTSVVVRESRSGEQFGTGSRVLRFNLPGALQRTGLNLGEFVAIRGEWDGQQLVGYFSPITLPEDLGTISLLVRADKGTLKEWICALRPGDSVEIKACGGLRIDQDPVKKCLLFRNRPITRFALVAAGTGVAPMLQVIRAALKKPYVDTLESIRLIYAAEEYDTLTYRSILQRFAEEFPDKFVCNFVLNNPPEGWTGGVGFVNKKSLQKVLQPPSSEPLIVVCGPPVMQRDVKNELLSMGYDKELVHTVDGESGTL

SEQ ID NO: 11 - DNA, 3429 bp, Trypanosoma brucei
  CDS (1)..(3429): Sequence coding for a fumarate reductase
atggtagacgggcgatcttctgcatcaattgttgccgttgatcccgaaagggctgcgcgtgagcgcgacgcagcagcgcgtgcccttcttcaagacagtccgctacacacgaccatgcaatatgcaacgtctggtcttgagcttaccgttccctatgcacttaaggtggttgccagtgctgacaccttcgatcgcgctaaggaggttgccgatgaggtgctacgctgcgcatggcaactcgccgacaccgtgttgaacagtttcaacccgaacagtgaggtttcactcgtgggtcgcctgcctgtggggcagaagcaccaaatgtctgctccactcaagcgtgtgatggcatgctgccagcgtgtgtataactcatcggctggatgttttgatccctccacagcacccgtcgcaaaggcgctgcgtgagattgcactggggaaggagcggaacaatgcttgtctggaggcacttactcaagcgtgtacgcttcccaacagttttgtgatcgatttcgaagctggaactatcagccgtaagcacgagcatgcgtctctggacctaggtggggttagcaaaggttatatcgttgattatgtcattgataatatcaatgctgctggatttcaaaacgttttttttgactggggtggagactgccgtgcgagtggtatgaatgcgcgcaataccccgtgggttgttggtataactcgccctccgtcccttgatatgctccctaacccgccaaaggaggcgtcgtatatcagcgttatctctctcgacaacgaggcccttgccacgagtggcgattatgaaaacttaatatacaccgctgatgataaaccccttacctgcacttatgactggaaggggaaggaactgatgaaaccttctcagtccaatatcgcgcaggtatcggttaaatgttatagcgccatgtacgctgacgcgcttgcgactgcgtgtttcataaagcgggatcccgcgaaggttcgacagctgctggacggttggcgttacgtgcgtgatacagtgagagattacagggtctacgttcgtgaaaatgagcgagtagcgaagatgtttgagatcgccacagaggatgcggaaatgaggaagaggcggatcagcaacacacttcccgctcgtgtcattgtggtgggcggtggtcttgcgggtttgtccgcggccatcgaagctgcaggatgcggtgctcaggttgtgcttatggagaaggaggcgaagctcggaggcaacagcgccaaggcgacatctggtatcaacggatggggcacacgtgctcaggcgaaggcaagcattgtggatggtgggaaatacttcgagcgtgacacatacaagtctggtatcgggggtaacaccgatcctgcccttgtgaagacactttctatgaaaagtgctgacgctattgggtggctgacctcgttgggtgtaccgctgacggtattgtcacagcttgggggtcacagccgcaagcgcacacatcgggcaccggataagaaagatggtacacctctacctatcggatttacaatcatgaaaaccctcgaggatcacgtgcgtggtaacctttctggccgcatcaccataatggaaaactgcagtgtaacgtcgttgctcagtgagacgaaggaacggccagatggcactaaacagatacgagttactggtgtggagttcacgcaggctggcagtgggaagacgaccatacttgcagatgctgtcatccttgccactggtggattttctaacgacaaaactgcagactccctgcttcgtgagcacgccccgcacttggtcaacttccctacgacgaatggcccgtgggcgacaggtgatggcgtgaaacttgcacagcgacttggcgctcaactggtggatatggacaaggtccagttgcatccgacaggcctcatcaacccgaaggatccagcgaaccctacaaagttccttggacctgaggcgctacgtggatccggtggcgttttgttgaacaagcaaggcaagcgcttcgttaatgaacttgacctccgttctgtggtatcgaaagccatcatggaacagggtgcggaatatcctggatcgggtggtagcatgttcgcctactgtgtgttgaatgctgcggcgcagaagctctttggtgtcagctcacacgagttctactggaagaagatgggtctcttcgtgaaggctgacaccatgagggacctcgctgcactcattgggtgcccagtggaatctgtgcagcagacgctggaggagtacgagcggctctccatatcacagcgttcctgccccatcacgcgcaaaagcgtctatccgtgcgtgctcggcactaagggcccctactacgtcgccttcgtgacaccttcgattcactacacaatgggtggatgtctcatctcgccttctgctgaaatacaaatgaagaacacatcatcacgcgctccactgagtcacagcaacccaatcctcgggttatttggtgccggtgaggtaacgggtggtgtgcacggtgggaaccggttgggcggcaattcgctgcttgagtgcgtcgtgtttgggagaattgcgggtgatcgggcctcgaccatccttcagaggaagtcctcagcactttccttcaaggtgtggacgaccgtggtgctgcgtgaagtacgcgaaggtggtgtgtacggtgctgggtcccgcgtgcttcgctttaatttacccggggcgctgcaacggtctggtctgagcctcggccaatttatcgcaattcgtggtgattgggacggtcagcagttgatcggttattacagtcccatcacgctgccagatgatcttggcatgatcgatatactcgcccgcagtgataaggggacgctgagggagtggatttccgctctggagccgggtgacgctgtggagatgaaggcatgcggtggtctggtgattgagcgccgcttaagcgataagcactttgtgttcatgggacacattatcaacaagctttgtctaattgctggtggaacgggtgtggcaccgatgctgcaaataatcaaagcagcctttatgaaacccttcattgacacattggagagcgttcatctcatctatgccgcggaggacgtgacggagttgacgtatcgcgaggtgctggaggagcgccgtcgtgagtcacgtggaaagttcaagaaaacgtttgtcctcaaccggcccccgcccctatggactgatggtgttggcttcatcgaccggggcatcctcacaaatcatgtgcagccgccatctgacaacctgctggtggccatatgcggaccaccggtaatgcagcgcattgtaaaggcgaccctgaagactttgggctacaacatgaaccttgtgaggactgtggatgaaacggagccgagcggctcatccaaaatttga

SEQ ID NO: 12 - protein, 1142 aa, Trypanosoma brucei
MVDGRSSASIVAVDPERAARERDAAARALLQDSPLHTTMQYATSGLELTVPYALKVVASADTFDRAKEVADEVLRCAWQLADTVLNSFNPNSEVSLVGRLPVGQKHQMSAPLKRVMACCQRVYNSSAGCFDPSTAPVAKALREIALGKERNNACLEALTQACTLPNSFVIDFEAGTISRKHEHASLDLGGVSKGYIVDYVIDNINAAGFQNVFFDWGGDCRASGMNARNTPWVVGITRPPSLDMLPNPPKEASYISVISLDNEALATSGDYENLIYTADDKPLTCTYDWKGKELMKPSQSNIAQVSVKCYSAMYADALATACFIKRDPAKVRQLLDGWRYVRDTVRDYRVYVRENERVAKMFEIATEDAEMRKRRISNTLPARVIVVGGGLAGLSAAIEAAGCGAQVVLMEKEAKLGGNSAKATSGINGWGTRAQAKASIVDGGKYFERDTYKSGIGGNTDPALVKTLSMKSADAIGWLTSLGVPLTVLSQLGGHSRKRTHRAPDKKDGTPLPIGFTIMKTLEDHVRGNLSGRITIMENCSVTSLLSETKERPDGTKQIRVTGVEFTQAGSGKTTILADAVILATGGFSNDKTADSLLREHAPHLVNFPTTNGPWATGDGVKLAQRLGAQLVDMDKVQLHPTGLINPKDPANPTKFLGPEALRGSGGVLLNKQGKRFVNELDLRSVVSKAIMEQGAEYPGSGGSMFAYCVLNAAAQKLFGVSSHEFYWKKMGLFVKADTMRDLAALIGCPVESVQQTLEEYERLSISQRSCPITRKSVYPCVLGTKGPYYVAFVTPSIHYTMGGCLISPSAEIQMKNTSSRAPLSHSNPILGLFGAGEVTGGVHGGNRLGGNSLLECVVFGRIAGDRASTILQRKSSALSFKVWTTVVLREVREGGVYGAGSRVLRFNLPGALQRSGLSLGQFIAIRGDWDGQQLIGYYSPITLPDDLGMIDILARSDKGTLREWISALEPGDAVEMKACGGLVIERRLSDKHFVFMGHIINKLCLIAGGTGVAPMLQIIKAAFMKPFIDTLESVHLIYAAEDVTELTYREVLEERRRESRGKFKKTFVLNRPPPLWTDGVGFIDRGILTNHVQPPSDNLLVAICGPPVMQRIVKATLKTLGYNMNLVRTVDETEPSGSSKI

SEQ ID NO: 13 - DNA, 2634 bp, Trypanosoma brucei
  CDS (1)..(2634): Sequence coding for a fumarate reductase
atgtgtcacacacgtgtatgggcatccatgcgtctacatggatgtcttttgtcgccgttaacttcagtatctatgtgtcaccctttctggctgcaattttttgtttgttcttttcatctcttccttgacaccgtggcgcgagtgtcattcaccggtttgaaaagcttaacccatcgtaatcgattggtgggtgtcgcaatgttttccggtcctgctgaagtttattacactaaaagcatacaaaacaatcatagacagttgcgccgcaacgttggaggagcacagccaggtcgcggtatatattactccctgcttgctgttttgctcgtagttacattcatcaagactgccagcgcgacgctagaaagagtgcgagtgggtatcgatatgataagatcaagggagatggccatggttgagtccgagaatcctcgtgtcattgtggtgggcggtggtcttgcgggtttgtccgcggccatcgaagctgcaggatgcggtgctcaggttgtgcttatggagaaggaggcgaagctcggaggcaacagcgccaaggcgacatctggtatcaacggatggggcacacgtgctcaggcgaaggcaagcattgtggatggtgggaaatacttcgagcgtgacacatacaagtctggtatcgggggtaacaccgatcctgcccttgtgaagacactttctatgaaaagtgctgacgctattgggtggctgacctcgttgggtgtaccgctgacggtattgtcacagcttgggggtcacagccgcaagcgcacacatcgggcaccggataagaaagatggtacacctctacctatcggatttgcgatcgtgaaaaccctcgaggatcacgtgcgtggtaacctttctggccgcatcaccataatggaaaactgcagtgtaacgtcgttgctcagtgagacgaaggaacggccagatggcactaaacagatacgagttactggtgtggagttcacgcaggctggcagtgggaagacgaccatacttgcagatgctgtcatccttgccactggtggattttctaacgacaaaactgcagactccctgcttcgtgagcacgccccgcacttggtcaacttccctacgacgaatggcccgtgggcgacaggtgatggcgtgaaacttgcacagcgacttggcgctcaactggtggatatggacaaggtccagttgcatccgacaggcctcatcaacccgaaggatccagcgaaccctacaaagttccttggacctgaggcgctacgtggatccggtggcgttttgttgaacaagcaaggcaagcgcttcgttaatgaacttgacctccgttctgtggtatcgaaagccatcatggaacagggtgcggaatatcctggatcgggtggtagcatgttcgcctactgtgtgttgaatgctgcggcgcagaagctctttggtgtcagctcacacgagttctactggaagaagatgggtctcttcgtgaaggctgacaccatgagggacctcgctgcactcattgggtgcccagtggaatctgtgcagcagacgctggaggagtacgagcggctctccatatcacagcgttcctgccccatcacgcgcaaaagcgtctatccgtgcgtgctcggcactaagggcccctactacgtcgccttcgtgacaccttcgattcactacacaatgggtggatgtctcatctcgccttctgctgaaatacaaatgaagaacacatcatcacgcgctccactgagccacagcaacccaatcctcgggttatttggtgccggtgaggtaacgggtggtgtgcacggtgggaaccggttgggcggcaattcgctgcttgagtgcgtcgtgtttgggagaattgcgggggcgagtgcagcgaatgctaaatatcacaatgagactcacctttggaacaaccgatgggctaagatggtagttgaaagcgttcttgacgacacgaacgggtttcagtggttatatttcaggcttccaagcacgctccagcgttcgggtgtgggaccactgcaagcagttgtgttgcgggagatggagggggaaggcaggttggatgtccgtattccatttacggtgcctggggacgttggcgttgttggtattatggtttattccaacgacgagaacagttccatggggtggttaactgcgctgctgcccggcggaatggtggagatgaaagcgggggtacaagtcgacgccaactatgaaaggatcctggcgcttccccggaaggtaattatcgccactcgtgatggcgtggcgccgatggtccaaatgattagagcagccttgcatgaagcgaaggacgaacctgcgcttcaaataatttacatcgcagaacgtgtcgcgacgattccccagcgtgaaaagttggagcagcttcaaagggatcatccgaacaagtttaagttcactttcgttgtgcacgatccgcccccgctttggaccggaggtgtcaacatcatggaggaaatatctaaatctgtgttccctgatgcgagtctgggcgttttcctatttgctagtgcgaccgagtccgcatcactgcaagttcagctgttggagttggggcataacaaatcaaacatagtcactctttga

SEQ ID NO: 14 - protein, 877 aa, Trypanosoma brucei
MCHTRVWASMRLHGCLLSPLTSVSMCHPFWLQFFVCSFHLFLDTVARVSFTGLKSLTHRNRLVGVAMFSGPAEVYYTKSIQNNHRQLRRNVGGAQPGRGIYYSLLAVLLVVTFIKTASATLERVRVGIDMIRSREMAMVESENPRVIVVGGGLAGLSAAIEAAGCGAQVVLMEKEAKLGGNSAKATSGINGWGTRAQAKASIVDGGKYFERDTYKSGIGGNTDPALVKTLSMKSADAIGWLTSLGVPLTVLSQLGGHSRKRTHRAPDKKDGTPLPIGFAIVKTLEDHVRGNLSGRITIMENCSVTSLLSETKERPDGTKQIRVTGVEFTQAGSGKTTILADAVILATGGFSNDKTADSLLREHAPHLVNFPTTNGPWATGDGVKLAQRLGAQLVDMDKVQLHPTGLINPKDPANPTKFLGPEALRGSGGVLLNKQGKRFVNELDLRSVVSKAIMEQGAEYPGSGGSMFAYCVLNAAAQKLFGVSSHEFYWKKMGLFVKADTMRDLAALIGCPVESVQQTLEEYERLSISQRSCPITRKSVYPCVLGTKGPYYVAFVTPSIHYTMGGCLISPSAEIQMKNTSSRAPLSHSNPILGLFGAGEVTGGVHGGNRLGGNSLLECVVFGRIAGASAANAKYHNETHLWNNRWAKMVVESVLDDTNGFQWLYFRLPSTLQRSGVGPLQAVVLREMEGEGRLDVRIPFTVPGDVGVVGIMVYSNDENSSMGWLTALLPGGMVEMKAGVQVDANYERILALPRKVIIATRDGVAPMVQMIRAALHEAKDEPALQIIYIAERVATIPQREKLEQLQRDHPNKFKFTFVVHDPPPLWTGGVNIMEEISKSVFPDASLGVFLFASATESASLQVQLLELGHNKSNIVTL

SEQ ID NO: 15 - DNA, 3444 bp, Leishmania braziliensis
  CDS (1)..(3444): Sequence coding for a fumarate reductase
atggcggacggaaagacctctgcgtctgttgtagcggttgaccccgagcgcgcggcgaaggaacgcgacgcagccgcccgcgcgatgctacaagacggcggcgtctcgccggtcggaaaggctcagctgttgaagaagggactcgcgtacgccgttccgtataccctcaagatcgtcgtggcagaccccaaggcaatggagaagacgacggcagatgtagagaaagtgctccagaccgccttccaggtggtcgacacccttctcaacaacttcaacgagaacagtgaggtgtcccgcatcaaccgaatgcccgttggtgaagaacaccagatgtctgcggcgctgaagcgtgtgatgggctgctgccagcgtgtgtacaactcgtcgcgcggcgcctttgaccctgcagtcggcccgctcgtccgtgagctacgcgaggccgctcgtgaggggaggacgctgccagcggagcgcatcaacgccctcctcagcaagtgcacgctgaacatcagcttctccattgacctaaaccgtggcacaatcgcccgtaagcatgcggacgcgatgctggaccttggtggtgtgaacaagggctacggtgtcgactacgtcgtcgaacacctcaacaatctcggctacgacgatgtcttctttgaatggggcggtgatgtgcgtgccagtgggaagaacccgtcaaatcaacactgggtcgtcggcatcgcgcgcccacccgctttagcggacattcgtacagtggtgccgcaggacaagcagtcctttatccgtgtggtgtgcctcaacgacgaggccatcgccaccagcggtgactacgagaacctcgttgagggccctgggtcaaaggtatattcatctaccttcaatgcaacgtccaagagcctgctggagccgaccgagacgaacatagcacaggtttccgtaaagtgctacagctgcatgtacgccgatgccctggccaccgctgcgctcctcaagaacaacccgacggccgttcgccgcatgctggacaactggcgctacgtgcgcgacaccgtcaccgactacaccacctacagccgtgagggtgagcgtgttgccaagatgttcgaaatcgccacggaggataaggagatgcgcgcgaagcgcatcagtggctcgctcccggcccgtgtgatcatcgttggtggcggtctggccggttgctcggctgcgatcgaagcagtcaactgtggcgcgcaggtaattctcctcgagaaagaggcaaagattggcggcaacagcgccaaggcaacatccggcatcaacgcctggggcactcgtgcgcaggcgaagcagggcgtcatggacggtggcaagttctttgagcgcgacacacaccgttccggcaagggtggtcactgcgatccgtgccttgtcaagacgctctctgtgaagagctctgacgcggtgaagtggctgtccgagctgggcgtgccgctgacggtgctgtcgcagctcggcggtgcaagtcgcaagcgctgccaccgcgccccagataagtcagatggtaccccagtcccgatcggcttcacgatcatgaagaccctggagaaccacatcatcaacgacctcagtcaccaggtcactgtaatgacaggcattaaagtgacagggctagagagcacgagccatgcccgccctgatggcgtccttgtgaagcacgtcacgggcgtccgcctcatccagggcgacgggcagtccagggtgctgaacgccgacgccgtcattctcgccaccggcggcttctcgaacgaccacacggccaactcgcttctgcagcagtacgcaccgcaactatcctccttccccacaaccaacggcgtgtgggccacaggcgatggcgtcaaggcggcgcgtgagcttggcgtggagcttgtcgacatggacaaggtgcagctgcacccgaccggcctgctggacccgaaggatccgtcgaatcgcaccaagtacctcggccccgaggcgctgcgtggctccggcggcgtgctgctgaacaagaacggtgagcgctttgtgaacgagctggacctgcgctccgtcgtgtcgcaggcgattattgagcagaacaacgtctaccccgggtccggcggcagcaaattcgcatactgtgtgctgaatgaggccgcggcgaagctcttcggaaagaacttcctgggcttctactggcaccgtcttggcctcttcgagaaggtggaggacgtcgctggcctggcgaagctgattggctgccccgaggagaatgtgacggcaactctgaaggagtacaaggagctctcctccaagaagctgcacgcctgcccgttgactaacaagaacgtgttcccgtgcaccctgggaaccgaggggccctactacgtagccttcgtcacgccgtcgatccactacaccatgggcggctgcctcatctccccttcggctgaaatgcagacgatagacaataccggcgtgacccccgttcgtcgtcccatccttgggctctttggcgctggcgaggtgactggtggcgtgcacggtggaaaccgcctcggcggcaactcgctgttggagtgcgtcgtctttggaaggatcgccggcgaccgtgcagccacaattctgcaaaaaaagaacgcggggctgtccatgacggagtggtcgacggtggtgctgcgtgaggtgcgcgagggtggcgtgtatggtaccggctcgcgcgtactgcgattcaacatgcccggcgcgctgcagaaaacgggccttgcgcttggtcagttcatcgccatgcgtggtgactgggacggtcagcagctgctcggctactatagccccatcacgctgccagacgacatcggtgtgattggtatcctcgcccgtgccgacaagggccgcctagcggagtggatctctgccctacagcctggcgacgcggtggagatgaaggcgtgcggtggtctcattattcaccgccgcttcgctgcccgccatctcttcttccgcagccacaagattcgcaagctggctctcatcggcggcggcacgggtgtcgcaccgatgctgcagattgtgcgggctgcggtgaagaagcccttcgtagactcgatcgagagcatccagttcatctacgccgccgaggacgtatcggagctgacgtatcgcacgctgcttgagagttacgaaaaggagtacggctctggtaagttcaagtgccacttcgtcctcaacaaccctccttctcagtggaccgagggcgtcggcttcgttgataccgctctgctgcgctccgccgtgcaggcgccgtccaacgaccttctggtggccatctgtggtccgccgatcatgcagcgtgctgtgaaaagtgcactcaagggcctcggctacaacatgaatctcgtgcgcacggtggatgagccagagcctctctcggccaagatttaa

SEQ ID NO: 16 - protein, 1147 aa, Leishmania braziliensis
MADGKTSASVVAVDPERAAKERDAAARAMLQDGGVSPVGKAQLLKKGLAYAVPYTLKIVVADPKAMEKTTADVEKVLQTAFQVVDTLLNNFNENSEVSRINRMPVGEEHQMSAALKRVMGCCQRVYNSSRGAFDPAVGPLVRELREAAREGRTLPAERINALLSKCTLNISFSIDLNRGTIARKHADAMLDLGGVNKGYGVDYVVEHLNNLGYDDVFFEWGGDVRASGKNPSNQHWVVGIARPPALADIRTVVPQDKQSFIRVVCLNDEAIATSGDYENLVEGPGSKVYSSTFNATSKSLLEPTETNIAQVSVKCYSCMYADALATAALLKNNPTAVRRMLDNWRYVRDTVTDYTTYSREGERVAKMFEIATEDKEMRAKRISGSLPARVIIVGGGLAGCSAAIEAVNCGAQVILLEKEAKIGGNSAKATSGINAWGTRAQAKQGVMDGGKFFERDTHRSGKGGHCDPCLVKTLSVKSSDAVKWLSELGVPLTVLSQLGGASRKRCHRAPDKSDGTPVPIGFTIMKTLENHIINDLSHQVTVMTGIKVTGLESTSHARPDGVLVKHVTGVRLIQGDGQSRVLNADAVILATGGFSNDHTANSLLQQYAPQLSSFPTTNGVWATGDGVKAARELGVELVDMDKVQLHPTGLLDPKDPSNRTKYLGPEALRGSGGVLLNKNGERFVNELDLRSVVSQAIIEQNNVYPGSGGSKFAYCVLNEAAAKLFGKNFLGFYWHRLGLFEKVEDVAGLAKLIGCPEENVTATLKEYKELSSKKLHACPLTNKNVFPCTLGTEGPYYVAFVTPSIHYTMGGCLISPSAEMQTIDNTGVTPVRRPILGLFGAGEVTGGVHGGNRLGGNSLLECVVFGRIAGDRAATILQKKNAGLSMTEWSTVVLREVREGGVYGTGSRVLRFNMPGALQKTGLALGQFIAMRGDWDGQQLLGYYSPITLPDDIGVIGILARADKGRLAEWISALQPGDAVEMKACGGLIIHRRFAARHLFFRSHKIRKLALIGGGTGVAPMLQIVRAAVKKPFVDSIESIQFIYAAEDVSELTYRTLLESYEKEYGSGKFKCHFVLNNPPSQWTEGVGFVDTALLRSAVQAPSNDLLVAICGPPIMQRAVKSALKGLGYNMNLVRTVDEPEPLSAKI

SEQ ID NO: 17 - DNA, 3585 bp, Leishmania braziliensis
  CDS (1)..(3585): Sequence coding for a fumarate reductase
atgggtgcctccgcaatggcgacgcagcggcgctgcacggtctccgactcgcatacgggtgcttccatagtcgtcgtgaaccctgaaaaggcagctcgtgagcgcgatcgcattgctcgcgacctactcaccaccaactttccagagctgcatgtcaaccagcgctcagtgctgttgtacaaggatgtggtgcacaccgttccgtacacgctttccatcgctgtagacggcagtgtcactcgccaagatgcagatcctgttgtgaaggccattctgagcaactgcttcaccatggttgatacgcacctcaactccttcaatccggacagcgaggtgtcgaaggtcaacaggatgccggtgggcgagaagcacgtcatgtcggagcatctctgcaaggtggtcaaatgctgcgaagagatctacaacatcagcggcagctgcttcgacccggccgctgcaccgctggtgcacaggcttcgcaatgccgctcgccggcaagattccaccgaggcggacttcatgatcacagcagaggtggcgggacgcttcaccctgacgaacagctttgccatcaatgtcaaggaaggcacgatcgcacgcaagcacgaagacgcgatgctggacctgggtggtctgaacaagggctacaccgtcgactgtgtggtggatcagctgaatgcagccaatttttctgacgtgctgtttgagtggggcggcgactgtcgcgcctcaggtgtgaacgtacagcgccagccgtgggcagtcggcgttgtgcgtccgccatcggtcgacgaggtcgtggcggcttccaagtccggcaagtcggtaacgatgaatgcccacagtctcggggagcgctcagatgagccggcgcagtcggcgtcggccgccgacggggcagccaagcctgggcacaagacgctcctacgcgtcatgtcgctcagcaacgaggcactctgcacgagcggcgactacgagaacgtcctcttcgttaacgcacttaaccgcgctgtttcgagcacgtacgactggcgtcaccgttgcctcattggaccctctcagggcggcctggcccaggttagcgtcaaatgctattcctgcctctatgccgacgcactcgccactgcgagcttcgtgaagcgcaaccccgtgcccatacggtacatgctcgagcactatcgccgtgattacaaccgcgtgaccgactacactgcctacacgcgtgagggggagcggctggcgcacatgtacgagatcgcgtgcgagagcccggcctgccggacagagcgcattgcaggctcactgccagcgcgggtagtcgtgatcggcggcggtcttgccgggtgtgccgctgcgattgaggcagccagctgcggtgccaccgtgattctcctagagaaggaagcgcgactgggtggcaacagtgccaaggccacgtctggcatcaacgcctggggcacccgcacgcaggccgtgaatcacgttctcgacaactgcaagttctttgaacgtgacacgttcctctccggtaagggtggtcactgcgacccagggcttgtgcgcaccctctctgtaaagtctgccgaggcaattagctggctcgagtccttcggcattccgctaaccgccctctaccaacttggtggggcgagccgcccgcgctgccaccgcgcaccagatcaaaaggacggctccccggtgcccattggcttcacaatcatgcgtcacctggagaactacatccacaccaagctgcagggaaaggtgaccatcttgtgcgaaacggcggtggtgagcctcctacacgacgtgagtgcgatgccggacggcagccgcgagattcgcgtgcgcggtgtccgctacaagtcgacgagcgatgcgtcggaggcggtgatcgatctgccggcggacgccgtcgtgcttgccaccggtggcttctcaaacgatcagacggtcaactcgctgctgcgcgagtacgcgccaaacgtgtacggcacccccaccaccaacggcgcgttcgccaccggcgatggcgtcaagatggcacgccagcttggcgccaggctggtggacatggacaaggtgcagctgcacccgaccggtctcattgaccccaaggatccgtcgaatcgcaccaaataccttggccccgaggcactgcgcggctccggcggtatcctactgaatcgcaacggcgagcgctttgtgaatgagctggacctgcgctccgtcgtgtcgcaggcaatcaacgcgcaggacgacgagtacccgaactcgggcggcagcaagtttgcgtactgtgtgctgagcgaggaggctgccaagctgtttggcaagaacgccctcacatactactggaaatcgcagggcctcttcacccgtgttgacaacataaaggcgctcgcggagctcatcggctgctcggttgacaacctgcatcgcactctcgagacatacgagcaccagagcaccgcgaaggtggcctgcccgctgactggcaaacttgtgtttccgagtgtggtgggcaccagtggcccctattacgtggcgtacgtcacgccgtcgattcactacaccatgggcggctgctccatctcaccggcggcggagctgctcatggaggatcactccgtcaacatcttcgaagacatgcgccctattcttggcctcttcggcgctggcgaggtgactggcggcgtgcacggccgcaaccgtctcggcggcaactctcttttggagtgcgtcgttttcggcaagatcgctggcgaccgcgcggctacaattctgcagaaggagaagtacgggctcagcaaggacaagtgggtgccggtggtggtgcgggagacgagggcaagtgaccagttcggtgtcggctcgcgcgtgctgcacttcaacctgcccggtgcgacacaaacatccggactgaccgtcggcgagttcatcagcatccgcggtgactgggacggccagcatctgatcggctactacagccccatcagcatgcccgatgacaagggccgcatctcaatcctggcacgtggtgacaagggcaacctgcaggaatgggtctcgtccatgcgtcctggcgactcagtcgagatgaaggcctgcggcggcctccgtatcgagctcaagcctcagcagaagcagatgatctaccgcaagacggtcatccgaaaactggccctcatcgccggtggctccggtgtggcaccgatgctgcagattatcaaggccgcgctcagtcgcccctatgtggacagcatagagacgatccgtctagtgtacgccgccgaggacgagtatgaactgacctaccgctcattgttgaagaagtaccgcaccgacaacacggagaagttcgactgcgacttcgtgctcaacaaccctcccgaaggttggacggagggtgtgggctacgtcgaccgcgccacgctgcagagctttctcccgcctccgtcgaagggcctgctggtagccatttgcggcccaccggtgatgcagcactccgttgtggcggacctgctggcactaggctatagcgccgagacggtgcgcacgatagatgaggatcgcatgctctag

SEQ ID NO: 18 - protein, 1194 aa, Leishmania braziliensis
MGASAMATQRRCTVSDSHTGASIVVVNPEKAARERDRIARDLLTTNFPELHVNQRSVLLYKDVVHTVPYTLSIAVDGSVTRQDADPVVKAILSNCFTMVDTHLNSFNPDSEVSKVNRMPVGEKHVMSEHLCKVVKCCEEIYNISGSCFDPAAAPLVHRLRNAARRQDSTEADFMITAEVAGRFTLTNSFAINVKEGTIARKHEDAMLDLGGLNKGYTVDCVVDQLNAANFSDVLFEWGGDCRASGVNVQRQPWAVGVVRPPSVDEVVAASKSGKSVTMNAHSLGERSDEPAQSASAADGAAKPGHKTLLRVMSLSNEALCTSGDYENVLFVNALNRAVSSTYDWRHRCLIGPSQGGLAQVSVKCYSCLYADALATASFVKRNPVPIRYMLEHYRRDYNRVTDYTAYTREGERLAHMYEIACESPACRTERIAGSLPARVVVIGGGLAGCAAAIEAASCGATVILLEKEARLGGNSAKATSGINAWGTRTQAVNHVLDNCKFFERDTFLSGKGGHCDPGLVRTLSVKSAEAISWLESFGIPLTALYQLGGASRPRCHRAPDQKDGSPVPIGFTIMRHLENYIHTKLQGKVTILCETAVVSLLHDVSAMPDGSREIRVRGVRYKSTSDASEAVIDLPADAVVLATGGFSNDQTVNSLLREYAPNVYGTPTTNGAFATGDGVKMARQLGARLVDMDKVQLHPTGLIDPKDPSNRTKYLGPEALRGSGGILLNRNGERFVNELDLRSVVSQAINAQDDEYPNSGGSKFAYCVLSEEAAKLFGKNALTYYWKSQGLFTRVDNIKALAELIGCSVDNLHRTLETYEHQSTAKVACPLTGKLVFPSVVGTSGPYYVAYVTPSIHYTMGGCSISPAAELLMEDHSVNIFEDMRPILGLFGAGEVTGGVHGRNRLGGNSLLECVVFGKIAGDRAATILQKEKYGLSKDKWVPVVVRETRASDQFGVGSRVLHFNLPGATQTSGLTVGEFISIRGDWDGQHLIGYYSPISMPDDKGRISILARGDKGNLQEWVSSMRPGDSVEMKACGGLRIELKPQQKQMIYRKTVIRKLALIAGGSGVAPMLQIIKAALSRPYVDSIETIRLVYAAEDEYELTYRSLLKKYRTDNTEKFDCDFVLNNPPEGWTEGVGYVDRATLQSFLPPPSKGLLVAICGPPVMQHSVVADLLALGYSAETVRTIDEDRML

SEQ ID NO: 19 - DNA, 1545 bp, Leishmania braziliensis
  CDS (1)..(1545): Sequence coding for a fumarate reductase
atgtctgacatagcctcttcactaaaccgtgtcgtcgtcatcggcagcgggcttgcagggcaatgcgcagcaatcgaggctgctcgccacggagctaaggaagttgtaataattgaaaaggaaacccggctgggaggcaacagcgctaaggcaacgtccggtatcaacgcctggggcacggcggtgcagaaagccgctggcgtgcacgacagcggtgagctctttgaaaaggatacgttcgcctctggcaagggcggcagctgccagccagagttggtacggacgctgtccgaccacagcgcagaagccatcgagtggctctcctcgttcggcatcccgctgactgcccttacgcagctcggcggtgcaagtcgcaagcgctgccaccgcgccccagacaacccagacgggaccccgctgccgatcggcttcaccattgtgcgtgccctggagaactacattcgcacgaacttgtccgacatcgtgcgcatcgaaaccaatgcgcgtctcatctcactgatgcacagtaaggaggacaacgcagtggtagtgcaaggcatcacgtacgccacgcaaactgcaagtggagaggggaagattcgcaaactacaggcccgtgccgtcattctcgccaccggcggcttctcgaacgaccacacggccaactcgcttctgcagcagtacgcaccgcaactatcctccttccccacaaccaacggcgtgtgggccacaggcgatggcgtcaaggcggcgcgtgagcttggcgtggagcttgtcgacatggacaaggtgcagctgcacccgactggcctgctggacccgaaggatccgtcgaatcgcaccaagtacctcggccccgaggcgctgcgtggctccggcggcgtgctgctgaacaagaacggtgagcgctttgtgaacgagctggacctgcgctccgtcgtgtcgcaggcgattattgagcagaacaacgtctaccccgggtccggcggcagcaaattcgcatactgtgtgctaaatgaggccgcggcgaagctcttcggaaagaacttcctgggcttctactggcaccgtcttggcctcttcgagaaggtggaggacgtcgctggcctggcgaagctgattggctgccccgaggagaatgtgacggcaactctgaaggagtacaaggagctctcctccaagaagctgcacgcctgcccgttgactaacaagaacgtgttcccgtgcaccctgggaaccgaggggccctactacgtagccttcgtcacgccgtcgatccactacaccatgggcggctgcctcatctccccttcggctgaaatgcagacgatagacaataccggcgtgacccccgttcgtcgtcccatccctgggctctttggcgctggcgaggtgactggtggcgtgcacggtggaaaccgcctcggcggcaactcgctgttggagtgcgtcgtctttggaaggatcgccggcgcccgcgcggctgcaattctgcaggagggggccgcggggttgcccctgcttgggggagggcaggccgggtga

SEQ ID NO: 20 - protein, 514 aa, Leishmania braziliensis
MSDIASSLNRVVVIGSGLAGQCAAIEAARHGAKEVVIIEKETRLGGNSAKATSGINAWGTAVQKAAGVHDSGELFEKDTFASGKGGSCQPELVRTLSDHSAEAIEWLSSFGIPLTALTQLGGASRKRCHRAPDNPDGTPLPIGFTIVRALENYIRTNLSDIVRIETNARLISLMHSKEDNAVVVQGITYATQTASGEGKIRKLQARAVILATGGFSNDHTANSLLQQYAPQLSSFPTTNGVWATGDGVKAARELGVELVDMDKVQLHPTGLLDPKDPSNRTKYLGPEALRGSGGVLLNKNGERFVNELDLRSVVSQAIIEQNNVYPGSGGSKFAYCVLNEAAAKLFGKNFLGFYWHRLGLFEKVEDVAGLAKLIGCPEENVTATLKEYKELSSKKLHACPLTNKNVFPCTLGTEGPYYVAFVTPSIHYTMGGCLISPSAEMQTIDNTGVTPVRRPIPGLFGAGEVTGGVHGGNRLGGNSLLECVVFGRIAGARAAAILQEGAAGLPLLGGGQAG

SEQ ID NO: 21 - DNA, 1632 bp, Leishmania braziliensis
  CDS (1)..(1632): Sequence coding for a fumarate reductase
atggtctccgggacgcatgcggcgcggctcgttttgaaaaaagggcacgccaaaccagcagcgccggggcgggggcgtccgcgtttcccggcccgttggcggggcatgatggccaccgcccgcggagcacggcggcttgcttcagacgaagcgggtgcgcctcacgcaaagctccgggtgttgcgtcgcgccgggcgttttctgcttcgcggggcgcctggcgagtgcgccgcccgcagccgagaaaacggcaacgcctggggcacggcggtgcagaaagccgctggcgtgcacgacagcggtgagctctttgaaaaggatacgttcgcctctggcaagggcggcagctgccagccagagttggtacggacgctgtccgaccacagcgcagaagccatcgagtggctctcctcgttcggcatcccgctgactgcccttacgcagctcggcggtgcaagtcgcaagcgctgccaccgcgccccagacaacccagacgggaccccgctgccgatcggcttcaccattgtgcgtgccctggagaactacattcgcacgaacttgtccgacatcgtgcgcatcgaaaccaatgcgcgtctcatctcactgatgcacagtaaggaggacaacgcagtggtagtgcaaggcatcacgtacgccacgcaaactgcaagtggagaggggaagattcgcaaactacaggcccgtgccgtcattctcgccaccggcggcttctcgaacgaccacacggccaactcgcttctgcagcagtacgcaccgcaactatcctccttccccacaaccaacggcgtgtgggccacaggcgatggcgtcaaggcggcgcgtgagcttggcgtggagcttgtcgacatggacaaggtgcagctgcacccgaccggcctgctggacccgaaggatccgtcgaatcgcaccaagtacctcggccccgaggcgctgcgtggctccggcggcgtgctgctgaacaagaacggtgagcgctttgtgaacgagctggacctgcgctccgtcgtgtcgcaggcgattattgagcagaacaacgtctaccccgggtccggcggcagcaaattcgcatactgtgtgctgaatgaggccgcggcgaagctcttcggaaagaacttcctgggcttctactggcaccgtcttggcctcttcgagaaggtggaggacgtcgctggcctggcgaagctgattggctgccccgaggagaatgtgacggcaactctgaaggagtacaaggagctctcctccaagaagctgcacgcctgcccgttgactaacaagaacgtgttcccgtgcaccctgggaaccgaggggccctactacgtagccttcgtcacgccgtcgatccactacaccatgggcggctgcctcatctccccttcggctgaaatgcagacgatagacaataccggcgtgacccccgttcgtcgtcccatccctgggctctttggcgctggcgaggtgactggtggcgtgcacggtggaaaccgcctcggcggcaactcgctgttggagtgcgtcgtctttggaaggatcgccggcgcccgcgcggctgcaattctgcaggagggggccgcggggttgcccctgcttgggggagggcaggccgggtga

SEQ ID NO: 22 - protein, 543 aa, Leishmania braziliensis
MVSGTHAARLVLKKGHAKPAAPGRGRPRFPARWRGMMATARGARRLASDEAGAPHAKLRVLRRAGRFLLRGAPGECAARSRENGNAWGTAVQKAAGVHDSGELFEKDTFASGKGGSCQPELVRTLSDHSAEAIEWLSSFGIPLTALTQLGGASRKRCHRAPDNPDGTPLPIGFTIVRALENYIRTNLSDIVRIETNARLISLMHSKEDNAVVVQGITYATQTASGEGKIRKLQARAVILATGGFSNDHTANSLLQQYAPQLSSFPTTNGVWATGDGVKAARELGVELVDMDKVQLHPTGLLDPKDPSNRTKYLGPEALRGSGGVLLNKNGERFVNELDLRSVVSQAIIEQNNVYPGSGGSKFAYCVLNEAAAKLFGKNFLGFYWHRLGLFEKVEDVAGLAKLIGCPEENVTATLKEYKELSSKKLHACPLTNKNVFPCTLGTEGPYYVAFVTPSIHYTMGGCLISPSAEMQTIDNTGVTPVRRPIPGLFGAGEVTGGVHGGNRLGGNSLLECVVFGRIAGARAAAILQEGAAGLPLLGGGQAG

SEQ ID NO: 23 - DNA, 3444 bp, Leishmania major strain Friedlin; identical to SEQ ID NO: 5
  CDS (1)..(3444): Sequence coding for a fumarate reductase

SEQ ID NO: 24 - protein, 1147 aa, Leishmania major strain Friedlin; identical to SEQ ID NO: 6

SEQ ID NO: 25 - DNA, 1488 bp, Leishmania major strain Friedlin; identical to SEQ ID NO: 7
  CDS (1)..(1488): Sequence coding for a fumarate reductase

SEQ ID NO: 26 - protein, 495 aa, Leishmania major strain Friedlin; identical to SEQ ID NO: 8

SEQ ID NO: 27 - DNA, 3444 bp, Leishmania infantum
  CDS (1)..(3444): Sequence coding for a fumarate reductase
atggcggatggaaagacctctgcatctgtggtggcggtcgacgccgagagcgcggcaaaggagcgcgacgcagccgcccgcgcgatgctgcaggacggcggcgtttcaccagtcggaaaggctcagctgttaaagaagggcctcgtgcacacggttccgtacaccctcaaggtcgtcgtggcggaccccaaggagatggagaaggccgcagcagatgcggagcaagtgctccaggctgccttccaggtggtcgacaccctcctcaataacttcaacgagaacagcgaggtgtcccgcatcaatcgaatgccggtcggtgaggaacaccagatgtctgcggctctgaagcatgtgatggcctgctgccagaaagtctacaactcgtcgcgcggcgccttcgaccccgccgtcggtcccctcgtccgagagctgcgcgaggccgcacataagggcaagacggtgccggcggagcacgtcaacgacctcctcagcaagtgcacgctgaacgctagcttctccattgacatgaaccgtggcatgatcgcccgcaagcacgcggacgcgatgctggatcttggtggtgtgaacaagggctacggcatcgactacatcgtcgagcgcctcaacagcctcggctacaacgacgtcttcttcgagtggggcggtgatgtgcgtgccagtggcaagaaccagtcgaaccagccctgggccgtcggcatcgtgcgcccgcccgctttggcggacattcgcactgtggtgccggaagacaagcggtccttcatccgggtggtgcgcctcaacaacgaagccatcgccaccagcggtgactacgagaacctcattgagggccccggctccaaggtgtactcgtcgaccttcgatccggcgtccaagaacctgctggagccaaccgaggcggacatggcacaagtctccgtgaagtgctacagctgcatgtacgccgatgccctggccaccgccgcgctcctcaagaacgacccggcggccgtccgccgcatgctggacaactggcgctacgtgcgcgataccgtcaccgactacaccacctacactcgtgagggcgagcgtgtcgccaagatgctcgaaatcgctacggaggatgcggagatgcgcgcgaagcgcatcaagggctcgctgccggcgcgcgtgatcatcgttggtggaggtctggccggttgctcggccgcgatcgaggcggccaactgtggcgcgcaggtgattctgctggagaaggagccaaagctcggcggcaacagcgccaaagcaacgtccggcatcaacgcctggggaacccgtgcgcaggcgaagcagggcgtcatggacggcggcaagttctttgagcgcgacacacaccgctccggcaagggtggcaactgcgatccgtgcctcgtcaagacgctatctgtaaagagctccgacgcggtgaagtggctgtccgagctgggcgtgccgctgacggtgctgtcgcagctcggcggcgcgagccgcaagcgctgccaccgcgcaccagataagtcggacggtaccccggtcccggtgggcttcaccatcatgaagaccctggagaaccatatcgtcaacaacctcagtcgccatgttactgtgatgacgggcattactgtgacagcgctggagagcacgagccgtgtccgccctgatggcgtccttgtgaagcacgtgacgggcgtccgcctcatccagtccagcgggcagtccatggtgctgaacgccgacgctgtcgttctcgccaccggcggcttctcgaacgaccacacgcccaactcgctcctgcagcagtacgcgccgcaactgtcgtccttccccaccaccaacggtgtatgggccaccggtgatggcgtgaagatggcgagcaagctgggcgtgacgctggtagacatggacaaggtgcagctgcacccgaccggtctgctagacccgaaggatccgtcgaatcgcaccaagtaccttggccccgaggcgctgcgtggctccggtggcgtgctgctgaacaagaacggcgagcgcttcgtgaacgagctggacctgcgctccgtcgtgtcgcaggcgattatcgcgcaggacaacgtctaccccgggtccggcggcagcaaattcgcgtactgtgtgctgaatgagaccgcggcgaagctcttcggcaagaatttcctcggcttctactggaaccgcctcggtctcttccagaaggtggatagcgtcgctggcctggcgaaactgattggctgccctgaggcgaatgtgatggcaaccctgaagcagtacgaggagctctcctccaagaagttaaacccctgcccgctgactggcaagaacgtgttcccttgtgtgctgggtactcaagggccctactacgttgccctcgtcacgccgtcgatccactacaccatgggtggctgcctcatctcgccctcggcggagatgcagaccatagacaatagcggtgtgacccccgttcgtcgtccgattcttggtctctttggcgctggcgaggtgacgggcggcgtgcacggtggcaaccgtctcggcggtaactcgctgctagagtgtgtcgtgtttggcaagatcgccggcgaccgcgcggccacgattctgcagaagaagagcacggggctatccacgacggagtggtcgaccgtggtgctgcgtgaggtgcgcgagggtggcgtgtacggtgcgggctcgcgcgtgctgcgcttcaacatgcccggcgcgctgcagaggactggccttgctcttggtcagtttatcggcattcgcggtgactgggacggccaccgactgatcggctactacagccccatcacgcttccggacgatgtcggtgtgattggcatcctcgcccgtgccgacaagggccgcctggcggagtggatctctgccctgcagcctggagacgcggtggagatgaaggcgtgcggtggccttatcatcgagcgccgcttcgccgaccgccacttctttttccgtggccacaagattcgcaagctcgccctcatcggtggcggcacgggtgtcgcgccgatgctgcagattgtgcgggctgcggtgaagaaacccttcgtggactcgattgagagcattcagttcatctacgccgccgaggacgtatcagagctgacgtaccgcacgctgcttgagagctacgagaaggagtatggctctgagaagttcaagtgtcacttcgttctcaacaaccctcctgctcagtggaccgacggcgtcggcttcgttgataccgctttgctacgctccgccgtgcaggcgccgtccaacgaccttctggtggccatctgcggtccgccgatcatgcagcgtgcggtcaagggtgccctcaagggtctcggctacaacatgaacctcgtgcgcacggtggatgaaacagagcccacctcggccaagatttga

SEQ ID NO: 28 - protein, 1147 aa, Leishmania infantum
MADGKTSASVVAVDAESAAKERDAAARAMLQDGGVSPVGKAQLLKKGLVHTVPYTLKVVVADPKEMEKAAADAEQVLQAAFQVVDTLLNNFNENSEVSRINRMPVGEEHQMSAALKHVMACCQKVYNSSRGAFDPAVGPLVRELREAAHKGKTVPAEHVNDLLSKCTLNASFSIDMNRGMIARKHADAMLDLGGVNKGYGIDYIVERLNSLGYNDVFFEWGGDVRASGKNQSNQPWAVGIVRPPALADIRTVVPEDKRSFIRVVRLNNEAIATSGDYENLIEGPGSKVYSSTFDPASKNLLEPTEADMAQVSVKCYSCMYADALATAALLKNDPAAVRRMLDNWRYVRDTVTDYTTYTREGERVAKMLEIATEDAEMRAKRIKGSLPARVIIVGGGLAGCSAAIEAANCGAQVILLEKEPKLGGNSAKATSGINAWGTRAQAKQGVMDGGKFFERDTHRSGKGGNCDPCLVKTLSVKSSDAVKWLSELGVPLTVLSQLGGASRKRCHRAPDKSDGTPVPVGFTIMKTLENHIVNNLSRHVTVMTGITVTALESTSRVRPDGVLVKHVTGVRLIQSSGQSMVLNADAVVLATGGFSNDHTPNSLLQQYAPQLSSFPTTNGVWATGDGVKMASKLGVTLVDMDKVQLHPTGLLDPKDPSNRTKYLGPEALRGSGGVLLNKNGERFVNELDLRSVVSQAIIAQDNVYPGSGGSKFAYCVLNETAAKLFGKNFLGFYWNRLGLFQKVDSVAGLAKLIGCPEANVMATLKQYEELSSKKLNPCPLTGKNVFPCVLGTQGPYYVALVTPSIHYTMGGCLISPSAEMQTIDNSGVTPVRRPILGLFGAGEVTGGVHGGNRLGGNSLLECVVFGKIAGDRAATILQKKSTGLSTTEWSTVVLREVREGGVYGAGSRVLRFNMPGALQRTGLALGQFIGIRGDWDGHRLIGYYSPITLPDDVGVIGILARADKGRLAEWISALQPGDAVEMKACGGLIIERRFADRHFFFRGHKIRKLALIGGGTGVAPMLQIVRAAVKKPFVDSIESIQFIYAAEDVSELTYRTLLESYEKEYGSEKFKCHFVLNNPPAQWTDGVGFVDTALLRSAVQAPSNDLLVAICGPPIMQRAVKGALKGLGYNMNLVRTVDETEPTSAKI

SEQ ID NO: 29 - DNA, 3585 bp, Leishmania infantum
  CDS (1)..(3585): Sequence coding for a fumarate reductase
atgggtggctgcgcaacgtcgctgtgtcgccgttgcgcagccaccgactcgcatacaggcgcttccgtagtcgtttcggaccccgaaaaggccgctcgtgagcgcgatcgcattgctcgcggcctgctcaccaccaactttcccgagctgcacgtcaaccagcgctcggtgctgcggtataaggacgtgatgcacacggtgccgtacacgctcaccatcgctgtagacggtaatgtcgctcgccaagatgtggatcctgtcgtcaaggcaattctaagcgactgcttcgcgatggtggacaagcacctcaactccttcaacccgggcagcgaggtgtcgcaggtcaacaggatgccggtgggaaagaagcacgtcatgtcggagcacctcttcgaggtggtcaagtgctgcgaggaggtctacagcagtagcggcagctgctttgacccggccgcagcaccgctggtgcacaagctgcgcgatgccgctcgccggcaggactccgccgagggggacttcgccatctctgcggaggaggcggggcgtttcaccctgacgaacagctttgccatcgacatcaaagaaggcaccatcgcgcgcaagcacgaagatgcgatgctagacctgggtggcctgaacaagggctacaccgtcgactgcgtggtggatcgtctgaatgcagccaacttcgccgacgtgctgttcgagtggggcggcgactgccgcgcctcgggtgtgaatgtgcagcgccagccgtgggcagtcggcgttgtgcgtccgccatcggtcgacgaggtcgtcgcggctgccaagtccggcaagtcggtgacaatgaatgcccacagccttggggatcacacggatgaaccggcgcagtcgacgtcggccgccgacggggcggccaaggctgagcacaaggcgctcctgcgcgtcatgtcgctcagcaatgaggcactctgcacgagcggcgactacgagaacgtgctcttcgccaacgcgcttggatgcgctctctcgagcacatacgactggcgtcgccgctgcctcattgagccctgccggaacgaactggcccaggttagcatcaaatgctactcgtgtctgtacgccgacgcactcgccaccgcgagcttcgtgaagcgcgaccccgtgcgcgtgcggtacatgctcgagcactaccgccacgattacaaccgcgtgaccgactacgccgcctacacgcgcgagggggagcggctggcgcacatgtacgagatcgcgcatgagagcccggcctgtcggatagagcgcattgccggctcgctgccagcgcgtgttgttgtgatcggcggcggccttgctggttgcgcggccgccattgaggcagccagctgcggcgctaccgtcattctcctggagaaggaagcccggctgggtggcaatagcgccaaggccacctctggcatcaacggctggggcacccgcacgcaggcggtgaatcacgtcctcgataactgcaagttttttgagcgagacacgttcctctccggcaagggtggccactgcgaccctggactcgtgcgcaccctctctgtaaaatccgctgaagcgattagctggctcgagtccttcggcatcccgctaaccgtcctctaccagcttggtggcgcgagccgcaggcgctgccatcgcgcgccggatcagaaagacggcactccggtgcccgttggcttcacgatcatgcgtcacctcgaggactacatccgcaccaagctgcaaggcaaggtgacgatcttgaacgagatggcggtggtgagcctcatgcacgacgtgagcgcgatgccggacggaaaccgcgagattcgcgtgcacggtgtccgctacaagtcgatgaccgatgcgtcggggacggttatggatctgccggcggacgccgtcgtgcttgccaccggcggcttctcgaacgaccacacgcccaactcgctgctgcgtgagtacgcgccaaacgtgtacggcacccccaccaccaacggcacgttcgccaccggcgacggtgtgaagatggcgcgcaagctgggcgccacgctggtagacatggacaaggtgcagctgcacccgaccggtctcattgaccccaaggacccgtcgaatcgcaccaagtaccttggccccgaggcgctgcgcggctccggcggcatcctgctgaacaagaacggcgagcgcttcgtgaacgagctggacctgcgctccgtcgtgtcgcaggcgattatcgcgcaggacaacgagtacccgaactcgggtggcagcaagttcgcatactgcgtgctgagcgaggaggcagcgacgctcttcggcaagaactccctcacgtactactggaagtcgcagggtctgttcacccgtgtggatgacatgaaggcgctcgccgagctcatcggctgctcggttgaaagcctgcatcgaaccctcgagacatacgagcgccagagcacggggaagaaggcctgcccgcggactggcaagctcgtgttccccagtgtggtgggcaccaaggggccctattacgtggcgtacgtcacaccgtcgatccactacaccatgggcggctgcttcatctctccggcggcggagctgctcatggaagatcactccgtcaacatattcgaagacatgcatcccattcttggcctcttcggtgcaggtgaggtaaccggcggcgtgcacggccgcaaccgtctcggcggcaactctctgctggagtgcgtcgtgttcggcaagatcgctggcgaccgcgcggccacaattctgcagaaggagaagcacgggctccgcaaggataagtgggtgccggtggtggtgcgggagtcgagggcgagtgatcagttcggtgttggctcgcgcgtgctgcgcttcaacctgcccggcgcgacgcagacatccggattgaccgttggcgagttcatcggtatccgcggtgactgggacggccagcaattgattggctactacagccccatcaacatgcccgacgacaagggccgcatctcgattctggcgcgtggtgacaagggcaacctgcaggagtggatctcgtccatgcgtcctggcgactcggtcgaaatgaaggcctgcggcggtctccgtatcgagctcaagccccaccagaagcagatggtgtaccgtaagacggtcattcggaaactgggcctcatcgccggcggctccggtgtggcgccgatgctgcagattattaaggccgcgctcaaccgcccatacgtggacagcatcgagacgatccgcctcgtgtacgctgccgaggacgagtatgagctgacctaccgctcgctgctgaagcaataccgcgccgacaacccggacaagttcgactgcggcttcgtgctgaataaccctcctgaaggctggacagagggtgtgggctacgtcgaccgtgccacgctgcagagccttctcccgcctccgtcgaagggcctgctcgtggccatttgcggcccgccggtgatgcagcgctccgtcgtggcggacctgctggcactaggctataacgccgaaatggtgcgcacggtggatgaggatggcgcgctctag

SEQ ID NO: 30 - protein, 1194 aa, Leishmania infantum
MGGCATSLCRRCAATDSHTGASVVVSDPEKAARERDRIARGLLTTNFPELHVNQRSVLRYKDVMHTVPYTLTIAVDGNVARQDVDPVVKAILSDCFAMVDKHLNSFNPGSEVSQVNRMPVGKKHVMSEHLFEVVKCCEEVYSSSGSCFDPAAAPLVHKLRDAARRQDSAEGDFAISAEEAGRFTLTNSFAIDIKEGTIARKHEDAMLDLGGLNKGYTVDCVVDRLNAANFADVLFEWGGDCRASGVNVQRQPWAVGVVRPPSVDEVVAAAKSGKSVTMNAHSLGDHTDEPAQSTSAADGAAKAEHKALLRVMSLSNEALCTSGDYENVLFANALGCALSSTYDWRRRCLIEPCRNELAQVSIKCYSCLYADALATASFVKRDPVRVRYMLEHYRHDYNRVTDYAAYTREGERLAHMYEIAHESPACRIERIAGSLPARVVVIGGGLAGCAAAIEAASCGATVILLEKEARLGGNSAKATSGINGWGTRTQAVNHVLDNCKFFERDTFLSGKGGHCDPGLVRTLSVKSAEAISWLESFGIPLTVLYQLGGASRRRCHRAPDQKDGTPVPVGFTIMRHLEDYIRTKLQGKVTILNEMAVVSLMHDVSAMPDGNREIRVHGVRYKSMTDASGTVMDLPADAVVLATGGFSNDHTPNSLLREYAPNVYGTPTTNGTFATGDGVKMARKLGATLVDMDKVQLHPTGLIDPKDPSNRTKYLGPEALRGSGGILLNKNGERFVNELDLRSVVSQAIIAQDNEYPNSGGSKFAYCVLSEEAATLFGKNSLTYYWKSQGLFTRVDDMKALAELIGCSVESLHRTLETYERQSTGKKACPRTGKLVFPSVVGTKGPYYVAYVTPSIHYTMGGCFISPAAELLMEDHSVNIFEDMHPILGLFGAGEVTGGVHGRNRLGGNSLLECVVFGKIAGDRAATILQKEKHGLRKDKWVPVVVRESRASDQFGVGSRVLRFNLPGATQTSGLTVGEFIGIRGDWDGQQLIGYYSPINMPDDKGRISILARGDKGNLQEWISSMRPGDSVEMKACGGLRIELKPHQKQMVYRKTVIRKLGLIAGGSGVAPMLQIIKAALNRPYVDSIETIRLVYAAEDEYELTYRSLLKQYRADNPDKFDCGFVLNNPPEGWTEGVGYVDRATLQSLLPPPSKGLLVAICGPPVMQRSVVADLLALGYNAEMVRTVDEDGAL

SEQ ID NO: 31 - DNA, 1488 bp, Leishmania infantum
  CDS (1)..(1488): Sequence coding for a fumarate reductase
atgtctcgagtagccccttcagtaaaccgggtcgtcatcatcggcagcggacttgcagggcagtccgcagcgatcgaggccgcccgcgagggcgctaaggaagttgtcctcattgagaaggaagcgcggctgggcggcaacagtgccaaggccacgtctggcatcaacggctggggcacggcagtgcagaaggccgccggcgtgcacgacagcggcgaactctttgaaaaggatacgttcgtctctggcaagggtggcacctgtcggccagagttggtgcggacgctgtccgaccacagtgcagaagccatcgagtggctctcttcgtttggcatcccgctgaccgccatcacgcaactcggcggtgcgagtcgcaagcgctgccaccgtgccccagacaagccggacgggactccgctgccgatcggttccacgattgtgcgtgcgctggagaactacatccgcacaaacctgtccggcaccgtgctcatcgaaaccaatgcgcgtctcatctcactgatacacagtaaggagggcggcgtggaggtggtgcaaggcatcacgtatgccacgcaaactggaagcggcgaggagcagactcgcgaactacaggcccgtgccgtcattctcgccacgggcggcttctcgaacgaccacacgcccaactcgctcctgcagcagtacgcgccgcaactgtcgtccttccccaccaccaacggtgtatgggccaccggcgacggtgtaaaggcggcacgtgagctgggcgtaaagctggtggacatggacaaggtgcagctgcacccgaccggtctgctgaatccgaaagacccgaacgccaagacactattcctcggccccgaggcgctgcgtggctccggtggcgtgctgctgaacaagaacggcgagcgcttcgtgaacgagctgggcctgcgctccgtcgtgtcgcaggcgattatcgcgcagaacaacgtctaccccggcacagagagtcgccgctacgcgtactgcgtactgaacgacgcggctgctgacgcgtttggtcgcagttcgctgaacttctactggaaaaagatggggctcttttctgaggttgccgacgtcgccgcgcttgcggctctcatcggatgtccggaagaaaccttgatgcacacgctctccgagtacgagaagatctccagcggccgaaaaccgtgcccgaagagtggaaaagaagtgttcccttgtgtgctgggtactcaagggccctactacgttgccctcgtcacgccgtcgatccactacaccatgggtggctgcctcatctcacctgcagcagagatcctgaatgagcagatgcacccgattcttggtctctttggcgctggcgaggtgacgggcggcgtgcacggtggcaaccgtctcggcggcaactcgctgctggagtgtgtcgtgttcggtcgcattgctgggcgtcaagctgcacgccacctcggcacagcggtgtcttag

SEQ ID NO: 32 - protein, 495 aa, Leishmania infantum
MSRVAPSVNRVVIIGSGLAGQSAAIEAAREGAKEVVLIEKEARLGGNSAKATSGINGWGTAVQKAAGVHDSGELFEKDTFVSGKGGTCRPELVRTLSDHSAEAIEWLSSFGIPLTAITQLGGASRKRCHRAPDKPDGTPLPIGSTIVRALENYIRTNLSGTVLIETNARLISLIHSKEGGVEVVQGITYATQTGSGEEQTRELQARAVILATGGFSNDHTPNSLLQQYAPQLSSFPTTNGVWATGDGVKAARELGVKLVDMDKVQLHPTGLLNPKDPNAKTLFLGPEALRGSGGVLLNKNGERFVNELGLRSVVSQAIIAQNNVYPGTESRRYAYCVLNDAAADAFGRSSLNFYWKKMGLFSEVADVAALAALIGCPEETLMHTLSEYEKISSGRKPCPKSGKEVFPCVLGTQGPYYVALVTPSIHYTMGGCLISPAAEILNEQMHPILGLFGAGEVTGGVHGGNRLGGNSLLECVVFGRIAGRQAARHLGTAVS

SEQ ID NO: 33 - DNA, 3648 bp, Trypanosoma cruzi strain CL Brene
atgttctcaacaatgcgcattttactccgcaccgccacgatcggcgtcgccacggggacaatggcgatgacaacaactttggtgtctggtgcacatcttgccgcagtacggtggtcctcctcctcgacgtcggacaattcaccggcactcttgtcgcagcgaagttttttttccggaggcgccgatggtcgctcctccgcgtcgattgtcgtggtcgatgctgagcttgccgcaaaggagcgtgaccgcatcgcacgtgagatgttgtcgcagaacatgcctagccctcatgccgaggagcgattggtggtgacgatgaggggactggagcacactgtgccgtatacccttcgtatcgttctgaacgggccaaacgaagcaaagaatggtgaagtcatagcggggaaagtattaaaagaggccttcgaggttgtggatcagcatttgaatcactataacccggagagcgaggtgtcggcaatcaaccagcttcctttaggggcgaagcacaccatgtcacagcacatgcgacgtgtgatggagtgttgcgtacgggtctacgcatccagtggagcctgctttgaccctgccacaggaccgctggttgaatttcttcgttctgtcatgaaggatgagaagagtgacgcagagtcaacacttacggaagaggaagtggagcgcttttgcctgcctcagagttttgatgtaaatatgacagacggcaccatcgcccgcaagcacgagggtgccaagttggatttgggtggtgtaaacaagggctacactgtggaccgtgtggtggaaaagcttaacgcagctgggatgcgggatgtgatgtttgaatggggaggtgattgccgtgcaacgggagtgaattatcagcatcagccatgggccattgccattgtgcgcccaccgcctgtcgaggtggtggaacagcacgccaaggaagggttggatgaaaaaaaggaggcatcacagttgcttcgactcatgtatcttgacgatgaggcactttgtacgagtggcgactatgaaaatgtcatgtacagtccaaaatatggtgtccgcagcaacatctttgactggaaaaagagaagtctgctggagccagttgaaagtgaactggcgcaggtttccatcaagtgctacagtgcaatgtacgctgacgcacttgcaacggcaagcctcatcaaacgtgacatttcaaaagtacggcatatgttggaggaatggcgtcactcgcgaaatcgcgtcacgaattacgttacctacacacgacagggcgaacgcgtagcccgtatgtttgaaatagcaacagaaaatgccgagattcgcaaaaaccgtattgccggttctcttcctgcacgcgtaattgttgtgggttgtggtcttgccgggttgtctgcagccatcgaggccaccgcctgcggagcccaagtcatcctgctggagaaggaaccgaaggttggtggcaacagcgccaaggcaacgtccggcatcaatggctggggcacacgcgcacaggcactggatgacatccaagataactgcaatatatttgagcgcgatacgcacaaatcgggtctcggcggcagcaccgtccccagtctggtgcgcacactctccgtaaagagcggggatgccatttcttggctttcgtcgcttggcgttccactgacagtgctttcgcagcttggcgggcacagccgcaagcgcacgcaccgtgcccccgataaggcagacggcactcctgtgcccattggcttcaccatcatgcggacgcttgagcagcacgttcgcacgaagctggcggatcgcgttacgatcatggaaagcactgtcgtcacttcgcttctaaatgagatcaaaggtacacccgatggtgggcgtgaagtgagagtcacgggcgttacttacaagaagtccgatgagaaggaggcaggttcgatgaaacttaccgcggatgccgtcattctcgccaccggcggcttttcaaatgatcacatgtcgcaatcactcattggcgagtttgcgccagagttgtccggcttccccacaaccaatggaccgtgggccacgggtgacggcgtcaagcttgcacgacgccttggtgccacacttgtcgacatggaaaaggtacaacttcacccgacaggcctcatcgacccgaaagaccccgcaaacccgaccaagtaccttggcccggaggcgctgcgtgggtcgggcggcgtcctgctaaacaagaagggtgagcggtttgtcaatgagcttgaactccgttccgtggtctccaacgccatcattgagcagggcgacgaatatccatattcaggcggtagcaagtttgccttctgtgtgctcaatgacgcagcggtgaagctctttggcgtcaatctgttgaacttttacgcaaatactttgggagtgtttaagcgtgtggatgacctgcaggggctggcaatgctcattggttgtgatgttttaactttgcagaatacgctggaaacatacgagtcaagcagtattgttacctccgcgtgtccatttacgggaaaggttgtctacccgtgtgtggtgggaccgcagggtcccttttatgttgcctttgtgacgccgtcgatccactacacaatgggcggctgcctcatctcgccctcggcagaaattcagcgtgaacactattctctgaaccttcttgagaatcaacgtcccattcttggcttgtttggtgcgggcgaggtgacgggcggggtgcatggtggaaaccggcttggtggcaattcgctgctggaatgcgtcgtctttggccgcattgctggggatcgtgccgcaacaatcctgcagaagcaagtgtatgcgctttcaaaggataagtggacatccgttgtggtccgtgaatcccgcagtggtgaacggtttgggaccggctctcgtgtgctgcggtttaaccttccgggtgccttgcagcgttctggactttacctcggccagtttattgccatccgcggtgagtgggatggccagcagctcattgggtactacagccccatcacattgccggatgaacgtggcgtcatatctatccttgcccgtggggacaaggggactttgaaggagtggatttctgccatgcgccctggtgactccgtagagattaaaagttgcggtggcattctcattgagcgcaatccggcgaagaagcaatttctcttccacggacatgttatacggcaattcgggcttattgcggggggctcaggcgtggcaccgatgctgcagatcattcgtgccgcgctggaacgcccatacgtggatacaacggagtccatccgtttagtttacaccgccgaggaatatgaagagttaacgtaccgtgaactgcttcatcactattccaaggagaatccggacaagttttccgttgaattttctcttaacaacccaccggagggatggaccgggggtgttggcttcgttgatcgtccgtcgctgcggaaaacactgcagcctccttcgaatgatctgcttattgccatctgcggcccgcccgctatgcagcgtgcaatgaagaacgacctgctggctatgggctataacccagcgcttgtgcacacggtggatgatgacatgcaagccgcactgtaa

SEQ ID NO: 34 - DNA, 3429 bp, Trypanosoma cruzi strain CL Brene
atggcagacggtcgatcttccgcctccgtcgttgctgttgatcctgagaaggccgcacgggaaagggatgaggcggcccgtgcccttcttcgggacagccccttgcaaacacacttgcaatacatgaccaacggcctcgagctcactgtgcctttcacattgaaggttgttgcagaggcggttgctttctcgcgcgccaaagaggtagcggatgaagttctccgctccgcttggcatcttgcagataccgttctgaacaattttaaccccaacagtgagatctccatgattggcaggcttccagttggtcaaaagcacacgatgtctgccacgcttaagagtgtcattacgtgctgtcaacacgtctttaactcttcccgtggggtgtttgaccctgccactggaccaatcattgaggcgttacgtgccaaagttgcagagaaagcctcagtgtcggatgaacagatggagaagcttttccgggtctgcaacttttccagcagcttcatcgttgacttggaaatgggcaccatcgcccgcaagcacgaagatgctcgctttgatctgggcggtgtgagcaagggctacatagtggactacgtcgtggagaggttaaacgcagctggaattgtcgatgtgtactttgaatggggcggtgactgtcgtgcaagtggtacgaacgcgcgacgcacaccatggatggtaggcattattcgcccgccgtccctggagcaattgcgaaatccgccaaaagacccttcatatatccgtgtcctgccactcaacgatgaggctctgtgtacgagtggggactacgagaacttgacagaaggctccaacaagaagctttacacttccatctttgactggaaaaagagaagtctgctggagcccgttgaaagtgaactggcgcaggtttccatcaggtgctacagcgcaatgtacgctgacgcacttgcaacggcaagcctcatcaaacgtgacattaaaaaagtacggcaaatgttggaggactggcgtcacgtgcgaaatcgcgtcacgaattacgttacctacacacgacagggcgaacgcgtagcccgtatgtttgaaatagccactgataacgccgagattcgcaaaaaacgtattgccggttctcttcctgcacgcgtaattgttgtgggtggtggtcttgccgggttgtctgcagccatcgaggccaccgcctgcggagcccaagtcatcctcctggagaaggaaccgaaggttggtggcaacagcgccaaggcaacgtccggcatcaatggctggggcacacgcgcacaggcagagcaggatgtctatgacagcgggaagtactttgagcgcgatacgcacaaatcgggtctcggcggcagcaccgaccccggtctggtgcgcacactctccgtaaagagcggggatgccatttcttggctttcgtcgcttggcgttccactgacagtgctttcgcagcttggcgggcacagccgcaagcgcacgcaccgtgcccccgataaggcagacggcactcctgtgcccattggtttcaccatcatgcaaacgcttgagcagcacgttcgcacgaagctggcggatcgcgtgacgatcatggaaaacactactgtcacttcactgctgagcaagtcccgggtgcgccatgatggtgccaaacaggtgagggtgtacggcgtggaagtactgcaggacgagggggtggtatcaaggatactggcggatgccgtcattctcgccaccggcggcttttcaaatgacaagacgcccaactcgcttctccaggagtttgcgccgcagttgtccggcttccccacaaccaatggaccgtgggccacgggtgacggcgtcaagcttgcacgtgaactgggggtgaagcttgtcgacatggacaaggtacaacttcacccgacaggcctcatcgacccgaaagaccccgcaaacccgaccaagtaccttggcccggaggcgctgcgtgggtcgggcggcgtcctgctaaacaagaagggtgagcggtttgtcaatgagcttgacctccgttccgtggtctccaacgccatcattgagcagggcgacgaataccccgatgcaggtggtagcaaatttgccttctgtgtgctcaatgacgcagcggtgaagctcttcggcgtgaattcccacggcttttattggaagcgtcttggtctttttgtaaaggctgacacagtggaaaagcttgcggcactcattggatgccctgtggagaatgtgcggaatacgctgggggactacgagcagctctcaaaagaaaaccgtcaatgccctaagactcgcaaggttgtctacccgtgtgtggtgggaccgcagggtcccttttatgttgcctttgtgacgccgtcgatccactacacaatgggcggctgcctcatctcgccctcggcagagatgcagttggaagaaaacacgacctctccctttggccatcgccgtcctatttttggcttgtttggtgcgggcgaggtgacgggcggagtgcatggtggaaatcggcttggtggcaattcgctgctggaatgcgtcgtctttggccgcattgctggggatcgtgccgcaacaatcctgcagaagaaacctgtgccgctgtcatttaaaacctggacgacagtgattttgcgcgaggtgcgggagggtggaatgtacggaactggctctcgtgtgctgcggtttaaccttccgggtgccttgcagcgttctgggcttcaactcggccagtttattgccatccgcggtgagtgggatggccagcagctcattgggtactacagccccatcacattgccggacgatcttggcgtcattggtatcctggcgaggagtgacaaggggactttgaaggagtggatttccgccctagaaccaggcgatgcggtggagatgaaaggttgcggtggcctggtgattgagcgccgtttcagcgaacgatacttgtacttttcaggccatgcgctgaaaaaactttgccttattgccggcggcacaggtgtggcaccgatgctgcagatcattcgtgccgcactcaagaagccgttccttgaaaatatcgagagcatccgcctcatctacgctgccgaagatgtctcggagctcacgtaccgcgaactgcttgaacatcaccagcgagattcaaagggaaagtttcgcagtatttttgttttgaatcgccctccaccaatttggactgacggcgttgggttcattgacaagaagcttctgagctcatccgtacaaccgccggcaaaagatctgttggtggccatctgcggcccgccaattatgcagcgcgttgtcaagacgtgcctgaagagtcttgggtacgacatgcaactagtgcgtactgtcgacgaagtggaaacccagaactcatccaagatgtaa

SEQ ID NO: 35 - DNA, 2370 bp, Trypanosoma cruzi strain CL Brener
atgcgtccgggggatatttttagtttcagagtcagtcacatcacaactcaatttttgcttgccaccttcctgctggcttcggtgtcttttaccttaatgaccgaacaaatgaacatgcagccacaacgagacgtcttctatgatccggcagaattgcagaaacagccagttgttattgttgtgggtggtggtcttgccgggttgtctgctgccatcgaggccaccgcctgcggagcccaagtcatcctgctggagaaggaaccgagggttggtggcaacagcgccaaggcaacgtccggcatcaatggctggggcacacgcgcacaggcagagcaggatgtctatgacagcgggaagtactttgagcgcgacacgcacaaatcgggtctcggtggcagcaccgaccccggtctggtgcgcacactctccgtaaagagcggggatgccatttcttggctttcgtcgcttggcgttcccctgacagtgctttcgcagcttggtgggcacagccgcaagcgcacgcaccgtgcccccgataaggccgacggcactcctgtgcccattggcttcaccatcatgcaaacgcttgagcagcacatccgcacgaagctgacggatcgcgtgacgatcatggaaaacactactgtcacttcgctgctgagcaaatcccgggtgcgccatgatggtgccaaacaggtgagggtgtacggcgtggaagtactgcaggacgagggggtggcatcaaggatactggcggatgccgtcattctcgccaccggcggcttttcaaatgacaagacgcccaactcgcttctccaggagtttgcgccgcagttgtccggcttccctacaaccaatggaccgtgggccacgggtgacggcgtcaagcttgcacgcgagctgggggtgaagcttgtcgacatggacaaggtacaacttcacccgacaggcctcatcgacccgaaagaccccgcaaacccgaccaagtaccttggcccggaggcgctgcgtgggtcgggcggcgtcctgctaaacaagaagggtgagcggtttgtcaatgagcttgacctccgttccgtggtctccaacgccatcattgagcagggagacgaataccccgattcaggcggtagcaagtttgccttctgtgtgctcaatgacgcagcggtgaggctcttcggcgtgaattcccacggcttttattggaagcgccgtggtctttttgtaaaggctgacacagtggaagagcttgcggcactcattggatgccctgtggagaatgtgcggaatacgctgggggactacgagcagctctcaaaagaaaaccgtcaatgccccaagactcgcaaggttgtctacccgtgtgtggtgggaccgcagggtcccttttatgttgcctttgtgacgccgtcgatccactacacaatgggcggctgcctcatttcgccctcggcagagatgcagttggaagaaaacacgacctccccctttggccaccgccgtcccatttttggcttgtttggtgcgggcgaggtgacgggcggggtgcatggcggaaaccggcttggtggcaattcgctgctggaatgcgtcgtctttggccgcattgctggggatcgtgccgcgactatcctgcagaataatgacacttacctctggggcgagaagtggtctcagctgaagctgcggaatgtcgtggaagaagagaatggatttatgtggttgcacttcactttcccaagcagttttcaagtttctggattggaagtcttacagggggtggtgttgcgttctgtctccggtgataaaagtgtggaagtttatactccatatactttacctgacgatgagggtgttgtgggcattgcgttgagtccattgctcactgggaatggggtgcattggttgcgtacactgcagccgggggatacggtggaaatgaaagctgctgaacgggtggatcgcggttatatgaatatgctgaatgctccacataaagttgtcatcgccacctctcgcgggattgctcccatgatgcaaatccttcgggccgccatggaagggccggagaaggatacaaccattcatttaatttacattgcggatcgagcatcttccattcctcatcgtgaaaagctgaaggcgttggcggaggcatcccctgaacgattccggtgtacatttgttctgcagcacccacagcccgggtggtcgggtgctgtgaactatgtggacgaggttgctgcatccgtgtttcccgaccccgcactcgtcatctttctctgtggtgccagcgaagagacaaggtctataaagagctctcttcttgacatggggcacgatgtcgccaccatcgcgactgtagaatag

SEQ ID NO: 36 - DNA, 1764 bp, Trypanosoma cruzi strain CL Brener
  CDS (1)..(1764): Sequence coding for a fumarate reductase
atggcagacggccgatcttccgcctccgtcgttgctgttgatcctgagaaggccgcacgggaaagggatgaggcggcccgtgcccttcttcgggacagccccctgcaaacacacctgcaatacatgaccaacggccttgagctcactgtgcctttcacattgaaggttgttgcagaggcggttgctttctcgcgcgccaaagaggtagcggatgaagttctccgctccgcttggcatcttgcagataccgttctgaacaattttaaccccaacagcgagatctccatgattggcaggcttccggttggtcaaaagcacactatgtctgccacgcttaagagtgtcattacgtgctgtcaacacgtcttcaactcttcccgtggggtgtttgatcctgccactggaccaatcattgaagcgttacgtgccaaagttgcagagaaagcctccgtgtcggatgaacagatggagaagcttttccgggtctgcaactttcccagcagcttcagcgttgacttggaaatgggcaccatcgcccgcaagcacgaagatgctcgctttgatctgggcggtgtgagcaagggctacatagtggactacgtcgtggagaggttaaacgcagctggaattgtcgatgtttactttgaatggggcggtgactgtcgtgcaagtggtacgaacgcgcgacgcacaccatggatggtaggcattattcggccgccgtccatggagcaattgcgaaatccaccaaaggatccttcatatatccgtgtcctgccactaaacgatgaggctctgtgtacgagtggggactacgagaacttgacagaaggctccaacaagaagctttacacttccatctttgactggaaaaagaaggatctgctggagcccgttgaaagtgaactggcgcaggtttccatcaagtgctacagcgcaatgtacgctgacgcacttgcaacggcaagcctcatcaaacgtgacattaaaaaagtacggcaaatgttggaggaatggcgtcacgtgcgaaatcgcgtcacgaattacgttacctacacacgacagggcgaacgcgtagcccgtatgtttgaaatagccactgataacgccgagattcgcaaaaaacgtattgccggttctcttcctgcacgcgtaattgttgtgggtggtggtcttgccgggttgtctgcagccatcgaggccaccgcctgcggagcccaagtcatcctgctggagaaggaaccgagggttggcggcaacagcgccaaggcaacgtccggcatcaatggctggggcacgcgcgcacaggcagagcaggatgtctatgacagcgggaagtactttgagcgcgacacgcacaaatcgggtctcggtggcagcaccgaccccggtctggtgcgcacactctccgtaaagagcggggatgccatttcttggctttcgtcgcttggcgttcccctgacagtgctttcgcagcttggtgggcacagccgcaagcgcacacaccgtgcccccgataaggccgacggcactcctgtgcccattggcttcaccatcatgcaaacgcttgagcagcacgttcgcacgaagctggcggatcgcgtgacgatcatggaaaacactactgtcacttcgctgctgagcaaatcccgggtgcgccatgatggtgccaaacaggtgagggtgtacggcgtggaagtactgcaggacgagggggtggcatcaaggatactggcggatgccgtcattctcgccaccggcggc

SEQ ID NO: 37 - protein, 588 aa, Trypanosoma cruzi strain CL Brener
MADGRSSASVVAVDPEKAARERDEAARALLRDSPLQTHLQYMTNGLELTVPFTLKVVAEAVAFSRAKEVADEVLRSAWHLADTVLNNFNPNSEISMIGRLPVGQKHTMSATLKSVITCCQHVFNSSRGVFDPATGPIIEALRAKVAEKASVSDEQMEKLFRVCNFPSSFSVDLEMGTIARKHEDARFDLGGVSKGYIVDYVVERLNAAGIVDVYFEWGGDCRASGTNARRTPWMVGIIRPPSMEQLRNPPKDPSYIRVLPLNDEALCTSGDYENLTEGSNKKLYTSIFDWKKKDLLEPVESELAQVSIKCYSAMYADALATASLIKRDIKKVRQMLEEWRHVRNRVTNYVTYTRQGERVARMFEIATDNAEIRKKRIAGSLPARVIVVGGGLAGLSAAIEATACGAQVILLEKEPRVGGNSAKATSGINGWGTRAQAEQDVYDSGKYFERDTHKSGLGGSTDPGLVRTLSVKSGDAISWLSSLGVPLTVLSQLGGHSRKRTHRAPDKADGTPVPIGFTIMQTLEQHVRTKLADRVTIMENTTVTSLLSKSRVRHDGAKQVRVYGVEVLQDEGVASRILADAVILATGG

SEQ ID NO: 38 - DNA, 1458 bp, Trypanosoma cruzi strain CL Brener
  CDS (1)..(1458): Sequence coding for a fumarate reductase
cttggcccggaggcgctgcgtgggtcgggcggcgttctgctaaacaagaagggtgagcggtttgtcaatgagcttgacctccgttccgtggtctccaacgccatcattgagcagggcgacgaataccccgattcaggcggtagcaagtttgccttctgtgtgctcaatgacgcagcggtgaggctcttcggcgtgaattcccacggcttttattggaatcgtcttggtctttttgtaaaggctgacacagtggaaaagcttgcggcactcattggatgccctgtggagaatgtgcggaatacgctgggggactacgagcagctctcaaaagagaaccgtcaatgccccaagactcgcaagattgtctacccgtgtgtggtgggaccgcagggtcccttttatgttgcctttgtgacgccgtcgatccactacacaatgggcggctgcctcatctcgccctcggcagagatgcagttggaagaaaacacgacctccccctttggccaccgccgtcctatttttggcttgtttggtgcgggcgaggtgacgggcggggtgcatggcggaaaccggcttggtggcaattcgctgctggaatgcgtcgtctttggccgcattgctggggatcgtgccgcgactatcctgcagaagaaacctgtgccgctgtcatttaaaacctggacgacagtgattttgcgcgaggtgcgggagggtggaatgtacggaactggttctcgtgtgctgcggtttaatcttccgggtgccttgcagcgttctgggcttcaactcggccagtttattgccatccgcggtgagtgggatggccagcagctcattgggtactacagccccatcacattgccggacgatcttggtgtcattggcatcctggcgaggagtgacaaggggactttgaaggagtggatttccgccctagaaccaggcgatgcggtggagatgaaaggttgcggtggcctggtgattgagcgccgtttcagcgaacgatacctgtacttttcaggtcatgcgctgaaaaaactttgccttattgccggcggcacaggtgtggcaccgatgctgcagatcattcgtgccgcactcaagaagccgttccttgaaaatatcgagagcatctgtctcatctacgctgccgaagatgtctcggagctcacgtaccgcgaactgcttgaacagcaccagcgagattcaaagggaaagtttcgcagtatttttgttttgaatcgccctccaccagtttggactgacggcgttgggttcattgacaagaagcttctgagctcatccgtacaaccgccggcaaaagatctgttggtggccatctgcggcccgccaattatgcagcgcgttgtcaagacgtgcctgaagagtcttgggtacgacatgcaactagtgcgtactgtcgacgaagtggaaacccagaactcatccaagatgtaa

SEQ ID NO: 39 - protein, 485 aa, Trypanosoma cruzi strain CL Brener
LGPEALRGSGGVLLNKKGERFVNELDLRSVVSNAIIEQGDEYPDSGGSKFAFCVLNDAAVRLFGVNSHGFYWNRLGLFVKADTVEKLAALIGCPVENVRNTLGDYEQLSKENRQCPKTRKIVYPCVVGPQGPFYVAFVTPSIHYTMGGCLISPSAEMQLEENTTSPFGHRRPIFGLFGAGEVTGGVHGGNRLGGNSLLECVVFGRIAGDRAATILQKKPVPLSFKTWTTVILREVREGGMYGTGSRVLRFNLPGALQRSGLQLGQFIAIRGEWDGQQLIGYYSPITLPDDLGVIGILARSDKGTLKEWISALEPGDAVEMKGCGGLVIERRFSERYLYFSGHALKKLCLIAGGTGVAPMLQIIRAALKKPFLENIESICLIYAAEDVSELTYRELLEQHQRDSKGKFRSIFVLNRPPPVWTDGVGFIDKKLLSSSVQPPAKDLLVAICGPPIMQRVVKTCLKSLGYDMQLVRTVDEVETQNSSKM

SEQ ID NO: 40 - DNA, 780 bp, Trypanosoma cruzi strain CL Brener
  CDS (1)..(780): Sequence coding for a fumarate reductase
tcgacccgaaagaccccgcaaacccgaccaagtaccttggcccggaggcgctgcgtgggtcgggcggcgtcctgctaaacaagaagggtgagcggtttgtcaatgagcttgacctccgttccgtggtctccaacgccatcattgagcagggcgacgaataccccgatgcaggtggtagcaaatttgccttctgtgtgctcaatgacgcagcggtgaagctcttcggcgtgaattcccacggcttttattggaagcgtcttggtctttttgtaaaggctgacacagtggaaaagcttgcggcactcattggatgccctgtggagaatgtgcggaatacgctgggggactacgagcagctctcaaaagaaaaccgtcaatgccccaagactcgcaaggttgtctacccgtgtgtggtgggaccgcagggtcccttttatgttgcctttgtgacgccgtcgatccactacacaatgggcggctgcctcatctcgccctcggcagagatgcagttggaagaaaacacgacctccccctttggccaccgccgtcctatttttggcttgtttggtgcgggcgaggtgacgggcggagtgcatggtggaaaccggcttggtggcaattcgctgctggaatgcgtcgtctttggccgcattgctggggatcgtgccgcaacaatcctgcagaataatgacacttacctctggggcgagaagtggtctcagctgaagctgcggaatgtcatggaagacgagaatggatttatgtggttgcacttcactt

SEQ ID NO: 41 - protein, 25 aa, Trypanosoma cruzi strain CL Brener
STRKTPQTRPSTLARRRCVGRAASC

SEQ ID NO: 42 - protein, 45 aa, Trypanosoma cruzi strain CL Brener
TRRVSGLSMSLTSVPWSPTPSLSRATNTPMQVVANLPSVCSMTQR

SEQ ID NO: 43 - protein, 4 aa, Trypanosoma cruzi strain CL Brener
SSSA

SEQ ID NO: 44 - protein, 13 aa, Trypanosoma cruzi strain CL Brener
IPTAFIGSVLVFL

SEQ ID NO: 45 - protein, 58 aa, Trypanosoma cruzi strain CL Brener
RLTQWKSLRHSLDALWRMCGIRWGTTSSSQKKTVNAPRLARLSTRVWWDRRVPFMLPL

SEQ ID NO: 46 - protein, 43 aa, Trypanosoma cruzi strain CL Brener
RRRSTTQWAAASSRPRQRCSWKKTRPPPLATAVLFLACLVRAR

SEQ ID NO: 47 - protein, 47 aa, Trypanosoma cruzi strain CL Brener
RAECMVETGLVAIRCWNASSLAALLGIVPQQSCRIMTLTSGARSGLS

SEQ ID NO: 48 - protein, 18 aa, Trypanosoma cruzi strain CL Brener
SCGMSWKTRMDLCGCTSL

SEQ ID NO: 49 - DNA, 60 bp, synthetic oligonucleotide
  misc_feature (1)..(60): Primer 1 NuoA_NR
catcagcggcattgccaaacgcacaatgctaatcagcggtattccggggatccgtcgacc

SEQ ID NO: 50 - DNA, 60 bp, synthetic oligonucleotide
  misc_feature (1)..(60): Primer 2nuoA_NF
ttcatcgcatcggacgatagataattcctgagacaatagtgtgtaggctggagctgcttc

SEQ ID NO: 51 - DNA, 60 bp, synthetic oligonucleotide
  misc_feature (1)..(60): Primer 3ndhF
atacacccctcactctatatcactctcacaaattcgctcagtgtaggctggagctgcttc

SEQ ID NO: 52 - DNA, 60 bp, synthetic oligonucleotide
  misc_feature (1)..(60): Primer 4ndhR
atgcaacttcaaacgcggacggataacgcggttaatactcattccggggatccgtcgacc

SEQ ID NO: 53 - DNA, 65 bp, synthetic oligonucleotide
  misc_feature (1)..(65): Primer 5ndh_BM3F
cattaattaacaattggttaataaatttaagggggtcacgatgacaattaaagaaatgcctcagc

SEQ ID NO: 54 - DNA, 65 bp, synthetic oligonucleotide
  misc_feature (1)..(65): Primer 6nuo_BM3F
gaagagcagtgaatctggcgctacttttgatgagtaagcaatgacaattaaagaaatgcctcagc

SEQ ID NO: 55 - DNA, 60 bp, synthetic oligonucleotide
  misc_feature (1)..(60): Primer 7nuo_tacBM3F
ttcatcgcatcggacgatagataattcctgagacaatagtgcttccggctcgtataatgt

SEQ ID NO: 56 - DNA, 60 bp, synthetic oligonucleotide
  misc_feature (1)..(60): Primer 8ndh_tacBM3F
atacacccctcactctatatcactctcacaaattcgctcagcttccggctcgtataatgt

SEQ ID NO: 57 - DNA, 40 bp, synthetic oligonucleotide
  misc_feature (1)..(40): Primer 9nuoA_NF
agacgtgtgggctgggtaaggtgtaggctggagctgcttc

SEQ ID NO: 58 - DNA, 40 bp, synthetic oligonucleotide
  misc_feature (1)..(40): Primer 10BM3R
gaagcagctccagcctacaccttacccagcccacacgtct

SEQ ID NO: 59 - DNA, 60 bp, synthetic oligonucleotide
  misc_feature (1)..(60): Primer 41ldhA_ko_f
tgtgattcaacatcactggagaaagtcttatgaaactcgcgtgtaggctggagctgcttc

SEQ ID NO: 60 - DNA, 60 bp, synthetic oligonucleotide
  misc_feature (1)..(60): Primer 42ldhA_ko_r
ttgcagcgtagtctgagaaatactggtcagagcttctgctattccggggatccgtcgacc

SEQ ID NO: 61 - DNA, 61 bp, synthetic oligonucleotide
  misc_feature (1)..(61): 47focApflB_ko_f
accatgcgagttacgggcctataagccaggcgagatatgatgtgtaggctggagctgcttc

SEQ ID NO: 62 - DNA, 60 bp, synthetic oligonucleotide
  misc_feature (1)..(60): Primer 48focApflB_ko_r
catagattgagtgaaggtacgagtaataacgtcctgctgcattccggggatccgtcgacc

SEQ ID NO: 63 - DNA, 62 bp, synthetic oligonucleotide
  misc_feature (1)..(62): Primer 49adhE_ko_f
gttatctagttgtgcaaaacatgctaatgtagccaccaaatcgtgtaggctggagctgcttc

SEQ ID NO: 64 - DNA, 61 bp, synthetic oligonucleotide
  misc_feature (1)..(61): Primer 50adhE_ko_r
gcagtttcaccttctacataatcacgaccgtagtaggtatcattccggggatccgtcgacc

SEQ ID NO: 65 - DNA, 60 bp, synthetic oligonucleotide
  misc_feature (1)..(60): Primer 51poxB_ko_f
gatggagaaccatgaaacaaacggttgcagcttatatcgcgtgtaggctggagctgcttc

SEQ ID NO: 66 - DNA, 60 bp, synthetic oligonucleotide
  misc_feature (1)..(60): Primer 52poxB_ko_r
ctgaaacctttggcctgttcgagtttgatctgcggtggaacatatgaatatcctccttag

SEQ ID NO: 67 - DNA, 60 bp, synthetic oligonucleotide
  misc_feature (1)..(60): Primer 53ackA_ko_f
ataggtacttccatgtcgagtaagttagtactggttctgagtgtaggctggagctgcttc

SEQ ID NO: 68 - DNA, 60 bp, synthetic oligonucleotide
  misc_feature (1)..(60): Primer 54ackA_ko_r
tgccgaaacgtgcagccaggttgcgttcatgatcaacttccatatgaatatcctccttag

SEQ ID NO: 69 - DNA, 61 bp, synthetic oligonucleotide
  misc_feature (1)..(61): Primer 55frd_ko_f
gacttatccatcagactatactgttgtacctataaaggagcgtgtaggctggagctgcttc

SEQ ID NO: 70 - DNA, 60 bp, synthetic oligonucleotide
  misc_feature (1)..(60): Primer 56frd_ko_r
gagcttcattggtcgcgtattcctgttcctgatgatcgttattccggggatccgtcgacc

SEQ ID NO: 71 - DNA, 28 bp, synthetic oligonucleotide
  misc_feature (1)..(28): Primer 773Tryp_kpn_f
cgggtaccatggtagacgggcgatcttc

SEQ ID NO: 72 - DNA, 28 bp, synthetic oligonucleotide
  misc_feature (1)..(28): Primer 775Tryp_sal_r
gagtcgacaattttggatgagccgctcg

SEQ ID NO: 73 - DNA, 414 bp, Pseudomonas putida KT2440
  CDS (1)..(414): Sequence coding for Subunit A of NADH dehydrogenase I
atgtccgattcggcaggactcatcgcccacaactggggctttgccatcttcctcctgggtgtcgtcggcctgtgcgccttcatgctcggcctgtccagcctgctcggtagcaaggcctggggccgcgccaagaacgaacccttcgaatccggcatgctgcccgtcggcagcgctcgcctgcgcctgtccgccaaattctatctggtcgcgatgctgttcgtgatcttcgatatcgaagccctctttctctttgcatggtctgtgtccgtccgcgaaagcggctggaccggattcgtcgaagcactcgttttcatagcaattctgttggcaggtcttgtctacctatggcgcgtcggggcacttgattgggctcccgaaggtcgccgcaagcggcaagcgaagctgaaacaatga

SEQ ID NO: 74 - protein, 137 aa, Pseudomonas putida KT2440
MSDSAGLIAHNWGFAIFLLGVVGLCAFMLGLSSLLGSKAWGRAKNEPFESGMLPVGSARLRLSAKFYLVAMLFVIFDIEALFLFAWSVSVRESGWTGFVEALVFIAILLAGLVYLWRVGALDWAPEGRRKRQAKLKQ

SEQ ID NO: 75 - DNA, 678 bp, Pseudomonas putida KT2440
  CDS (1)..(678): Sequence coding for Subunit B of NADH dehydrogenase I
atgcaatacaatctcaccagaatcgatccggatgcgcccaacgagcagtacccggtcggtgaacgggaaaccgtcaccgatcaactgctggaggaccaggtccacaagaacatcttcatgggcaaacttgaagatgtgctgcgtggcgcggtcaactggggccgcaagaactccctctggccgtacaacttcggcctgtcctgctgctacgtggaaatgaccacggccttcacggcaccccacgacatcgcccgcttcggcgccgaagtcatccgggcctcgccgcgtcaggccgacttcatggtcattgccggtacctgcttcgtgaagatggcgccgatcatccagcgcctctacgagcagatgctcgagccgaaatgggtcatttccatgggttcgtgcgccaactccggtggcatgtacgacatctactcggtcgttcagggggtcgacaagttcctccccgtggacgtctatgtgcctggctgcccgccacgccctgaggctttcctgcaaggcttgatgctgctgcaggagtcgatcggccaagaacgacgcccgctttcctgggttgttggtgatcaaggtatttaccgtgccgagatgccagcccagaaagacctgcgtcgtgagcagcgcattgcagtaaccaacctgcgcagccccgacgaagtctga

SEQ ID NO: 76 - protein, 225 aa, Pseudomonas putida KT2440
MQYNLTRIDPDAPNEQYPVGERETVTDQLLEDQVHKNIFMGKLEDVLRGAVNWGRKNSLWPYNFGLSCCYVEMTTAFTAPHDIARFGAEVIRASPRQADFMVIAGTCFVKMAPIIQRLYEQMLEPKWVISMGSCANSGGMYDIYSVVQGVDKFLPVDVYVPGCPPRPEAFLQGLMLLQESIGQERRPLSWVVGDQGIYRAEMPAQKDLRREQRIAVTNLRSPDEV

SEQ ID NO: 77 - DNA, 1782 bp, Pseudomonas putida KT2440
  CDS (1)..(1782): Sequence coding for Subunit C,D of NADH dehydrogenase I
atgacagcggacaacgctattttcattccgccctacaaggctgacgaccaggatgtggtcgtcgaactgaacaaccgttttggcgccgatgcattcgtcgcccaggaaacccgcaccggcatgcccgtgctgtgggtaaaacgcgcccagctcaaagaggtgctcagcttcctgcgcggcgtcgccaagccttacagcatgctgtacgacctgcatggcgtcgacgagcgcctgcgcacccagcgccgtggcctgccagccgccgatttcagcgtgttctaccacctgttgtcgatcgagcgtaacagcgatgtgatgatcaaggtgtcgctcagcgaaggcgacctgaacctgccgaccgtgacgggcatctggccaaacgccaactggtacgagcgcgaagtctgggacatgttcggcatcgactttgccggccacccgcacctcagccgcatcatgatgccgccgacctgggaaggtcacccgctgcgcaaggactaccctgctcgtgcgaccgagttcgacccctacagcctgaccctggccaagcagcagcttgaagaggaatcggcacgcttcaacccggaagcctggggcatgaagcgtcagggcgccaatgaggactacatgttcctcaacctcggccccaaccacccttcggcgcacggtgcgttccgtatcgtcctgcagctggacggtgaagaaatcgtcgactgcgtaccggacatcggctaccaccaccgtggcgccgaaaaaatggccgagcgccagtcctggcacagcttcatcccctataccgaccgtatcgattacctcggcggggtgatgaacaacctgccgtacgtgcttgcggtagagaagctggccggcatcaaggtgccgcagaaggtcgacgtgattcgcatcatgctggccgagttcttccgcatcaccagccacctgctgttcctgggtacctacatccaggacgtcggcgccatgaccccggtgttcttcaccttcaccgaccgccagcgcgcctacaccgtgatcgaagcgatcaccggcttccgcctgcacccggcctggtaccgcatcggtggcgtcgcccacgacctgccacgtggctgggacaagctggtcaaggacttcgtcgaatggctgcccaagcgcctcgacgaatacaccaaggccgccctgcaaaacagcatcctcaagggccgtaccatcggcgttgccgcgtacaacaccaaggaagcgctggaatggggcaccaccggtgccggcttgcgtgccaccggttgcaacttcgacctgcgtaaagcccgcccttactccggctacgagaacttcgagttcgaagtaccgctggcccacaacggcgatgcctacgatcgctgcatggtccgtgtcgaggagatgcgtcagagtatccgcatcatcgaccagtgcctgcgcaacatgccggaaggcccgtacaaggcggatcacccgctgaccacgccgccgccgaaagagcgcaccctgcagcacatcgaaaccctgatcacgcacttcctgcaagtctcgtggggcccggtcatgccggccaacgagtcgttccagatgatcgaagcgaccaagggcatcaacagttactacctgacgagcgatggcggcaccatgagctaccgcacccggatccgcaccccgagctacccgcacctgcagcagatcccttcggtgatcaaaggcagcatggttgccgacctcattgcgtacctgggcagtatcgacttcgttatggctgacgtggaccgctaa

SEQ ID NO: 78 - protein, 593 aa, Pseudomonas putida KT2440
MTADNAIFIPPYKADDQDVVVELNNRFGADAFVAQETRTGMPVLWVKRAQLKEVLSFLRGVAKPYSMLYDLHGVDERLRTQRRGLPAADFSVFYHLLSIERNSDVMIKVSLSEGDLNLPTVTGIWPNANWYEREVWDMFGIDFAGHPHLSRIMMPPTWEGHPLRKDYPARATEFDPYSLTLAKQQLEEESARFNPEAWGMKRQGANEDYMFLNLGPNHPSAHGAFRIVLQLDGEEIVDCVPDIGYHHRGAEKMAERQSWHSFIPYTDRIDYLGGVMNNLPYVLAVEKLAGIKVPQKVDVIRIMLAEFFRITSHLLFLGTYIQDVGAMTPVFFTFTDRQRAYTVIEAITGFRLHPAWYRIGGVAHDLPRGWDKLVKDFVEWLPKRLDEYTKAALQNSILKGRTIGVAAYNTKEALEWGTTGAGLRATGCNFDLRKARPYSGYENFEFEVPLAHNGDAYDRCMVRVEEMRQSIRIIDQCLRNMPEGPYKADHPLTTPPPKERTLQHIETLITHFLQVSWGPVMPANESFQMIEATKGINSYYLTSDGGTMSYRTRIRTPSYPHLQQIPSVIKGSMVADLIAYLGSIDFVMADVDR

SEQ ID NO: 79 - DNA, 498 bp, Pseudomonas putida KT2440
  CDS (1)..(498): Sequence coding for Subunit E of NADH dehydrogenase I
atgaacagcacgcttatccagacagaccgtttcgccctgagcgaaaccgagcgctcggccatcgagcacgaaatgcatcactacgaggacccgcgcgcggcgtccatcgaagccctgaagatcgtccagaaggagcgtggctgggtgccggacggcgccattcacgccatcggcgaagtgctgggcattccggccagcgacgtcgagggtgtcgccaccttctacagccagatcttccgccaaccggtcggccgccacatcatccgcgtgtgcgacagcatggtctgctacatcggcggccatgagtcggtggtcagccagatccagagcgagctgggcatcggcctcggccagaccactgccgacggccgtttcaccctgctgcccgtgtgctgcctgggcaactgcgacaaggctccggcactgatgatcgacgacgacaccttcggtgacgtgcagccggctggcgtttccaaactgctggagggttacgtatga

SEQ ID NO: 80 - protein, 165 aa, Pseudomonas putida KT2440
MNSTLIQTDRFALSETERSAIEHEMHHYEDPRAASIEALKIVQKERGWVPDGAIHAIGEVLGIPASDVEGVATFYSQIFRQPVGRHIIRVCDSMVCYIGGHESVVSQIQSELGIGLGQTTADGRFTLLPVCCLGNCDKAPALMIDDDTFGDVQPAGVSKLLEGYV

SEQ ID NO: 81 - DNA, 1362 bp, Pseudomonas putida KT2440
  CDS (1)..(1362): Sequence coding for Subunit F of NADH dehydrogenase I
atgaccattacttccttcggcccggccaaccgcatcgcgcgctcggccgaaacccacccgctgacctggcgcctgcgtgacgacggcgagccggtctggctggccgagtacgaatcgaagaacggctacgccgcagcccgcaaggcgctggcgcaaatgtccgccgacgacatcgtgcagagcgtcaaggactccggcctcaaaggccgtggcggtgcaggtttccccactggcgtgaagtggggcctgatgcccaaagacgaatccatgaacatccgctacctgctgtgcaacgcggacgaaatggagccgaacacctggaaggaccgcatgctgatggagcaacagccccatctgctggtcgagggcatgctgatcagcgcccgcgccctgaaggcctaccgcggctacatcttcctgcgtggcgagtacaccaccgcagcgaaaaacctcaaccgcgccatcgatgaagccaaggccgccggcctgctgggcaagaacatcctgggcagcggtttcgatttcgagctgttcgtgcacaccggtgcaggccgctacatctgcggtgaagaaaccgcgctgatcaactcgctggaaggccgccgcgccaacccgcgctccaagccgcccttccctgccgccgttggcgtgtggggcaagccgacgtgcgtgaacaacgtcgagaccctgtgcaacgtcccggccatcgtcgccaacggcaacgactggtacaagtcgctggcccgcgaaggcagcgaggaccacggcaccaagctgatgggcttctccggcaaggtgaagaacccaggcctgtgggaactgccatttggcgttaccgcccgcgaattgttcgaagactacgccggtggcatgcgcgatggcttcaagctcaagtgctggcagccaggcggcgccggtaccggcttcctgctgcctgagcacctcgatgcgcaaatgtacgccggtggcatcgccaaggtcggcacccgtatgggtactggcctggccatggcggtcgacgacagcatcaacatggtgtcgctgctgcgcaacatggaagagttctttgcccgcgaatcgtgcggctggtgcaccccatgccgtgacggcctgccgtggagcgtgaagatgctgcgcgcgctggaaaacggccaaggccgcgccgaggacatcgagacgctgctggggctggtcaacttcctcggcccgggccgtaccttctgtgctcacgcaccgggtgccgtcgagccgctgggcagtgccatcaaatacttccgctcggagttcgaggccggcgtggcgccagaaagcgctgccaccctgcgccccgacctggcgaagccgatcgtggtcggcgcataa

SEQ ID NO: 82 - protein, 453 aa, Pseudomonas putida KT2440
MTITSFGPANRIARSAETHPLTWRLRDDGEPVWLAEYESKNGYAAARKALAQMSADDIVQSVKDSGLKGRGGAGFPTGVKWGLMPKDESMNIRYLLCNADEMEPNTWKDRMLMEQQPHLLVEGMLISARALKAYRGYIFLRGEYTTAAKNLNRAIDEAKAAGLLGKNILGSGFDFELFVHTGAGRYICGEETALINSLEGRRANPRSKPPFPAAVGVWGKPTCVNNVETLCNVPAIVANGNDWYKSLAREGSEDHGTKLMGFSGKVKNPGLWELPFGVTARELFEDYAGGMRDGFKLKCWQPGGAGTGFLLPEHLDAQMYAGGIAKVGTRMGTGLAMAVDDSINMVSLLRNMEEFFARESCGWCTPCRDGLPWSVKMLRALENGQGRAEDIETLLGLVNFLGPGRTFCAHAPGAVEPLGSAIKYFRSEFEAGVAPESAATLRPDLAKPIVVGA

SEQ ID NO: 83 - DNA, 2715 bp, Pseudomonas putida KT2440
  CDS (1)..(2715): Sequence coding for Subunit G of NADH dehydrogenase I
atggccactatccacgtagacggcaaagcgctcgaagtcaacggtgcagacaacctgttacaggcatgtctgtcgctcggcctcgacatcccttatttctgctggcacccggcgcttggtagcgttggtgcctgccggcaatgcgcagtcaagcagtacaccgacgagaacgacacccgtggtcgtatcgtcatgtcctgcatgacccctgcttccgacggcacctggatttccatcgacgatgaagagtcgaaggcgttccgcgccagcgtcgtcgaatggctgatgaccaaccacccgcacgactgcccggtgtgcgaggaaggcggtcactgccacctgcaggacatgacggtaatgaccggccacaacgagcgccgctaccgtttcaccaagcgtacccaccagaaccaggacctcggcccgttcatcgcccatgagatgaaccgctgcatcgcctgctaccgctgcgtgcgctattacaaggactacgccggtggtaccgacctgggcgtctacggcgcccacgacaacgtgtacttcggccgcgtcgaagacggtgtgctggaaagcgagttctccggcaacctgaccgaggtctgcccgaccggcgtattcaccgacaagacccactccgaacgctacaaccgcaagtgggacatgcagttcgccccaagcatctgccatggctgctccagcggctgcaacatcagcccgggtgagcgctacggcgaactacgccgtatcgaaaaccgcttcaacggttcggtcaaccagtacttcctgtgcgaccgcggccgcttcggctacggctacgtcaaccgcaaggaccgcccacgccagccacaactggccgacggcaccaagctgggcctggacgccgccctggacaaggccgccgacctgctacgcggccgtaccatcgtcggtatcggctcgccacgcgccagcctcgaaagcaactacggcctgcgtgagctggtcggcgccgagtacttctactcgggcatggaagctggcgaactggcccgcgtacgcctggccctgaacgtgctgaacaacagcccgctgcccgtgccgaccctgcgcgacatcgaagaccacgacgccgtgttcgtgctcggtgaagacctgacccagaccgctgcccgcgttgccctggccgtgcgccaggccaccaaaggcaaggccgaggccatggccgaagccatgaaagtgcagccttggctcgacgctgccgtgaagaacattggccagcacgcgctgtacccgctgttcatcgcatccctggctgaaaccaagctggacgacgtcgccgaagagtgcgtacacgccgcgccggccgacctggcccgcatcggtttcgccgtggcccacgccatcgacccgagcgcccctgccgtcgccggcctggacgacgaagcccaggccctggcccagcgcatcgccgacgccctggtcgccgccaagcgcccactggtcgtagccggtacttcgctggccgacccggcgctgatcgaagcggctgccaacatcgccaaggccctgaagctgcgcgagaaaaacggctcgctgagcctggtggtgcctgaggccaacagcctcggcctggccatgctcggtggcgagtcggtcgatgccgccctagacgcggtcatcagcggcaaagccgatgccatcgtggtgctggaaaacgacctgtacgcccgcgtaccggccgccaaggtcgatgctgccctggctgcggccaaggtggtgatcgttgccgaccactccaaaaccgccaccgtcgaccgcgcccacctggtgctgccggccgcctcgttcgccgaaggcgacggtaccctggtcagccaggaaggccgtgcccagcgcttcttccaggtgttcgacccgcagtacctggacagcagcatccagatccacgaaggctggcgctggatgcacgccctgcgtgccaccctgctgaacaagccggtcgactggacccagctggaccacgtcaccagcgcctgcgccgaagccgctccgcaactggctggcatcgtcaacgccgcgccgagcgctgcgttccgcatcaagggcatgaagctggcccgtgagccgctgcgctactccggccgtaccgccatgcgtgccaacatcagtgtgcacgagccgcgcaccccgcaagacaaggacaccgcgttcgccttctccatggaaggctactcgggttcggccgaaccgcgccagcaggtgccattcgcctggtcgccgggctggaactccccacaagcctggaacaagtttcaggacgaggtcggtggccacctgcgtgccggtgacccaggcgtacgcctgatcgaatcgcaaggcgaccgcctgaactggttcaacgccattccgggggccttcaacccggcccgtggcatctggactgccgtgccgttcttccacctgttcggcagcgaagaaagctcctcgcgcgccgccccggttcaagaacgcatcccggctgcctacgtggccctagccaagtccgaagccgaccgcctgggcgtcaacgacggcgccctgctgagcctgaacgtcgccggtgtggccctgcgcctgccgctgcgtatcaatgaagagctgggcgctggcctggtcgcgctgccgaaaggcctggctggcattccgcctgccatcttcggtgcatccgtcgaaggtctgcaggaggcagcacaatga

SEQ ID NO: 84 - protein, 904 aa, Pseudomonas putida KT2440
MATIHVDGKALEVNGADNLLQACLSLGLDIPYFCWHPALGSVGACRQCAVKQYTDENDTRGRIVMSCMTPASDGTWISIDDEESKAFRASVVEWLMTNHPHDCPVCEEGGHCHLQDMTVMTGHNERRYRFTKRTHQNQDLGPFIAHEMNRCIACYRCVRYYKDYAGGTDLGVYGAHDNVYFGRVEDGVLESEFSGNLTEVCPTGVFTDKTHSERYNRKWDMQFAPSICHGCSSGCNISPGERYGELRRIENRFNGSVNQYFLCDRGRFGYGYVNRKDRPRQPQLADGTKLGLDAALDKAADLLRGRTIVGIGSPRASLESNYGLRELVGAEYFYSGMEAGELARVRLALNVLNNSPLPVPTLRDIEDHDAVFVLGEDLTQTAARVALAVRQATKGKAEAMAEAMKVQPWLDAAVKNIGQHALYPLFIASLAETKLDDVAEECVHAAPADLARIGFAVAHAIDPSAPAVAGLDDEAQALAQRIADALVAAKRPLVVAGTSLADPALIEAAANIAKALKLREKNGSLSLVVPEANSLGLAMLGGESVDAALDAVISGKADAIVVLENDLYARVPAAKVDAALAAAKVVIVADHSKTATVDRAHLVLPAASFAEGDGTLVSQEGRAQRFFQVFDPQYLDSSIQIHEGWRWMHALRATLLNKPVDWTQLDHVTSACAEAAPQLAGIVNAAPSAAFRIKGMKLAREPLRYSGRTAMRANISVHEPRTPQDKDTAFAFSMEGYSGSAEPRQQVPFAWSPGWNSPQAWNKFQDEVGGHLRAGDPGVRLIESQGDRLNWFNAIPGAFNPARGIWTAVPFFHLFGSEESSSRAAPVQERIPAAYVALAKSEADRLGVNDGALLSLNVAGVALRLPLRINEELGAGLVALPKGLAGIPPAIFGASVEGLQEAAQ

SEQ ID NO: 85 - DNA, 1008 bp, Pseudomonas putida KT2440
  CDS (1)..(1008): Sequence coding for Subunit H of NADH dehydrogenase I
atgagctggttcacccccgaagtgatcgatgtgatcctcaccgtgctgcgggccatcgtggtcctgctggcggtggtggtctgcggtgcgctgctcagcttcgtcgagcgccgcctgctgggctggtggcaggaccgttacggtccgaaccgcgtcggcccgttcggcatgttccagatcgctgccgacatgctgaagatgttcttcaaggaagactggaacccacccttcgtcgatcgcgtgatcttcaccctggcaccggtagtggccatgagcgccctgctgatcgccttcgtggtcatcccgatcaccccgacctggggcgttgccgacctgaacatcggcctgctgttcttcttcgccatggccggcctgtcggtctacgcggtgctgttcgccggctggtcgtcgaacaacaagtacgccctgctgggcagcttgcgtgcttcggcacagaccgtgtcgtacgaagtgttcctgggcctggcgctgatgggcgtggtggtgcaggtgggttcgttcaacatgcgcgacatcgttgagtaccaggcgcagaacctgtggttcatcattccgcagttcttcggcttctgcaccttcttcatcgctggcgtcgccgtgactcaccgtcacccgttcgaccagccggaagcagaacaggaactggccgacggctaccacatcgagtatgccggcatgaagtggggcatgttcttcgtcggtgagtacatcggcatcatcctcatctcggcgctgctggtaaccctgttcttcggcggctggcacggcccgttcggcatcctgccgcaactgtcgttcctgtggttcgccctgaagaccgcgttcttcatcatgctgttcatcctgctgcgcgcctcgatcccgcgcccacgctatgaccaggtgatggacttcagctggaagttctgcctgccgctgaccctgatcaatttgctggtgaccgctgcgatcgtgctctacaacacgccagccgtcgcggcccagtga

SEQ ID NO: 86 - protein, 335 aa, Pseudomonas putida KT2440
MSWFTPEVIDVILTVLRAIVVLLAVVVCGALLSFVERRLLGWWQDRYGPNRVGPFGMFQIAADMLKMFFKEDWNPPFVDRVIFTLAPVVAMSALLIAFVVIPITPTWGVADLNIGLLFFFAMAGLSVYAVLFAGWSSNNKYALLGSLRASAQTVSYEVFLGLALMGVVVQVGSFNMRDIVEYQAQNLWFIIPQFFGFCTFFIAGVAVTHRHPFDQPEAEQELADGYHIEYAGMKWGMFFVGEYIGIILISALLVTLFFGGWHGPFGILPQLSFLWFALKTAFFIMLFILLRASIPRPRYDQVMDFSWKFCLPLTLINLLVTAAIVLYNTPAVAAQ

SEQ ID NO: 87 - DNA, 549 bp, Pseudomonas putida KT2440
  CDS (1)..(549): Sequence coding for Subunit I of NADH dehydrogenase I
atgttcaagtatatcggcgacatcgttaagggcaccggcacccagctgcgcagcctggcaatggtgttctcccacgggttccgcaagcgcgacaccctgcaataccccgaagaacccgtgtacctgccgccgcgctaccgcggccgcatcgtcctcacccgcgaccccgatggcgaggagcgctgcgtagcgtgcaacctctgcgcggtggcctgcccggtgggctgcatttcgctgcagaaggccgagacagaggacggccgctggtacccggagttcttccgcatcaacttctcgcgttgcattttctgcggcctgtgtgaagaagcgtgcccgaccaccgcgatccagctgactccggatttcgaaatggccgagttcaagcgtcaggacctggtgtacgagaaagaagatctgctgatctccggccccggcaagaaccctgactacaacttctaccgtgttgcgggtatggcaatcgctggcaagccgaagggctctgcacagaacgaagccgagccgatcaacgtgaagagcttgctcccataa

SEQ ID NO: 88 - protein, 182 aa, Pseudomonas putida KT2440
MFKYIGDIVKGTGTQLRSLAMVFSHGFRKRDTLQYPEEPVYLPPRYRGRIVLTRDPDGEERCVACNLCAVACPVGCISLQKAETEDGRWYPEFFRINFSRCIFCGLCEEACPTTAIQLTPDFEMAEFKRQDLVYEKEDLLISGPGKNPDYNFYRVAGMAIAGKPKGSAQNEAEPINVKSLLP

SEQ ID NO: 89 - DNA, 501 bp, Pseudomonas putida KT2440
  CDS (1)..(501): Sequence coding for Subunit J of NADH dehydrogenase I
atggaattcgctttctacttcgcatccgggatcgccgtggtctccacccttcgggtggtaactggcaccaaccccgtgcacgccttgctttacctgatcatttcgctgatttccgtggccatgatcttcttcgccctgggtgcgccgtttgccggcgccctggaagtgatcgcctacgccggcgccatcatggtgctgttcgtgttcgtggtgatgatgctcaacctcgggccggcttcggtcgcccaggaacggggctggctcaagcccggtatctgggcagggccagtgatcctcggcaccctgctgctggcggagctgctgtacgtcctgttcgtcgccccgagcggcgccggcatcagcggtaccaccgtgggcccgaaagccgtgggcatcagcctgttcggcccgtacctgctggtggtcgaactggcgtcgatgctgctgctggctgcagccgtcaccgccttccacctgggccgcaacgaggcgaaggagtaa

SEQ ID NO: 90 - protein, 166 aa, Pseudomonas putida KT2440
MEFAFYFASGIAVVSTLRVVTGTNPVHALLYLIISLISVAMIFFALGAPFAGALEVIAYAGAIMVLFVFVVMMLNLGPASVAQERGWLKPGIWAGPVILGTLLLAELLYVLFVAPSGAGISGTTVGPKAVGISLFGPYLLVVELASMLLLAAAVTAFHLGRNEAKE

SEQ ID NO: 91 - DNA, 1854 bp, Pseudomonas putida KT2440
  CDS (1)..(1854): Sequence coding for Subunit L of NADH dehydrogenase I
atgaaccttctcttcctgactttcgtctttcccctcatcggcttcctgctgctgtcgttctcgcgcggacggttctcggagaacctgtccgccctgatcggcgtcggctcggtaggcctgtcggccgccacggccgcctacgtcatctggcagttcaacgtcgccccgcctgagggcggcgcgtacagccagctgctgtggcagtggatgtcggtggacggcttcgcgccgaacttcaccctgtacctggacggcctgtcggtcaccatgctcggcgtggtgaccggtgtcggcttcctgatccacctgttcgcatcctggtacatgcgtggcgaagccggctactcgcgcttcttctcgtacaccaacctgttcatcgccagcatgctgttcctgatccttggcgataacctgctgttcatctacttcggctgggaaggcgtgggcctgtgctcgtacctgttgatcggtttctactacagcaaccgcaacaacggtaacgcggcactcaaggcattcatcgtcacccgtatcggcgacgtgttcatggccatcggcctgttcatcctgttcgcccagctgggcaccctgaacgtgcaggaactgctggtgttggcaccgcagaagttccaggctggcgacacctggatggtgctggccacgctgatgctgctgggtggtgcggtcggtaaatcggcgcagctgccattgcagacctggctggccgacgccatggcgggcccgactccggtttcggcactgatccacgccgcaaccatggtgaccgcgggcgtgtacctgatcgcccgtaccaacggcctgttcctgctggcgccggacatcctgcaccttgtaggtgtggtcggtggcttgaccctggtactggcaggcttcgctgcgctggtacagaccgatatcaagcgtatcctcgcctactcgaccatgagccagatcggctacatgttcctggccctgggcgtgggtgcctgggacgcggcgatcttccacctgatgacccacgccttcttcaaggccctgctgttccttgcctccggtgcggtgatcgttgcctgccaccacgagcagaacatcttcaagatgggcggcctgtggaagaaactgccgctggcctacgccagcttcgtggtcggtggtgcggcactggcggccctgccgatcgtgaccgtaggcttctactccaaggacgagatcctctgggaagccttcgccagcggcaacaccggcctgctgtatgccggcctggtaggcgcgttcatgacctcgctgtacaccttccgcctgatcttcatcgccttccacggcgaagccaagaccgaagcccacgctggccacggcatcagccactggctgccgctgggcgtgctgatcgtgctgtcgactttcgtcggcgcctggatcaccccgccgctggccggtgtgctgccggaaagcgccggccatgccggtggcgaagccaagcacgcgctggagatcacctcgggtgccatcgccatcgccggtatcctgctggccgccctgctgttcctgggcaagcgccgcttcgtcagcgccgtcgccaacagtggcatcggtcgcgtcctgtcggcctggtggttcgctgcctggggcttcgactggatctacgacaagcttttcgtcaaaccgtacctgctgatcagccacatcctgcgcaaggacccggttgaccgcaccatcggcctgattcctcggatggcgcgtggtggccacgtggccatgagcaagaccgagactggccaactgcgctggtacaccgcctctatcgccgtgggtgccgtgctggtgctcggtgccgtggtagtggctgcggtatga

SEQ ID NO: 92 - protein, 617 aa, Pseudomonas putida KT2440
MNLLFLTFVFPLIGFLLLSFSRGRFSENLSALIGVGSVGLSAATAAYVIWQFNVAPPEGGAYSQLLWQWMSVDGFAPNFTLYLDGLSVTMLGVVTGVGFLIHLFASWYMRGEAGYSRFFSYTNLFIASMLFLILGDNLLFIYFGWEGVGLCSYLLIGFYYSNRNNGNAALKAFIVTRIGDVFMAIGLFILFAQLGTLNVQELLVLAPQKFQAGDTWMVLATLMLLGGAVGKSAQLPLQTWLADAMAGPTPVSALIHAATMVTAGVYLIARTNGLFLLAPDILHLVGVVGGLTLVLAGFAALVQTDIKRILAYSTMSQIGYMFLALGVGAWDAAIFHLMTHAFFKALLFLASGAVIVACHHEQNIFKMGGLWKKLPLAYASFVVGGAALAALPIVTVGFYSKDEILWEAFASGNTGLLYAGLVGAFMTSLYTFRLIFIAFHGEAKTEAHAGHGISHWLPLGVLIVLSTFVGAWITPPLAGVLPESAGHAGGEAKHALEITSGAIAIAGILLAALLFLGKRRFVSAVANSGIGRVLSAWWFAAWGFDWIYDKLFVKPYLLISHILRKDPVDRTIGLIPRMARGGHVAMSKTETGQLRWYTASIAVGAVLVLGAVVVAAV

SEQ ID NO: 93 - DNA, 1533 bp, Pseudomonas putida KT2440
  CDS (1)..(1533): Sequence coding for Subunit M of NADH dehydrogenase I
atgattttgccttggctgatcctgatccccttcatcggcggcttcctctgctggctgggtgagcgcttcggcgccaccctgccgcgctggatcgcgctgcttaccatgtccctgctgcttggcatcggcctgtggctgtggggtaccggcgactacacccttgctcccgccccgggcgccgaaccggcctgggctctcgaatacaaagtcgagtggatcaagcgtttcggcatcagcatccacctggccctggacggcctgtcgctgctgatgatcctgctcaccggcctgctcggtgtgctgtcggtactgtgttcctggaaagagatccagcgccacgtcggcttcttccacctcaacctgatgtggatcctcggcggcgtggtcggtgtgttcctggccctggacctgttcctgttcttcttcttctgggaaatgatgctggtgccgatgtacttcctcatcgcgctctggggtcacagctcggcagacggcaagaagacccggatctacgcggcgaccaagttcttcatcttcacccaggccagcggcctgatcatgctggtggcgatcctgggcctggtgctggtcaactacaacaccactggcgtgctcaccttcaactacagcgacctgctcaaggccgagttgccggccggtatcgagtacgtgctgatgctgggcttcttcatcgccttcgcggtgaagctgccagtggtgccgttccactcctggctgcctgacgctcacgcccaggcaccgaccgcaggctcggtggacctggcgggtatcttgctgaagaccgcggcctacggcctgctgcgcttcgctctgccgctgttcccgaacgcctcggccgagttcgcgccgatcgccatgaccctgggcctgatcggtatcttctacggtgccttcctggccttcgcacaaaccgacatcaagcgcctgatcgccttctccagcgtctcgcacatgggcttcgtgctgatcggtatctactccggcagccagcaggccctgcaaggtgcggtgatccagatgctggcccacggcctgtcggctgcggcgctgttcatcctgtccggccagctgtacgagcgcctgcacacccgtgacatgcgtcagatgggtggcctgtggcaccgcatcgcctacctgccggccatcagcctgttcttcgcagcggcatcgctgggcctgccaggcaccggcaacttcgtcggcgagttcctgatcctgatcggcagcttcgtgcatgtaccgtggatcaccgtgatcgccactaccggcctggtgttcggttctgtttactcgctgatcatgatccaccgtgcctacttcggcccggccaagaccgacaccgtgctggccggcatggacggtcgcgaactgatcatggttctgggtctggcggtattgctgatcctgctgggcgtgtatccgcagccgttcctcgacacctctgccgccaccatgagtggtgtgcagcagtggctcggatccgctttcactcaactcgcttcggcccggtaa

SEQ ID NO: 94 - protein, 510 aa, Pseudomonas putida KT2440
MILPWLILIPFIGGFLCWLGERFGATLPRWIALLTMSLLLGIGLWLWGTGDYTLAPAPGAEPAWALEYKVEWIKRFGISIHLALDGLSLLMILLTGLLGVLSVLCSWKEIQRHVGFFHLNLMWILGGVVGVFLALDLFLFFFFWEMMLVPMYFLIALWGHSSADGKKTRIYAATKFFIFTQASGLIMLVAILGLVLVNYNTTGVLTFNYSDLLKAELPAGIEYVLMLGFFIAFAVKLPVVPFHSWLPDAHAQAPTAGSVDLAGILLKTAAYGLLRFALPLFPNASAEFAPIAMTLGLIGIFYGAFLAFAQTDIKRLIAFSSVSHMGFVLIGIYSGSQQALQGAVIQMLAHGLSAAALFILSGQLYERLHTRDMRQMGGLWHRIAYLPAISLFFAAASLGLPGTGNFVGEFLILIGSFVHVPWITVIATTGLVFGSVYSLIMIHRAYFGPAKTDTVLAGMDGRELIMVLGLAVLLILLGVYPQPFLDTSAATMSGVQQWLGSAFTQLASAR

SEQ ID NO: 95 - DNA, 1470 bp, Pseudomonas putida KT2440
  CDS (1)..(1470): Sequence coding for Subunit N of NADH dehydrogenase I
atggaattcaccactcaacacttcatcgcattggcgccgatgctgatcaccaccatcaccacggtggtggtgatgctggcgatcgcctggaagcgcaaccactcgcagaccttcctgctgtccaccgtgggcctgaacctggccctgctgtcgctcctgccggcgctgaaggtcgcgccgctggcggtcacatcgctgatcaccatcgacaagttcgcctgcctgtacatggcgatcatcctcgtggcgacgctggcctgcgtcaccctcgcccacgcctacctcggcgagggcgccaagggtttcccgggcaaccgtgaagaactgtacctgttgctgctgatgtcggccctcggtggcctggtgctggtcagcgccaaccacctagccggcctgttcatcggcctggagctgttgtcggtaccggtctacggcctggtggcgtatgccttcttcaacaagcgatcgctggaagctggcatcaagtacatggtgctgtcggccgcaggctcggctttcctgctgttcggcatggccctgctgtacgccgacgccggcagcctgagcttcgaccagatcggcaaggccctggctaccaccagcatgccaagcctggtggcccagctgggcctgggcatgatgctggttggcctggccttcaagctgtcgctggtaccgttccatctgtggaccccggacgtgtacgaaggcgctccggcgccggtcgccgcgttcctggcaaccgccagcaaggtggcagtattcgccgttgtcgtacgcctgttcatgctctcccctgctgccagcagcggcgtgctcagcaccgtattggcggtgattgccgtagcctcgatcctgattggtaacctgctggcgctgacccagagcaacctcaagcgtctgctgggttactcgtccatcgcccacttcggttacctggtcatcgcgttggtcgccagcaagggcctggccctggaagccatgggcgtttacctggtcacctacgtgatcaccagcctgggcgccttcggcgtcatcaccctcatgtcctcgccatacggcggccgtgacgccgatgccctgtacgagtaccgcggcctgttctggcgccgcccgtacctgacagcggtactgaccgtgatgatgctgtcgctggcgggtatcccgctgaccgccggcttcatcggcaagttctacatcatcgctaccggtgtcgaatcgcacctgtggtggttggtcggtgcgctggtgatcggtagcgccatcggcgtgtactactacctgcgcgtcatggttaccctgtacctggtcgagccgaacctgcgtcgtcacgacgccccgctcaagtgggaacagcgcaccggcggtgtcatgctgctggctatcgccattctcgctttcgtactgggtgtgtacccgcagcccctgctggagatggttcagcaagcgggcctgcaactgatcggttga

SEQ ID NO: 96 - protein, 489 aa, Pseudomonas putida KT2440
MEFTTQHFIALAPMLITTITTVVVMLAIAWKRNHSQTFLLSTVGLNLALLSLLPALKVAPLAVTSLITIDKFACLYMAIILVATLACVTLAHAYLGEGAKGFPGNREELYLLLLMSALGGLVLVSANHLAGLFIGLELLSVPVYGLVAYAFFNKRSLEAGIKYMVLSAAGSAFLLFGMALLYADAGSLSFDQIGKALATTSMPSLVAQLGLGMMLVGLAFKLSLVPFHLWTPDVYEGAPAPVAAFLATASKVAVFAVVVRLFMLSPAASSGVLSTVLAVIAVASILIGNLLALTQSNLKRLLGYSSIAHFGYLVIALVASKGLALEAMGVYLVTYVITSLGAFGVITLMSSPYGGRDADALYEYRGLFWRRPYLTAVLTVMMLSLAGIPLTAGFIGKFYIIATGVESHLWWLVGALVIGSAIGVYYYLRVMVTLYLVEPNLRRHDAPLKWEQRTGGVMLLAIAILAFVLGVYPQPLLEMVQQAGLQLIG

SEQ ID NO: 97 - DNA, 1296 bp, Pseudomonas putida KT2440
  CDS (1)..(1296): Sequence coding for a NADH dehydrogenase
ttagtgcaacttcaggcgcggctcggtaccccggccaatcttgctgcccagcatcatcaacgccgtgcggaagaacccgtacagcgccatctggtgcatgcggtacagcgacacatagaacatgcgcgccagccagccttccagcttcacgctgcccatcaggttacccatcaggttgcccaccgccgagaagcgcgacagcgacaccagcgagccatagtccttgtactcgtacgtcggcagggccttgttctccaggcgcgccttcaggctttgcgccagcatcgaagcctgctggtgcgccgcctgtgcgcgcggcggcacgttacgatcgctgccgggttgcgggcaggcggcgcagtcaccgaaggcgaagatgttgtcgtcgcgggtggtttgcagggtagggcgcacgaccagctggttgatgcggttggtttccaggccatcgatgtccttgaggaaacctggcgcgcggataccggctgcccacaccttcaggctggcctggatcacttcaccgtccttggttttcaggccgtcctcggtcacttcactgactgcggcgttggtcatcaccttcaccccgagtttttccagggttttgtgaaccggtacgctgatgcgctccggcagcgctggcagaacgcgcgggcccgcctcgataagggtgatgtgcatgtccttgggctggatgcggtccaggccataggccgccagctcgtgggcagcgtggtgcagctcggccgccagctctacaccggtggcaccggcgccgacaatggctacgctgattttctcgctggccacgtcgccggcgtgggcacgcaggtagtggttgagcagttgctggtggaaacgctcggcctgcttgcgggtatccaggaacaggcagtgctgcgctgcgcccagggtgccgaagtcgttggtgttggagcccacggcaatcaccagggtgtcgtagcccagggtgcgcgcaggcagcagctcgcggccctcttcgtcgagggtggcagccagttgaatctgcttgccttcacggtccaggccgctcatgcgccccagctggaaattgaagtggttccacttggcctgggccacgtagttcagttcgtcttccgaggagttcagcgagccggcggccacttcgtgcagcagtggcttccagatgtgcgtgaggttggtgtcgaccagggtgatttcggcctgcttgcgcttgcccaggcttttacccaggcgggtcgccagttccaggccgccggcgccgccgccgacaatcacgatgcgatgagtcat

SEQ ID NO: 98 - protein, 432 aa, Pseudomonas putida KT2440
LVQLQARLGTPANLAAQHHQRRAEEPVQRHLVHAVQRHIEHARQPAFQLHAAHQVTHQVAHRREARQRHQRAIVLVLVRRQGLVLQARLQALRQHRSLLVRRLCARRHVTIAAGLRAGGAVTEGEDVVVAGGLQGRAHDQLVDAVGFQAIDVLEETWRADTGCPHLQAGLDHFTVLGFQAVLGHFTDCGVGHHLHPEFFQGFVNRYADALRQRWQNARARLDKGDVHVLGLDAVQAIGRQLVGSVVQLGRQLYTGGTGADNGYADFLAGHVAGVGTQVVVEQLLVETLGLLAGIQEQAVLRCAQGAEVVGVGAHGNHQGVVAQGARRQQLAALFVEGGSQLNLLAFTVQAAHAPQLEIEVVPLGLGHVVQFVFRGVQRAGGHFVQQWLPDVREVGVDQGDFGLLALAQAFTQAGRQFQAAGAAADNHDAMSH